(19) United States
(12) Patent Application Publication
(10) Pub. No.: US 2026/0283915 A1
(43) Pub. Date: Sep. 24, 2026

(54) METHODS AND COMPOSITIONS FOR COSMETIC APPLICATIONS

(71) Applicant: Glo Pharma, Inc., Carlsbad, CA (US)

(72) Inventors: Robert A. LOVE, San Diego, CA (US); Marianna FOLDVARI, Kitchener (CA)

(21) Appl. No.: 19/630,257

(22) Filed: Mar. 26, 2026

Related U.S. Application Data
(63) Continuation of application No. 18/052,490, filed on Nov. 3, 2022, now abandoned.
(60) Provisional application No. 63/275,878, filed on Nov. 4, 2021.
(60) Provisional application No. 63/406,873, filed on Sep. 15, 2022.

(51) Int. Cl. A61K 8/14; A61K 8/64; A61Q 19/00

(52) U.S. Cl. CPC A61K 8/14; A61K 8/64; A61Q 19/00

(57) ABSTRACT

The present invention relates to compositions comprising and methods for use of formulations for the delivery of cosmetic agents, including anionic polymers such as hyaluronic acid and peptides with cosmetic activity. They are useful in, e.g., cosmetics and pharmaceuticals that prevent or improve the appearance of undesirable skin features including wrinkles.

### CROSS-REFERENCE

This application is a continuation of U.S. Ser. No. 18/052,490, filed Nov. 3, 2022, which claims the benefit of U.S. provisional patent application No. 63/406,873 filed on Sep. 15, 2022, and U.S. provisional patent application No. 63/275,878 filed on Nov. 4, 2021, which are incorporated by reference in their entirety.

### SEQUENCE LISTING

The instant application contains a Sequence Listing which has been submitted electronically in XML format and is hereby incorporated by reference in its entirety. Said XML copy, created on Mar. 26, 2026, is named 54780-712.301_SL.xml and is 96,290 bytes in size.

### BACKGROUND OF THE INVENTION

Certain cosmetic agents are desirably delivered below the surface of the skin. There exists a need for improved methods of delivery and compositions for such cosmetic agents for various cosmetic and pharmaceutical purposes, including the prevention, reduction, or elimination of wrinkles.

### SUMMARY OF THE INVENTION

The present invention relates to compositions for delivery of cosmetic agents. In some embodiments, the compositions are lipid vesicle formulations of cosmetic agents which allow the cosmetic agents to be delivered below the surface of the skin upon topical application. In some embodiments, the cosmetic agent comprises an anionic polymer material, such as hyaluronic acid, a peptide which is beneficial for the appearance of the skin, such as the skin of the lips, the face, around the eyes, or neck of a subject, or all of the above. In some embodiments, the cosmetic agents are delivered to a preferred or pre-selected layer of the skin or surrounding tissue, such as the epidermis, dermis, subcutaneous tissue, or muscle tissue.

In one aspect, provided herein, is a lipid vesicle composition comprising; (a) lipid vesicles each comprising a lipid bilayer comprising vesicle forming lipids; and (b) an oil-in-water emulsion entrapped in the lipid vesicles, and stabilized by one or more surfactants; wherein the lipid bilayer, the oil-in-water emulsion, or a combination thereof comprises one or more peptides, wherein the one or more peptides comprises an amino acid sequence: Xaa1-Xaa2-Xaa3-Xaa4-Xaa5-Xaa6-Xaa7-Xaa8, wherein: Xaa1 is absent or selected from Ala, Arg, Gly, Lys, Pro, Tyr, Val and a derivative of Ala, Arg, Gly, Lys, Pro, Tyr, or Val; Xaa2 is absent or selected from: Ala, Arg, Cys, Gly, Ile, Lys, Pro, Tyr, Val and a derivative of Ala, Arg, Cys, Gly, Ile, Lys, Pro, Tyr, or Val; Xaa3 is absent or selected from: Ala, Arg, Gln, Gly, Lys, Phe, Pro, Tyr, Val and a derivative of Ala, Arg, Gln, Gly, Lys, Phe, Pro, Tyr, or Val; Xaa4 is absent or selected from: Ala, Arg, Glu, Gly, Lys, Pro, Tyr, Val and a derivative of Ala, Arg, Glu, Gly, Lys, Pro, Tyr, or Val; Xaa5 is absent or selected from: Ala, Arg, Gln, Gly, Lys, Leu, Met, Pro, Thr, Tyr, Val and a derivative of Ala, Arg, Gln, Gly, Lys, Leu, Met, Pro, Thr, Tyr, or Val; Xaa6 is absent or selected from: Ala, Arg, Asp, Gln, Gly, His, Lys, Phe, Pro, Ser, Thr, Tyr, Val and a derivative of Ala, Arg, Asp, Gln, Gly, His, Lys, Phe, Pro, Ser, Thr, Tyr, or Val; Xaa7 is selected from: Ala, Arg, Asn, Asp, Cys, Gly, His, Lys, Phe, Pro, Thr, Tyr, Val and a derivative of Ala, Arg, Asn, Asp, Cys, Gly, His, Lys, Phe, Pro, Thr, Tyr, or Val; Xaa8 is selected from: Ala, Arg, Cys, Glu, Gly, His, Lys, Phe, Pro, Ser, Thr, Trp, Tyr, Val and a derivative of Ala, Arg, Cys, Glu, Gly, His, Lys, Phe, Pro, Ser, Thr, Trp, Tyr, or Val. In some embodiments, the one or more peptides are entrapped in the lipid bilayer, the oil-in-water emulsion, or in the combination thereof. In some embodiments, the one or more peptides are unmodified. In some embodiments, the one or more peptides is present at a concentration of from about 0.1 mg/mL to about 10 mg/mL. In some embodiments, the one or more peptides comprise tetrapeptides, pentapeptides, hexapeptides, or any combination thereof. In some embodiments, the one or more peptides comprises an amino acid sequence at least about 50%, at least about 60%, at least about 70%, at least about 80%, or at least about 90% sequence homology to the amino acid sequence of any one of SEQ ID NOs: 1-51. In some embodiments, the one or more peptides comprise an amino acid sequence identical to the amino acid sequence of any one of SEQ ID NOs: 1-51. In some embodiments, the one or more peptides have an amino acid sequence consisting of an identical sequence to the amino acid sequence of any one of SEQ ID NOs: 1-51. In some embodiments, the one or more peptides have an amino acid sequence consisting of an identical sequence to the amino acid sequence of SEQ ID NO: 31, 36, or 37. In some embodiments, the one or more peptides have an amino acid sequence consisting of an identical sequence to the amino acid sequence of SEQ ID NO: 21. In some embodiments, the one or more peptides have an amino acid sequence consisting of an identical sequence to the amino acid sequence of SEQ ID NO: 24. In some embodiments, the one or more peptides have an amino acid sequence consisting of an identical sequence to the amino acid sequence of SEQ ID NO: 30. In some embodiments, the one or more peptides have an amino acid sequence consisting of an identical sequence to the amino acid sequence of SEQ ID NO: 31. In some embodiments, the one or more peptides have an amino acid sequence consisting of an identical sequence to the amino acid sequence of SEQ ID NO: 36. In some embodiments, the one or more peptides have an amino acid sequence consisting of an identical sequence to the amino acid sequence of SEQ ID NO: 37. In some embodiments, the one or more peptides have an amino acid sequence consisting of an identical sequence to the amino acid sequence of SEQ ID NO: 38. In some embodiments, the one or more peptides have an amino acid sequence consisting of an identical sequence to the amino acid sequence of SEQ ID NO: 40. In some embodiments, the lipid bilayer, the oil-in-water emulsion, or the combination thereof further comprises an anionic polymer material. In some embodiments, the anionic polymer material is entrapped in the lipid bilayer, the oil-in-water emulsion, or in a combination thereof. In some embodiments, the anionic polymer material comprises an anionic polysaccharide. In some embodiments, the anionic polymer is present in an amount of about 0.01 mg/mL to about 10 mg/mL of the composition. In some embodiments, the anionic polysaccharide comprises hyaluronic acid or a salt thereof. In some embodiments, the anionic polymer material has a molecular weight of from about 5 kDa to about 500 kDa. In some embodiments, the anionic polymer material comprises a first and a second anionic polymer material, each anionic polymer material having a different molecular weight. In some embodiments, the first and the second anionic polymer material are the same material. In some embodiments, the first anionic polymer material has a molecular weight of up to about 75 kDa and the second anionic polymer material has a molecule weight of greater than about 75 kDa. In some embodiments, the first anionic polymer material has a molecular weight of from about 5 kDa to about 50 kDa, and wherein the second anionic polymer material has a molecular weight of from about 100 kDa to about 500 kDa. In some embodiments, the vesicle forming lipids comprise phospholipids, glycolipids, lecithins, ceramides, lysolecithin, lysophosphatidylethanolamine, phosphatidylserine, phosphatidylinositol, sphingomyelin, cardiolipin, phosphatidic acid, cerebroside, or any combination thereof. In some embodiments, the vesicle forming lipids comprise phospholipids. In some embodiments, the composition comprises vesicle forming lipids in an amount of from about 0.5% to about 25% (w/w) of the composition. In some embodiments, the oil-in-water emulsion comprises a triglyceride in the oil component. In some embodiments, the triglyceride comprises a medium-chain triglyceride. In some embodiments, the triglyceride is present in an amount of from about 1% to about 35% (w/w) of the composition. In some embodiments, the composition comprises a sterol. In some embodiments, the sterol is present in an amount of from about 1% to about 5% (w/w) of the composition. In some embodiments, the composition comprises propylene glycol. In some embodiments, the propylene glycol is present in an amount of from about 1% to about 25% (w/w) of the composition. In some embodiments, the composition comprises one or more viscosity enhancing agents. In some embodiments, the one or more viscosity enhancing agents are present in an amount of from about 0.5% to about 10% (w/w) of the composition. In some embodiments, the composition further comprises one or more penetration enhancing agents. In some embodiments, the one or more penetration enhancing agents comprises a non-ionic surfactant or a combination of non-ionic surfactants. In some embodiments, the non-ionic surfactant or a combination of non-ionic surfactants is selected from polyethylene glycol ethers of fatty alcohols, sorbitan esters, polysorbates, sorbitan esters and polyethylene glycol fatty acid esters and combinations thereof. In some embodiments, the polyethylene glycol ethers of fatty alcohols comprise a C₈-C₂₂ fatty alcohol and a polyethylene glycol group having from about 2 to about 8 ethylene glycol subunits. In some embodiments, the polyethylene glycol ethers of fatty alcohols comprise diethylene glycol hexadecyl ether, 2-(2-octadecoxyethoxy)ethanol, diethylene glycol monooleyl ether, polyoxyethylene (2) pleyl ether, polyoxyethylene (3) pleyl ether, or polyoxyethylene (5) oleyl ether, or any combination thereof. In some embodiments, the sorbitan esters comprise sorbitan monolaurate, sorbitan monopalmitate, sorbitan monostearate, sorbitan monooleate, sorbitan trioleate, sorbitan sesquioleate, or sorbitan isostearate, or any combinations thereof. In some embodiments, the polyethylene glycol fatty acid ester comprises PEG-8 dilaurate, PEG-4 dilaurate, PEG-4 laurate, PEG-8 dioleate, PEG-8 distearate, PEG-8 distearate, PEG-7 glyceryl cocoate, and PEG-20 almond glycerides, or any combination thereof. In some embodiments, the polysorbate comprises polysorbate 20, polysorbate 21, polysorbate 40, polysorbate 60, polysorbate 80, polysorbate 85, or any combination thereof. In some embodiments, the non-ionic surfactant or a combination of non-ionic surfactants has a hydrophobic-lipophilic balance (HLB) of about 10 or less. In some embodiments, the non-ionic surfactant or a combination of non-ionic surfactants is present in an amount of from about 0.5% to about to about 5% (w/w) of the composition. In some embodiments, the non-ionic surfactant or combination of non-ionic surfactants is present in an amount of from about 0.5% to about to about 10% (w/w) of the composition. In some embodiments, at least one non-ionic surfactant is present in the oil-in-water emulsion. In some embodiments, at least one non-ionic surfactant is present in the lipid bilayer. In some embodiments, the one or more penetration enhancing agents comprises a combination of a sorbitan ester, a polysorbate, and a polyethylene glycol fatty acid esters. In some embodiments, the one or more penetration enhancing agents comprises a combination of a polyethylene glycol ether of a fatty alcohol, a sorbitan esters, and a polysorbate. In some embodiments, the one or more penetration enhancing agents comprises monolauroyllysine or dipalmitoyllysine, or a combination thereof. In some embodiments, wherein the composition comprises a cationic surfactant. In some embodiments, the cationic surfactant is a mono-cationic surfactant. In some embodiments, the cationic surfactant comprises a fatty amide derived propylene glycol-diammonium phosphate ester. In some embodiments, the cationic surfactant is present in an amount of from about 1% to about 20%. In some embodiments, the cationic surfactant is present in an amount of from about 1% to about 10%. In some embodiments, the cationic penetration enhancing agent comprises a di-cationic penetration enhancing agent. In some embodiments, the di-cationic penetration enhancing agent is a gemini cationic surfactant. In some embodiments, wherein the cationic penetration agent comprises a cationic polymer. In some embodiments, the cationic penetration enhancing agent is present in an amount of from about 0.01% to about 1% (w/w) of the composition. In some embodiments, the penetration enhancing agent comprises a salicylate ester or a nicotinate ester. In some embodiments, the ester is a C₁-C₆ alkyl ester or a benzyl ester. In some embodiments, the penetration enhancing agent comprises methyl salicylate or benzyl nicotinate. In some embodiments, the composition further comprises one or more additional agents. In some embodiments, the additional agents comprise one or more of a thickener, a preservative, a moisturizer, an emollient, a humectant, an antimicrobial, a biological extract, or any combination thereof. In some embodiments, the composition is formulated for topical application to the skin of a subject. In some embodiments, the composition is formulated to deliver the anionic polymer to a specified layer of the skin of a subject. In some embodiments, the composition is formulated to deliver the one or more peptides to a specified layer of the skin of a subject. In some embodiments, the composition is formulated as a cream, a lotion, a suspension, or an emulsion.

In one aspect, provided herein, is a lipid vesicle composition comprising; (a) lipid vesicles each comprising a lipid bilayer comprising vesicle forming lipids; and (b) an oil-in-water emulsion entrapped in the lipid vesicles, and stabilized by one or more surfactants; wherein the lipid bilayer, the oil-in-water emulsion, or a combination thereof comprises one or more peptides, wherein the one or more peptides comprises an amino acid sequence at least about 50%, at least about 60%, at least about 70%, at least about 80%, or at least about 90% sequence homology to any one of SEQ ID NOs: 1-51. In some embodiments, the one or more peptides are entrapped in the lipid bilayer, the oil-in-water emulsion, or in the combination thereof. In some embodiments, the one or more peptides are unmodified. In some embodiments, the one or more peptides is present at a concentration of from about 0.1 mg/mL to about 10 mg/mL. In some embodiments, the one or more peptides comprise tetrapeptides, pentapeptides, hexapeptides, or any combination thereof. In some embodiments, the one or more peptides comprise an amino acid sequence identical to the amino acid sequence of any one of SEQ ID NOs: 1-51. In some embodiments, the one or more peptides comprise at least two peptides each with an amino acid sequence identical to the amino acid sequence of any one of SEQ ID NOs: 1-51. In some embodiments, the one or more peptides comprise an amino acid sequence identical to the amino acid sequence of any one of SEQ ID NOs: 21-40. In some embodiments, the one or more peptides comprise at least two peptides each with an amino acid sequence identical to the amino acid sequence of any one of SEQ ID NOs: 21-40. In some embodiments, the one or more peptides have an amino acid sequence consisting of an identical sequence to the amino acid sequence of any one of SEQ ID NOs: 1-51. In some embodiments, the one or more peptides have an amino acid sequence consisting of an identical sequence to the amino acid sequence of SEQ ID NO: 31, 36, or 37. In some embodiments, the one or more peptides have an amino acid sequence consisting of an identical sequence to the amino acid sequence of SEQ ID NO: 21. In some embodiments, the one or more peptides have an amino acid sequence consisting of an identical sequence to the amino acid sequence of SEQ ID NO: 24. In some embodiments, the one or more peptides have an amino acid sequence consisting of an identical sequence to the amino acid sequence of SEQ ID NO: 30. In some embodiments, the one or more peptides have an amino acid sequence consisting of an identical sequence to the amino acid sequence of SEQ ID NO: 31. In some embodiments, the one or more peptides have an amino acid sequence consisting of an identical sequence to the amino acid sequence of SEQ ID NO: 36. In some embodiments, the one or more peptides have an amino acid sequence consisting of an identical sequence to the amino acid sequence of SEQ ID NO: 37. In some embodiments, the one or more peptides have an amino acid sequence consisting of an identical sequence to the amino acid sequence of SEQ ID NO: 38. In some embodiments, the one or more peptides have an amino acid sequence consisting of an identical sequence to the amino acid sequence of SEQ ID NO: 40. In some embodiments, the lipid bilayer, the oil-in-water emulsion, or the combination thereof further comprises an anionic polymer material. In some embodiments, the anionic polymer material is entrapped in the lipid bilayer, the oil-in-water emulsion, or in a combination thereof. In some embodiments, the anionic polymer material comprises an anionic polysaccharide. In some embodiments, the anionic polymer is present in an amount of about 0.1 mg/mL to about 10 mg/mL of the composition. In some embodiments, the anionic polysaccharide comprises hyaluronic acid or a salt thereof. In some embodiments, the anionic polymer material has a molecular weight of from about 5 kDa to about 500 kDa. In some embodiments, wherein the anionic polymer material comprises a first and a second anionic polymer material, each anionic polymer material having a different molecular weight. In some embodiments, the first and the second anionic polymer material are the same material. In some embodiments, the first anionic polymer material has a molecular weight of up to about 75 kDa and the second anionic polymer material has a molecule weight of greater than about 75 kDa. In some embodiments, the first anionic polymer material has a molecular weight of from about 5 kDa to about 50 kDa, and wherein the second anionic polymer material has a molecular weight of from about 100 kDa to about 500 kDa. In some embodiments, the vesicle forming lipids comprise phospholipids, glycolipids, lecithins, ceramides, lysolecithin, lysophosphatidylethanolamine, phosphatidylserine, phosphatidylinositol, sphingomyelin, cardiolipin, phosphatidic acid, cerebroside, or any combination thereof. In some embodiments, the vesicle forming lipids comprise phospholipids. In some embodiments, the composition comprises vesicle forming lipids in an amount of from about 0.5% to about 25% (w/w) of the composition. In some embodiments, the oil-in-water emulsion comprises a triglyceride in the oil component. In some embodiments, the triglyceride comprises a medium-chain triglyceride. In some embodiments, the triglyceride is present in an amount of from about 1% to about 35% (w/w) of the composition. In some embodiments, the composition comprises a sterol. In some embodiments, the sterol is present in an amount of from about 1% to about 5% (w/w) of the composition. In some embodiments, the composition comprises propylene glycol. In some embodiments, the propylene glycol is present in an amount of from about 1% to about 25% (w/w) of the composition. In some embodiments, the composition comprises one or more viscosity enhancing agents. In some embodiments, the one or more viscosity enhancing agents are present in an amount of from about 0.5% to about 10% (w/w) of the composition. In some embodiments, the composition further comprises one or more penetration enhancing agents. In some embodiments, the one or more penetration enhancing agents comprises a non-ionic surfactant or a combination of non-ionic surfactants. In some embodiments, the non-ionic surfactant or a combination of non-ionic surfactants is selected from polyethylene glycol ethers of fatty alcohols, sorbitan esters, polysorbates, sorbitan esters and polyethylene glycol fatty acid esters and combinations thereof. In some embodiments, the polyethylene glycol ethers of fatty alcohols comprise a C₈-C₂₂ fatty alcohol and a polyethylene glycol group having from about 2 to about 8 ethylene glycol subunits. In some embodiments, the polyethylene glycol ethers of fatty alcohols comprise diethylene glycol hexadecyl ether, 2-(2-octadecoxyethoxy)ethanol, diethylene glycol monooleyl ether, polyoxyethylene (2) oleyl ether, polyoxyethylene (3) oleyl ether, or polyoxyethylene (5) oleyl ether, or any combination thereof. In some embodiments, the sorbitan esters comprise sorbitan monolaurate, sorbitan monopalmitate, sorbitan monostearate, sorbitan monooleate, sorbitan trioleate, sorbitan sesquioleate, or sorbitan isostearate, or any combinations thereof. In some embodiments, the polyethylene glycol fatty acid ester comprises PEG-8 dilaurate, PEG-4 dilaurate, PEG-4 laurate, PEG-8 dioleate, PEG-8 distearate, PEG-8 distearate, PEG-7 glyceryl cocoate, and PEG-20 almond glycerides, or any combination thereof. In some embodiments, the polysorbate comprises polysorbate 20, polysorbate 21, polysorbate 40, polysorbate 60, polysorbate 80, polysorbate 85, or any combination thereof. In some embodiments, the non-ionic surfactant or a combination of non-ionic surfactants has a hydrophobic-lipophilic balance (HLB) of about 10 or less. In some embodiments, the non-ionic surfactant or a combination of non-ionic surfactants is present in an amount of from about 0.5% to about to about 5% (w/w) of the composition. In some embodiments, the non-ionic surfactant or combination of non-ionic surfactants is present in an amount of from about 0.5% to about to about 10% (w/w) of the composition. In some embodiments, at least one non-ionic surfactant is present in the oil-in-water emulsion. In some embodiments, at least one non-ionic surfactant is present in the lipid bilayer. In some embodiments, the one or more penetration enhancing agents comprises a combination of a sorbitan ester, a polysorbate, and a polyethylene glycol fatty acid esters. In some embodiments, the one or more penetration enhancing agents comprises a combination of a polyethylene glycol ether of a fatty alcohol, a sorbitan esters, and a polysorbate. In some embodiments, the one or more penetration enhancing agents comprises monolauroyllysine or dipalmitoyllysine, or a combination thereof. In some embodiments, wherein the composition comprises a cationic surfactant. In some embodiments, the cationic surfactant is a mono-cationic surfactant. In some embodiments, the cationic surfactant comprises a fatty amide derived propylene glycol-diammonium phosphate ester. In some embodiments, the cationic surfactant is present in an amount of from about 1% to about 20%. In some embodiments, the cationic surfactant is present in an amount of from about 1% to about 10%. In some embodiments, the cationic penetration enhancing agent comprises a di-cationic penetration enhancing agent. In some embodiments, the di-cationic penetration enhancing agent is a gemini cationic surfactant. In some embodiments, the cationic penetration agent comprises a cationic polymer. In some embodiments, the cationic penetration enhancing agent is present in an amount of from about 0.01% to about 1% (w/w) of the composition. In some embodiments, the penetration enhancing agent comprises a salicylate ester or a nicotinate ester. In some embodiments, the ester is a C₁-C₆ alkyl ester or a benzyl ester. In some embodiments, the penetration enhancing agent comprises methyl salicylate or benzyl nicotinate. In some embodiments, the composition further comprises one or more additional agents. In some embodiments, wherein the additional agents comprise one or more of a thickener, a preservative, a moisturizer, an emollient, a humectant, an antimicrobial, a biological extract, or any combination thereof. In some embodiments, the composition is formulated for topical application to the skin of a subject. In some embodiments, the composition is formulated to deliver the anionic polymer to a specified layer of the skin of a subject. In some embodiments, the composition is formulated to deliver the one or more peptides to a specified layer of the skin of a subject. In some embodiments, the composition is formulated as a cream, a lotion, a suspension, or an emulsion.

In one aspect, provided herein, is a composition comprising a peptide comprising an amino acid sequence of SEQ ID NO: 37. In some embodiments, the composition further comprises one or more additional agents. In some embodiments, the additional agents comprise one or more of a thickener, a preservative, a moisturizer, an emollient, a humectant, an antimicrobial, a biological extract, or any combination thereof. In some embodiments, the additional agents comprises an anionic polymer material. In some embodiments, the anionic polymer material comprises hyaluronic acid. In some embodiments, the composition is formulated for topical application to the skin of a subject. In some embodiments, the composition is formulated to deliver the one or more peptides to a specified layer of the skin of a subject. In some embodiments, the composition is formulated as a cream, a lotion, a suspension, or an emulsion.

In one aspect, provided herein, is a lipid vesicle composition comprising; (a) lipid vesicles each comprising a lipid bilayer comprising vesicle forming lipids; and (b) an oil-in-water emulsion entrapped in the lipid vesicles, and stabilized by one or more surfactants; wherein the lipid bilayer, the oil-in-water emulsion, or a combination thereof comprises one or more peptides and an anionic polymer material, wherein the one or more peptides comprises an amino acid sequence: Xaa1-Xaa2-Xaa3-Xaa4-Xaa5-Xaa6-Xaa7-Xaa8, wherein: Xaa1 is absent or selected from Ala, Arg, Gly, Lys, Pro, Tyr, Val and a derivative of Ala, Arg, Gly, Lys, Pro, Tyr, or Val; Xaa2 is absent or selected from: Ala, Arg, Cys, Gly, Ile, Lys, Pro, Tyr, Val and a derivative of Ala, Arg, Cys, Gly, Ile, Lys, Pro, Tyr, or Val; Xaa3 is absent or selected from: Ala, Arg, Gln, Gly, Lys, Phe, Pro, Tyr, Val and a derivative of Ala, Arg, Gln, Gly, Lys, Phe, Pro, Tyr, or Val; Xaa4 is absent or selected from: Ala, Arg, Glu, Gly, Lys, Pro, Tyr, Val and a derivative of Ala, Arg, Glu, Gly, Lys, Pro, Tyr, or Val; Xaa5 is absent or selected from: Ala, Arg, Gln, Gly, Lys, Leu, Met, Pro, Thr, Tyr, Val and a derivative of Ala, Arg, Gln, Gly, Lys, Leu, Met, Pro, Thr, Tyr, or Val; Xaa6 is absent or selected from: Ala, Arg, Asp, Gln, Gly, His, Lys, Phe, Pro, Ser, Thr, Tyr, Val and a derivative of Ala, Arg, Asp, Gln, Gly, His, Lys, Phe, Pro, Ser, Thr, Tyr, or Val; Xaa7 is selected from: Ala, Arg, Asn, Asp, Cys, Gly, His, Lys, Phe, Pro, Thr, Tyr, Val and a derivative of Ala, Arg, Asn, Asp, Cys, Gly, His, Lys, Phe, Pro, Thr, Tyr, or Val; Xaa8 is selected from: Ala, Arg, Cys, Glu, Gly, His, Lys, Phe, Pro, Ser, Thr, Trp, Tyr, Val and a derivative of Ala, Arg, Cys, Glu, Gly, His, Lys, Phe, Pro, Ser, Thr, Trp, Tyr, or Val. In some embodiments, the one or more peptides are entrapped in the lipid bilayer, the oil-in-water emulsion, or in the combination thereof. In some embodiments, the one or more peptides are unmodified. In some embodiments, the one or more peptides is present at a concentration of from about 0.1 mg/mL to about 10 mg/mL. In some embodiments, the one or more peptides comprise tetrapeptides, pentapeptides, hexapeptides, or any combination thereof. In some embodiments, the one or more peptides comprises an amino acid sequence at least about 50%, at least about 60%, at least about 70%, at least about 80%, or at least about 90% sequence homology to the amino acid sequence of any one of SEQ ID NOs: 1-51. In some embodiments, the one or more peptides comprise an amino acid sequence identical to the amino acid sequence of any one of SEQ ID NOs: 1-51. In some embodiments, the one or more peptides comprise at least two peptides each with an amino acid sequence identical to the amino acid sequence of any one of SEQ ID NOs: 1-51. In some embodiments, the one or more peptides comprise an amino acid sequence identical to the amino acid sequence of any one of SEQ ID NOs: 21-40. In some embodiments, the one or more peptides comprise at least two peptides each with an amino acid sequence identical to the amino acid sequence of any one of SEQ ID NOs: 21-40. In some embodiments, the one or more peptides have an amino acid sequence consisting of an identical sequence to the amino acid sequence of any one of SEQ ID NOs: 1-51. In some embodiments, the one or more peptides have an amino acid sequence consisting of an identical sequence to the amino acid sequence of SEQ ID NO: 31, 36, or 37. In some embodiments, the one or more peptides have an amino acid sequence consisting of an identical sequence to the amino acid sequence of SEQ ID NO: 21. In some embodiments, the one or more peptides have an amino acid sequence consisting of an identical sequence to the amino acid sequence of SEQ ID NO: 24. In some embodiments, the one or more peptides have an amino acid sequence consisting of an identical sequence to the amino acid sequence of SEQ ID NO: 30. In some embodiments, the one or more peptides have an amino acid sequence consisting of an identical sequence to the amino acid sequence of SEQ ID NO: 31. In some embodiments, the one or more peptides have an amino acid sequence consisting of an identical sequence to the amino acid sequence of SEQ ID NO: 36. In some embodiments, the one or more peptides have an amino acid sequence consisting of an identical sequence to the amino acid sequence of SEQ ID NO: 37. In some embodiments, the one or more peptides have an amino acid sequence consisting of an identical sequence to the amino acid sequence of SEQ ID NO: 38. In some embodiments, the one or more peptides have an amino acid sequence consisting of an identical sequence to the amino acid sequence of SEQ ID NO: 40. In some embodiments, the anionic polymer material is entrapped in the lipid bilayer, the oil-in-water emulsion, or in a combination thereof. In some embodiments, the anionic polymer material comprises an anionic polysaccharide. In some embodiments, the anionic polymer is present in an amount of about 0.1 mg/mL to about 10 mg/mL of the composition. In some embodiments, the anionic polysaccharide comprises hyaluronic acid, or a salt thereof. In some embodiments, the anionic polymer material has a molecular weight of from about 5 kDa to about 500 kDa. In some embodiments, wherein the anionic polymer material comprises a first and a second anionic polymer material, each anionic polymer material having a different molecular weight. In some embodiments, the first and the second anionic polymer material are the same material. In some embodiments, the first anionic polymer material has a molecular weight of up to about 75 kDa and the second anionic polymer material has a molecule weight of greater than about 75 kDa. In some embodiments, the first anionic polymer material has a molecular weight of from about 5 kDa to about 50 kDa, and wherein the second anionic polymer material has a molecular weight of from about 100 kDa to about 500 kDa. In some embodiments, the vesicle forming lipids comprise phospholipids, glycolipids, lecithins, ceramides, lysolecithin, lysophosphatidylethanolamine, phosphatidylserine, phosphatidylinositol, sphingomyelin, cardiolipin, phosphatidic acid, cerebroside, or any combination thereof. In some embodiments, the vesicle forming lipids comprise phospholipids. In some embodiments, the composition comprises vesicle forming lipids in an amount of from about 0.5% to about 25% (w/w) of the composition. In some embodiments, the oil-in-water emulsion comprises a triglyceride in the oil component. In some embodiments, the triglyceride comprises a medium-chain triglyceride. In some embodiments, the triglyceride is present in an amount of from about 1% to about 35% (w/w) of the composition. In some embodiments, the composition comprises a sterol. In some embodiments, the sterol is present in an amount of from about 1% to about 5% (w/w) of the composition. In some embodiments, the composition comprises propylene glycol. In some embodiments, the propylene glycol is present in an amount of from about 1% to about 25% (w/w) of the composition. In some embodiments, the composition comprises one or more viscosity enhancing agents. In some embodiments, the one or more viscosity enhancing agents are present in an amount of from about 0.5% to about 10% (w/w) of the composition. In some embodiments, the composition further comprises one or more penetration enhancing agents. In some embodiments, the one or more penetration enhancing agents comprises a non-ionic surfactant or a combination of non-ionic surfactants. In some embodiments, the non-ionic surfactant or a combination of non-ionic surfactants is selected from polyethylene glycol ethers of fatty alcohols, sorbitan esters, polysorbates, sorbitan esters and polyethylene glycol fatty acid esters and combinations thereof. In some embodiments, the polyethylene glycol ethers of fatty alcohols comprise a C₈-C₂₂ fatty alcohol and a polyethylene glycol group having from about 2 to about 8 ethylene glycol subunits. In some embodiments, the polyethylene glycol ethers of fatty alcohols comprise diethylene glycol hexadecyl ether, 2-(2-octadecoxyethoxy)ethanol, diethylene glycol monooleyl ether, polyoxyethylene (2) oleyl ether, polyoxyethylene (3) oleyl ether, or polyoxyethylene (5) oleyl ether, or any combination thereof. In some embodiments, the sorbitan esters comprise sorbitan monolaurate, sorbitan monopalmitate, sorbitan monostearate, sorbitan monooleate, sorbitan trioleate, sorbitan sesquioleate, or sorbitan isostearate, or any combinations thereof. In some embodiments, the polyethylene glycol fatty acid ester comprises PEG-8 dilaurate, PEG-4 dilaurate, PEG-4 laurate, PEG-8 dioleate, PEG-8 distearate, PEG-8 distearate, PEG-7 glyceryl cocoate, and PEG-20 almond glycerides, or any combination thereof. In some embodiments, the polysorbate comprises polysorbate 20, polysorbate 21, polysorbate 40, polysorbate 60, polysorbate 80, polysorbate 85, or any combination thereof. In some embodiments, the non-ionic surfactant or a combination of non-ionic surfactants has a hydrophobic-lipophilic balance (HLB) of about 10 or less. In some embodiments, the non-ionic surfactant or a combination of non-ionic surfactants is present in an amount of from about 0.5% to about to about 5% (w/w) of the composition. In some embodiments, the non-ionic surfactant or combination of non-ionic surfactants is present in an amount of from about 0.5% to about to about 10% (w/w) of the composition. In some embodiments, at least one non-ionic surfactant is present in the oil-in-water emulsion. In some embodiments, at least one non-ionic surfactant is present in the lipid bilayer. In some embodiments, the one or more penetration enhancing agents comprises a combination of a sorbitan ester, a polysorbate, and a polyethylene glycol fatty acid esters. In some embodiments, the one or more penetration enhancing agents comprises a combination of a polyethylene glycol ether of a fatty alcohol, a sorbitan esters, and a polysorbate. In some embodiments, the one or more penetration enhancing agents comprises monolauroyllysine or dipalmitoyllysine, or a combination thereof. In some embodiments, wherein the composition comprises a cationic surfactant. In some embodiments, the cationic surfactant is a mono-cationic surfactant. In some embodiments, the cationic surfactant comprises a fatty amide derived propylene glycol-diammonium phosphate ester. In some embodiments, the cationic surfactant is present in an amount of from about 1% to about 20%. In some embodiments, the cationic surfactant is present in an amount of from about 1% to about 10%. In some embodiments, the cationic penetration enhancing agent comprises a di-cationic penetration enhancing agent. In some embodiments, the di-cationic penetration enhancing agent is a gemini cationic surfactant. In some embodiments, wherein the cationic penetration agent comprises a cationic polymer. In some embodiments, the cationic penetration enhancing agent is present in an amount of from about 0.01% to about 1% (w/w) of the composition. In some embodiments, the penetration enhancing agent comprises a salicylate ester or a nicotinate ester. In some embodiments, the ester is a C₁-C₆ alkyl ester or a benzyl ester. In some embodiments, the penetration enhancing agent comprises methyl salicylate or benzyl nicotinate. In some embodiments, the composition further comprises one or more additional agents. In some embodiments, wherein the additional agents comprise one or more of a thickener, a preservative, a moisturizer, an emollient, a humectant, an antimicrobial, a biological extract, or any combination thereof. In some embodiments, the composition is formulated for topical application to the skin of a subject. In some embodiments, the composition is formulated to deliver the anionic polymer to a specified layer of the skin of a subject. In some embodiments, the composition is formulated to deliver the one or more peptides to a specified layer of the skin of a subject. In some embodiments, the composition is formulated as a cream, a lotion, a suspension, or an emulsion.

In one aspect, provided herein, is a lipid vesicle composition comprising; (a) lipid vesicles each comprising a lipid bilayer comprising vesicle forming lipids; and (b) an oil-in-water emulsion entrapped in the lipid vesicles, and stabilized by one or more surfactants; wherein the lipid bilayer, the oil-in-water emulsion, or a combination thereof comprises one or more peptides and an anionic polymer material, wherein the one or more peptides comprises an amino acid sequence at least about 50%, at least about 60%, at least about 70%, at least about 80%, or at least about 90% sequence homology to the amino acid sequence of any one of SEQ ID NOs: 1-51. In some embodiments, the one or more peptides are entrapped in the lipid bilayer, the oil-in-water emulsion, or in the combination thereof. In some embodiments, the one or more peptides are unmodified. In some embodiments, the one or more peptides is present at a concentration of from about 0.1 mg/mL to about 10 mg/mL. In some embodiments, the one or more peptides are tetrapeptides, pentapeptides, hexapeptides, or any combination thereof. In some embodiments, the one or more peptides comprise an amino acid sequence identical to the amino acid sequence of any one of SEQ ID NOs: 1-51. In some embodiments, the one or more peptides comprise at least two peptides each with an amino acid sequence identical to the amino acid sequence of any one of SEQ ID NOs: 1-51. In some embodiments, the one or more peptides comprise an amino acid sequence identical to the amino acid sequence of any one of SEQ ID NOs: 21-40. In some embodiments, the one or more peptides comprise at least two peptides each with an amino acid sequence identical to the amino acid sequence of any one of SEQ ID NOs: 21-40. In some embodiments, the one or more peptides have an amino acid sequence consisting of an identical sequence to the amino acid sequence of any one of SEQ ID NOs: 1-51. In some embodiments, the one or more peptides have an amino acid sequence consisting of an identical sequence to the amino acid sequence of SEQ ID NO: 31, 36, or 37. In some embodiments, the one or more peptides have an amino acid sequence consisting of an identical sequence to the amino acid sequence of SEQ ID NO: 21. In some embodiments, the one or more peptides have an amino acid sequence consisting of an identical sequence to the amino acid sequence of SEQ ID NO: 24. In some embodiments, the one or more peptides have an amino acid sequence consisting of an identical sequence to the amino acid sequence of SEQ ID NO: 30. In some embodiments, the one or more peptides have an amino acid sequence consisting of an identical sequence to the amino acid sequence of SEQ ID NO: 31. In some embodiments, the one or more peptides have an amino acid sequence consisting of an identical sequence to the amino acid sequence of SEQ ID NO: 36. In some embodiments, the one or more peptides have an amino acid sequence consisting of an identical sequence to the amino acid sequence of SEQ ID NO: 37. In some embodiments, the one or more peptides have an amino acid sequence consisting of an identical sequence to the amino acid sequence of SEQ ID NO: 38. In some embodiments, the one or more peptides have an amino acid sequence consisting of an identical sequence to the amino acid sequence of SEQ ID NO: 40. In some embodiments, the anionic polymer material is entrapped in the lipid bilayer, the oil-in-water emulsion, or in a combination thereof. In some embodiments, the anionic polymer material comprises an anionic polysaccharide. In some embodiments, the anionic polymer is present in an amount of about 0.1 mg/mL to about 10 mg/mL of the composition. In some embodiments, the anionic polysaccharide comprises hyaluronic acid, or a salt thereof. In some embodiments, the anionic polymer material has a molecular weight of from about 5 kDa to about 500 kDa. In some embodiments, wherein the anionic polymer material comprises a first and a second anionic polymer material, each anionic polymer material having a different molecular weight. In some embodiments, the first and the second anionic polymer material are the same material. In some embodiments, the first anionic polymer material has a molecular weight of up to about 75 kDa and the second anionic polymer material has a molecule weight of greater than about 75 kDa. In some embodiments, the first anionic polymer material has a molecular weight of from about 5 kDa to about 50 kDa, and wherein the second anionic polymer material has a molecular weight of from about 100 kDa to about 500 kDa. In some embodiments, the vesicle forming lipids comprise phospholipids, glycolipids, lecithins, ceramides, lysolecithin, lysophosphatidylethanolamine, phosphatidylserine, phosphatidylinositol, sphingomyelin, cardiolipin, phosphatidic acid, cerebroside, or any combination thereof. In some embodiments, the vesicle forming lipids comprise phospholipids. In some embodiments, the composition comprises vesicle forming lipids in an amount of from about 0.5% to about 25% (w/w) of the composition. In some embodiments, the oil-in-water emulsion comprises a triglyceride in the oil component. In some embodiments, the triglyceride comprises a medium-chain triglyceride. In some embodiments, the triglyceride is present in an amount of from about 1% to about 35% (w/w) of the composition. In some embodiments, the composition comprises a sterol. In some embodiments, the sterol is present in an amount of from about 1% to about 5% (w/w) of the composition. In some embodiments, the composition comprises propylene glycol. In some embodiments, the propylene glycol is present in an amount of from about 1% to about 25% (w/w) of the composition. In some embodiments, the composition comprises one or more viscosity enhancing agents. In some embodiments, the one or more viscosity enhancing agents are present in an amount of from about 0.5% to about 10% (w/w) of the composition. In some embodiments, the composition further comprises one or more penetration enhancing agents. In some embodiments, the one or more penetration enhancing agents comprises a non-ionic surfactant or a combination of non-ionic surfactants. In some embodiments, the non-ionic surfactant or a combination of non-ionic surfactants is selected from polyethylene glycol ethers of fatty alcohols, sorbitan esters, polysorbates, sorbitan esters and polyethylene glycol fatty acid esters and combinations thereof. In some embodiments, the polyethylene glycol ethers of fatty alcohols comprise a C₈-C₂₂ fatty alcohol and a polyethylene glycol group having from about 2 to about 8 ethylene glycol subunits. In some embodiments, the polyethylene glycol ethers of fatty alcohols comprise diethylene glycol hexadecyl ether, 2-(2-octadecoxyethoxy)ethanol, diethylene glycol monooleyl ether, polyoxyethylene (2) oleyl ether, polyoxyethylene (3) oleyl ether, or polyoxyethylene (5) oleyl ether, or any combination thereof. In some embodiments, the sorbitan esters comprise sorbitan monolaurate, sorbitan monopalmitate, sorbitan monostearate, sorbitan monooleate, sorbitan trioleate, sorbitan sesquioleate, or sorbitan isostearate, or any combinations thereof. In some embodiments, the polyethylene glycol fatty acid ester comprises PEG-8 dilaurate, PEG-4 dilaurate, PEG-4 laurate, PEG-8 dioleate, PEG-8 distearate, PEG-8 distearate, PEG-7 glyceryl cocoate, and PEG-20 almond glycerides, or any combination thereof. In some embodiments, the polysorbate comprises polysorbate 20, polysorbate 21, polysorbate 40, polysorbate 60, polysorbate 80, polysorbate 85, or any combination thereof. In some embodiments, the non-ionic surfactant or a combination of non-ionic surfactants has a hydrophobic-lipophilic balance (HLB) of about 10 or less. In some embodiments, the non-ionic surfactant or a combination of non-ionic surfactants is present in an amount of from about 0.5% to about to about 5% (w/w) of the composition. In some embodiments, the non-ionic surfactant or combination of non-ionic surfactants is present in an amount of from about 0.5% to about to about 10% (w/w) of the composition. In some embodiments, at least one non-ionic surfactant is present in the oil-in-water emulsion. In some embodiments, at least one non-ionic surfactant is present in the lipid bilayer. In some embodiments, the one or more penetration enhancing agents comprises a combination of a sorbitan ester, a polysorbate, and a polyethylene glycol fatty acid esters. In some embodiments, the one or more penetration enhancing agents comprises a combination of a polyethylene glycol ether of a fatty alcohol, a sorbitan esters, and a polysorbate. In some embodiments, the one or more penetration enhancing agents comprises monolauroyllysine or dipalmitoyllysine, or a combination thereof. In some embodiments, wherein the composition comprises a cationic surfactant. In some embodiments, the cationic surfactant is a mono-cationic surfactant. In some embodiments, the cationic surfactant comprises a fatty amide derived propylene glycol-diammonium phosphate ester. In some embodiments, the cationic surfactant is present in an amount of from about 1% to about 20%. In some embodiments, the cationic surfactant is present in an amount of from about 1% to about 10%. In some embodiments, the cationic penetration enhancing agent comprises a di-cationic penetration enhancing agent. In some embodiments, the di-cationic penetration enhancing agent is a gemini cationic surfactant. In some embodiments, wherein the cationic penetration agent comprises a cationic polymer. In some embodiments, the cationic penetration enhancing agent is present in an amount of from about 0.01% to about 1% (w/w) of the composition. In some embodiments, the penetration enhancing agent comprises a salicylate ester or a nicotinate ester. In some embodiments, the ester is a C₁-C₆ alkyl ester or a benzyl ester. In some embodiments, the penetration enhancing agent comprises methyl salicylate or benzyl nicotinate. In some embodiments, the composition further comprises one or more additional agents. In some embodiments, wherein the additional agents comprise one or more of a thickener, a preservative, a moisturizer, an emollient, a humectant, an antimicrobial, a biological extract, or any combination thereof. In some embodiments, the composition is formulated for topical application to the skin of a subject. In some embodiments, the composition is formulated to deliver the anionic polymer to a specified layer of the skin of a subject. In some embodiments, the composition is formulated to deliver the one or more peptides to a specified layer of the skin of a subject. In some embodiments, the composition is formulated as a cream, a lotion, a suspension, or an emulsion.

In one aspect, provided herein, is a method of preparing a lipid vesicle composition provided herein, comprising a) preparing an oil-in-water emulsion comprising the one or more peptides, by mixing oil components of the oil-in-water emulsion with aqueous components of the oil-in-water emulsion, wherein the oil components and/or the aqueous components of the oil-in-water emulsion comprises the one or more surfactants; b) solubilizing vesicle forming lipids in an acceptable solvent other than water; c) adding the oil-in-water emulsion to the solubilized vesicle forming lipids; and d) mixing the oil-in-water emulsion and the solubilized vesicle forming lipids under mixing conditions effective to form the lipid vesicles comprising a lipid bilayer comprising vesicle forming lipids, and an oil-in-water emulsion entrapped in the lipid vesicles. In some embodiments, a) further comprises the anionic polymer material.

In one aspect, provided herein, is a method of producing one or more cosmetic effects by delivering a cosmetic agent below a skin surface of a subject, comprising applying to the skin surface a lipid vesicle composition provided herein. In some embodiments, the cosmetic agent is delivered to the dermis of the subject. In some embodiments, the cosmetic agent is delivered to the epidermis of the subject. In some embodiments, the cosmetic agent is delivered to contiguous muscles or subcutaneous tissue, including adipose tissue, of the subject. In some embodiments, the one or more cosmetic effect comprises prevention or temporary improvement of the appearance of one or more of skin wrinkles. In some embodiments, the one or more skin wrinkles comprises moderate to severe glabellar lines associated with corrugator and/or procerus muscle activity, moderate to severe lateral canthal lines associated with orbicularis oculi activity (crow's feet lines), or moderate to severe forehead lines associated with frontalis muscle activity.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C shows physicochemical characterization of multisome formulations F1, F2 and F3. FIG. 1A shows confocal microscopic images of multisome formulations F1 (top), F2 (middle) and F3 (bottom) containing a rhodamine red labelled HA250K and green FITC-HA10K; FI tracings show co-localization of the two labels in the vesicles. FIG. 1B shows light microscopic images of multisome formulations F1 (top), F2 (middle) and F3 (bottom). FIG. 1C shows particle size distribution of multisome formulations F1 (top), F2 (middle) and F3 (bottom). The figures are labeled size distribution by intensity. The x-axis is the size (d nm) from 0.1 to 10000 labeled in increments of a factor of 10. The y-axis for F1 (top) shows the intensity (percent) from 0 to 20 labeled in increments of 5. The y-axis for F2 (middle) shows the intensity (percent) from 0 to 30 labeled in increments of 10. The y-axis for F3 (bottom) shows the intensity (percent) from 0 to 12 labeled in increments of 2.

FIG. 2 shows confocal microscopic images of human skin to which cationic multisome formulations were applied. Cationic multisome formulations were prepared with a rhodamine red labelled HA250K and green FITC-HA10K or FITC-HA50K. FI tracing show the levels of rhodamine red labelled HA250K and green FITC-HA10K or FITC-HA50K in the skin layers from the surface of the skin to the upper dermis. For the FI tracing for F1 (top) the x-axis is distance (nm) from 0 to 300000 labeled in increments of 50000; the y-axis is intensity from 0 to 66 in labeled increments of 10 up to 60. For the FI tracing for F2 (middle) the x-axis is distance (nm) from 0 to 198107 labeled in increments of 20000 up to 180000; the y-axis is intensity from 0 to 90 in labeled increments of 10. For the FI tracing for F3 (bottom) the x-axis is distance (nm) from 0 to 375190 labeled in increments of 50000 up to 350000; the y-axis is intensity from 0 to 65 in labeled increments of 10 up to 60. The plain of the tracing direction is shown on each micrograph.

FIGS. 3A-3C show confocal microscopic images of human skin to which multisome formulations were applied. Multisome formulations were prepared with a rhodamine red labelled HA250K and green FITC-HA10K or FITC-HA50K. FI tracing show the levels of rhodamine red labelled HA250K and green FITC-HA10K or FITC-HA50K in the skin layers from the surface of the skin to the upper dermis. The x-axis shown are distance (nm) and the y-axis are intensity in the FI tracings. The plane of the tracing direction is shown on each micrograph. FIG. 3A shows results for E1 (F4HA250+10 1 mg), E4 (F4HA250+50 1 mg), E7 (F3-2HA250+50 1.5 mg), and E10 (F1-F6-COSM6BN-HA250+50 1.5 mg) from top to bottom. FIG. 3B shows results for E2 (F4HA250+10 1.5 mg), E5 (F4HA250+50 1.5 mg), E8 (F1-F6-COSM6-HA250+10 1.5 mg), and E11 (Gel-HA250+50 1.5 mg) from top to bottom. FIG. 3C shows results for E3 (COSMF4HA250+10 1.5 mg), E6 (COSMF4HA250+50 1.5 mg), E9 (F1-F6-COSM6-HA250+50 1.5 mg), and E12 (Gel-HA250+10 1.5 mg) from top to bottom.

FIGS. 4A-4B show confocal microscopic images of human skin to which multisome formulations were applied. Multisome formulations were prepared with a rhodamine red labelled HA250K and green FITC-HA10K or FITC-HA50K. FI tracing show the levels of rhodamine red labelled HA250K and green FITC-HA10K or FITC-HA50K in the skin layers from the surface of the skin to the upper dermis. The x-axis shown are distance (nm) and the y-axis are intensity in the FI tracings. The plane of the tracing direction is shown on each micrograph. FIG. 4A shows results for G1 (F1-F5-COSM4-HA250+10), G2 (F1-F5-COSM4+HA250+10+BN), G3 (F1-F6-COSM5-HA250+10), and G4 (F1-F6-COSM5-HA250+10+BN) from top to bottom. FIG. 4B shows results for G5 (F1-F5-COSM4-HA250+50), G6 (F1-F5-COSM4+HA250+50+BN), G7 (F1-F6-COSM5-HA250+50), and G8 (F1-F6-COSM5-HA250+50+BN) from top to bottom.

FIG. 5 shows an exemplary pictorial workflow for the preparation of lipid vesicles provided herein.

FIG. 6 shows an exemplary workflow for the preparation of lipid vesicles comprising hyaluronic acid (HA) as provided herein.

FIGS. 7A-D show physicochemical characterization of multisome formulations containing various HA+functional ingredient packs (FIPs). FIG. 7A shows confocal microscopic images of multisome formulations and fluorescence intensity (FI) tracings (Panel A) and light microscopic images of the multisome formulations (Panel B). In the FI tracings, the x-axis are distance (μm) from 0 to 50 in increments of 5; the y-axis are intensity from 0 to 250 shown in increments of 50. FIG. 7B shows confocal microscopic images of further multisome formulations and FI tracings (Panel A) and corresponding light microscopic images (Panel B). In the FI tracings for F5P2.6-F6COSM5V2-H6+P2 (top), the axis is distance (μm) from 0 to 55 in increments of 5; the y-axis is intensity shows from 0 to 250 in increments of 50. In the FI tracings for F1-F6-COSM5V2-H6+P2 (bottom), the axis is distance (μm) from 0 to about 130 in increments of 20; the y-axis is intensity shows from 0 to 250 in increments of 50. FIG. 7C shows a confocal microscopic image of a selected multisome formulation and FI tracing (Panel A), as well as light microscopic images of the selected formulation for various batch sized (Panel B). In the FI tracing, the x-axis is distance (nm) from 0 to about 35000 in increments of 5000; the y-axis shows intensity from 0 to 57 in increments of 10. FIG. 7D shows a confocal microscopic image of another selected multisome formulation and FI tracing (Panel A), as well as light microscopic images of the selected formulation before and after mixing of additional ingredients to the formulation (Panel B). In the FI tracing, the x-axis is distance (nm) from 0 to about 12000 in increments of 2000; the y-axis shows intensity from 0 to 170 in increments of 20. The confocal microscopic images in Panels A in FIGS. 7A-D comprise a rhodamine red labelled HA250K and green FITC-HA10K and the FI tracings show co-localization of the two labels in the vesicles.

FIGS. 8A-8B show confocal microscopic images and FI tracing of human skin treated with two formulations, F5P2.6-F6COSM5V2-HA5-1+P2 (FIG. 8A) and F1-F6COSM5V2-H5-1+P2 (FIG. 8B), at two different HA250/50 ratios with HA+FIP formulations: HA5-1 and 5-2. Multisome formulations were prepared with a rhodamine red labelled HA250K and green FITC-HA50K. FI tracing show the levels of rhodamine red labelled HA250K and green FITC-HA10K or FITC-HA50K in the skin layers from the surface of the skin to the upper dermis. The plain of the tracing direction is shown on each micrograph. In the FI tracings in FIG. 8A, the x-axis is distance (nm) from 0 to 133123 in increments of 20000; the y-axis is intensity from 0 to 109 in increments of 10 for HA5-1 (top), while the x-axis is distance (nm) from 0 to 120000 in increments of 20000; the y-axis is intensity from 0 to 80 in increments of 10 for HA 5-2 (bottom). In the FI tracings in FIG. 8B, the x-axis is distance (nm) from 0 to 112730 in increments of 20000; the y-axis is intensity from 0 to 99 in increments of 10 for HA5-1 (top), while the x-axis is distance (nm) from 0 to 100000 in increments of 10000; the y-axis is intensity from 0 to 97 in increments of 10 for HA 5-2 (bottom).

FIG. 9 shows confocal microscopic images and FI tracing of human skin treated with the formulation F5P2.6-F6COSM5V2 with three different HA+FIPs formulations: HA5-1+P2 (top), HA6+P2 (middle) and HA8+P2 (bottom). Multisome formulations were prepared with a rhodamine red labelled HA250K and green FITC-HA50K.FI tracing show the levels of rhodamine red labelled HA250K and green FITC-HA10K or FITC-HA50K in the skin layers from the surface of the skin to the upper dermis. The plain of the tracing direction is shown on each micrograph. For the FI tracings for HA5-1+P2 (top), the x-axis is distance (nm) from 0 to 133123 in increments of 20000; the y-axis is intensity from 0 to 109 in increments of 10. For the FI tracings for HA6+P2 (middle), the x-axis is distance (nm) from 0 to 130138 in increments of 20000; the y-axis is intensity from 0 to 70 in increments of 10. For the FI tracings for HA8+P2 (bottom), the x-axis is distance (nm) from 0 to 118512 in increments of 20000; the y-axis is intensity from 0 to 23 in increments of 2.

FIG. 10 shows confocal microscopic images and FI tracing of human skin treated with the formulation F5P2.6-F6COSM5V2 formulated with either propylene glycol (top) or butylene glycol (bottom). Multisome formulations were prepared with a rhodamine red labelled HA250K and green FITC-HA50K. FI tracing show the levels of rhodamine red labelled HA250K and green FITC-HA10K or FITC-HA50K in the skin layers from the surface of the skin to the upper dermis. The plain of the tracing direction is shown on each micrograph. For the FI tracing for F5P2.6-F6COSM5V2 formulated with propylene glycol (top), the x-axis is distance (nm) from 0 to 133123 in increments of 20000; the y-axis is intensity from 0 to 109 in increments of 10. For FI tracings for F5P2.6-F6COSM5V2 formulated with butylene glycol (bottom), the x-axis is distance (nm) from 0 to 120000 in increments of 20000; the y-axis is intensity from 0 to 17 in increments of 2.

FIGS. 11A-11C show confocal microscopic images and FI tracing of human skin treated with the formulation F1-F6COSM5NC-HA4-2P. Multisome formulations were prepared with a rhodamine red labelled HA250K and green FITC-HA50K. FI tracing show the levels of rhodamine red labelled HA250K and green FITC-HA10K or FITC-HA50K in the skin layers from the surface of the skin to the upper dermis. The plain of the tracing direction is shown on each micrograph. Measurements shown on 12 different areas, where FIG. 11A shows areas 1, 4, 7, and 10; FIG. 11B shows areas 2, 5, 8, and 11; and FIG. 11C shows areas 3, 6, 9, and 12 (from top to bottom). For each of the FI tracings, the x-axis is distance (nm) and the y-axis is intensity. In FIG. 11A, for area 1 the x-axis is shown from 0 to greater than 80000 in increments of 10000 and y-axis is shown from 0 to 120 in increments of 20; for area 4 the x-axis is shown from 0 to greater than 140000 in increments of 20000 and y-axis is shown from 0 to 100 in increments of 10; for area 7 the x-axis is shown from 0 to greater than 100000 in increments of 20000 and y-axis is shown from 0 to 120 in increments of 20; and for area 10 the x-axis is shown from 0 to greater than 120000 in increments of 20000 and y-axis is shown from 0 to 50 in increments of 5. In FIG. 11B, for area 2 the x-axis is shown from 0 to greater than 120000 in increments of 20000 and y-axis is shown from 0 to 110 in increments of 10; for area 5 the x-axis is shown from 0 to greater than 120000 in increments of 20000 and y-axis is shown from 0 to 75 in increments of 10; for area 8 the x-axis is shown from 0 to about 100000 in increments of 20000 and y-axis is shown from 0 to 170 in increments of 20; and for area 11 the x-axis is shown from 0 to greater than 150000 in increments of 20000 and y-axis is shown from 0 to 60 in increments of 10. In FIG. 11C, for area 3 the x-axis is shown from 0 to greater than 120000 in increments of 20000 and y-axis is shown from 0 to greater than 250 in increments of 50; for area 6 the x-axis is shown from 0 to greater than 140000 in increments of 20000 and y-axis is shown from 0 to greater than 90 in increments of 10; for area 9 the x-axis is shown from 0 to greater than 100000 in increments of 10000 and y-axis is shown from 0 to 55 in increments of 5; and for area 12 the x-axis is shown from 0 to greater than 120000 in increments of 20000 and y-axis is shown from 0 to 80 in increments of 10.

FIG. 12 shows confocal microscopic images and FI tracing of human skin treated with a gel formulation (non-vesicle formulation) as a control formulation. The gel was prepared with a rhodamine red labelled HA250K and green FITC-HA50K. FI tracing show the levels of rhodamine red labelled HA250K and green FITC-HA10K or FITC-HA50K in the skin layers from the surface of the skin to the upper dermis. The plain of the tracing direction is shown on each micrograph. Measurements shown on 2 different areas. For the FI tracing for area 1 (top), the x-axis is distance (nm) from 0 to 140000 in increments of 20000; the y-axis is intensity from 0 to 80 in increments of 10. For the FI tracing for area 2 (bottom), the x-axis is distance (nm) from 0 to 112386 in increments of 20000; the y-axis is intensity from 0 to 80 in increments of 10.

### DETAILED DESCRIPTION OF THE INVENTION

### Definitions

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of” or “consisting of.” The phrase “consisting essentially of” is used herein to require the specified feature(s) as well as those which do not materially affect the character or function of the claimed invention. As used herein, the term “consisting” is used to indicate the presence of the recited feature alone.

As used in the specification and appended claims, unless specified to the contrary, the following terms have the meaning indicated below.

As used herein, the term “pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

As used herein, the term “unmodified peptides” refers to peptides that have not been terminally modified to include additional functional groups. In some embodiments, the peptides have not been modified to include functional groups to enhance transdermal penetration, such as an octanoyl, decanoyl, lauroyl, myristoyl, palmitoyl, stearoyl, biotinoyl, elaidoyl, oleoyl or lipoyl group. In some embodiments, the peptides have not been modified to include oleanolic acid.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

As used herein, “conservative substitution” means an exchange of one amino acid for another amino acid with similar properties, such as size, charge, and polarity. The substitution can be for a natural or modified (e.g., unnatural amino acid). Non-limiting of examples which can be interchanged in conservative substitutions include the following groupings: Large Hydrophobics (Valine, Leucine, Isoleucine, Phenylalanine, Tryptophan, Tyrosine, Methionine), Small Non-Polar (Alanine, Glycine), Polar (Serine, Threonine, Glutamine, Asparagine, Cysteine, histidine), Positively Charged (Lysine, Arginine), and Negatively Charged (Glutamate, Aspartate).

When a % is used herein to refer to an amount of a component, unless otherwise specified, it is intended that the % be the % w/w.

The term “penetration enhancing agents” and “penetration enhancers” are used herein interchangeably. As used herein, it refers to one or more ingredients which facilitate or increase the penetration of one or more active ingredients (e.g., anionic polymeric materials such as hyaluronic acid or one or more peptides) through one or more layers of the skin of a subject. In some embodiments, the penetration enhancing agent is a surfactant, including, for example, non-ionic surfactants having a hydrophilic-lipophilic balance (HLB) of about 10 or less, a cationic group, or another agent such as a terpene, alkaloid, salicylate derivative, nicotinate derivative, or any combination thereof.

The term “multisome” as used herein refers lipid vesicle (such as a biphasic lipid vesicle) which comprises one or more penetration enhancers, which in preferred embodiments include multiple penetration enhancers which work in a synergistic fashion. In some embodiments, multisomes include vesicle whose central core compartments are occupied by an oil-in-water emulsion composed of an aqueous continuous phase and a dispersed hydrophobic, hydrophilic or oil phase. In an embodiment, the spaces between adjacent bilayers of lipid vesicles may also be occupied by the emulsion.

The term “lipid vesicle composition” as used herein refers to a composition which includes one or more lipid vesicles (e.g., multisomal lipid vesicles, lipid bilayer vesicles, etc.). When a lipid vesicle composition is described as “comprising” one or more additional components (e.g., an anionic polymer material or one or more peptides provided herein), it is intended that the composition includes the additional component in any manner within the composition (e.g., encapsulated within a lipid vesicle. For example, a lipid vesicle composition comprising an anionic polymer material can include the anionic polymer material encapsulated within a lipid bilayer of the lipid vesicle composition.

The term “emulsion” as used herein refers to a mixture of two immiscible substances.

The term “bilayer” as used herein refers to a structure composed of amphiphilic lipid molecules arranged in two molecular layers, with the hydrophobic tails on the interior and the polar head groups on the exterior surfaces.

The term “topical application” or “topical delivery” as used herein means intradermal, transdermal and/or transmucosal delivery of a compound by administration of a composition comprising the compound or compounds to skin and/or a mucosal membrane.

The term “gemini surfactant” as used herein refers to a surfactant molecule which contains more than one hydrophobic tail, and each hydrophobic tail having a hydrophilic head wherein the hydrophobic tails or hydrophilic heads are linked together by a spacer moiety. The hydrophobic tails can be identical or differ. Likewise, the hydrophilic heads can be identical or differ, the hydrophilic heads may be anionic, cationic, or neutral.

The term “HLB” or “Hydrophilic-Lipophilic Balance” value refers to standard HLB according to Griffin, J. Soc. Cosm. Chem., vol. 5, 249 (1954), which indicates the degrees of hydrophilicity and lipophilicity of a surfactant.

### Lipid Vesicle Compositions of Anionic Polymer Materials Such as Hyaluronic Acid for Intradermal Delivery

In one aspect, provided herein, is a lipid vesicle composition comprising an anionic polymer material. In some embodiments, the lipid vesicle composition comprises lipid vesicles each comprising a lipid bilayer comprising vesicle forming lipids. In some embodiments, the lipid vesicle composition comprises an oil-in-water emulsion entrapped in the lipid vesicles. In some embodiments, the oil-in-water emulsion is stabilized by one or more surfactants. In some embodiments, the anionic polymer material is entrapped in the lipid bilayer, the oil-in-water emulsion, or a combination thereof. In some embodiments, the anionic polymer material is hyaluronic acid.

### Anionic Polymer Materials

In some aspects, the lipid vesicle compositions provided herein comprise an anionic polymer material. The anionic polymer material is desirably one which is compatible with delivery beneath the surface of the skin of a subject. In some embodiments, the anionic polymer material is one which acts as a volumizer or filler after delivery beneath the surface of the skin. In some embodiments, the anionic polymer material acts as a support for another layer of skin (e.g., the epidermis) in order to correct depressions of the skin or restore facial volume. In some embodiments, the anionic polymer material is not crosslinked. In some embodiments, the anionic polymer material is crosslinked (e.g., crosslinked hyaluronic acid, such as Hyacross™ TL100).

In some embodiments, the anionic polymer material comprises an anionic polysaccharide. In some embodiments, the anionic polysaccharide is non-sulfated glycosaminoglycan. In some embodiments, the anionic polymeric material is a naturally occurring substance. In some embodiments, the anionic polymeric material naturally occurs in a human. In some embodiments, the anionic polymer material naturally occurs in connective or epithelial tissue in a human. In some embodiments, the anionic polymeric material is hyaluronic acid, or a pharmaceutically acceptable salt thereof.

In some embodiments, the hyaluronic acid is a pharmaceutically acceptable salt of hyaluronic acid. In some embodiments, the salt is the sodium salt, the potassium salt, the magnesium salt, or any combination thereof. In some embodiments, the salt is the sodium salt.

In some embodiments, the anionic polymer material has a molecular weight of from about 5 kDa to about 500 kDa. In some embodiments, the molecular weight is the weight average molecular weight. In some embodiments, the anionic polymeric material has a molecular weight of about 5 kDa to about 500 kDa. In some embodiments, the anionic polymeric material has a molecular weight of about 5 kDa to about 10 kDa, about 5 kDa to about 20 kDa, about 5 kDa to about 50 kDa, about 5 kDa to about 100 kDa, about 5 kDa to about 200 kDa, about 5 kDa to about 250 kDa, about 5 kDa to about 300 kDa, about 5 kDa to about 400 kDa, about 5 kDa to about 500 kDa, about 10 kDa to about 20 kDa, about 10 kDa to about 50 kDa, about 10 kDa to about 100 kDa, about 10 kDa to about 200 kDa, about 10 kDa to about 250 kDa, about 10 kDa to about 300 kDa, about 10 kDa to about 400 kDa, about 10 kDa to about 500 kDa, about 20 kDa to about 50 kDa, about 20 kDa to about 100 kDa, about 20 kDa to about 200 kDa, about 20 kDa to about 250 kDa, about 20 kDa to about 300 kDa, about 20 kDa to about 400 kDa, about 20 kDa to about 500 kDa, about 50 kDa to about 100 kDa, about 50 kDa to about 200 kDa, about 50 kDa to about 250 kDa, about 50 kDa to about 300 kDa, about 50 kDa to about 400 kDa, about 50 kDa to about 500 kDa, about 100 kDa to about 200 kDa, about 100 kDa to about 250 kDa, about 100 kDa to about 300 kDa, about 100 kDa to about 400 kDa, about 100 kDa to about 500 kDa, about 200 kDa to about 250 kDa, about 200 kDa to about 300 kDa, about 200 kDa to about 400 kDa, about 200 kDa to about 500 kDa, about 250 kDa to about 300 kDa, about 250 kDa to about 400 kDa, about 250 kDa to about 500 kDa, about 300 kDa to about 400 kDa, about 300 kDa to about 500 kDa, or about 400 kDa to about 500 kDa. In some embodiments, the anionic polymeric material has a molecular weight of about 5 kDa, about 10 kDa, about 20 kDa, about 50 kDa, about 100 kDa, about 200 kDa, about 250 kDa, about 300 kDa, about 400 kDa, or about 500 kDa. In some embodiments, the anionic polymeric material has a molecular weight of at least about 5 kDa, about 10 kDa, about 20 kDa, about 50 kDa, about 100 kDa, about 200 kDa, about 250 kDa, about 300 kDa, or about 400 kDa. In some embodiments, the anionic polymeric material has a molecular weight of at most about 10 kDa, about 20 kDa, about 50 kDa, about 100 kDa, about 200 kDa, about 250 kDa, about 300 kDa, about 400 kDa, or about 500 kDa.

In some embodiments, an anionic polymer material is present in an amount of about 0.01 wt % to about 1 wt %. In some embodiments, the anionic polymer material is present in an amount of about 0.01 wt % to about 0.02 wt %, about 0.01 wt % to about 0.05 wt %, about 0.01 wt % to about 0.08 wt %, about 0.01 wt % to about 0.1 wt %, about 0.01 wt % to about 0.15 wt %, about 0.01 wt % to about 0.2 wt %, about 0.01 wt % to about 0.25 wt %, about 0.01 wt % to about 0.3 wt %, about 0.01 wt % to about 0.4 wt %, about 0.01 wt % to about 0.5 wt %, about 0.01 wt % to about 1 wt %, about 0.02 wt % to about 0.05 wt %, about 0.02 wt % to about 0.08 wt %, about 0.02 wt % to about 0.1 wt %, about 0.02 wt % to about 0.15 wt %, about 0.02 wt % to about 0.2 wt %, about 0.02 wt % to about 0.25 wt %, about 0.02 wt % to about 0.3 wt %, about 0.02 wt % to about 0.4 wt %, about 0.02 wt % to about 0.5 wt %, about 0.02 wt % to about 1 wt %, about 0.05 wt % to about 0.08 wt %, about 0.05 wt % to about 0.1 wt %, about 0.05 wt % to about 0.15 wt %, about 0.05 wt % to about 0.2 wt %, about 0.05 wt % to about 0.25 wt %, about 0.05 wt % to about 0.3 wt %, about 0.05 wt % to about 0.4 wt %, about 0.05 wt % to about 0.5 wt %, about 0.05 wt % to about 1 wt %, about 0.08 wt % to about 0.1 wt %, about 0.08 wt % to about 0.15 wt %, about 0.08 wt % to about 0.2 wt %, about 0.08 wt % to about 0.25 wt %, about 0.08 wt % to about 0.3 wt %, about 0.08 wt % to about 0.4 wt %, about 0.08 wt % to about 0.5 wt %, about 0.08 wt % to about 1 wt %, about 0.1 wt % to about 0.15 wt %, about 0.1 wt % to about 0.2 wt %, about 0.1 wt % to about 0.25 wt %, about 0.1 wt % to about 0.3 wt %, about 0.1 wt % to about 0.4 wt %, about 0.1 wt % to about 0.5 wt %, about 0.1 wt % to about 1 wt %, about 0.15 wt % to about 0.2 wt %, about 0.15 wt % to about 0.25 wt %, about 0.15 wt % to about 0.3 wt %, about 0.15 wt % to about 0.4 wt %, about 0.15 wt % to about 0.5 wt %, about 0.15 wt % to about 1 wt %, about 0.2 wt % to about 0.25 wt %, about 0.2 wt % to about 0.3 wt %, about 0.2 wt % to about 0.4 wt %, about 0.2 wt % to about 0.5 wt %, about 0.2 wt % to about 1 wt %, about 0.25 wt % to about 0.3 wt %, about 0.25 wt % to about 0.4 wt %, about 0.25 wt % to about 0.5 wt %, about 0.25 wt % to about 1 wt %, about 0.3 wt % to about 0.4 wt %, about 0.3 wt % to about 0.5 wt %, about 0.3 wt % to about 1 wt %, about 0.4 wt % to about 0.5 wt %, about 0.4 wt % to about 1 wt %, or about 0.5 wt % to about 1 wt %. In some embodiments, the anionic polymer material is present in an amount of about 0.01 wt %, about 0.02 wt %, about 0.05 wt %, about 0.08 wt %, about 0.1 wt %, about 0.15 wt %, about 0.2 wt %, about 0.25 wt %, about 0.3 wt %, about 0.4 wt %, about 0.5 wt %, or about 1 wt %. In some embodiments, the anionic polymer material is present in an amount of at least about 0.01 wt %, about 0.02 wt %, about 0.05 wt %, about 0.08 wt %, about 0.1 wt %, about 0.15 wt %, about 0.2 wt %, about 0.25 wt %, about 0.3 wt %, about 0.4 wt %, or about 0.5 wt %. In some embodiments, the anionic polymer material is present in an amount of at most about 0.02 wt %, about 0.05 wt %, about 0.08 wt %, about 0.1 wt %, about 0.15 wt %, about 0.2 wt %, about 0.25 wt %, about 0.3 wt %, about 0.4 wt %, about 0.5 wt %, or about 1 wt %.

In some embodiments, the lipid vesicle composition comprises a first and a second anionic polymer material. In some embodiments, the first and the second anionic polymer are a different type. In some embodiments, each of the first and the second anionic polymer material is an anionic polysaccharide of a different type. In some embodiments, the first and the second anionic polymer material are the same type. In some embodiments, each of the first and the second anionic polymer material is an anionic polysaccharide. In some embodiments, each of the first and the second anionic polymer is hyaluronic acid.

In cases where the first and second anionic polymer materials are the same type, each anionic polymer material has a different molecular weight. In some embodiments, the first anionic polymer material has a molecular weight of up to about 75 kDa and the second anionic polymer material has a molecule weight of greater than about 75 kDa. In some embodiments, the first anionic polymer material has a molecular weight of up to about 75 kDa and the second anionic polymer material has a molecular weight of greater than about 75 kDa. In some embodiments, the first anionic polymer comprises sodium hyaluronate with a molecular weight of 50 kDa and the second anionic polymer comprises sodium hyaluronate with a molecular weight of 250 kDa.

In cases where the lipid vesicle comprises a first and second anionic polymer material, each component may be included in a different amount. In some embodiments, the first and second anionic polymer material are present in about the same amount. In some embodiments, the ratios provided herein comprise weight ratios. In some embodiments, the ratio of the first and the second anionic material is about 10:1, 9:1. 8:1, 7:1, 6:1, 5:1, 4:1, 3:1, 3:2, 2:1, 1:1, 1:2, 1:3, 1:4, 1:5, 1:6, 1:7, 1:8, 1:9 or 1:10. In some embodiments, the weight ratio of the first and the second anionic material is about 10:1, 9:1. 8:1, 7:1, 6:1, 5:1, 4:1, 3:1, 3:2, 2:1, 1:1, 1:2, 1:3, 1:4, 1:5, 1:6, 1:7, 1:8, 1:9 or 1:10. In some embodiments, the first and the second anionic polymer comprising sodium hyaluronate with a molecular weight of 50 kDa and 250 kDa, respectively, is present at a ratio of about 1:2. In some cases, sodium hyaluronate with a molecular weight of 250 kDa is present at 0.1 wt % and sodium hyaluronate with a molecular weight of 50 kDa is present at 0.05 wt %. In some embodiments, the first and the second anionic polymer comprising sodium hyaluronate with a molecular weight of 50 kDa and 250 kDa, respectively, is present at a ratio of about 1:1. In some cases, sodium hyaluronate with a molecular weight of 250 kDa is present at 0.1 wt % and sodium hyaluronate with a molecular weight of 50 kDa is present at 0.1 wt %.

In some embodiments, the combination of the first anionic polymer and the second anionic polymer is present in the amount of about 0.01 wt % to about 1 wt %. In some embodiments, the combination is present in an amount of about 0.01 wt % to about 0.02 wt %, about 0.01 wt % to about 0.05 wt %, about 0.01 wt % to about 0.08 wt %, about 0.01 wt % to about 0.1 wt %, about 0.01 wt % to about 0.15 wt %, about 0.01 wt % to about 0.2 wt %, about 0.01 wt % to about 0.25 wt %, about 0.01 wt % to about 0.3 wt %, about 0.01 wt % to about 0.4 wt %, about 0.01 wt % to about 0.5 wt %, about 0.01 wt % to about 1 wt %, about 0.02 wt % to about 0.05 wt %, about 0.02 wt % to about 0.08 wt %, about 0.02 wt % to about 0.1 wt %, about 0.02 wt % to about 0.15 wt %, about 0.02 wt % to about 0.2 wt %, about 0.02 wt % to about 0.25 wt %, about 0.02 wt % to about 0.3 wt %, about 0.02 wt % to about 0.4 wt %, about 0.02 wt % to about 0.5 wt %, about 0.02 wt % to about 1 wt %, about 0.05 wt % to about 0.08 wt %, about 0.05 wt % to about 0.1 wt %, about 0.05 wt % to about 0.15 wt %, about 0.05 wt % to about 0.2 wt %, about 0.05 wt % to about 0.25 wt %, about 0.05 wt % to about 0.3 wt %, about 0.05 wt % to about 0.4 wt %, about 0.05 wt % to about 0.5 wt %, about 0.05 wt % to about 1 wt %, about 0.08 wt % to about 0.1 wt %, about 0.08 wt % to about 0.15 wt %, about 0.08 wt % to about 0.2 wt %, about 0.08 wt % to about 0.25 wt %, about 0.08 wt % to about 0.3 wt %, about 0.08 wt % to about 0.4 wt %, about 0.08 wt % to about 0.5 wt %, about 0.08 wt % to about 1 wt %, about 0.1 wt % to about 0.15 wt %, about 0.1 wt % to about 0.2 wt %, about 0.1 wt % to about 0.25 wt %, about 0.1 wt % to about 0.3 wt %, about 0.1 wt % to about 0.4 wt %, about 0.1 wt % to about 0.5 wt %, about 0.1 wt % to about 1 wt %, about 0.15 wt % to about 0.2 wt %, about 0.15 wt % to about 0.25 wt %, about 0.15 wt % to about 0.3 wt %, about 0.15 wt % to about 0.4 wt %, about 0.15 wt % to about 0.5 wt %, about 0.15 wt % to about 1 wt %, about 0.2 wt % to about 0.25 wt %, about 0.2 wt % to about 0.3 wt %, about 0.2 wt % to about 0.4 wt %, about 0.2 wt % to about 0.5 wt %, about 0.2 wt % to about 1 wt %, about 0.25 wt % to about 0.3 wt %, about 0.25 wt % to about 0.4 wt %, about 0.25 wt % to about 0.5 wt %, about 0.25 wt % to about 1 wt %, about 0.3 wt % to about 0.4 wt %, about 0.3 wt % to about 0.5 wt %, about 0.3 wt % to about 1 wt %, about 0.4 wt % to about 0.5 wt %, about 0.4 wt % to about 1 wt %, or about 0.5 wt % to about 1 wt %. In some embodiments, the combination is present in an amount of about 0.01 wt %, about 0.02 wt %, about 0.05 wt %, about 0.08 wt %, about 0.1 wt %, about 0.15 wt %, about 0.2 wt %, about 0.25 wt %, about 0.3 wt %, about 0.4 wt %, about 0.5 wt %, or about 1 wt %. In some embodiments, the combination is present in an amount of at least about 0.01 wt %, about 0.02 wt %, about 0.05 wt %, about 0.08 wt %, about 0.1 wt %, about 0.15 wt %, about 0.2 wt %, about 0.25 wt %, about 0.3 wt %, about 0.4 wt %, or about 0.5 wt %. In some embodiments, the combination is present in an amount of at most about 0.02 wt %, about 0.05 wt %, about 0.08 wt %, about 0.1 wt %, about 0.15 wt %, about 0.2 wt %, about 0.25 wt %, about 0.3 wt %, about 0.4 wt %, about 0.5 wt %, or about 1 wt %. For example, the first anionic polymer may be present in an amount of about 0.1 wt % and the second anionic polymer may be present in an amount of about 0.05 wt %. As a further example, the second anionic polymer may be present in an amount of about 0.1 wt % and the first anionic polymer may be present in an amount of about 0.05 wt %.

In some embodiments, the composition comprises a first, second, and third anionic polymer material. In some embodiments, the anionic polymer materials can be of the same type (e.g., three different molecular weights of hyaluronic acid). In some embodiments, two of the three anionic polymer materials are of the same type. In some embodiments, each of the anionic polymer materials are of a different type. In some embodiments, the first anionic polymer material, the second anionic polymer material, the third anionic polymer material or any combination thereof are not crosslinked. In some embodiments, the first anionic polymer material, the second anionic polymer material, or both are not crosslinked. In some embodiments, the third anionic polymer material is crosslinked. In some embodiments, the third anionic polymer material comprises a sodium hyaluronate crosspolymer. The sodium hyaluronate crosspolymer may be in a composition comprising one or more solvents, such as water and/or pentylene glycol. In some embodiments, the crosslinked polymer material is in a gel form. In some embodiments, the first anionic polymer material has a molecular weight of from about 5 kDa to about 20 kDa, the second anionic polymer has a molecular weight of from about 20 kDa to about 75 kDa, and the third anionic polymer material has a molecular weight of greater than about 75 kDa.

In some embodiments, each of the three anionic polymer materials is present in about the same amount. In some embodiments, each of the three anionic polymer materials is present in about a different amount. In some embodiments, the first anionic polymer material, the second anionic polymer material, the third anionic polymer material, or any combination thereof is each present in an amount of about 0.1 mg/g to about 10 mg/g. In some embodiments, the first anionic polymer material, the second anionic polymer material, the third anionic polymer material, or any combination thereof is each present in an amount of about 0.01% to about 1%.

In some embodiments, an anionic polymer material is present in an amount of from about 0.01 mg/mL to about 10 mg/mL. In some embodiments, the anionic polymer material is present in an amount of about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 1.25 mg/mL, about 0.01 mg/mL to about 1.5 mg/mL, about 0.01 mg/mL to about 1.75 mg/mL, about 0.01 mg/mL to about 2 mg/mL, about 0.01 mg/mL to about 5 mg/mL, about 0.01 mg/mL to about 10 mg/mL, about 0.05 mg/mL to about 0.1 mg/mL, about 0.05 mg/mL to about 0.5 mg/mL, about 0.05 mg/mL to about 1 mg/mL, about 0.05 mg/mL to about 1.25 mg/mL, about 0.05 mg/mL to about 1.5 mg/mL, about 0.05 mg/mL to about 1.75 mg/mL, about 0.05 mg/mL to about 2 mg/mL, about 0.05 mg/mL to about 5 mg/mL, about 0.05 mg/mL to about 10 mg/mL, about 0.1 mg/mL to about 0.5 mg/mL, about 0.1 mg/mL to about 1 mg/mL, about 0.1 mg/mL to about 1.25 mg/mL, about 0.1 mg/mL to about 1.5 mg/mL, about 0.1 mg/mL to about 1.75 mg/mL, about 0.1 mg/mL to about 2 mg/mL, about 0.1 mg/mL to about 5 mg/mL, about 0.1 mg/mL to about 10 mg/mL, about 0.5 mg/mL to about 1 mg/mL, about 0.5 mg/mL to about 1.25 mg/mL, about 0.5 mg/mL to about 1.5 mg/mL, about 0.5 mg/mL to about 1.75 mg/mL, about 0.5 mg/mL to about 2 mg/mL, about 0.5 mg/mL to about 5 mg/mL, about 0.5 mg/mL to about 10 mg/mL, about 1 mg/mL to about 1.25 mg/mL, about 1 mg/mL to about 1.5 mg/mL, about 1 mg/mL to about 1.75 mg/mL, about 1 mg/mL to about 2 mg/mL, about 1 mg/mL to about 5 mg/mL, about 1 mg/mL to about 10 mg/mL, about 1.25 mg/mL to about 1.5 mg/mL, about 1.25 mg/mL to about 1.75 mg/mL, about 1.25 mg/mL to about 2 mg/mL, about 1.25 mg/mL to about 5 mg/mL, about 1.25 mg/mL to about 10 mg/mL, about 1.5 mg/mL to about 1.75 mg/mL, about 1.5 mg/mL to about 2 mg/mL, about 1.5 mg/mL to about 5 mg/mL, about 1.5 mg/mL to about 10 mg/mL, about 1.75 mg/mL to about 2 mg/mL, about 1.75 mg/mL to about 5 mg/mL, about 1.75 mg/mL to about 10 mg/mL, about 2 mg/mL to about 5 mg/mL, about 2 mg/mL to about 10 mg/mL, or about 5 mg/mL to about 10 mg/mL. In some embodiments, the anionic polymer material is present in an amount of about 0.01 mg/mL, about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, about 5 mg/mL, or about 10 mg/mL. In some embodiments, the anionic polymer material is present in an amount of at least about 0.01 mg/mL, about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, or about 5 mg/mL. In some embodiments, the anionic polymer material is present in an amount of at most about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, about 5 mg/mL, or about 10 mg/mL.

In some embodiments, an anionic polymer material is present in an amount of from about 0.01 mg/g to about 10 mg/g. In some embodiments, an anionic polymer material is present in an amount of from about 0.01 mg/g to about 0.05 mg/g, about 0.01 mg/g to about 0.1 mg/g, about 0.01 mg/g to about 0.5 mg/g, about 0.01 mg/g to about 1 mg/g, about 0.01 mg/g to about 2.5 mg/g, about 0.01 mg/g to about 5 mg/g, about 0.01 mg/g to about 7.5 mg/g, about 0.01 mg/g to about 10 mg/g, about 0.05 mg/g to about 0.1 mg/g, about 0.05 mg/g to about 0.5 mg/g, about 0.05 mg/g to about 1 mg/g, about 0.05 mg/g to about 2.5 mg/g, about 0.05 mg/g to about 5 mg/g, about 0.05 mg/g to about 7.5 mg/g, about 0.05 mg/g to about 10 mg/g, about 0.1 mg/g to about 0.5 mg/g, about 0.1 mg/g to about 1 mg/g, about 0.1 mg/g to about 2.5 mg/g, about 0.1 mg/g to about 5 mg/g, about 0.1 mg/g to about 7.5 mg/g, about 0.1 mg/g to about 10 mg/g, about 0.5 mg/g to about 1 mg/g, about 0.5 mg/g to about 2.5 mg/g, about 0.5 mg/g to about 5 mg/g, about 0.5 mg/g to about 7.5 mg/g, about 0.5 mg/g to about 10 mg/g, about 1 mg/g to about 2.5 mg/g, about 1 mg/g to about 5 mg/g, about 1 mg/g to about 7.5 mg/g, about 1 mg/g to about 10 mg/g, about 2.5 mg/g to about 5 mg/g, about 2.5 mg/g to about 7.5 mg/g, about 2.5 mg/g to about 10 mg/g, about 5 mg/g to about 7.5 mg/g, about 5 mg/g to about 10 mg/g, or about 7.5 mg/g to about 10 mg/g. In some embodiments, an anionic polymer material is present in an amount of from about 0.01 mg/g, about 0.05 mg/g, about 0.1 mg/g, about 0.5 mg/g, about 1 mg/g, about 2.5 mg/g, about 5 mg/g, about 7.5 mg/g, or about 10 mg/g. In some embodiments, an anionic polymer material is present in an amount of from at least about 0.01 mg/g, about 0.05 mg/g, about 0.1 mg/g, about 0.5 mg/g, about 1 mg/g, about 2.5 mg/g, about 5 mg/g, or about 7.5 mg/g. In some embodiments, an anionic polymer material is present in an amount of from at most about 0.05 mg/g, about 0.1 mg/g, about 0.5 mg/g, about 1 mg/g, about 2.5 mg/g, about 5 mg/g, about 7.5 mg/g, or about 10 mg/g. As an example, per 100 g of product, the anionic polymer material is present in an amount of from about 1 mg to about 1000 mg.

In some embodiments, a combination of the first and the second anionic polymer material is present in an amount of from about 0.01 mg/g to about 10 mg/g. In some embodiments, a combination of the first and the second anionic polymer material is present in an amount of from about 0.01 mg/mL to about 10 mg/mL. In some embodiments, a combination of the first and the second anionic polymer material is present in an amount of from about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 2.5 mg/mL, about 0.01 mg/mL to about 5 mg/mL, about 0.01 mg/mL to about 7.5 mg/mL, about 0.01 mg/mL to about 10 mg/mL, about 0.05 mg/mL to about 0.1 mg/mL, about 0.05 mg/mL to about 0.5 mg/mL, about 0.05 mg/mL to about 1 mg/mL, about 0.05 mg/mL to about 2.5 mg/mL, about 0.05 mg/mL to about 5 mg/mL, about 0.05 mg/mL to about 7.5 mg/mL, about 0.05 mg/mL to about 10 mg/mL, about 0.1 mg/mL to about 0.5 mg/mL, about 0.1 mg/mL to about 1 mg/mL, about 0.1 mg/mL to about 2.5 mg/mL, about 0.1 mg/mL to about 5 mg/mL, about 0.1 mg/mL to about 7.5 mg/mL, about 0.1 mg/mL to about 10 mg/mL, about 0.5 mg/mL to about 1 mg/mL, about 0.5 mg/mL to about 2.5 mg/mL, about 0.5 mg/mL to about 5 mg/mL, about 0.5 mg/mL to about 7.5 mg/mL, about 0.5 mg/mL to about 10 mg/mL, about 1 mg/mL to about 2.5 mg/mL, about 1 mg/mL to about 5 mg/mL, about 1 mg/mL to about 7.5 mg/mL, about 1 mg/mL to about 10 mg/mL, about 2.5 mg/mL to about 5 mg/mL, about 2.5 mg/mL to about 7.5 mg/mL, about 2.5 mg/mL to about 10 mg/mL, about 5 mg/mL to about 7.5 mg/mL, about 5 mg/mL to about 10 mg/mL, or about 7.5 mg/mL to about 10 mg/mL. In some embodiments, a combination of the first and the second anionic polymer material is present in an amount of from about 0.01 mg/mL, about 0.05 mg/mL, about 0.1 mg/mL, about 0.5 mg/mL, about 1 mg/mL, about 2.5 mg/mL, about 5 mg/mL, about 7.5 mg/mL, or about 10 mg/mL. In some embodiments, a combination of the first and the second anionic polymer material is present in an amount of from at least about 0.01 mg/mL, about 0.05 mg/mL, about 0.1 mg/mL, about 0.5 mg/mL, about 1 mg/mL, about 2.5 mg/mL, about 5 mg/mL, or about 7.5 mg/mL. In some embodiments, a combination of the first and the second anionic polymer material is present in an amount of from at most about 0.05 mg/mL, about 0.1 mg/mL, about 0.5 mg/mL, about 1 mg/mL, about 2.5 mg/mL, about 5 mg/mL, about 7.5 mg/mL, or about 10 mg/mL. As an example, per 100 g of product, the combination of the first and the second anionic polymer material is present in an amount of from about 1 mg to about 1000 mg.

In some embodiments, the first anionic polymer material is present in an amount of from about 0.01 mg/g to about 10 mg/g. In some embodiments, the first anionic polymer material is present in an amount of from about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 2.5 mg/mL, about 0.01 mg/mL to about 5 mg/mL, about 0.01 mg/mL to about 7.5 mg/mL, about 0.01 mg/mL to about 10 mg/mL, about 0.05 mg/mL to about 0.1 mg/mL, about 0.05 mg/mL to about 0.5 mg/mL, about 0.05 mg/mL to about 1 mg/mL, about 0.05 mg/mL to about 2.5 mg/mL, about 0.05 mg/mL to about 5 mg/mL, about 0.05 mg/mL to about 7.5 mg/mL, about 0.05 mg/mL to about 10 mg/mL, about 0.1 mg/mL to about 0.5 mg/mL, about 0.1 mg/mL to about 1 mg/mL, about 0.1 mg/mL to about 2.5 mg/mL, about 0.1 mg/mL to about 5 mg/mL, about 0.1 mg/mL to about 7.5 mg/mL, about 0.1 mg/mL to about 10 mg/mL, about 0.5 mg/mL to about 1 mg/mL, about 0.5 mg/mL to about 2.5 mg/mL, about 0.5 mg/mL to about 5 mg/mL, about 0.5 mg/mL to about 7.5 mg/mL, about 0.5 mg/mL to about 10 mg/mL, about 1 mg/mL to about 2.5 mg/mL, about 1 mg/mL to about 5 mg/mL, about 1 mg/mL to about 7.5 mg/mL, about 1 mg/mL to about 10 mg/mL, about 2.5 mg/mL to about 5 mg/mL, about 2.5 mg/mL to about 7.5 mg/mL, about 2.5 mg/mL to about 10 mg/mL, about 5 mg/mL to about 7.5 mg/mL, about 5 mg/mL to about 10 mg/mL, or about 7.5 mg/mL to about 10 mg/mL. In some embodiments, the first anionic polymer material is present in an amount of from about 0.01 mg/mL, about 0.05 mg/mL, about 0.1 mg/mL, about 0.5 mg/mL, about 1 mg/mL, about 2.5 mg/mL, about 5 mg/mL, about 7.5 mg/mL, or about 10 mg/mL. In some embodiments, the first anionic polymer material is present in an amount of from at least about 0.01 mg/mL, about 0.05 mg/mL, about 0.1 mg/mL, about 0.5 mg/mL, about 1 mg/mL, about 2.5 mg/mL, about 5 mg/mL, or about 7.5 mg/mL. In some embodiments, the first anionic polymer material is present in an amount of from at most about 0.05 mg/mL, about 0.1 mg/mL, about 0.5 mg/mL, about 1 mg/mL, about 2.5 mg/mL, about 5 mg/mL, about 7.5 mg/mL, or about 10 mg/mL. In some embodiments, the second anionic polymer material is present in an amount of from about 0.01 mg/g to about 10 mg/g. In some embodiments, the second anionic polymer material is present in an amount of from about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 2.5 mg/mL, about 0.01 mg/mL to about 5 mg/mL, about 0.01 mg/mL to about 7.5 mg/mL, about 0.01 mg/mL to about 10 mg/mL, about 0.05 mg/mL to about 0.1 mg/mL, about 0.05 mg/mL to about 0.5 mg/mL, about 0.05 mg/mL to about 1 mg/mL, about 0.05 mg/mL to about 2.5 mg/mL, about 0.05 mg/mL to about 5 mg/mL, about 0.05 mg/mL to about 7.5 mg/mL, about 0.05 mg/mL to about 10 mg/mL, about 0.1 mg/mL to about 0.5 mg/mL, about 0.1 mg/mL to about 1 mg/mL, about 0.1 mg/mL to about 2.5 mg/mL, about 0.1 mg/mL to about 5 mg/mL, about 0.1 mg/mL to about 7.5 mg/mL, about 0.1 mg/mL to about 10 mg/mL, about 0.5 mg/mL to about 1 mg/mL, about 0.5 mg/mL to about 2.5 mg/mL, about 0.5 mg/mL to about 5 mg/mL, about 0.5 mg/mL to about 7.5 mg/mL, about 0.5 mg/mL to about 10 mg/mL, about 1 mg/mL to about 2.5 mg/mL, about 1 mg/mL to about 5 mg/mL, about 1 mg/mL to about 7.5 mg/mL, about 1 mg/mL to about 10 mg/mL, about 2.5 mg/mL to about 5 mg/mL, about 2.5 mg/mL to about 7.5 mg/mL, about 2.5 mg/mL to about 10 mg/mL, about 5 mg/mL to about 7.5 mg/mL, about 5 mg/mL to about 10 mg/mL, or about 7.5 mg/mL to about 10 mg/mL. In some embodiments, the second anionic polymer material is present in an amount of from about 0.01 mg/mL, about 0.05 mg/mL, about 0.1 mg/mL, about 0.5 mg/mL, about 1 mg/mL, about 2.5 mg/mL, about 5 mg/mL, about 7.5 mg/mL, or about 10 mg/mL. In some embodiments, the second anionic polymer material is present in an amount of from at least about 0.01 mg/mL, about 0.05 mg/mL, about 0.1 mg/mL, about 0.5 mg/mL, about 1 mg/mL, about 2.5 mg/mL, about 5 mg/mL, or about 7.5 mg/mL. In some embodiments, the second anionic polymer material is present in an amount of from at most about 0.05 mg/mL, about 0.1 mg/mL, about 0.5 mg/mL, about 1 mg/mL, about 2.5 mg/mL, about 5 mg/mL, about 7.5 mg/mL, or about 10 mg/mL. In some examples, the first anionic polymer is present in an amount of 0.25 mg/g and the second anionic polymer is present in an amount of about 0.5 mg/g. In some examples, the first anionic polymer is present in an amount of 0.5 mg/g and the second anionic polymer is present in an amount of about 1 mg/g. In some examples, the first anionic polymer is present in an amount of 0.5 mg/g and the second anionic polymer is present in an amount of about 0.5 mg/g. In some examples, the first anionic polymer is present in an amount of 1 mg/g and the second anionic polymer is present in an amount of about 1 mg/g. In some examples, the first anionic polymer is present in an amount of 1 mg/g and the second anionic polymer is present in an amount of about 2 mg/g. In some examples, per 100 g of product, the first anionic polymer material is present in an amount of from about 1 mg to about 500 mg. In some examples, per 100 g of product, the second anionic polymer material is present in an amount of from about 5 mg to about 1000 mg. In some embodiments, the composition further comprises the third anionic polymer that is present in an amount of from about 0.01 mg/g to about 10 mg/g. In some embodiments, the third anionic polymer material is present in an amount of from about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 2.5 mg/mL, about 0.01 mg/mL to about 5 mg/mL, about 0.01 mg/mL to about 7.5 mg/mL, about 0.01 mg/mL to about 10 mg/mL, about 0.05 mg/mL to about 0.1 mg/mL, about 0.05 mg/mL to about 0.5 mg/mL, about 0.05 mg/mL to about 1 mg/mL, about 0.05 mg/mL to about 2.5 mg/mL, about 0.05 mg/mL to about 5 mg/mL, about 0.05 mg/mL to about 7.5 mg/mL, about 0.05 mg/mL to about 10 mg/mL, about 0.1 mg/mL to about 0.5 mg/mL, about 0.1 mg/mL to about 1 mg/mL, about 0.1 mg/mL to about 2.5 mg/mL, about 0.1 mg/mL to about 5 mg/mL, about 0.1 mg/mL to about 7.5 mg/mL, about 0.1 mg/mL to about 10 mg/mL, about 0.5 mg/mL to about 1 mg/mL, about 0.5 mg/mL to about 2.5 mg/mL, about 0.5 mg/mL to about 5 mg/mL, about 0.5 mg/mL to about 7.5 mg/mL, about 0.5 mg/mL to about 10 mg/mL, about 1 mg/mL to about 2.5 mg/mL, about 1 mg/mL to about 5 mg/mL, about 1 mg/mL to about 7.5 mg/mL, about 1 mg/mL to about 10 mg/mL, about 2.5 mg/mL to about 5 mg/mL, about 2.5 mg/mL to about 7.5 mg/mL, about 2.5 mg/mL to about 10 mg/mL, about 5 mg/mL to about 7.5 mg/mL, about 5 mg/mL to about 10 mg/mL, or about 7.5 mg/mL to about 10 mg/mL. In some embodiments, the third anionic polymer material is present in an amount of from about 0.01 mg/mL, about 0.05 mg/mL, about 0.1 mg/mL, about 0.5 mg/mL, about 1 mg/mL, about 2.5 mg/mL, about 5 mg/mL, about 7.5 mg/mL, or about 10 mg/mL. In some embodiments, the third anionic polymer material is present in an amount of from at least about 0.01 mg/mL, about 0.05 mg/mL, about 0.1 mg/mL, about 0.5 mg/mL, about 1 mg/mL, about 2.5 mg/mL, about 5 mg/mL, or about 7.5 mg/mL. In some embodiments, the third anionic polymer material is present in an amount of from at most about 0.05 mg/mL, about 0.1 mg/mL, about 0.5 mg/mL, about 1 mg/mL, about 2.5 mg/mL, about 5 mg/mL, about 7.5 mg/mL, or about 10 mg/mL. In some examples, the third anionic polymer is present in an amount of about 0.1 mg/g. In some examples, the third anionic polymer is present in an amount of about 0.2 mg/g. In some examples, the third anionic polymer is present in an amount of about 0.5 mg/g. In some examples, the third anionic polymer is present in an amount of about 1 mg/g. In some examples, the third anionic polymer is present in an amount of about 2 mg/g. In some examples, the third anionic polymer is present in an amount of about 5 mg/g. In some examples, per 100 g of product, the third anionic polymer material is present in an amount of from about 1 mg to 500 mg.

### Vesicle Forming Lipids

In some embodiments, the vesicle composition comprises one or more vesicle forming lipids. The vesicle forming lipids act to encapsulate portions of the oil-in-water emulsions. In some embodiments, this allows the oil-in-water emulsion to remain stable for a period of time.

The vesicle forming lipids may be any suitable lipids for such a purpose. In some embodiments, the vesicle forming lipids comprise phospholipids, glycolipids, lecithins, ceramides, lysolecithin, lysophosphatidylethanolamine, phosphatidylserine, phosphatidylinositol, sphingomyelin, cardiolipin, phosphatidic acid, cerebroside, or any combination thereof. In some embodiments, the vesicle forming lipids comprise a combination of lipids.

In some embodiments, the vesicle forming lipids comprise phospholipids. In some embodiments, the phospholipids are naturally occurring, semisynthetic, or synthetically prepared, or a mixture thereof. In an embodiment, the phospholipids are one or more esters of glycerol with one or two (equal or different) residues of fatty adds and with phosphoric acid, wherein the phosphoric acid residue is in turn bound to a hydrophilic group, such as, for instance, choline (phosphatidylcholines—PC), serine (phosphatidylserines—PS), glycerol (phosphatidylglycerols—PG), ethanolamine (phosphatidylethanolamines—PE), or inositol (phosphatidylinositol). Esters of phospholipids with only one residue of fatty acid are generally referred to in the art as the “lyso” forms of the phospholipid or “lysophospholipids”. Fatty acids residues present in the phospholipids are in general long chain aliphatic acids, typically containing 12 to 24 carbon atoms, or 14 to 22 carbon atoms; the aliphatic chain may contain one or more unsaturations or is completely saturated. Examples of suitable fatty acids included in the phospholipids are, for instance, lauric acid, myristic acid, palmitic acid, stearic acid, arachidic acid, behenic acid, oleic acid, linoleic acid, and linolenic acid. Saturated fatty acids such as myristic acid, palmitic acid, stearic acid and arachidic acid may be employed.

In some embodiments, the phospholipid comprises one or more natural phospholipids. In some embodiments, the phospholipid comprises one or more semisynthetic phospholipids. In some embodiments, the semisynthetic phospholipids are the partially or fully hydrogenated derivatives of the naturally occurring lecithins. In some embodiments, the phospholipids include fatty acids di-esters of phosphatidylcholine, ethylphosphatidylcholine, phosphatidylglycerol, phosphatidic acid, phosphatidylethanolamine, phosphatidylserine or of sphingomyelin. In some embodiments, the phospholipids include hydrogenated phosphatidylcholine (e.g., Sunlipon 90H). In some embodiments, the phospholipids are, for instance, dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroylphosphatidylglycerol (DLPG) and its alkali metal salts, diarachidoylphosphatidylglycerol (DAPG) and its alkali metal salts, dimyristoylphosphatidylglycerol (DMPG) and its alkali metal salts, dipalmitoylphosphatidylglycerol (DPPG) and its alkali metal salts, distearoylphosphatidylglycerol (DSPG) and its alkali metal salts, dioleoyl-phosphatidylglycerol (DOPG) and its alkali metal salts, dimyristoyl phosphatidic acid DMPA) and its alkali metal salts, dipalmitoyl phosphatidic acid (DPPA) and its alkali metal salts, distearoyl phosphatidic acid (DSPA), diarachidoylphosphatidic acid (DAPA) and its alkali metal salts, dimyristoylphosphatidylethanolamine (DMPE), dipalmitoylphosphatidylethanolamine (DPPE), distearoyl phosphatidyl-ethanolamine (DSPE), dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), dioleoyl-phosphatidylinositol (DOPI).

In some embodiments, the vesicle forming lipids are present in an amount of about 0.5% to about 25% (w/w) of the composition. In some embodiments, the vesicle forming lipids are present in an amount of about 0.5% to about 2%, about 0.5% to about 5%, about 0.5% to about 8%, about 0.5% to about 10%, about 0.5% to about 12%, about 0.5% to about 15%, about 0.5% to about 20%, about 0.5% to about 25%, about 2% to about 5%, about 2% to about 8%, about 2% to about 10%, about 2% to about 12%, about 2% to about 15%, about 2% to about 20%, about 2% to about 25%, about 5% to about 8%, about 5% to about 10%, about 5% to about 12%, about 5% to about 15%, about 5% to about 20%, about 5% to about 25%, about 8% to about 10%, about 8% to about 12%, about 8% to about 15%, about 8% to about 20%, about 8% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25% (w/w) of the composition. In some embodiments, the vesicle forming lipids are present in an amount of about 0.5%, about 2%, about 5%, about 8%, about 10%, about 12%, about 15%, about 20%, or about 25%. In some embodiments, the vesicle forming lipids are present in an amount of at least about 0.5%, about 2%, about 5%, about 8%, about 10%, about 12%, about 15%, or about 20% (w/w) of the composition. In some embodiments, the vesicle forming lipids are present in an amount of at most about 2%, about 5%, about 8%, about 10%, about 12%, about 15%, about 20%, or about 25% (w/w) of the composition.

In some embodiments, the vesicle forming lipids are present in an amount of about 5% to about 15% (w/w) of the composition. In some embodiments, the vesicle forming lipids are present in an amount of about 5% to about 8%, about 5% to about 9%, about 5% to about 10%, about 5% to about 11%, about 5% to about 12%, about 5% to about 13%, about 5% to about 14%, about 5% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or about 14% to about 15%. In some embodiments, the vesicle forming lipids are present in an amount of about 5%, about 8%, about 9%, about 10%, about 11%, about 12%, about 13%, about 14%, or about 15% (w/w) of the composition. In some embodiments, the vesicle forming lipids are present in an amount of at least about 5%, about 8%, about 9%, about 10%, about 11%, about 12%, about 13%, or about 14% (w/w) of the composition. In some embodiments, the vesicle forming lipids are present in an amount of at most about 8%, about 9%, about 10%, about 11%, about 12%, about 13%, about 14%, or about 15% (w/w) of the composition.

In some embodiments, the composition comprises a short chain polyol. In some embodiments, the short chain polyol acts to enhance the stability of the resulting lipid vesicles. In some embodiments, the short chain polyol is a C₂-C₄ polyol comprising two or three alcohol groups. In some embodiments, the short chain polyol is propylene glycol. In some embodiments, the composition comprises propylene glycol. In some embodiments, the composition comprises butanediol (e.g., 2,3 butanediol). In some embodiments, the composition comprises propylene glycol, butanediol, or both.

In some embodiments, the propylene glycol is present in an amount of about 0.5% to about 25% (w/w) of the composition. In some embodiments, the propylene glycol is present in an amount of about 0.5% to about 2%, about 0.5% to about 5%, about 0.5% to about 8%, about 0.5% to about 10%, about 0.5% to about 12%, about 0.5% to about 15%, about 0.5% to about 20%, about 0.5% to about 25%, about 2% to about 5%, about 2% to about 8%, about 2% to about 10%, about 2% to about 12%, about 2% to about 15%, about 2% to about 20%, about 2% to about 25%, about 5% to about 8%, about 5% to about 10%, about 5% to about 12%, about 5% to about 15%, about 5% to about 20%, about 5% to about 25%, about 8% to about 10%, about 8% to about 12%, about 8% to about 15%, about 8% to about 20%, about 8% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25%. In some embodiments, the propylene glycol is present in an amount of about 0.5%, about 2%, about 5%, about 8%, about 10%, about 10.5%, about 12%, about 15%, about 20%, or about 25%. In some embodiments, the propylene glycol is present in an amount of at least about 0.5%, about 2%, about 5%, about 8%, about 10%, about 12%, about 15%, or about 20%. In some embodiments, the propylene glycol is present in an amount of at most about 2%, about 5%, about 8%, about 10%, about 12%, about 15%, about 20%, or about 25%. In some embodiments, the propylene glycol is present in an amount of about 1% to about 10%. In some embodiments, the propylene glycol is present in an amount of about 1% to about 2%, about 1% to about 4%, about 1% to about 6%, about 1% to about 8%, about 1% to about 10%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10%. In some embodiments, the propylene glycol is present in an amount of about 1%, about 2%, about 4%, about 6%, about 8%, or about 10%. In some embodiments, the propylene glycol is present in an amount of at least about 1%, about 2%, about 4%, about 6%, or about 8%. In some embodiments, the propylene glycol is present in an amount of at most about 2%, about 4%, about 6%, about 8%, or about 10%. In some embodiments, propylene glycol is present in about the same amount as the vesicle forming lipid. In some embodiments, the ratio of propylene glycol to vesicle forming lipid in the composition is form about 2:1 to about 1:2 (w/w).

In some embodiments, the butanediol (e.g., 2,3-butanediol) is present in an amount of about 0.5% to about 25% (w/w) of the composition. In some embodiments, the butanediol is present in an amount of about 0.5% to about 2%, about 0.5% to about 5%, about 0.5% to about 8%, about 0.5% to about 10%, about 0.5% to about 12%, about 0.5% to about 15%, about 0.5% to about 20%, about 0.5% to about 25%, about 2% to about 5%, about 2% to about 8%, about 2% to about 10%, about 2% to about 12%, about 2% to about 15%, about 2% to about 20%, about 2% to about 25%, about 5% to about 8%, about 5% to about 10%, about 5% to about 12%, about 5% to about 15%, about 5% to about 20%, about 5% to about 25%, about 8% to about 10%, about 8% to about 12%, about 8% to about 15%, about 8% to about 20%, about 8% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25%. In some embodiments, the butanediol is present in an amount of about 0.5%, about 2%, about 5%, about 8%, about 10%, about 10.5%, about 12%, about 15%, about 20%, or about 25%. In some embodiments, the butanediol is present in an amount of at least about 0.5%, about 2%, about 5%, about 8%, about 10%, about 12%, about 15%, or about 20%. In some embodiments, the butanediol is present in an amount of at most about 2%, about 5%, about 8%, about 10%, about 12%, about 15%, about 20%, or about 25%. In some embodiments, the butanediol is present in an amount of about 1% to about 10%. In some embodiments, the butanediol is present in an amount of about 1% to about 2%, about 1% to about 4%, about 1% to about 6%, about 1% to about 8%, about 1% to about 10%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10%. In some embodiments, the butanediol is present in an amount of about 1%, about 2%, about 4%, about 6%, about 8%, or about 10%. In some embodiments, the butanediol is present in an amount of at least about 1%, about 2%, about 4%, about 6%, or about 8%. In some embodiments, the butanediol is present in an amount of at most about 2%, about 4%, about 6%, about 8%, or about 10%. In some embodiments, butanediol is present in about the same amount as the vesicle forming lipid. In some embodiments, the ratio of butanediol to vesicle forming lipid in the composition is form about 2:1 to about 1:2 (w/w). In some embodiments, in a composition comprising butanediol and propylene glycol, the ratio of butanediol to propylene glycol is about 1:1, 2:1, 3:1, 4:1, 5:1, 6:1, 7:1, 8:1, 9:1, 10:1, 11:1, or 12:1.

### Oil Phases

The lipid vesicle compositions provided herein comprise an oil-in-water emulsion. The oil component is selected such that the material is a liquid at operative temperatures (e.g., room temperature) and is non-miscible with water.

Any suitable oil may be used as the oil phase. In some embodiments, the oil comprises a naturally occurring oil. In some embodiments, the naturally occurring oil is derived from one or more plants or plant parts (e.g., seeds or nuts). In some embodiments, the oil is a naturally occurring oil such as olive oil, vegetable oil, sunflower oil, or other similar plant derived oil.

In some embodiments, the oil phase is selected from the group consisting of vegetable oils, mono-, di-, and triglycerides, silicone fluids, mineral oils, and combinations thereof.

In some embodiments, the oil comprises a silicon oil or derivative, such as dimethicone. In some embodiments, the silicon oil comprises a siloxane polymer. In some embodiments, the siloxane polymer comprises C₁-C₃ substituents. In some embodiments, the siloxane is polydimethylsiloxane (PDMS). In some embodiments, the silicon oil is polymethylsilsesquioxane (e.g., Botanisil™ SP-360). In some embodiments, the oil is a mixture which comprises a silicon oil (e.g., dimethicone) as a smaller component. In some embodiments, the oil is a mixture which comprises a natural emollient as a substitute for dimethicone (e.g., LexFeel™ N350 MB). In some embodiments, the silicon oil is incorporated in order to enhance the feel of the resulting composition or as a moisturizer. In some embodiments, the oil comprises a silicon oil in an amount of up to about 5%, up to about 4%, up to about 3%, up to about 2%, or up to about 1% (w/w) of the composition. In some embodiments, the silicon oil is present in an amount of from about 0.1% to about 2%. In some embodiments, the silicon oil is present in an amount of from about 0.1% to about 0.5%, 0.1% to about 0.7%, 0.1% to about 1%, 0.1% to about 1.5%, 0.15% to about 2%, 0.5% to about 0.7%, 0.5% to about 1%, 0.5% to about 1.5%, 0.5% to about 2%, 0.7% to about 1%, 0.7% to about 1.5%, 0.7% to about 2%, 1% to about 1.5%, or 1% to about 2% (w/w) of the composition. In some embodiments, the silicon oil is present in an amount of about 0.1%, 0.5%, 0.6%, 0.7%, 1%, 1.5%, or 2% of the composition.

In some embodiments, the oils are present in an amount of about 1% to about 35% (w/w) of the composition. In some embodiments, the oils are present in an amount of about 1% to about 5%, about 1% to about 10%, about 1% to about 15%, about 1% to about 20%, about 1% to about 25%, about 1% to about 30%, about 1% to about 35%, about 5% to about 10%, about 5% to about 15%, about 5% to about 20%, about 5% to about 25%, about 5% to about 30%, about 5% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30%, about 25% to about 35%, or about 30% to about 35%. In some embodiments, the oils are present in an amount of about 1%, about 5%, about 10%, about 15%, about 20%, about 25%, about 30%, or about 35%. In some embodiments, the oils are present in an amount of at least about 1%, about 5%, about 10%, about 15%, about 20%, about 25%, or about 30%. In some embodiments, the oils are present in an amount of at most about 5%, about 10%, about 15%, about 20%, about 25%, about 30%, or about 35%. In some embodiments, the oils are present in an amount of about 5% to about 15%. In some embodiments, the oils are present in an amount of about 5% to about 8%, about 5% to about 9%, about 5% to about 10%, about 5% to about 11%, about 5% to about 12%, about 5% to about 13%, about 5% to about 14%, about 5% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or about 14% to about 15%. In some embodiments, the oils are present in an amount of about 5%, about 8%, about 9%, about 10%, about 11%, about 12%, about 13%, about 14%, or about 15%. In some embodiments, the oils are present in an amount of at least about 5%, about 8%, about 9%, about 10%, about 11%, about 12%, about 13%, or about 14%. In some embodiments, the oils are present in an amount of at most about 8%, about 9%, about 10%, about 11%, about 12%, about 13%, about 14%, or about 15%.

In some embodiments, the oil comprises one or more triglycerides. In some embodiments the triglyceride is a medium chain triglyceride. In some embodiments, the medium chain triglyceride comprises fatty acid esters having a chain length of C₆-C₁₂.

In some embodiments, the triglyceride is present in an amount of about 1% to about 35% (w/w) of the composition. In some embodiments, the triglyceride is present in an amount of about 1% to about 5%, about 1% to about 10%, about 1% to about 15%, about 1% to about 20%, about 1% to about 25%, about 1% to about 30%, about 1% to about 35%, about 5% to about 10%, about 5% to about 15%, about 5% to about 20%, about 5% to about 25%, about 5% to about 30%, about 5% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30%, about 25% to about 35%, or about 30% to about 35%. In some embodiments, the triglyceride is present in an amount of about 1%, about 1.5%, about 2%, about 2.5%, about 3%, about 3.5%, about 4%, about 4.5%, about 5%, about 10%, about 15%, about 20%, about 25%, about 30%, or about 35%. In some embodiments, the triglyceride is present in an amount of at least about 1%, about 1.5%, about 2%, about 2.5%, about 3%, about 3.5%, about 4%, about 4.5%, about 5%, about 10%, about 15%, about 20%, about 25%, or about 30%. In some embodiments, the triglyceride is present in an amount of at most about 1.5%, about 2%, about 2.5%, about 3%, about 3.5%, about 4%, about 4.5%, about 5%, about 10%, about 15%, about 20%, about 25%, about 30%, or about 35%.

In some embodiments, the oil phase of the lipid vesicle and/or the lipid vesicle portion of the composition comprises a sterol. In some embodiments, the sterol is a naturally derived sterol. In some embodiments, the sterol is a synthetic sterol. In some embodiments, the sterol is cholesterol. In some embodiments, the cholesterol may be plant-derived cholesterol. In some embodiments, the cholesterol may be synthetic cholesterol. In some embodiments the plant-derived cholesterol may be cholest-5-en-3-ol (e.g., PhytoChol®, SyntheChol®, Avanti #700100), or any other plant-derived cholesterol, or any combination thereof. In some embodiments, the sterol may be phytosterol or a derivative thereof. In some embodiments, the phytosterol or derivative thereof may be phytosterol MM, Advasterol™ 90 IP or 95 IP F, NET Sterol-ISO, Canola Sterols, sitosterol 700095, lanosterol-95, brassicasterol, or any combination thereof.

In some embodiments, the sterol is present in an amount of about 0.1% to about 5% (w/w) of the composition. In some embodiments, the sterol is present in an amount of about 0.1% to about 5%. In some embodiments, the sterol is present in an amount of about 0.1% to about 0.2%, about 0.1% to about 0.5%, about 0.1% to about 0.8%, about 0.1% to about 1%, about 0.1% to about 2%, about 0.1% to about 3%, about 0.1% to about 4%, about 0.1% to about 5%, about 0.2% to about 0.5%, about 0.2% to about 0.8%, about 0.2% to about 1%, about 0.2% to about 2%, about 0.2% to about 3%, about 0.2% to about 4%, about 0.2% to about 5%, about 0.5% to about 0.8%, about 0.5% to about 1%, about 0.5% to about 2%, about 0.5% to about 3%, about 0.5% to about 4%, about 0.5% to about 5%, about 0.8% to about 1%, about 0.8% to about 2%, about 0.8% to about 3%, about 0.8% to about 4%, about 0.8% to about 5%, about 1% to about 2%, about 1% to about 3%, about 1% to about 4%, about 1% to about 5%, about 2% to about 3%, about 2% to about 4%, about 2% to about 5%, about 3% to about 4%, about 3% to about 5%, or about 4% to about 5%. In some embodiments, the sterol is present in an amount of about 0.1%, about 0.2%, about 0.5%, about 0.8%, about 1%, about 2%, about 3%, about 4%, or about 5%. In some embodiments, the sterol is present in an amount of at least about 0.1%, about 0.2%, about 0.5%, about 0.8%, about 1%, about 2%, about 3%, or about 4%. In some embodiments, the sterol is present in an amount of at most about 0.2%, about 0.5%, about 0.8%, about 1%, about 2%, about 3%, about 4%, or about 5%. In some embodiments, the sterol is present in an amount of about 1% to about 1.5%, about 1% to about 2%, about 1% to about 2.5%, about 1% to about 3%, about 1% to about 4%, about 1% to about 5%, about 1.5% to about 2%, about 1.5% to about 2.5%, about 1.5% to about 3%, about 1.5% to about 4%, about 1.5% to about 5%, about 2% to about 2.5%, about 2% to about 3%, about 2% to about 4%, about 2% to about 5%, about 2.5% to about 3%, about 2.5% to about 4%, about 2.5% to about 5%, about 3% to about 4%, about 3% to about 5%, or about 4% to about 5% (w/w) of the composition. In some embodiments, the sterol is present in an amount of about 1%, about 1.5%, about 2%, about 2.5%, about 3%, about 4%, or about 5% (w/w) of the composition. In some embodiments, the sterol is present in an amount of at least about 1%, about 1.5%, about 2%, about 2.5%, about 3%, or about 4% (w/w) of the composition. In some embodiments, the sterol is present in an amount of at most about 1.5%, about 2%, about 2.5%, about 3%, about 4%, or about 5% (w/w) of the composition.

In some embodiments, the lipid vesicle compositions comprise one or more penetration enhancers. In some embodiments, a penetration enhancer acts to increase the amount of penetration of the anionic polymer material through one or more layers of skin when applied to the skin of an individual. In some embodiments, a penetration enhancer acts to increase the amount of penetration of one or more peptides through one or more layers of skin when applied to the skin of an individual.

In some embodiments, a composition comprising one or more penetration enhancers has an increase in penetration through one or more layers of the skin when applied to a skin sample or the skin of an individual by at least 5%, 10%, 15%, 20%, 25%, 30%, 40%, 50%, 60%, 70%, 80%, 90%, 100%, 120%, 150%, or 200% compared to an otherwise same or similar composition without the one or more penetration enhancers when applied one or more layers of the skin of a similar or same skin sample or skin of an individual. In some embodiments, a composition comprising one or more penetration enhancers has an increase in penetration through one or more layers of the skin when applied to a skin sample or the skin of an individual by greater than 5%, 10%, 15%, 20%, 25%, 30%, 40%, 50%, 60%, 70%, 80%, 90%, 100%, 120%, 150%, or 200% compared to an otherwise same or similar composition without the one or more penetration enhancers when applied one or more layers of the skin of a similar or same skin sample or skin of an individual. In some embodiments, a composition comprising one or more penetration enhancers has an increase in penetration through one or more layers of the skin when applied to a skin sample or the skin of an individual by about 5%, 10%, 15%, 20%, 25%, 30%, 40%, 50%, 60%, 70%, 80%, 90%, 100%, 120%, 150%, or 200% compared to an otherwise same or similar composition without the one or more penetration enhancers when applied one or more layers of the skin of a similar or same skin sample or skin of an individual.

In some embodiments, a composition comprising one or more non-ionic surfactants with an HLB value of about 10 or less has an increase in penetration through one or more layers of the skin when applied to a skin sample or the skin of an individual by at least 5%, 10%, 15%, 20%, 25%, 30%, 40%, 50%, 60%, 70%, 80%, 90%, 100%, 120%, 150%, or 200% compared to an otherwise same or similar composition without the one or more non-ionic surfactants with an HLB value of about 10 or less when applied one or more layers of the skin of a similar or same skin sample or skin of an individual. In some embodiments, a composition comprising one or more non-ionic surfactants with an HLB value of about 10 or less has an increase in penetration through one or more layers of the skin when applied to a skin sample or the skin of an individual by greater than 5%, 10%, 15%, 20%, 25%, 30%, 40%, 50%, 60%, 70%, 80%, 90%, 100%, 120%, 150%, or 200% compared to an otherwise same or similar composition without the one or more non-ionic surfactants with an HLB value of about 10 or less when applied one or more layers of the skin of a similar or same skin sample or skin of an individual. In some embodiments, a composition comprising one or more non-ionic surfactants with an HLB value of about 10 or less has an increase in penetration through one or more layers of the skin when applied to a skin sample or the skin of an individual by about 5%, 10%, 15%, 20%, 25%, 30%, 40%, 50%, 60%, 70%, 80%, 90%, 100%, 120%, 150%, or 200% compared to an otherwise same or similar composition without the one or more non-ionic surfactants with an HLB value of about 10 or less when applied one or more layers of the skin of a similar or same skin sample or skin of an individual.

In some embodiments, the penetration enhancer is included in the oil-in-water emulsion of the composition. In some embodiments, the penetration enhancer is included in the lipid bilayer of the composition.

There are many types of penetration enhancing agents that may be employed. In some embodiments, the penetration enhancing agent comprising an ionic surfactant, a nonionic surfactant, or a combination thereof. In some embodiments, an ionic surfactant, a non-ionic surfactant, or a combination thereof is not a penetration enhancing agent.

In some embodiments, the penetration enhancing agent comprises a non-ionic surfactant or a combination of non-ionic surfactants. In some embodiments, the penetration enhancing agent is a single non-ionic surfactant. In some embodiments, the penetration enhancing agent is a combination of at least 2, 3, 4, or more non-ionic surfactants. In some embodiments, the penetration enhancing agent is a combination 2 non-ionic surfactants. In some embodiments, the penetration enhancing agent is a combination 3 non-ionic surfactants.

In some embodiments, the non-ionic surfactant or combination of non-ionic surfactants is selected from polyethylene glycol ethers of fatty alcohols, sorbitan esters, polysorbates, and polyethylene glycol fatty acid esters and combinations thereof.

In some embodiments, the non-ionic surfactant comprises a polyethylene glycol (PEG) ethers of a fatty alcohol. In some embodiments, the PEG ether of the fatty alcohol comprises from about 2 to about 8 PEG groups and a C₁₂-C₂₂ fatty alcohol. In some embodiments, the polyethylene glycol ether of a fatty alcohol comprises diethylene glycol hexadecyl ether, 2-(2-octadecoxyethoxy)ethanol, diethylene glycol monooleyl ether, polyoxyethylene (2) oleyl ether, polyoxyethylene (3) oleyl ether, or polyoxyethylene (5) oleyl ether, or any combination thereof. In some embodiments, the polyethylene glycol ether of a fatty alcohol comprises 2-(2-octadecoxyethoxy)ethanol. In some embodiments, the PEG ether of a fatty alcohol is super refined Brij® 02 or a derivative thereof.

In some embodiments, the PEG ether of the fatty alcohol is present in an amount of from about 0.5% to about 10%, about 0.5% to about 5%, about 0.5% to about 4%, or about 0.05% to about 3% (w/w) of the composition. In some embodiments, the PEG ether of the fatty alcohol is present in an amount of about 0.5% to about 2.5%. In some embodiments, the PEG ether of the fatty alcohol is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the PEG ether of the fatty alcohol is present in an amount of about 0.5%, about 0.8%, about 1%, about 1.2%, about 1.5%, about 2%, or about 2.5%. In some embodiments, the PEG ether of the fatty alcohol is present in an amount of at least about 0.5%, about 0.8%, about 1%, about 1.2%, about 1.5%, or about 2%. In some embodiments, the PEG ether of the fatty alcohol is present in an amount of at most about 0.8%, about 1%, about 1.2%, about 1.5%, about 2%, or about 2.5%.

In some embodiments, the non-ionic surfactant comprises a sorbitan ester. In some embodiments, the sorbitan ester is a fatty acid ester. In some embodiments, the sorbitan ester is a C₁₂-C₂₂ fatty acid ester. In some embodiments, the sorbitan ester comprises sorbitan monolaurate, sorbitan monopalmitate, sorbitan monostearate, sorbitan monooleate, sorbitan trioleate, sorbitan sesquioleate, or sorbitan isostearate, or any combinations thereof. In some embodiments, the sorbitan ester comprises sorbitan monolaurate. In some embodiments, the sorbitan ester comprises sorbitan monopalmitate. In some embodiments, the sorbitan ester comprises sorbitan monostearate. In some embodiments, the sorbitan ester comprises sorbitan monooleate. In some embodiments, the sorbitan ester comprises sorbitan trioleate. In some embodiments, the sorbitan ester comprises sorbitan sesquioleate. In some embodiments, the sorbitan ester comprises sorbitan isostearate.

In some embodiments, the sorbitan ester is present in an amount of up to about 5% (w/w) of the composition. In some embodiments, the sorbitan ester is present in an amount of from about 0.5% to about 5%, about 0.5% to about 4%, or about 0.5% to about 3%. In some embodiments, the sorbitan ester is present in an amount of about 0.5% to about 2.5%. In some embodiments, the sorbitan ester is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the sorbitan ester is present in an amount of about 0.5%, about 0.8%, about 1%, about 1.2%, about 1.5%, about 2%, or about 2.5%. In some embodiments, the sorbitan ester is present in an amount of at least about 0.5%, about 0.8%, about 1%, about 1.2%, about 1.5%, or about 2%. In some embodiments, the sorbitan ester is present in an amount of at most about 0.8%, about 1%, about 1.2%, about 1.5%, about 2%, or about 2.5%.

In some embodiments, the non-ionic surfactant comprises a polysorbate. In some embodiments, the polysorbate comprises polysorbate 20, polysorbate 21, polysorbate 40, polysorbate 60, polysorbate 80, polysorbate 85, or any combination thereof. In some embodiments, the polysorbate is polysorbate 80. In some embodiments, the polysorbate is polysorbate 20. In some embodiments, the polysorbate is added to the aqueous phase of the composition.

In some embodiments, the polysorbate is present in an amount of up to about 5%. In some embodiments, the polysorbate is present in an amount of from about 0.5% to about 5%, about 0.5% to about 4%, or about 0.5% to about 3% (w/w) of the composition. In some embodiments, the polysorbate is present in an amount of about 0.5% to about 2.5%. In some embodiments, the polysorbate is present in an amount of about 0.2% to about 0.5%, about 0.2% to about 0.8%, about 0.2% to about 1%, about 0.2% to about 1.2%, about 0.2% to about 1.5%, about 0.2% to about 2%, about 0.2% to about 2.5%, about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the polysorbate is present in an amount of about 0.2%, 0.5%, about 0.8%, about 1%, about 1.2%, about 1.5%, about 2%, or about 2.5%. In some embodiments, the polysorbate is present in an amount of at least about 0.2%, 0.5%, about 0.8%, about 1%, about 1.2%, about 1.5%, or about 2%. In some embodiments, the polysorbate is present in an amount of at most about 0.5%, about 0.8%, about 1%, about 1.2%, about 1.5%, about 2%, or about 2.5%.

In some embodiments, the non-ionic surfactant comprises a polyethylene glycol (PEG) fatty acid ester. In some embodiments, the PEG fatty acid ester is a PEG chain of about 2-8 subunits comprising C₈-C₂₂ fatty acids affixed to each terminal hydroxyl to form the fatty acid ester. In some embodiments, the PEG fatty acid ester comprises PEG-8 dilaurate, PEG-4 dilaurate, PEG-4 laurate, PEG-8 dioleate, PEG-8 distearate, PEG-8 distearate, PEG-7 glyceryl cocoate, and PEG-20 almond glycerides, or any combination thereof. In some embodiments, the PEG fatty acid ester is PEG-4 dilaurate.

In some embodiments, the PEG fatty acid ester is present in an amount of up to about 5% (w/w) of the composition. In some embodiments, the PEG fatty acid ester is present in an amount of from about 0.5% to about 5%, about 0.5% to about 4%, or about 0.5% to about 3%. In some embodiments, the PEG fatty ester is present in an amount of about 0.5% to about 2.5%. In some embodiments, the PEG fatty ester is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the PEG fatty ester is present in an amount of about 0.5%, about 0.8%, about 1%, about 1.2%, about 1.5%, about 2%, or about 2.5%. In some embodiments, the PEG fatty ester is present in an amount of at least about 0.5%, about 0.8%, about 1%, about 1.2%, about 1.5%, or about 2%. In some embodiments, the PEG fatty ester is present in an amount of at most about 0.8%, about 1%, about 1.2%, about 1.5%, about 2%, or about 2.5%.

In some embodiments, the non-ionic surfactant has a hydrophobic-lipophilic balance (HLB) of about 10 or less. In some embodiments, the non-ionic surfactant comprises glyceryl monostearate (e.g., Cithrol GMS 40). In some embodiments, the non-ionic surfactant comprises an oleyl alcohol (e.g., Lipocol O-95). In some embodiments, the non-ionic surfactant comprises a polyoxyethylene oleyl ether (e.g., Oleth-2). In some embodiments, the non-ionic surfactant comprises a propylene glycol monocaprylate (e.g., Capryol® 90). In some embodiments, the composition comprises a plurality of non-ionic surfactants, each having an HLB of about 10 or less. In some embodiments, the non-ionic surfactant with an HLB of 10 or less is selected from the Table 1 below, or any combination thereof.

TABLE 1

| Surfactants (INCI/Chemical name) | Properties |
|---|---|
| Ceteth-2 (Diethylene glycol hexadecyl ether) | HLB = 5.3 |
| Steareth-2 (2-(2-octadecoxyethoxy)ethanol) | HLB = 4.9 |
| Oleth-2 (Polyoxyethylene (2) Oleyl Ether) | HLB = 4.9 |
| Oleth-3 (Polyoxyethylene (3) Oleyl Ether) | HLB = 6.6 |
| Oleth-5 (Polyoxyethylene (5) Oleyl Ether) | HLB = 9 |
| Polysorbate 61 | HLB = 9.6 |
| Sorbitan monolaurate | HLB = 8.6 |
| Sorbitan monopalmitate | HLB = 6.7 |
| Sorbitan monostearate | HLB = 4.7 |
| Sorbitan monooleate | HLB = 4.3 |
| Sorbitan trioleate | HLB = 1.8 |
| Sorbitan sesquioleate | HLB = 3.7 |
| Sorbitan Isostearate | HLB = 4.7 |
| PEG-8 dilaurate (Polyoxyethylene (8) dilaurate) | HLB = 10 |
| PEG-4 dilaurate (Polyoxyethylene (4) dilaurate) | HLB = 6 |
| PEG-4 laurate | HLB = 9 |
| PEG-8 dioleate | HLB = 7.2 |
| PEG-8 distearate | HLB = 8 |
| PEG-7 glyceryl cocoate | HLB = 10 |
| PEG-20 almond glycerides | HLB = 10 |
| Propylene glycol isostearate | HLB = 2.5 |
| Glycol stearate | HLB = 2.9 |
| Glyceryl stearate | HLB = 3.8 |
| Glyceryl stearate SE | HLB = 5.8 |
| Glyceryl laurate | HLB = 5.2 |
| Glyceryl caprylate | HLB = 5-6 |
| PEG-30 dipolyhydroxy-stearate | HLB = 5.5 |
| Glycol distearate | HLB = 1 |
| Phospholipid/lecithin | HLB = 4-10 |
| Propylene glycol monocaprylate | HLB = 5 |
| Propylene glycol monolaurate | HLB = 3 |

In some embodiments, the non-ionic surfactant has a hydrophobic-lipophilic balance (HLB) of about 10 or more. In some embodiments, the composition comprises a plurality of non-ionic surfactants, each having an HLB of about 10 or more.

In some embodiments, the non-ionic surfactant or combination of non-ionic surfactants are present in an amount of about 0.5% to about 10% (w/w) of the composition. In some embodiments, the non-ionic surfactant or combination of non-ionic surfactants are present in an amount of about 0.5% to about 1%, about 0.5% to about 1.5%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 10%, about 1% to about 1.5%, about 1% to about 2%, about 1% to about 3%, about 1% to about 4%, about 1% to about 5%, about 1% to about 6%, about 1% to about 7%, about 1% to about 8%, about 1% to about 10%, about 1.5% to about 2%, about 1.5% to about 3%, about 1.5% to about 4%, about 1.5% to about 5%, about 1.5% to about 6%, about 1.5% to about 7%, about 1.5% to about 8%, about 1.5% to about 10%, about 2% to about 3%, about 2% to about 4%, about 2% to about 5%, about 2% to about 6%, about 2% to about 7%, about 2% to about 8%, about 2% to about 10%, about 3% to about 4%, about 3% to about 5%, about 3% to about 6%, about 3% to about 7%, about 3% to about 8%, about 3% to about 10%, about 4% to about 5%, about 4% to about 6%, about 4% to about 7%, about 4% to about 8%, about 4% to about 10%, about 5% to about 6%, about 5% to about 7%, about 5% to about 8%, about 5% to about 10%, about 6% to about 7%, about 6% to about 8%, about 6% to about 10%, about 7% to about 8%, about 7% to about 10%, or about 8% to about 10%. In some embodiments, the non-ionic surfactant or combination of non-ionic surfactants are present in an amount of about 0.5%, about 0.6%, about 0.7%, about 1%, about 1.5%, about 2%, about 3%, about 4%, about 5%, about 6%, about 7%, about 8%, or about 10%. In some embodiments, the non-ionic surfactant or combination of non-ionic surfactants are present in an amount of about 0.6% (e.g., 0.54% to about 0.66%). In some embodiments, the non-ionic surfactant or combination of non-ionic surfactants are present in an amount of about 0.7% (e.g, 0.63% to about 0.77%). In some embodiments, the non-ionic surfactant or combination of non-ionic surfactants are present in an amount of at least about 0.5%, about 0.7%, about 1%, about 1.5%, about 2%, about 3%, about 4%, about 5%, about 6%, about 7%, or about 8%. In some embodiments, the non-ionic surfactant or combination of non-ionic surfactants are present in an amount of at most about 0.7%, about 1%, about 1.5%, about 2%, about 3%, about 4%, about 5%, about 6%, about 7%, about 8%, or about 10%.

In some embodiments, the composition comprises a non-ionic surfactant in the oil-in-water emulsion, the lipid bilayer, or both. In some embodiments, the composition comprises a non-ionic surfactant in the oil-in-water emulsion. In some embodiments, the composition comprises a non-ionic surfactant in the lipid bilayer. In some embodiments, the composition comprises a non-ionic surfactant in the oil-in-water emulsion and the lipid bilayer, wherein the composition comprises two or more different non-ionic surfactants.

In some embodiments, the penetration enhancing agent comprises a salicylate ester or a nicotinate ester. In some embodiments, the ester is a C₁-C₆ alkyl ester or a benzyl ester. In some embodiments, the penetration enhancing agent comprises methyl salicylate or benzyl nicotinate. In some embodiments, the penetration enhancing agent is a nicotinate ester present in an amount of up to about 0.1%, 0.5%, 1%, 2%, or 3% (w/w) of the composition. In some embodiments, the nicotinate ester is present in an amount of from about 0.1% to about 3%, about 0.1% to about 2%, or about 0.1% to about 1%. In some embodiments, benzyl nicotinate is present at an amount of about 0.5%.

### Cationic Surfactants

In some embodiments, the composition comprises an ionic surfactant. In some embodiments, the ionic surfactant is a cationic surfactant. In some embodiments, the cationic surfactant is a mono-cationic surfactant, a di-cationic surfactant, or a poly-cationic surfactant.

In some embodiments, the mono-cationic surfactant is used in the composition to form a submicron emulsion prior to formation of a final lipid vesicle composition provided herein (e.g., before the lipid forming vesicles are added). In some embodiments, the mono-cationic surfactant is net-mono-cationic (e.g., a phosphate salt comprising two side chains each with a single cationic functionality, which is partially neutralized by a phosphate anion).

In some embodiments, the mono-cationic surfactant is a fatty-amide derived propylene glycol-diammonium phosphate ester. Fatty-amide derived propylene glycol-diammonium phosphate esters are phospholipids which comprise at least one propylene glycol phosphoester linked to a quaternary ammonium group, which is in turn linked with a fatty acid amide. One non-limiting example of a fatty-amide derived propylene glycol-diammonium phosphate ester is linoleamidopropyl PG-dimonium chloride phosphate. Similar compounds with different fatty acid amide groups attached are also known. In some embodiments, the fatty-amide derived propylene glycol-diammoniom phosphate ester has the structure:

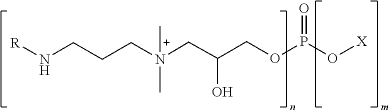

wherein n is an integer from 1 to 3, m is an integer from 0 to 2, wherein the sum of m and n is 3; X is a cation selected from a proton, sodium, potassium, magnesium, and calcium; and R is an acyl group of a C₈-C₃₀ fatty acid.

In some embodiments, the fatty acid is a C₁₂-C₂₄ fatty acid. In some embodiments, the fatty acid is an unsaturated fatty acid. In some embodiments, the fatty acid is linoleic acid. In some embodiments, the mono-cationic penetration enhancing agent is linoleamidopropyl PG-dimonium chloride phosphate (e.g., Arlasilk™ PTM, Arlasilk™ EFA).

In some embodiments, the fatty amide derived propylene glycol-diammonium phosphate ester is present in an amount of about 1% to about 20% (w/w) of the composition. In some embodiments, the fatty amide derived propylene glycol-diammonium phosphate ester is present in an amount of about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 12%, about 1% to about 15%, about 1% to about 20%, about 2% to about 3%, about 2% to about 4%, about 2% to about 5%, about 2% to about 6%, about 2% to about 7%, about 2% to about 8%, about 2% to about 9%, about 2% to about 10%, about 2% to about 12%, about 2% to about 15%, about 2% to about 20%, about 3% to about 4%, about 3% to about 5%, about 3% to about 6%, about 3% to about 7%, about 3% to about 8%, about 3% to about 9%, about 3% to about 10%, about 3% to about 12%, about 3% to about 15%, about 3% to about 20%, about 4% to about 5%, about 4% to about 6%, about 4% to about 7%, about 4% to about 8%, about 4% to about 9%, about 4% to about 10%, about 4% to about 12%, about 4% to about 15%, about 4% to about 20%, about 5% to about 6%, about 5% to about 7%, about 5% to about 8%, about 5% to about 9%, about 5% to about 10%, about 5% to about 12%, about 5% to about 15%, about 5% to about 20%, about 6% to about 7%, about 6% to about 8%, about 6% to about 9%, about 6% to about 10%, about 6% to about 12%, about 6% to about 15%, about 6% to about 20%, about 7% to about 8%, about 7% to about 9%, about 7% to about 10%, about 7% to about 12%, about 7% to about 15%, about 7% to about 20%, about 8% to about 9%, about 8% to about 10%, about 8% to about 12%, about 8% to about 15%, about 8% to about 20%, about 9% to about 10%, about 9% to about 12%, about 9% to about 15%, about 9% to about 20%, about 10% to about 12%, about 10% to about 15%, about 10% to about 20%, about 12% to about 15%, about 12% to about 20%, or about 15% to about 20%. In some embodiments, the fatty amide derived propylene glycol-diammonium phosphate ester is present in an amount of about 1%, about 2%, about 3%, about 4%, about 5%, about 6%, about 6.5%, about 7%, about 7.5%, about 8%, about 9%, about 10%, about 12%, about 15%, or about 20%. In some embodiments, the fatty amide derived propylene glycol-diammonium phosphate ester is present in an amount of at least about 1%, about 2%, about 3%, about 4%, about 5%, about 6%, about 6.5%, about 7%, about 7.5%, about 8%, about 9%, about 10%, about 12%, about 15%, or about 20%. In some embodiments, the fatty amide derived propylene glycol-diammonium phosphate ester is present in an amount of at most about 2%, about 3%, about 4%, about 5%, about 6%, about 6.5%, about 7%, about 7.5%, about 8%, about 9%, about 10%, about 12%, about 15%, or about 20%.

In some embodiments, the cationic surfactant is a di-cationic penetration enhancing agent. In some embodiments, the di-cationic surfactant is a gemini surfactant. In some embodiments, a gemini surfactant is a surfactant comprising two quaternary amines represented by the formula A-N(R)₂—B—N(R)₂—C, wherein each of A and C is independently an optionally substituted C₆-C₂₄ alkyl group, each R is independently optionally substituted C₁-C₆ alkyl, and B is an optionally substituted C₂-C₁₀ alkylene chain. In some embodiments, the each of A and C is a C₆-C₂₄ saturated or unsaturated hydrocarbon. In some embodiments, the each of A and C is a C₆-C₂₄ saturated hydrocarbon. In some embodiments, each R is methyl. In some embodiments, B is a saturated C₂-C₁₀ alkylene chain. In some cases, gemini surfactants follow the nomenclature X—Y—Z, wherein each of X, Y, and Z is an integer representing the number of carbon atoms of each substituent, and Y is the spacer between the two quaternary amines. Thus, for example, a Dec. 3, 2012 gemini surfactant has the formula CH₃(CH₂)₁₁—[N⁺(CH₃)₂]—(CH₂)₃—[N⁺(CH₃)₂]—(CH₂)₁₁CH₃. In some embodiments, the gemini surfactant is a 10-2, 10, 12-2-12, 14-2-14, 10-3-10, 12-3-12, 14-3-14, 10-4-10, 12-412, or 14-4-14 gemini surfactant. In some embodiments, the gemini surfactant is a 12-3-12 gemini surfactant.

In some embodiments, the gemini surfactant is present in an amount of about 0.1% to about 1.5% (w/w) of the composition. In some embodiments, the gemini surfactant is present in an amount of about 0.1% to about 0.2%, about 0.1% to about 0.3%, about 0.1% to about 0.5%, about 0.1% to about 0.7%, about 0.1% to about 0.9%, about 0.1% to about 1%, about 0.1% to about 1.2%, about 0.1% to about 1.5%, about 0.2% to about 0.3%, about 0.2% to about 0.5%, about 0.2% to about 0.7%, about 0.2% to about 0.9%, about 0.2% to about 1%, about 0.2% to about 1.2%, about 0.2% to about 1.5%, about 0.3% to about 0.5%, about 0.3% to about 0.7%, about 0.3% to about 0.9%, about 0.3% to about 1%, about 0.3% to about 1.2%, about 0.3% to about 1.5%, about 0.5% to about 0.7%, about 0.5% to about 0.9%, about 0.5% to about 1%, about 0.5% to about 1.2%, about 0.5% to about 1.5%, about 0.7% to about 0.9%, about 0.7% to about 1%, about 0.7% to about 1.2%, about 0.7% to about 1.5%, about 0.9% to about 1%, about 0.9% to about 1.2%, about 0.9% to about 1.5%, about 1% to about 1.2%, about 1% to about 1.5%, or about 1.2% to about 1.5%. In some embodiments, the gemini surfactant is present in an amount of about 0.1%, about 0.2%, about 0.3%, about 0.5%, about 0.7%, about 0.9%, about 1%, about 1.2%, or about 1.5%. In some embodiments, the gemini surfactant is present in an amount of at least about 0.1%, about 0.2%, about 0.3%, about 0.5%, about 0.7%, about 0.9%, about 1%, or about 1.2%. In some embodiments, the gemini surfactant is present in an amount of at most about 0.2%, about 0.3%, about 0.5%, about 0.7%, about 0.9%, about 1%, about 1.2%, or about 1.5%.

In some embodiments, the cationic surfactant comprises a polycationic group. In some embodiments, the polycationic group is a polymer wherein each monomer of the polymer comprises a charged group (e.g., an amino group). In some embodiments, the polycationic group is polylysine. In some embodiments, the polycationic group is polyarginine.

In some embodiments, the polylysine has a molecular weight of from about 1 kDa to about 10 kDa, from about 1 kDa to about 5 kDa, or from about 3 kDa to about 5 kDa. In some embodiments, the polylysine is present in an amount of from about 0.01% to about 1%, from about 0.01% to about 0.5%, from about 0.01% to about 0.2%, from about 0.05% to about 1%, from about 0.05% to about 0.5%, or from about 0.05% to about 0.2% (w/w) of the composition.

### Additional Components

In some embodiments, the vesicle composition comprises additional components. In some embodiments, these additional components improve one or more properties of the vesicles without dramatically altering the delivery of the anionic polymer material.

In some embodiments, the vesicle composition further comprises one or more viscosity enhancing agents. In some embodiments, the viscosity enhancing agents thicken the composition for increased stability and/or feel to a user of the vesicle composition. In some embodiments, the viscosity enhancing agents also act as surfactants. In some embodiments, the viscosity enhancing agent comprises one or more of a fatty alcohol, a wax, a fatty ester of glycerol, or any combination thereof. In some embodiments, the fatty alcohol is a C₈-C₂₀ fatty alcohol. In some embodiments, the fatty alcohol is cetyl alcohol. In some embodiments, the wax is a naturally occurring or synthetic wax. In some embodiments, the wax is beeswax. In some embodiment, the wax is synthetic beeswax. In some embodiments, the synthetic beeswax is Syncrowax™ BB4. In some embodiments, the synthetic beeswax is non-animal derived beeswax. In some embodiments, the non-animal derived beeswax is Syncrowax™ SB1. In some embodiments, the fatty ester of glycerol is a monoester. In some embodiments, the monoester is an ester of a C₈-C₂₄ fatty acid. In some embodiments, the fatty ester of glycerol is glycerol monostearate.

In some embodiments, the viscosity enhancing agents are present in an amount of from about 0.1% to about 10% (w/w) of the composition. In some embodiments, the viscosity enhancing agents are present in an amount of from about 0.1% to about 5%. In some embodiments, the viscosity enhancing agents are present in an amount of from about 0.1% to about 0.3%, about 0.1% to about 0.5%, about 0.1% to about 1%, about 0.1% to about 1%, about 0.1% to about 2%, about 0.1% to about 3%, about 0.1% to about 4%, about 0.1% to about 5%, about 0.3% to about 0.5%, about 0.3% to about 1%, about 0.3% to about 1%, about 0.3% to about 2%, about 0.3% to about 3%, about 0.3% to about 4%, about 0.3% to about 5%, about 0.5% to about 1%, about 0.5% to about 1%, about 0.5% to about 2%, about 0.5% to about 3%, about 0.5% to about 4%, about 0.5% to about 5%, about 1% to about 1%, about 1% to about 2%, about 1% to about 3%, about 1% to about 4%, about 1% to about 5%, about 1% to about 2%, about 1% to about 3%, about 1% to about 4%, about 1% to about 5%, about 2% to about 3%, about 2% to about 4%, about 2% to about 5%, about 3% to about 4%, about 3% to about 5%, or about 4% to about 5%. In some embodiments, the viscosity enhancing agents are present in an amount of from about 0.1%, about 0.3%, about 0.5%, about 1%, about 1%, about 2%, about 3%, about 4%, or about 5%. In some embodiments, the viscosity enhancing agents are present in an amount of from at least about 0.1%, about 0.3%, about 0.5%, about 1%, about 1%, about 2%, about 3%, or about 4%. In some embodiments, the viscosity enhancing agents are present in an amount of from at most about 0.3%, about 0.5%, about 1%, about 1%, about 2%, about 3%, about 4%, or about 5%. In some embodiments, the viscosity enhancing agents comprise a fatty alcohol in an amount of up to about 2%, a wax in an amount of up to about 2%, and a fatty ester of glycerol in an amount of up to about 5%. In some embodiments, the fatty alcohol is present in an amount of from about 0.1 to about 1.5%. In some embodiments, the fatty alcohol is present in an amount of about 0.4% (e.g., 0.36% to about 0.44%). In some embodiments, the fatty alcohol is present in an amount of about 0.6%. In some embodiments, the wax is present in an amount of from about 0.1% to about 1%. In some embodiments, the wax is present in an amount of about 0.2% (e.g., about 0.18% to about 0.22%). In some embodiments, the wax is present in an amount of about 0.3% (e.g., about 0.27% to about 0.33%). In some embodiments, the fatty ester of glycerol is present in an amount of from about 0.5% to about 2%. In some embodiments, the fatty ester of glycerol is present in an amount of about 0.8% (e.g., 0.72% to about 0.88%). In some embodiments, the fatty ester of glycerol is present in an amount of about 0.9%. In some embodiments, the fatty ester of glycerol is present in an amount of about 1%. In some embodiments, the fatty ester of glycerol is present in an amount of about 1.2%.

In some embodiments, the vesicle composition further comprises one or more of a thickener, a preservative, a moisturizer, an emollient, a humectant, or any combination thereof. In some embodiments, the vesicle composition further comprises a thickener. In some embodiments, the vesicle composition further comprises a preservative. In some embodiments, the vesicle composition further comprises a moisturizer. In some embodiments, the vesicle composition further comprises an emollient. In some embodiments, the vesicle composition further comprises a humectant. In some embodiments, the vesicle composition further comprises a fragrance (e.g., Mentha piperita). In some embodiments, the fragrance (e.g., Mentha piperita) is present in an amount of about 0.01% to about 0.1%. In some embodiments, the fragrance (e.g., Mentha piperita) is present in an amount of about 0.05%.

In some embodiments, the vesicle composition further comprises an antimicrobial. In some embodiments, the antimicrobial is a paraben ester. In some embodiments, the antimicrobial is methylparaben or propylparaben, or a combination thereof. In some embodiments, the antimicrobial is present in an amount of up to about 1%, up to about 0.9%, up to about 0.8%, up to about 0.7%, up to about 0.6%, up to about 0.5%, up to about 0.4%, up to about 0.3%, up to about 0.2% (w/w) of the composition.

In some embodiments, the vesicle composition further comprises a thickener. In some embodiments, the thickener is an inert polymer material. In some embodiments, the thickener is a siloxane polymer. In some embodiments, the thickener polydimethyl siloxane (PDMS). In some embodiments, the PDMS is present in an amount of up to about 5%, up to about 4%, up to about 3%, up to about 2%, or up to about 1%. In some embodiments, the PDMS is present in an amount of from about 0.1% to about 2% (w/w) of the composition.

In some embodiments, the composition further comprises a humectant. In some embodiments, the composition comprises glycerol. In some embodiments, the glycerol is present in an amount of from about 0.5% to about 25%, about 0.5% to about 20%, about 0.5% to about 15%, or about 0.5% to about 10%. In some embodiments, the glycerol is present in an amount of about 1% to about 10%. In some embodiments, the glycerol is present in an amount of about 1% to about 2%, about 1% to about 4%, about 1% to about 6%, about 1% to about 8%, about 1% to about 10%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10%. In some embodiments, the glycerol is present in an amount of about 1%, about 2%, about 4%, about 6%, about 8%, or about 10%. In some embodiments, the glycerol is present in an amount of at least about 1%, about 2%, about 4%, about 6%, or about 8%. In some embodiments, the glycerol is present in an amount of at most about 2%, about 4%, about 6%, about 8%, or about 10% (w/w) of the composition.

In some embodiments, the vesicle composition comprises a preservative. In some embodiments, the preservative is a cosmetic preservative, such as Euxyl® PE 9010 or Spectrastat®. In some embodiments, the preservative comprises a phenoxyethanol/ethylhexylglycerin mixture. In some embodiments, the preservative comprises a blend of caprylhydroxamic acid, caprylyl glycol, and glycerin. In some embodiments, the preservative is present in an amount of up to about 2%, up to about 1.5%, or up to about 1% (w/w) of the composition. In some embodiments, the preservative is present in an amount of from about 0.1% to about 2%, from about 0.1% to about 1.5%, or from about 0.1% to about 1%. In some embodiments, the preservative is present in an amount of about 0.1%, 0.2%, 0.3%, 0.4%, 0.5%, 0.55%, 0.6%, 0.65%, 0.7%, 0.75%, 0.8%, 0.85%, 0.9%, 0.95%, 1%, 1.05%, 1.1%, 1.2%, 1.3%, 1.4%, 1.5%, 1.6%, 1.7%, 1.8%, 1.9%, or 2%.

In some embodiments, the composition further comprises material to promote a desirable change in the skin, such as smoothing wrinkles. In some embodiments, the composition comprises a carbohydrate or an acceptable salt thereof. In some embodiments, the carbohydrate is a monosaccharide, disaccharide, or polysaccharide. In some embodiments, the carbohydrate or acceptable salt thereof comprises N-Acetyl-α-D-glucosamine 6-phosphate disodium salt (NovHyal™). In some embodiments, carbohydrate comprises a saccharide isomerate (e.g., Hyanify™). In some embodiments, the carbohydrate or an acceptable salt thereof is present in the composition in an amount of from about 0.1% to about 0.5%. In some embodiments, the carbohydrate or an acceptable salt thereof is present in the composition in an amount of from about 0.1% to about 0.2%, about 0.1% to about 0.3%, about 0.1% to about 0.4%, about 0.1% to about 0.5%, about 0.2% to about 0.3%, about 0.2% to about 0.4%, about 0.2% to about 0.5%, about 0.3% to about 0.4%, about 0.3% to about 0.5%, or about 0.4% to about 0.5%. In some embodiments, the carbohydrate or an acceptable salt thereof is present in the composition in an amount of about 0.1%, 0.2%, 0.3%, 0.4%, or 0.5%. In some embodiments, the carbohydrate or an acceptable salt thereof is present in the composition in an amount of at least about 0.1%, 0.2%, 0.3%, 0.4%, or 0.5%. In some embodiments, the carbohydrate or an acceptable salt thereof is present in the composition in an amount of at most about 0.1%, 0.2%, 0.3%, 0.4%, or 0.5%.

In some embodiments, the composition comprises a polymer of an amino acid. In some embodiments, the polymer of an amino acid comprises a high molecular weight (MW) polymer of the amino acid, a low MW polymer of the amino acid, or both. In some embodiments, the polymer of an amino acid comprises polyglutamate. In some cases, the polyglutamate comprises low MW polyglutamate (e.g., Hyafactor™ PGA LM), high MW polyglutamate (e.g., Hyafactor™ PGA HM), or both.

In some embodiments, the ratio of high MW polymer of the amino acid and low MW polymer of the amino acid is about 10:1, 9:1. 8:1, 7:1, 6:1, 5:1, 4:1, 3:1, 3:2, 2:1, 1:1, 1:2, 1:3, 1:4, 1:5, 1:6, 1:7, 1:8, 1:9 or 1:10.

In some embodiments, the polymer of an amino acid is present in an amount from about 0.01% to about 0.2%. In some embodiments, the polymer of an amino acid is present in an amount from about 0.01% to about 0.05%, about 0.01% to about 0.1%, about 0.01% to about 0.15%, about 0.01% to about 0.2%, about 0.05% to about 0.1%, about 0.05% to about 0.15%, about 0.05% to about 0.2%, about 0.1% to about 0.15%, about 0.1% to about 0.2%, or about 0.15% to about 0.2%. In some embodiments, the polymer of an amino acid is present in an amount of about 0.01%, about 0.05%, about 0.1%, about 0.15%, or about 0.2%. In some embodiments, the polymer of an amino acid is present in an amount of at least about 0.01%, about 0.05%, about 0.1%, or about 0.15%. In some embodiments, the polymer of an amino acid is present in an amount of at most about 0.05%, about 0.1%, about 0.15%, or about 0.2%.

In some embodiments, the combination of the high MW polymer and the low MW polymer is present in an amount from about 0.01% to about 0.2%. In some embodiments, the combination is present in an amount from about 0.01% to about 0.05%, about 0.01% to about 0.1%, about 0.01% to about 0.15%, about 0.01% to about 0.2%, about 0.05% to about 0.1%, about 0.05% to about 0.15%, about 0.05% to about 0.2%, about 0.1% to about 0.15%, about 0.1% to about 0.2%, or about 0.15% to about 0.2%. In some embodiments, the combination is present in an amount of about 0.01%, about 0.05%, about 0.1%, about 0.15%, or about 0.2%. In some embodiments, the combination is present in an amount of at least about 0.01%, about 0.05%, about 0.1%, or about 0.15%. In some embodiments, the combination is present in an amount of at most about 0.05%, about 0.1%, about 0.15%, or about 0.2%.

In some embodiments, the high MW polymer of the amino acid is present in an amount of about 0.01% to about 0.2%. In some embodiments, the high MW polymer is present in an amount from about 0.01% to about 0.05%, about 0.01% to about 0.1%, about 0.01% to about 0.15%, about 0.01% to about 0.2%, about 0.05% to about 0.1%, about 0.05% to about 0.15%, about 0.05% to about 0.2%, about 0.1% to about 0.15%, about 0.1% to about 0.2%, or about 0.15% to about 0.2%. In some embodiments, the high MW polymer is present in an amount of about 0.01%, about 0.05%, about 0.1%, about 0.15%, or about 0.2%. In some embodiments, the high MW polymer is present in an amount of at least about 0.01%, about 0.05%, about 0.1%, or about 0.15%. In some embodiments, the high MW polymer is present in an amount of at most about 0.05%, about 0.1%, about 0.15%, or about 0.2%. In some embodiments, the low MW polymer of the amino acid is present in an amount of about 0.01% to about 0.2%. In some embodiments, the low MW polymer of the amino acid is present in an amount from about 0.01% to about 0.05%, about 0.01% to about 0.1%, about 0.01% to about 0.15%, about 0.01% to about 0.2%, about 0.05% to about 0.1%, about 0.05% to about 0.15%, about 0.05% to about 0.2%, about 0.1% to about 0.15%, about 0.1% to about 0.2%, or about 0.15% to about 0.2%. In some embodiments, the low MW polymer of the amino acid is present in an amount of about 0.01%, about 0.05%, about 0.1%, about 0.15%, or about 0.2%. In some embodiments, the low MW polymer of the amino acid is present in an amount of at least about 0.01%, about 0.05%, about 0.1%, or about 0.15%. In some embodiments, the low MW polymer of the amino acid is present in an amount of at most about 0.05%, about 0.1%, about 0.15%, or about 0.2%. In some examples, the high MW polymer is present in an amount of about 0.01% and the low MW polymer is present in an amount of about 0.01%. In some examples, the high MW polymer is present in an amount of about 0.02% and the low MW polymer is present in an amount of about 0.01%. In some examples, the high MW polymer is present in an amount of about 0.05% and the low MW polymer is present in an amount of about 0.025%. In some examples, the high MW polymer is present in an amount of about 0.05%. In some embodiments, the low MW polymer is present in an amount of about 0.05%. In some examples, the high MW polymer is present in an amount of about 0.05% and the low MW polymer is present in an amount of about 0.05%. In some examples, the high MW polymer is present in an amount of about 0.1% and the low MW polymer is present in an amount of about 0.1%. In some examples, the high MW polymer is present in an amount of about 0.2% and the low MW polymer is present in an amount of about 0.1%. In some examples, the high MW polymer is present in an amount of about 0.2% and the low MW polymer is present in an amount of about 0.2%.

In some embodiments, the composition further comprises collagen. In some cases, the collagen in human collagen or vegan human collagen (e.g., HumaColl21®). In some cases, the molecular weight is about 15 kDa, 18 kDa, 20 kDa, 25 kDa, or 30 kDa. In some embodiments, the collagen in present in an amount from about 0.01% to about 0.2%. In some embodiments, the collagen is present in an amount from about 0.01% to about 0.05%, about 0.01% to about 0.1%, about 0.01% to about 0.15%, about 0.01% to about 0.2%, about 0.05% to about 0.1%, about 0.05% to about 0.15%, about 0.05% to about 0.2%, about 0.1% to about 0.15%, about 0.1% to about 0.2%, or about 0.15% to about 0.2%. In some embodiments, the collagen is present in an amount of about 0.01%, about 0.02%, about 0.05%, about 0.1%, about 0.15%, or about 0.2%. In some embodiments, the collagen is present in an amount of at least about 0.01%, about 0.02%, about 0.05%, about 0.1%, or about 0.15%. In some embodiments, the collagen is present in an amount of at most about 0.2%, about 0.05%, about 0.1%, about 0.15%, or about 0.2%.

In some embodiments, the composition further comprises material to promote a desirable change in the skin, such as reducing darkness in one or more areas of the skin. In some embodiments, the material comprises one or more biological extracts. The one or more biological extracts, each or in total, may be present in an amount of from about 0.01% to about 0.2%. In some instances, one or more biological extracts may be present in an amount of from about 0.01% to about 0.02%, about 0.01% to about 0.04%, about 0.01% to about 0.06%, about 0.01% to about 0.08%, about 0.01% to about 0.1%, about 0.01% to about 0.12%, about 0.01% to about 0.14%, about 0.01% to about 0.16%, about 0.01% to about 0.18%, about 0.01% to about 0.2%, about 0.02% to about 0.04%, about 0.02% to about 0.06%, about 0.02% to about 0.08%, about 0.02% to about 0.1%, about 0.02% to about 0.12%, about 0.02% to about 0.14%, about 0.02% to about 0.16%, about 0.02% to about 0.18%, about 0.02% to about 0.2%, about 0.04% to about 0.06%, about 0.04% to about 0.08%, about 0.04% to about 0.1%, about 0.04% to about 0.12%, about 0.04% to about 0.14%, about 0.04% to about 0.16%, about 0.04% to about 0.18%, about 0.04% to about 0.2%, about 0.06% to about 0.08%, about 0.06% to about 0.1%, about 0.06% to about 0.12%, about 0.06% to about 0.14%, about 0.06% to about 0.16%, about 0.06% to about 0.18%, about 0.06% to about 0.2%, about 0.08% to about 0.1%, about 0.08% to about 0.12%, about 0.08% to about 0.14%, about 0.08% to about 0.16%, about 0.08% to about 0.18%, about 0.08% to about 0.2%, about 0.1% to about 0.12%, about 0.1% to about 0.14%, about 0.1% to about 0.16%, about 0.1% to about 0.18%, about 0.1% to about 0.2%, about 0.12% to about 0.14%, about 0.12% to about 0.16%, about 0.12% to about 0.18%, about 0.12% to about 0.2%, about 0.14% to about 0.16%, about 0.14% to about 0.18%, about 0.14% to about 0.2%, about 0.16% to about 0.18%, about 0.16% to about 0.2%, or about 0.18% to about 0.2%. In some instances, one or more biological extracts, each or in total, may be present in an amount of from about 0.01%, about 0.02%, about 0.04%, about 0.06%, about 0.08%, about 0.1%, about 0.12%, about 0.14%, about 0.16%, about 0.18%, or about 0.2%. In some instances, one or more biological extracts, each or in total, may be present in an amount of from at least about 0.01%, about 0.02%, about 0.04%, about 0.06%, about 0.08%, about 0.1%, about 0.12%, about 0.14%, about 0.16%, or about 0.18%. In some instances, one or more biological extracts, each or in total, may be present in an amount of from at most about 0.02%, about 0.04%, about 0.06%, about 0.08%, about 0.1%, about 0.12%, about 0.14%, about 0.16%, about 0.18%, or about 0.2%.

In some embodiments, the one or more biological extracts are natural extracts or synthetic extracts. In some embodiments, the one or more biological extracts comprises a carbohydrate. In some embodiments, the carbohydrate comprises fucoidan. Examples of biological extracts comprise, but are not limited to, an algae extract, a marine extract, a biotechnology extract, root extract, or a flower extract (e.g., Seanactiv, Shadownyl Clear, Eyedeline, Eye'fective, Meiview, Rootness Awake, etc.). In some examples, the flower extract comprises a Jasminum Sambac flower extract (e.g., jasmine flower extract). In some examples, the flower extract comprises a Crataegus Monogyna flower extract (e.g., Hawthorn flower extract). In some examples, the extract is obtained by fermentation of bacteria. As an example, Bacillus ferment may be obtained by fermentation of Bacillus subtilis. In some examples, the root extract is from a plant belonging to the family Convolvulaceae. As an example, the root extract may be obtained from the roots of Ipomoea batatas. Each of the algae extract, marine extract, biotechnology extract, root extract, or flower extract may be present in in a biological extract composition added to the formulation in an amount from about 0.1%, 0.2%, 0.3%, 0.4%, 0.5%, 0.6%, 0.7%, or 0.08%. A biological extract composition added to the formulation provided herein may further comprise water, propanediol, xanthum gum, sodium sulfate, glycerin, glyceryl caprylate, or another other suitable solvent, or any combination thereof.

In some embodiments, an additional component may comprise one or more vitamins or derivatives thereof. In some cases, the one or more vitamins or derivatives thereof may provide one or more desired effects (e.g., smoothing, toning, moisturizing, brightening, reducing acne, eczema, wrinkles, etc.) when applied to the skin or epidermis. In some instances, the additional component may comprise vitamin B, vitamin C (ascorbic acid), vitamin A (retinol), vitamin E, vitamin D, vitamin F, vitamin K or any derivative thereof. In some examples, the additional component may comprise vitamin B3 (niacin). In some instances, a vitamin B3 derivative may comprise niacinamide. In some examples, the additional component may comprise provitamin B5 (panthenol). In some embodiments, the one or more vitamins or derivatives thereof are present in the composition in an about from about 0.1% to about 4% (w/w). In some embodiments, the one or more vitamins or derivatives thereof is present in an amount of about 0.1% to about 0.2%, about 0.1% to about 0.3%, about 0.1% to about 0.5%, about 0.1% to about 0.8%, about 0.1% to about 1%, about 0.1% to about 1.2%, about 0.1% to about 1.5%, about 0.1% to about 2%, about 0.1% to about 2.5%, about 0.1% to about 3%, about 0.1% to about 3.5%, about 0.1% to about 4%, about 0.2% to about 0.3%, about 0.2% to about 0.5%, about 0.2% to about 0.8%, about 0.2% to about 1%, about 0.2% to about 1.2%, about 0.2% to about 1.5%, about 0.2% to about 2%, about 0.2% to about 2.5%, about 0.2% to about 3%, about 0.2% to about 3.5%, about 0.2% to about 4%, about 0.3% to about 0.5%, about 0.3% to about 0.8%, about 0.3% to about 1%, about 0.3% to about 1.2%, about 0.3% to about 1.5%, about 0.3% to about 2%, about 0.3% to about 2.5%, about 0.3% to about 3%, about 0.3% to about 3.5%, about 0.3% to about 4%, about 0.5% to about 0.8%, about 0.5% to about 1%, about 0.5% to about 1.2%, about 0.5% to about 1.5%, about 0.5% to about 2%, about 0.5% to about 2.5%, about 0.5% to about 3%, about 0.5% to about 3.5%, about 0.5% to about 4%, about 0.8% to about 1%, about 0.8% to about 1.2%, about 0.8% to about 1.5%, about 0.8% to about 2%, about 0.8% to about 2.5%, about 0.8% to about 3%, about 0.8% to about 3.5%, about 0.8% to about 4%, about 1% to about 1.2%, about 1% to about 1.5%, about 1% to about 2%, about 1% to about 2.5%, about 1% to about 3%, about 1% to about 3.5%, about 1% to about 4%, about 1.2% to about 1.5%, about 1.2% to about 2%, about 1.2% to about 2.5%, about 1.2% to about 3%, about 1.2% to about 3.5%, about 1.2% to about 4%, about 1.5% to about 2%, about 1.5% to about 2.5%, about 1.5% to about 3%, about 1.5% to about 3.5%, about 1.5% to about 4%, about 2% to about 2.5%, about 2% to about 3%, about 2% to about 3.5%, about 2% to about 4%, about 2.5% to about 3%, about 2.5% to about 3.5%, about 2.5% to about 4%, about 3% to about 3.5%, about 3% to about 4%, or about 3.5% to about 4%. In some embodiments, the one or more vitamins or derivatives thereof is present in an amount of about 0.1%, about 0.2%, about 0.3%, about 0.5%, about 0.8%, about 1%, about 1.2%, about 1.5%, about 2%, about 2.05%, about 2.5%, about 3%, about 3.5%, or about 4%. In some embodiments, the one or more vitamins or derivatives thereof is present in an amount of at least about 0.1%, about 0.2%, about 0.3%, about 0.5%, about 0.8%, about 1%, about 1.2%, about 1.5%, about 2%, about 2.5%, about 3%, about 3.5%, or about 4%. In some embodiments, the one or more vitamins or derivatives thereof is present in an amount of at most about 0.2%, about 0.3%, about 0.5%, about 0.8%, about 1%, about 1.2%, about 1.5%, about 2%, about 2.5%, about 3%, about 3.5%, or about 4%.

In some embodiments, an additional component in the lipid vesicle composition may comprise an antioxidant. In some cases, the antioxidant may comprise vitamin C or a derivative thereof. In some instances, the antioxidant may comprise ascorbic acid, ascorbyl palmitate, sodium ascorbyl phosphate, magnesium ascorbyl phosphate, retinyl ascorbate, aminopropyl ascorbyl phosphate (K3 Vita-C), tetrahexyldecyl ascorbate, ascorbyl methylsilanol pectinate (ascorbosilane SP), or a combination thereof. In some embodiments, the antioxidant, such as a vitamin C or derivative thereof, may also aid in collagen synthesis. In some embodiments, the additional component may comprise resorcinol (e.g., 4-butyl resorcinol). In some embodiments, antioxidant is present in the composition in an amount of from about 0.005% to about 1%. In some embodiments, antioxidant is present in the composition in an amount of from about 0.005% to about 0.01%, about 0.005% to about 0.02%, about 0.005% to about 0.05%, about 0.005% to about 0.07%, about 0.005% to about 0.1%, about 0.005% to about 0.3%, about 0.005% to about 0.5%, about 0.005% to about 0.8%, about 0.005% to about 1%, about 0.01% to about 0.02%, about 0.01% to about 0.05%, about 0.01% to about 0.07%, about 0.01% to about 0.1%, about 0.01% to about 0.3%, about 0.01% to about 0.5%, about 0.01% to about 0.8%, about 0.01% to about 1%, about 0.02% to about 0.05%, about 0.02% to about 0.07%, about 0.02% to about 0.1%, about 0.02% to about 0.3%, about 0.02% to about 0.5%, about 0.02% to about 0.8%, about 0.02% to about 1%, about 0.05% to about 0.07%, about 0.05% to about 0.1%, about 0.05% to about 0.3%, about 0.05% to about 0.5%, about 0.05% to about 0.8%, about 0.05% to about 1%, about 0.07% to about 0.1%, about 0.07% to about 0.3%, about 0.07% to about 0.5%, about 0.07% to about 0.8%, about 0.07% to about 1%, about 0.1% to about 0.3%, about 0.1% to about 0.5%, about 0.1% to about 0.8%, about 0.1% to about 1%, about 0.3% to about 0.5%, about 0.3% to about 0.8%, about 0.3% to about 1%, about 0.5% to about 0.8%, about 0.5% to about 1%, or about 0.8% to about 1%. In some embodiments, antioxidant is present in the composition in an amount of about 0.005%, about 0.01%, about 0.02%, about 0.05%, about 0.07%, about 0.1%, about 0.3%, about 0.5%, about 0.51%, about 0.8%, or about 1%. In some embodiments, antioxidant is present in the composition in an amount of at least about 0.005%, about 0.01%, about 0.02%, about 0.05%, about 0.07%, about 0.1%, about 0.3%, about 0.5%, about 0.8%, or about 1%. In some embodiments, antioxidant is present in the composition in an amount of at most about 0.01%, about 0.02%, about 0.05%, about 0.07%, about 0.1%, about 0.3%, about 0.5%, about 0.8%, or about 1%.

In some embodiments, the composition comprises a silicon oil or derivative, such as dimethicone. In some embodiments, the oil silicon oil comprises a siloxane polymer. In some embodiments, the siloxane polymer comprises C1-C3 substituents. In some embodiments, the siloxane is polydimethylsiloxane (PDMS). In some embodiments, the silicon oil is polymethylsilsesquioxane (e.g., Botanisil™ SP-360). In some embodiments, the oil is a mixture which comprises a silicon oil (e.g., dimethicone) as a smaller component. In some embodiments, the silicon oil is incorporated in order to enhance the feel of the resulting composition or as a moisturizer. In some embodiments, the oil comprises a silicon oil in an amount of up to about 5%, up to about 4%, up to about 3%, up to about 2%, or up to about 1%. In some embodiments, the silicon oil is present in an amount of from about 0.1% to about 2% (w/w) of the composition. In some embodiments, the silicon oil is present in an amount of from about 0.1% to about 0.5%, 0.1% to about 0.7%, 0.1% to about 1%, 0.1% to about 1.5%, 0.15% to about 2%, 0.5% to about 0.7%, 0.5% to about 1%, 0.5% to about 1.5%, 0.5% to about 2%, 0.7% to about 1%, 0.7% to about 1.5%, 0.7% to about 2%, 1% to about 1.5%, or 1% to about 2% (w/w) of the composition. In some embodiments, the silicon oil is present in an amount of about 0.1%, 0.5%, 0.6%, 0.7%, 1%, 1.5%, or 2% of the composition.

In some embodiments, the composition comprises an emollient. In some embodiments, the emollient comprises caprylic and/or capric triglycerides (e.g., Labrafac CC). In some embodiments, caprylic and/or capric triglycerides is present in an amount of about 1% to about 10%. In some embodiments, caprylic and/or capric triglycerides is present in an amount of about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10%. In some embodiments, caprylic and/or capric triglycerides is present in an amount of about 1%, about 2%, about 3%, about 3.5%, about 4%, about 5%, about 6%, about 7%, about 8%, about 9%, or about 10%. In some embodiments, caprylic and/or capric triglycerides is present in an amount of at least about 1%, about 2%, about 3%, about 3.5%, about 4%, about 5%, about 6%, about 7%, about 8%, or about 9%. In some embodiments, caprylic and/or capric triglycerides is present in an amount of at most about 2%, about 3%, about 3.5%, about 4%, about 5%, about 6%, about 7%, about 8%, about 9%, or about 10%.

In some embodiments, the additional components comprise purified water. In some embodiments, purified water is present in an amount of about 50% to 80% (w/w). In some embodiments, purified water is present in an amount of about 50% to about 55%, about 50% to about 60%, about 50% to about 65%, about 50% to about 70%, about 50% to about 75%, about 50% to about 80%, about 55% to about 60%, about 55% to about 65%, about 55% to about 70%, about 55% to about 75%, about 55% to about 80%, about 60% to about 65%, about 60% to about 70%, about 60% to about 75%, about 60% to about 80%, about 65% to about 70%, about 65% to about 75%, about 65% to about 80%, about 70% to about 75%, about 70% to about 80%, or about 75% to about 80%. In some embodiments, purified water is present in an amount of about 50%, about 55%, about 60%, about 65%, about 70%, about 75%, or about 80%. In some embodiments, purified water is present in an amount of at least about 50%, about 55%, about 60%, about 65%, about 70%, or about 75%. In some embodiments, purified water is present in an amount of at most about 55%, about 60%, about 65%, about 70%, about 75%, or about 80%.

### Exemplary Compositions for Delivery of Anionic Polymer Materials

Provided below are exemplary compositions for the delivery of anionic polymer materials. The embodiments below may additionally comprise any of the other ingredients or components provided herein. In some embodiments, the other ingredients or components may be one or more peptides provided herein. In some embodiments, the one or more peptides can be dipeptides, tripeptides, tetrapeptides, pentapeptides, hexapeptides, heptapeptides, octapeptides, or a combination thereof. In some embodiments, the one or more peptides are unmodified. In some embodiments, the one or more peptides are entrapped in the lipid bilayer, the oil-in-water emulsion, or a combination thereof.

Hyaluronic Acid Composition 1: In one aspect, provided herein, is a lipid vesicle composition comprising
(a) lipid vesicles each comprising a lipid bilayer comprising vesicle forming lipids, wherein the vesicle forming lipids are present in an amount of from about 5% to about 20%;
(b) an oil-in-water emulsion entrapped in the lipid vesicles, and stabilized by one or more surfactants, wherein the one or more surfactants comprises a cationic surfactant;
(c) hyaluronic acid in an amount of from about 0.01 mg/mL to about 10 mg/mL entrapped in the lipid bilayer, the oil-in-water emulsion, or a combination thereof; and
(d) one or more peptides in an amount of from about 0.1 mg/mL to about 50 mg/mL entrapped in the lipid bilayer, the oil-in-water emulsion, or a combination thereof.

In some embodiments, the oil component is present in an amount of from about 2.5% to about 20%.

In some embodiments, the lipid vesicle composition comprises hyaluronic acid in an amount of about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 1.25 mg/mL, about 0.01 mg/mL to about 1.5 mg/mL, about 0.01 mg/mL to about 1.75 mg/mL, about 0.01 mg/mL to about 2 mg/mL, about 0.01 mg/mL to about 5 mg/mL, about 0.01 mg/mL to about 10 mg/mL, about 0.1 mg/mL to about 0.5 mg/mL, about 0.1 mg/mL to about 1 mg/mL, about 0.1 mg/mL to about 1.25 mg/mL, about 0.1 mg/mL to about 1.5 mg/mL, about 0.1 mg/mL to about 1.75 mg/mL, about 0.1 mg/mL to about 2 mg/mL, about 0.1 mg/mL to about 5 mg/mL, about 0.1 mg/mL to about 10 mg/mL, about 0.5 mg/mL to about 1 mg/mL, about 1 mg/mL to about 1.5 mg/mL, about 1 mg/mL to about 1.75 mg/mL, about 1 mg/mL to about 2 mg/mL, about 1 mg/mL to about 5 mg/mL, about 1 mg/mL to about 10 mg/mL. In some embodiments, the lipid vesicle composition comprises hyaluronic acid in an amount of about 0.01 mg/mL, about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, about 5 mg/mL, or about 10 mg/mL.

In some embodiments, the lipid vesicle composition comprises one or more peptides in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL. In some embodiments the lipid vesicle composition comprises one or more peptides in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the lipid vesicle composition comprises one or more peptides in an amount of about 1 mg/mL, about 2 mg/mL, about 3 mg/mL, about 4 mg/mL, or about 5 mg/mL.

In some embodiments, the lipid vesicle composition further comprises viscosity enhancing agents in an amount of from about 0.5% to about 5%. In some embodiments, the viscosity enhancing agents comprise one or more of a fatty alcohol, a wax, a fatty ester of glycerol, or any combination thereof.

In some embodiments, the lipid vesicle composition further comprises a non-ionic surfactant in an amount of from about 0.1% to about 3%. In some embodiments, the non-ionic surfactant is a PEG ether of a fatty alcohol.

In some embodiments, the one or more peptides of (d) comprise a tetrapeptide, pentapeptide, a hexapeptide, or any combination thereof. In some embodiments, the one or more peptides comprises an amino acid sequence at least about 50%, at least about 60%, at least about 70%, at least about 80%, or at least about 90% sequence homology to the amino acid sequence of any one of SEQ ID NO: 1-51. In some embodiments, the one or more peptides comprises an amino acid sequence at least about 50%, at least about 60%, at least about 70%, at least about 80%, or at least about 90% sequence homology to the amino acid sequence of any one of SEQ ID NO: 31, 36, or 37. In some embodiments, the one or more peptides comprises an amino acid sequence identical to the amino acids of any one of SEQ ID NO: 31, 36, or 37. In some embodiments, the one or more peptides comprises an amino acid sequence at least about 50%, at least about 60%, at least about 70%, at least about 80%, or at least about 90% sequence homology to the amino acid sequence of SEQ ID NO: 37. In some embodiments, the one or more peptides comprises an amino acid sequence identical to the amino acids of SEQ ID NO: 37.

In some embodiments, the cationic surfactant is a fatty amide derived propylene glycol-diammonium phosphate ester. In some embodiments, the cationic surfactant is present in an amount of from about 1% to about 10%.

Hyaluronic Acid Composition 2: In one aspect, provided herein, is a lipid vesicle composition comprising
(a) lipid vesicles each comprising a lipid bilayer comprising vesicle forming lipids, wherein the vesicle forming lipids are present in an amount of from about 2% to about 20%;
(b) an oil-in-water emulsion entrapped in the lipid vesicles, and stabilized by one or more surfactants;
(c) hyaluronic acid in an amount of from about 0.01 mg/mL to about 10 mg/mL entrapped in the lipid bilayer, the oil-in-water emulsion, or a combination thereof,
wherein the composition further comprises:
   a gemini surfactant in an amount of from about 0.01% to about 0.5%; and
   a polysorbate in an amount of from about 0.1% to about 2%; and
(d) one or more peptides in an amount of from about 0.1 mg/mL to about 50 mg/mL entrapped in the lipid bilayer, the oil-in-water emulsion, or a combination thereof.

In some embodiments, the oil component is present in an amount of from about 2.5% to about 20%.

In some embodiments, the lipid vesicle composition comprises hyaluronic acid in an amount of about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 1.25 mg/mL, about 0.01 mg/mL to about 1.5 mg/mL, about 0.01 mg/mL to about 1.75 mg/mL, about 0.01 mg/mL to about 2 mg/mL, about 0.01 mg/mL to about 5 mg/mL, about 0.01 mg/mL to about 10 mg/mL, about 0.1 mg/mL to about 0.5 mg/mL, about 0.1 mg/mL to about 1 mg/mL, about 0.1 mg/mL to about 1.25 mg/mL, about 0.1 mg/mL to about 1.5 mg/mL, about 0.1 mg/mL to about 1.75 mg/mL, about 0.1 mg/mL to about 2 mg/mL, about 0.1 mg/mL to about 5 mg/mL, about 0.1 mg/mL to about 10 mg/mL, about 0.5 mg/mL to about 1 mg/mL, about 1 mg/mL to about 1.5 mg/mL, about 1 mg/mL to about 1.75 mg/mL, about 1 mg/mL to about 2 mg/mL, about 1 mg/mL to about 5 mg/mL, about 1 mg/mL to about 10 mg/mL. In some embodiments, the lipid vesicle composition comprises hyaluronic acid in an amount of about 0.01 mg/mL, about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, about 5 mg/mL, or about 10 mg/mL.

In some embodiments, the lipid vesicle composition comprises one or more peptides in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL. In some embodiments the lipid vesicle composition comprises one or more peptides in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the lipid vesicle composition comprises one or more peptides in an amount of about 1 mg/mL, about 2 mg/mL, about 3 mg/mL, about 4 mg/mL, or about 5 mg/mL.

In some embodiments, the lipid vesicle composition further comprises viscosity enhancing agents in an amount of from about 0.5% to about 5%. In some embodiments, the viscosity enhancing agents comprise one or more of a fatty alcohol, a wax, a fatty ester of glycerol, or any combination thereof.

In some embodiments, the one or more peptides of (d) comprise a tetrapeptide, pentapeptide, hexapeptide, or any combination thereof. In some embodiments, the one or more peptides comprises an amino acid sequence at least about 50%, at least about 60%, at least about 70%, at least about 80%, or at least about 90% sequence homology to the amino acid sequence of any one of SEQ ID NO: 1-51. In some embodiments, the one or more peptides comprises an amino acid sequence at least about 50%, at least about 60%, at least about 70%, at least about 80%, or at least about 90% sequence homology to the amino acid sequence of any one of SEQ ID NO: 31, 36, or 37. In some embodiments, the one or more peptides comprises an amino acid sequence identical to the amino acids of any one of SEQ ID NO: 31, 36, or 37. In some embodiments, the one or more peptides comprises an amino acid sequence at least about 50%, at least about 60%, at least about 70%, at least about 80%, or at least about 90% sequence homology to the amino acid sequence of SEQ ID NO: 37. In some embodiments, the one or more peptides comprises an amino acid sequence identical to the amino acids of SEQ ID NO: 37.

In some embodiments, the polysorbate is polysorbate 80.

### Lipid Vesicle Compositions of One or More Peptides for Intradermal Delivery

In one aspect, provided herein, is a lipid vesicle composition comprising one or more peptides. In some embodiments, the one or more peptides can be dipeptides, tripeptides, tetrapeptides, pentapeptides, hexapeptides, heptapeptides, octapeptides or a combination thereof. In some embodiments, the one or more peptides are unmodified. In some embodiments, the lipid vesicle composition comprises lipid vesicles each comprising a lipid bilayer comprising vesicle forming lipids. In some embodiments, the lipid vesicle composition comprises an oil-in-water emulsion entrapped in the lipid vesicles. In some embodiments, the oil-in-water emulsion is stabilized by one or more surfactants. In some embodiments, the one or more peptides are entrapped in the lipid bilayer, the oil-in-water emulsion, or a combination thereof. In some embodiments, the lipid vesicle may further comprise other ingredients or components. In some embodiments, the other ingredients or components may be an anionic polymer material. In some embodiments, the anionic polymer material may be entrapped in the lipid bilayer, the oil-in-water emulsion, or a combination thereof. In some embodiments, the anionic polymer material is hyaluronic acid.

In one aspect, the present disclosure relates to a lipid vesicle composition comprising one or more peptides, which may provide biological activity. Peptides which may provide biological activity may be referred to as bioactive peptides. In some embodiments, bioactive peptides may play a role in biological processes related to skin care, including, but not limited to, modulation of cell proliferation, inflammation, angiogenesis, melanogenesis, extracellular matrix synthesis, etc. See e.g., Falla, et. al., 2009, “Cosmeceuticals and peptides,” Clinics in Dermatology 27:485-494, which is incorporated herein by reference in its entirety.

### Peptides in a Lipid Vesicle Composition

The present disclosure provides lipid vesicle compositions comprising one or more peptides in a lipid vesicle composition. In some embodiments, the one or more peptides are unmodified. In some embodiments, the one or more peptides comprise dipeptides, tripeptides, tetrapeptides, pentapeptides, hexapeptides, heptapeptides, octapeptides, or a combination thereof. In some embodiments, the one or more peptides are tetrapeptides, pentapeptides, hexapeptides, or a combination thereof. In some embodiments, the one or more tetrapeptides, pentapeptides, hexapeptides, or a combination thereof, may be in a lipid vesicle composition comprising an anionic polymer material (e.g., hyaluronic acid). In some embodiments, the one or more peptides may exhibit a high affinity to a metal to form a complex. In some embodiments, the metal may be copper.

In some embodiments, the one or more peptides in the lipid vesicle composition provided herein has a desirable property, or an improved property relative to peptides known in the art. Such a property can include, e.g., a pharmacokinetic property (including but not limited to absorption, bioavailability, distribution, metabolism, and excretion), a pharmacodynamic property (including but not limited to: receptor binding characteristics, e.g., binding half-life; postreceptor effects; and chemical interactions), enhanced activity (e.g., represented by IC₅₀), stability (e.g., represented by half-life), solubility (e.g., in a formulation), or permeability (e.g., permeability of the skin by a formulation containing the one or more peptides). In some embodiments, a formulation of the lipid vesicle containing one or more peptides of the disclosure has a desirable property, or an improved property relative to a formulation known in the art. In some embodiments, a desirable or improved property of a formulation of the disclosure is a property relating to the use of the formulation for an indication as described elsewhere herein, e.g., use for reducing or improving the appearance of skin wrinkles.

### Peptides

In some embodiments, the one or more peptides of the lipid vesicle composition comprises dipeptides, tripeptides, tetrapeptides, pentapeptides, hexapeptides, heptapeptides, octapeptides, or a combination thereof. In some embodiments, the one or more peptides comprises a tetrapeptide. In some embodiments, the one or more peptides comprises a pentapeptide. In some embodiments, the one or more peptides comprises a hexapeptide. In some embodiments, the one or more peptides comprises a pentapeptide and a hexapeptide. In some embodiments, the one or more peptides are unmodified. In some embodiments, the one or more peptides comprises an amino acid sequence which has at least about 50%, at least about 60%, at least about 70%, at least about 80%, or at least about 90% sequence homology to the amino acid sequence of any one of SEQ ID NOs: 1-51. In some embodiments, the one or more peptides comprise one or more amino acid substitutions relative to any one SEQ ID NO: 1-51. In some embodiments, the one or more peptides comprise 1, 2, or 3, 4 amino acid substitutions relative any one of SEQ ID NO: 1-51. In some embodiments, at least 1 of the 1, 2, or 3 amino acid substitutions is a conservative substitution. In some embodiments, the one or more peptides comprise an amino acid sequence identical to the amino acid sequence of any one of SEQ ID NOs: 1-51. In some embodiments, the one or more peptides have an amino acid sequence consisting of an identical sequence of any one of SEQ ID NOs: 1-51.

In some embodiments, the one or more peptides comprise an amino acid sequence which has at least about 50%, at least about 60%, at least about 70%, at least about 80%, or at least about 90% sequence homology to the amino acid sequence SEQ ID NO: 13. In some embodiments, the one or more peptides comprise one or more amino acid substitutions relative to SEQ ID NO: 13. In some embodiments, the one or more amino acid substitutions are selected from the amino acids defined as Xaa1-Xaa3. In some embodiments, the one or more peptides comprise 1 or 2 amino acid substitutions relative to SEQ ID NO: 13. In some embodiments, at least 1 of the 1 or 2 amino acid substitutions is a conservative substitution. In some embodiments, the one or more peptides have an amino acid sequence consisting of an identical sequence of SEQ ID NO: 13.

In some embodiments, the one or more peptides comprise an amino acid sequence which has at least about 50%, at least about 60%, at least about 70%, at least about 80%, or at least about 90% sequence homology to the amino acid sequence SEQ ID NO: 21. In some embodiments, the one or more peptides comprise one or more amino acid substitutions relative to SEQ ID NO: 21. In some embodiments, the one or more amino acid substitutions are selected from the amino acids defined as Xaa1-Xaa5. In some embodiments, the one or more peptides comprise 1, or 2 amino acid substitutions relative to SEQ ID NO: 21. In some embodiments, at least 1 of the 1 or 2 amino acid substitutions is a conservative substitution. In some embodiments, the one or more peptides have an amino acid sequence consisting of an identical sequence of SEQ ID NO: 21.

In some embodiments, the one or more peptides comprise an amino acid sequence which has at least about 50%, at least about 60%, at least about 70%, at least about 80%, or at least about 90% sequence homology to the amino acid sequence SEQ ID NO: 31. In some embodiments, the one or more peptides comprise one or more amino acid substitutions relative to SEQ ID NO: 31. In some embodiments, the one or more amino acid substitutions are selected from the amino acids defined as Xaa1-Xaa5. In some embodiments, the one or more peptides comprise 1, or 2 amino acid substitutions relative to SEQ ID NO: 31. In some embodiments, at least 1 of the 1 or 2 amino acid substitutions is a conservative substitution. In some embodiments, the one or more peptides have an amino acid sequence consisting of an identical sequence of SEQ ID NO: 31.

In some embodiments, the one or more peptides comprise an amino acid sequence which has at least about 50%, at least about 60%, at least about 70%, at least about 80%, or at least about 90% sequence homology to the amino acid sequence of SEQ ID NO: 36. In some embodiments, the one or more peptides comprise one or more amino acid substitutions relative to SEQ ID NO: 36. In some embodiments, the one or more amino acid substitutions are selected from the amino acids defined as Xaa1-Xaa6. In some embodiments, the one or more peptides comprise 1, 2, or 3amino acid substitutions relative to SEQ ID NO: 36. In some embodiments, at least 1 of the 1, 2, or 3 amino acid substitutions is a conservative substitution. In some embodiments, the one or more peptides have an amino acid sequence consisting of an identical sequence of SEQ ID NO: 36.

In some embodiments, the one or more peptides comprise an amino acid sequence which has at least about 50%, at least about 60%, at least about 70%, at least about 80%, or at least about 90% sequence homology to the amino acid sequence of SEQ ID NO: 37. In some embodiments, the one or more peptides comprise one or more amino acid substitutions relative to SEQ ID NO: 37. In some embodiments, the one or more amino acid substitutions are selected from the amino acids defined as Xaa1-Xaa6. In some embodiments, the one or more peptides comprise 1, 2, or 3 amino acid substitutions relative to SEQ ID NO: 37. In some embodiments, at least 1 of the 1, 2, or 3 amino acid substitutions is a conservative substitution. In some embodiments, the one or more peptides have an amino acid sequence consisting of an identical sequence of SEQ ID NO: 37.

In some embodiments, the one or more peptides comprise an amino acid sequence which has at least about 50%, at least about 60%, at least about 70%, at least about 80%, or at least about 90% sequence homology to the amino acid sequence of SEQ ID NO: 40. In some embodiments, the one or more peptides comprise one or more amino acid substitutions relative to SEQ ID NO: 37. In some embodiments, the one or more amino acid substitutions are selected from the amino acids defined as Xaa1-Xaa6. In some embodiments, the one or more peptides comprise 1, 2, or 3 amino acid substitutions relative to SEQ ID NO: 40. In some embodiments, at least 1 of the 1, 2, or 3 amino acid substitutions is a conservative substitution. In some embodiments, the one or more peptides have an amino acid sequence consisting of an identical sequence of SEQ ID NO: 40.

In some embodiments, the one or more peptides comprises up to about 8 amino acids up to about 7 amino acids, up to 6 amino acids, up to 5 amino acids, up to 4 amino acids, up to 3 amino acids, or up to 2 amino acids. In some embodiments, the one or more peptides has a molecular weight of up to about 1500 Da, up to about 1100 Da, up to about 950 Da, up to about 800 Da, up to about 650 Da, up to about 500 Da, up to about to about 350 Da, or up to about 250 Da.

In some embodiments, one or more peptides of the lipid vesicle composition has 2-8 residues. In some embodiments, one or more peptides of the lipid vesicle comprises the amino acid sequence:

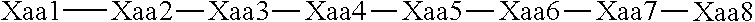

wherein:
Xaa1 is absent or selected from Ala, Gly, Gln, Glu, Val, Leu, Cys, Met, Sec, Ser, Thr, Tyr, Trp, Arg, Asn, Asp, His, Pro, Phe, Lys, Ile and a derivative of Ala, Gly, Gln, Glu, Val, Leu, Cys, Met, Sec, Ser, Thr, Tyr, Trp, Arg, Asn, Asp, His, Pro, Phe, Lys, or Ile;
Xaa2 is absent or selected from: Ala, Gly, Gln, Glu, Val, Leu, Cys, Met, Sec, Ser, Thr, Tyr, Trp, Arg, Asn, Asp, His, Pro, Phe, Lys, Ile and a derivative of Ala, Gly, Gln, Glu, Val, Leu, Cys, Met, Sec, Ser, Thr, Tyr, Trp, Arg, Asn, Asp, His, Pro, Phe, Lys, or Ile;
Xaa3 is absent or selected from: Ala, Gly, Gln, Glu, Val, Leu, Cys, Met, Sec, Ser, Thr, Tyr, Trp, Arg, Asn, Asp, His, Pro, Phe, Lys, Ile and a derivative of Ala, Gly, Gln, Glu, Val, Leu, Cys, Met, Sec, Ser, Thr, Tyr, Trp, Arg, Asn, Asp, His, Pro, Phe, Lys, or Ile;
Xaa4 is absent or selected from: Ala, Gly, Gln, Glu, Val, Leu, Cys, Met, Sec, Ser, Thr, Tyr, Trp, Arg, Asn, Asp, His, Pro, Phe, Lys, Ile and a derivative of Ala, Gly, Gln, Glu, Val, Leu, Cys, Met, Sec, Ser, Thr, Tyr, Trp, Arg, Asn, Asp, His, Pro, Phe, Lys, or Ile;
Xaa5 is absent or selected from: Ala, Gly, Gln, Glu, Val, Leu, Cys, Met, Sec, Ser, Thr, Tyr, Trp, Arg, Asn, Asp, His, Pro, Phe, Lys, Ile and a derivative of Ala, Gly, Gln, Glu, Val, Leu, Cys, Met, Sec, Ser, Thr, Tyr, Trp, Arg, Asn, Asp, His, Pro, Phe, Lys, or Ile;
Xaa6 is absent or selected from: Ala, Gly, Gln, Glu, Val, Leu, Cys, Met, Sec, Ser, Thr, Tyr, Trp, Arg, Asn, Asp, His, Pro, Phe, Lys, Ile and a derivative of Ala, Gly, Gln, Glu, Val, Leu, Cys, Met, Sec, Ser, Thr, Tyr, Trp, Arg, Asn, Asp, His, Pro, Phe, Lys, or Ile;
Xaa7 is selected from: Ala, Gly, Gln, Glu, Val, Leu, Cys, Met, Sec, Ser, Thr, Tyr, Trp, Arg, Asn, Asp, His, Pro, Phe, Lys, Ile and a derivative of Ala, Gly, Gln, Glu, Val, Leu, Cys, Met, Sec, Ser, Thr, Tyr, Trp, Arg, Asn, Asp, His, Pro, Phe, Lys, or Ile;
Xaa8 is selected from: Ala, Gly, Gln, Glu, Val, Leu, Cys, Met, Sec, Ser, Thr, Tyr, Trp, Arg, Asn, Asp, His, Pro, Phe, Lys, Ile and a derivative of Ala, Gly, Gln, Glu, Val, Leu, Cys, Met, Sec, Ser, Thr, Tyr, Trp, Arg, Asn, Asp, His, Pro, Phe, Lys, or Ile;
the N-terminus is unmodified; and
the C-terminus is unmodified.

In some embodiments, one or more peptides of the lipid vesicle composition has 2-8 residues. In some embodiments, one or more peptides of the lipid vesicle comprises the amino acid sequence:

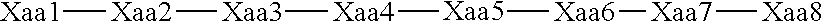

wherein:
Xaa1 is absent or selected from Ala, Arg, Gly, Lys, Pro, Tyr, Val and a derivative of Ala, Arg, Gly, Lys, Pro, Tyr, or Val;
Xaa2 is absent or selected from: Ala, Arg, Cys, Gly, Ile, Lys, Pro, Tyr, Val and a derivative of Ala, Arg, Cys, Gly, Ile, Lys, Pro, Tyr, or Val;
Xaa3 is absent or selected from: Ala, Arg, Gln, Gly, Lys, Phe, Pro, Tyr, Val and a derivative of Ala, Arg, Gln, Gly, Lys, Phe, Pro, Tyr, or Val;
Xaa4 is absent or selected from: Ala, Arg, Glu, Gly, Lys, Pro, Tyr, Val and a derivative of Ala, Arg, Glu, Gly, Lys, Pro, Tyr, or Val;
Xaa5 is absent or selected from: Ala, Arg, Gln, Gly, Lys, Leu, Met, Pro, Thr, Tyr, Val and a derivative of Ala, Arg, Gln, Gly, Lys, Leu, Met, Pro, Thr, Tyr, or Val;
Xaa6 is absent or selected from: Ala, Arg, Asp, Gln, Gly, His, Lys, Phe, Pro, Ser, Thr, Tyr, Val and a derivative of Ala, Arg, Asp, Gln, Gly, His, Lys, Phe, Pro, Ser, Thr, Tyr, or Val;
Xaa7 is selected from: Ala, Arg, Asn, Asp, Cys, Gly, His, Lys, Phe, Pro, Thr, Tyr, Val and a derivative of Ala, Arg, Asn, Asp, Cys, Gly, His, Lys, Phe, Pro, Thr, Tyr, or Val;
Xaa8 is selected from: Ala, Arg, Cys, Glu, Gly, His, Lys, Phe, Pro, Ser, Thr, Trp, Tyr, Val and a derivative of Ala, Arg, Cys, Glu, Gly, His, Lys, Phe, Pro, Ser, Thr, Trp, Tyr, or Val; the N-terminus is unmodified; and
the C-terminus is unmodified.

Non-limiting examples of one or more peptides of the disclosure are shown in Table 2.

TABLE 2
Peptide Examples

| SEQ | |
|---|---|
| ID | |
| NO | SEQUENCE |
| 1 | Lys-Lys |
| 2 | Lys-Pro |
| 3 | Cys-Lys |
| 4 | Lys-Cys |
| 5 | Lys-Thr |
| 6 | Asp-Phe |
| 7 | Asn-Phe |
| 8 | Val-Trp |
| 9 | Tyr-Arg |
| 10 | Thr-Thr |
| 11 | His-Gly-Gly |
| 12 | Arg-Lys-Arg |
| 13 | Gly-His-Lys |
| 14 | Gly-Gly-His |
| 15 | Gly-His-Gly |
| 16 | Lys-Phe-Gly |
| 17 | Lys-Phe-Lys |
| 18 | Lys-Gly-His |
| 19 | Lys-His-Gly |
| 20 | Lys-Phe-Lys |
| 21 | Gly-Gln-Pro-Arg |
| 22 | Lys-Thr-Phe-Lys |
| 23 | Ala-Gln-Thr-Arg |
| 24 | Ala-His-Ser-His |
| 25 | Arg-Ser-Arg-Lys |
| 26 | Lys-Asp-Val-Tyr |
| 27 | Lys-Thr-Ala-Lys |
| 28 | Lys-Phe-Tyr-Lys |
| 29 | Lys-Ala-Tyr-Lys |
| 30 | Thr-Thr-Lys-Ser |
| 31 | Lys-Thr-Thr-Lys-Ser |
| 32 | Lys-Leu-Ala-Ala-Lys |
| 33 | Lys-Gly-Gly-Pro-Gly |
| 34 | Lys-Ala-Gly-Gly-Pro |
| 35 | Gly-Ala-Gly-Pro-Gly |
| 36 | Val-Gly-Val-Ala-Pro-Gly |
| 37 | Gly-Val-Ala-Pro-Gly-Val |
| 38 | Gly-Lys-Thr-Thr-Lys-Ser |
| 39 | Gly-Lys-Thr-Ser-Lys-Ser |
| 40 | Phe-Val-Ala-Pro-Phe-Pro |
| 41 | Ala-Gly-Gly-Ala-Pro-Gly |
| 42 | Lys-Gly-Gly-Gly-Pro-Gly |
| 43 | Lys-Ala-Gly-Gly-Pro-Gly |
| 44 | Tyr-Tyr-Arg-Ala-Asp-Ala |
| 45 | Gln-Gly-Gln-Ly-Pro-Gly |
| 46 | Gln-Gly-Val-Ly-Pro-Ala |
| 47 | Pro-Gly-Ala-Tyr-Pro-Gly |
| 48 | Pro-Lys-Gly-Ser-Pro-Gly |
| 49 | Arg-Gly-Tyr-Tyr-Lys-Lys-Glu |
| 50 | Cys-Gly-Gly-Pro-Gly-Ala-Gly |
| 51 | Gly-Gly-Gly-Pro-Gly-Ala-Gly |
| 52 | Val-Ile-Gly-Tyr-Lys-Thr-Thr-Lys |

Unless otherwise indicated in the table, peptides listed in Table 2 can comprise all L-amino acids or all D-amino acids. In some embodiments, peptides listed in Table 2 are unmodified. In some embodiments, peptides listed in Table 2 are synthetic human peptides (e.g., sh-polypeptide). In some embodiments, peptides listed in Table 2 are recombinant human peptides (e.g., rh-polypeptide). In some embodiments, the peptides listed in Table 2 comprise a counter ion (e.g., acetate). In some embodiments, the peptides listed in Table 2 are modified with an acetyl group (e.g., Ac).

### Amino Acid Derivatives

The present disclosure contemplates the use of an amino acid derivative or analog of any amino acid in any of the one or more peptides of the disclosure, such as those listed in Table 2. In some embodiments, amino acid modifications can be made chemically using any known method. Selective protein modifications are described in the literature, e.g., by Spicer and Davis, 2014, “Selective chemical protein modification,” Nature Communications 5:4740, incorporated herein by reference.

In some embodiments, an amino acid derivative is a non-canonical amino acid. In some embodiments, a non-canonical amino acid has an(S) configuration at the alpha position. In some embodiments, a non-canonical amino acid has an (R) configuration at the alpha position. In some embodiments, a non-canonical amino acid is an alpha amino acid. In some embodiments, a non-canonical amino acid is a beta or gamma amino acid (e.g., beta-alanine). In some embodiments, a non-canonical amino acid is selected from the group consisting of: an aromatic side chain amino acid; a non-aromatic side chain amino acid; an aliphatic side chain amino acid; a side chain amide amino acid; a side chain ester amino acid; a heteroaromatic side chain amino acid; a side chain thiol amino acid; a beta amino acid; and a backbone-modified amino acid. In some embodiments, a non-canonical amino acid is a derivative of tyrosine, histidine, tryptophan, or phenylalanine. In some embodiments, a derivative of an amino acid comprises an ester, amide, disulfide, carbamate, urea, phosphate, ether of the amino acid. In some embodiments, a non-aromatic side chain amino acid is a derivative of serine, threonine, cysteine, methionine, arginine, asparagine, glutamine, aspartic acid, glutamic acid, lysine, proline, glycine, alanine, valine, isoleucine, or leucine. In some embodiments, a non-canonical amino acid is selected from the group consisting of 2-aminoadipic acid; 3-aminoadipic acid; beta-alanine; beta-aminoproprionic acid; 2-aminobutyric acid; 4-aminobutyric acid; piperidinic acid; 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminoproprionic acid; N-ethylglycine; N-ethylasparagine; hydroxylysine; allo-hydroxylysine; 3-hydroxyproline; 4-hydroxyproline; isodesmosine; allo-isoleucine; N-methylglycine; sarcosine; n-methylisoleucine; 6-N-methyllysine; N-methylvaline; norvaline; norleucine; and ornithine. In some embodiments, a non-canonical amino acid is a proline derivative. In some embodiments, a proline derivative is 3-fluoroproline, 4-fluoroproline, 3-hydroxyproline, 4-hydroxyproline, 3-aminoproline, 4-aminoproline, 3,4-dehydroproline, aziridine-2-carboxylic acid, azetidine-2-carboxylic acid, pipecolic acid, 4-oxa-proline, 3-thiaproline, or 4-thiaproline. In some embodiments, a non-canonical amino acid comprises a lipid.

In some embodiments, one or more peptides of the disclosure comprises one or more amino acid derivative or analog, e.g., as known to those of skill in the art and described in the literature or herein. In some embodiments, one or more peptides of the disclosure comprises 2, 3, 4, 5, 6, 7, 8, 2-3, 2-4, 2-5, 2-6, 2-7, or 2-8 amino acid derivatives. In some embodiments, the one or more peptides are unmodified.

In some embodiments, each amino acid derivative present in the one or more peptides of the disclosure is a non-canonical amino acid independently selected from the group consisting of: an aromatic side chain amino acid; a non-aromatic side chain amino acid; an aliphatic side chain amino acid; a side chain amide amino acid; a side chain ester amino acid; a heteroaromatic side chain amino acid; a side chain thiol amino acid; a beta amino acid; and a backbone-modified amino acid, selected from e.g., the non-canonical amino acids described herein or known in the art and described in the published literature.

In some embodiments, the one or more peptides comprises one or more amino acids that have the D-amino acid configuration, and the remaining amino acids in the peptide have the L-amino acid configuration.

In some embodiments, a non-canonical amino acid is a proline derivative. In some embodiments, a proline derivative comprises one or more substitutions on the pyrrolidine ring. In some embodiments, a proline derivative comprises one or more substitutions on the pyrrolidine ring, wherein the substitutions comprise halogen, alkoxy, amino, hydroxyl, alkyl (methyl, ethyl), thiol, or alkylthio. In some embodiments, a proline derivative comprises one or more substitutions on the pyrrolidine ring, wherein the substitutions comprise halogen, or alkyl (methyl, ethyl). In some embodiments, a proline derivative comprises one or more substitutions on the pyrrolidine ring, wherein the substitutions comprise halogen. In some embodiments, a proline derivative comprises one or more substitutions on the pyrrolidine ring, wherein the substitutions comprise alkoxy, hydroxyl, amino. In some embodiments, a proline derivative comprises one or more substitutions on the pyrrolidine ring, wherein the substitutions comprise halogen, alkoxy, alkyl (methyl, ethyl), thiol, or alkylthio.

### Concentrations of Peptides in the Composition

The composition can comprise one or more peptides. In some embodiments, the one or more peptides comprise one or more peptides provided in Table 2. In some embodiments, the one or more peptides are present in the vesicle composition in an amount of about 0.01 mg/mL to about 50 mg/mL. In some embodiments, the one or more peptides is present in the vesicle composition in an amount of about 0.005 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 2 mg/mL, about 0.01 mg/mL to about 3 mg/mL, about 0.01 mg/mL to about 4 mg/mL, about 0.01 mg/mL to about 5 mg/mL, about 0.01 mg/mL to about 10 mg/mL, about 0.01 mg/mL to about 20 mg/mL, about 0.01 mg/mL to about 50 mg/mL, about 0.05 mg/mL to about 0.1 mg/mL, about 0.05 mg/mL to about 0.5 mg/mL, about 0.05 mg/mL to about 1 mg/mL, about 0.05 mg/mL to about 2 mg/mL, about 0.05 mg/mL to about 3 mg/mL, about 0.05 mg/mL to about 4 mg/mL, about 0.05 mg/mL to about 5 mg/mL, about 0.05 mg/mL to about 10 mg/mL, about 0.01 mg/mL to about 20 mg/mL, about 0.05 mg/mL to about 50 mg/mL, 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL, about 0.5 mg/mL to about 1 mg/mL, about 0.5 mg/mL to about 2 mg/mL, about 0.5 mg/mL to about 3 mg/mL, about 0.5 mg/mL to about 4 mg/mL, about 0.5 mg/mL to about 5 mg/mL, about 0.5 mg/mL to about 10 mg/mL, about 0.5 mg/mL to about 20 mg/mL, about 0.5 mg/mL to about 50 mg/mL, about 1 mg/mL to about 2 mg/mL, about 1 mg/mL to about 3 mg/mL, about 1 mg/mL to about 4 mg/mL, about 1 mg/mL to about 5 mg/mL, about 1 mg/mL to about 10 mg/mL, about 1 mg/mL to about 20 mg/mL, about 1 mg/mL to about 50 mg/mL, about 2 mg/mL to about 3 mg/mL, about 2 mg/mL to about 4 mg/mL, about 2 mg/mL to about 5 mg/mL, about 2 mg/mL to about 10 mg/mL, about 2 mg/mL to about 20 mg/mL, about 2 mg/mL to about 50 mg/mL, about 3 mg/mL to about 4 mg/mL, about 3 mg/mL to about 5 mg/mL, about 3 mg/mL to about 10 mg/mL, about 3 mg/mL to about 20 mg/mL, about 3 mg/mL to about 50 mg/mL, about 4 mg/mL to about 5 mg/mL, about 4 mg/mL to about 10 mg/mL, about 4 mg/mL to about 20 mg/mL, about 4 mg/mL to about 50 mg/mL, about 5 mg/mL to about 10 mg/mL, about 5 mg/mL to about 20 mg/mL, about 5 mg/mL to about 50 mg/mL, about 10 mg/mL to about 20 mg/mL, about 10 mg/mL to about 50 mg/mL, or about 20 mg/mL to about 50 mg/mL. In some embodiments, the one or more peptides is present in the vesicle composition in an amount of about 0.005 mg/mL, 0.01 mg/mL, 0.02 mg/mL, about 0.05 mg/mL, about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the one or more peptides is present in the vesicle composition in an amount of at least about 0.005 mg/mL, 0.01 mg/mL, 0.02 mg/mL, about 0.05 mg/mL, about 0.1 mg/mL, about 0.5 mg/mL, about 1 mg/mL, about 2 mg/mL, about 3 mg/mL, about 4 mg/mL, about 5 mg/mL, about 10 mg/mL, or about 20 mg/mL. In some embodiments, the one or more peptides is present in the vesicle composition in an amount of at most about 0.01 mg/mL, 0.02 mg/mL, about 0.05 mg/mL, about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the one or more peptides is present in the composition in an amount of about 1 mg/mL, about 2 mg/mL, about 3 mg/mL, about 4 mg/mL, or about 5 mg/mL.

In some embodiments, each of the one or more peptides is present in the vesicle composition in an amount of about 0.001 mg/mL to about 50 mg/mL. In some embodiments, the one or more peptides is present in the vesicle composition in an amount of about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 2 mg/mL, about 0.01 mg/mL to about 3 mg/mL, about 0.01 mg/mL to about 4 mg/mL, about 0.01 mg/mL to about 5 mg/mL, about 0.01 mg/mL to about 10 mg/mL, about 0.01 mg/mL to about 20 mg/mL, about 0.01 mg/mL to about 50 mg/mL, about 0.05 mg/mL to about 0.1 mg/mL, about 0.05 mg/mL to about 0.5 mg/mL, about 0.05 mg/mL to about 1 mg/mL, about 0.05 mg/mL to about 2 mg/mL, about 0.05 mg/mL to about 3 mg/mL, about 0.05 mg/mL to about 4 mg/mL, about 0.05 mg/mL to about 5 mg/mL, about 0.05 mg/mL to about 10 mg/mL, about 0.01 mg/mL to about 20 mg/mL, about 0.05 mg/mL to about 50 mg/mL, about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL, about 0.5 mg/mL to about 1 mg/mL, about 0.5 mg/mL to about 2 mg/mL, about 0.5 mg/mL to about 3 mg/mL, about 0.5 mg/mL to about 4 mg/mL, about 0.5 mg/mL to about 5 mg/mL, about 0.5 mg/mL to about 10 mg/mL, about 0.5 mg/mL to about 20 mg/mL, about 0.5 mg/mL to about 50 mg/mL, about 1 mg/mL to about 2 mg/mL, about 1 mg/mL to about 3 mg/mL, about 1 mg/mL to about 4 mg/mL, about 1 mg/mL to about 5 mg/mL, about 1 mg/mL to about 10 mg/mL, about 1 mg/mL to about 20 mg/mL, about 1 mg/mL to about 50 mg/mL, about 2 mg/mL to about 3 mg/mL, about 2 mg/mL to about 4 mg/mL, about 2 mg/mL to about 5 mg/mL, about 2 mg/mL to about 10 mg/mL, about 2 mg/mL to about 20 mg/mL, about 2 mg/mL to about 50 mg/mL, about 3 mg/mL to about 4 mg/mL, about 3 mg/mL to about 5 mg/mL, about 3 mg/mL to about 10 mg/mL, about 3 mg/mL to about 20 mg/mL, about 3 mg/mL to about 50 mg/mL, about 4 mg/mL to about 5 mg/mL, about 4 mg/mL to about 10 mg/mL, about 4 mg/mL to about 20 mg/mL, about 4 mg/mL to about 50 mg/mL, about 5 mg/mL to about 10 mg/mL, about 5 mg/mL to about 20 mg/mL, about 5 mg/mL to about 50 mg/mL, about 10 mg/mL to about 20 mg/mL, about 10 mg/mL to about 50 mg/mL, or about 20 mg/mL to about 50 mg/mL. In some embodiments, the one or more peptides is present in the vesicle composition in an amount of about 0.001 mg/mL, 0.002 mg/mL, 0.005 mg/mL, 0.01 mg/mL, 0.02 mg/mL, 0.03 mg/mL, 0.05 mg/mL, 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, each of the one or more peptides is present in the vesicle composition in an amount of at least about 0.001 mg/mL, 0.002 mg/mL, 0.005 mg/mL, 0.01 mg/mL, about 0.05 mg/mL, about 0.1 mg/mL, about 0.5 mg/mL, about 1 mg/mL, about 2 mg/mL, about 3 mg/mL, about 4 mg/mL, about 5 mg/mL, about 10 mg/mL, or about 20 mg/mL. In some embodiments, each of the one or more peptides is present in the vesicle composition in an amount of at most about 0.002 mg/mL, 0.005 mg/mL, 0.01 mg/mL, about 0.05 mg/mL, about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, each of the one or more peptides is present in the composition in an amount of about 0.001 mg/mL, 0.002 mg/mL, 0.005 mg/mL, 0.01 mg/mL, about 0.05 mg/mL, about 0.1 mg/mL, about 0.5 mg/mL, about 1 mg/mL, about 2 mg/mL, about 3 mg/mL, about 4 mg/mL, or about 5 mg/mL.

In some embodiments, the one or more peptides are present in the vesicle composition in a total amount of about 0.005% to about 0.2%. In some embodiments, the one or more peptides is present in the vesicle composition in a total amount of about 0.005% to about 0.01%, about 0.005% to about 0.05%, about 0.005% to about 0.1%, about 0.005% to about 0.15%, about 0.005% to about 0.2%, about 0.01% to about 0.05%, about 0.01% to about 0.1%, about 0.01% to about 0.15%, about 0.01% to about 0.2%, about 0.02% to about 0.03%, about 0.02% to about 0.05%, about 0.05% to about 0.1%, about 0.05% to about 0.15%, about 0.05% to about 0.2%, about 0.1% to about 0.15%, about 0.1% to about 0.2%, or about 0.15% to about 0.2%. In some embodiments, the one or more peptides is present in the vesicle composition in a total amount of about 0.01%, about 0.02%, about 0.021%, about 0.022%, about 0.023%, about 0.024%, about 0.025%, about 0.026%, about 0.028%, about 0.03%, about 0.05%, about 0.1%, about 0.15%, or about 0.2%. In some embodiments, the one or more peptides is present in the vesicle composition in a total amount of at least about 0.005%, 0.01%, about 0.05%, about 0.1%, or about 0.15%. In some embodiments, the one or more peptides is present in the vesicle composition in a total amount of at most about 0.01%, 0.05%, about 0.1%, about 0.15%, or about 0.2%.

In some embodiments, each of the one or more peptides are present in the vesicle composition in an amount of about 0.001% to about 0.2%. In some embodiments, each of the one or more peptides is present in the vesicle composition in an amount of about 0.001% to about 0.002%, about 0.001% to about 0.005%, about 0.001% to about 0.01%, about 0.001% to about 0.05%, about 0.001% to about 0.1%, about 0.001% to about 0.15%, about 0.001% to about 0.2%, about 0.002% to about 0.005%, about 0.002% to about 0.01%, about 0.002% to about 0.05%, about 0.002% to about 0.1%, about 0.002% to about 0.15%, about 0.002% to about 0.2%, about 0.005% to about 0.01%, about 0.005% to about 0.05%, about 0.005% to about 0.1%, about 0.005% to about 0.15%, about 0.005% to about 0.2%, about 0.01% to about 0.05%, about 0.01% to about 0.1%, about 0.01% to about 0.15%, about 0.01% to about 0.2%, about 0.05% to about 0.1%, about 0.05% to about 0.15%, about 0.05% to about 0.2%, about 0.1% to about 0.15%, about 0.1% to about 0.2%, or about 0.15% to about 0.2%. In some embodiments, each of the one or more peptides is present in the vesicle composition in an amount of about 0.001%, 0.002%, 0.005%, 0.01%, about 0.05%, about 0.1%, about 0.15%, or about 0.2%. In some embodiments, each of the one or more peptides is present in the vesicle composition in an amount of at least about 0.001%, 0.002%, 0.005%, 0.01%, about 0.05%, about 0.1%, or about 0.15%. In some embodiments, each of the one or more peptides is present in the vesicle composition in an amount of at most about 0.002%, 0.005%, 0.05%, about 0.1%, about 0.15%, or about 0.2%.

### Vesicle Forming Lipids

In some embodiments, the vesicle composition comprises one or more vesicle forming lipids. The vesicle forming lipids act to encapsulate portions of the oil-in-water emulsions. In some embodiments, this allows the oil-in-water emulsion to remain stable for a period of time.

The vesicle forming lipids may be any suitable lipids for such a purpose. In some embodiments, the vesicle forming lipids comprise phospholipids, glycolipids, lecithins, ceramides, lysolecithin, lysophosphatidylethanolamine, phosphatidylserine, phosphatidylinositol, sphingomyelin, cardiolipin, phosphatidic acid, cerebroside, or any combination thereof. In some embodiments, the vesicle forming lipids comprise a combination of lipids.

In some embodiments, the vesicle forming lipids comprise phospholipids. In some embodiments, the phospholipids are naturally occurring, semisynthetic, or synthetically prepared, or a mixture thereof. In an embodiment, the phospholipids are one or more esters of glycerol with one or two (equal or different) residues of fatty adds and with phosphoric acid, wherein the phosphoric acid residue is in turn bound to a hydrophilic group, such as, for instance, choline (phosphatidylcholines—PC), serine (phosphatidylserines—PS), glycerol (phosphatidylglycerols—PG), ethanolamine (phosphatidylethanolamines—PE), or inositol (phosphatidylinositol). Esters of phospholipids with only one residue of fatty acid are generally referred to in the art as the “lyso” forms of the phospholipid or “lysophospholipids”. Fatty acids residues present in the phospholipids are in general long chain aliphatic acids, typically containing 12 to 24 carbon atoms, or 14 to 22 carbon atoms; the aliphatic chain may contain one or more unsaturations or is completely saturated. Examples of suitable fatty acids included in the phospholipids are, for instance, lauric acid, myristic acid, palmitic acid, stearic acid, arachidic acid, behenic acid, oleic acid, linoleic acid, and linolenic acid. Saturated fatty acids such as myristic acid, palmitic acid, stearic acid and arachidic acid may be employed.

In some embodiments, the phospholipid comprises one or more natural phospholipids. In some embodiments, the phospholipid comprises one or more semisynthetic phospholipids. In some embodiments, the semisynthetic phospholipids are the partially or fully hydrogenated derivatives of the naturally occurring lecithins. In some embodiments, the phospholipids include fatty acids di-esters of phosphatidylcholine, ethylphosphatidylcholine, phosphatidylglycerol, phosphatidic acid, phosphatidylethanolamine, phosphatidylserine or of sphingomyelin. In some embodiments, the phospholipids include hydrogenated phosphatidylcholine (e.g., Sunlipon 90H). In some embodiments, the phospholipids are, for instance, dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroylphosphatidylglycerol (DLPG) and its alkali metal salts, diarachidoylphosphatidylglycerol (DAPG) and its alkali metal salts, dimyristoylphosphatidylglycerol (DMPG) and its alkali metal salts, dipalmitoylphosphatidylglycerol (DPPG) and its alkali metal salts, distearoylphosphatidylglycerol (DSPG) and its alkali metal salts, dioleoyl-phosphatidylglycerol (DOPG) and its alkali metal salts, dimyristoyl phosphatidic acid DMPA) and its alkali metal salts, dipalmitoyl phosphatidic acid (DPPA) and its alkali metal salts, distearoyl phosphatidic acid (DSPA), diarachidoylphosphatidic acid (DAPA) and its alkali metal salts, dimyristoylphosphatidylethanolamine (DMPE), dipalmitoylphosphatidylethanolamine (DPPE), distearoyl phosphatidyl-ethanolamine (DSPE), dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), dioleoyl-phosphatidylinositol (DOPI).

In some embodiments, the vesicle forming lipids are present in an amount of about 0.5% to about 25% (w/w) of the composition. In some embodiments, the vesicle forming lipids are present in an amount of about 0.5% to about 2%, about 0.5% to about 5%, about 0.5% to about 8%, about 0.5% to about 10%, about 0.5% to about 12%, about 0.5% to about 15%, about 0.5% to about 20%, about 0.5% to about 25%, about 2% to about 5%, about 2% to about 8%, about 2% to about 10%, about 2% to about 12%, about 2% to about 15%, about 2% to about 20%, about 2% to about 25%, about 5% to about 8%, about 5% to about 10%, about 5% to about 12%, about 5% to about 15%, about 5% to about 20%, about 5% to about 25%, about 8% to about 10%, about 8% to about 12%, about 8% to about 15%, about 8% to about 20%, about 8% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25% (w/w) of the composition. In some embodiments, the vesicle forming lipids are present in an amount of about 0.5%, about 2%, about 5%, about 8%, about 10%, about 12%, about 15%, about 20%, or about 25%. In some embodiments, the vesicle forming lipids are present in an amount of at least about 0.5%, about 2%, about 5%, about 8%, about 10%, about 12%, about 15%, or about 20% (w/w) of the composition. In some embodiments, the vesicle forming lipids are present in an amount of at most about 2%, about 5%, about 8%, about 10%, about 12%, about 15%, about 20%, or about 25% (w/w) of the composition.

In some embodiments, the vesicle forming lipids are present in an amount of about 5% to about 15% (w/w) of the composition. In some embodiments, the vesicle forming lipids are present in an amount of about 5% to about 8%, about 5% to about 9%, about 5% to about 10%, about 5% to about 11%, about 5% to about 12%, about 5% to about 13%, about 5% to about 14%, about 5% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or about 14% to about 15%. In some embodiments, the vesicle forming lipids are present in an amount of about 5%, about 8%, about 9%, about 10%, about 11%, about 12%, about 13%, about 14%, or about 15% (w/w) of the composition. In some embodiments, the vesicle forming lipids are present in an amount of at least about 5%, about 8%, about 9%, about 10%, about 11%, about 12%, about 13%, or about 14% (w/w) of the composition. In some embodiments, the vesicle forming lipids are present in an amount of at most about 8%, about 9%, about 10%, about 11%, about 12%, about 13%, about 14%, or about 15% (w/w) of the composition.

In some embodiments, the composition comprises a short chain polyol. In some embodiments, the short chain polyol acts to enhance the stability of the resulting lipid vesicles. In some embodiments, the short chain polyol is a C₂-C₄ polyol comprising two or three alcohol groups. In some embodiments, the short chain polyol is propylene glycol. In some embodiments, the composition comprises propylene glycol. In some embodiments, the composition comprises a butanediol. In some embodiments, the butanediol is 1,3-butanediol. In some embodiments, the butanediol is 2,3-butanediol. In some embodiments, the composition comprises propylene glycol, butanediol (e.g., 2,3-butanediol), or both.

In some embodiments, the propylene glycol is present in an amount of about 0.5% to about 25% (w/w) of the composition. In some embodiments, the propylene glycol is present in an amount of about 0.5% to about 2%, about 0.5% to about 5%, about 0.5% to about 8%, about 0.5% to about 10%, about 0.5% to about 12%, about 0.5% to about 15%, about 0.5% to about 20%, about 0.5% to about 25%, about 2% to about 5%, about 2% to about 8%, about 2% to about 10%, about 2% to about 12%, about 2% to about 15%, about 2% to about 20%, about 2% to about 25%, about 5% to about 8%, about 5% to about 10%, about 5% to about 12%, about 5% to about 15%, about 5% to about 20%, about 5% to about 25%, about 8% to about 10%, about 8% to about 12%, about 8% to about 15%, about 8% to about 20%, about 8% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25%. In some embodiments, the propylene glycol is present in an amount of about 0.5%, about 2%, about 5%, about 8%, about 10%, about 10.5%, about 12%, about 15%, about 20%, or about 25%. In some embodiments, the propylene glycol is present in an amount of at least about 0.5%, about 2%, about 5%, about 8%, about 10%, about 12%, about 15%, or about 20%. In some embodiments, the propylene glycol is present in an amount of at most about 2%, about 5%, about 8%, about 10%, about 12%, about 15%, about 20%, or about 25%. In some embodiments, the propylene glycol is present in an amount of about 1% to about 10%. In some embodiments, the propylene glycol is present in an amount of about 1% to about 2%, about 1% to about 4%, about 1% to about 6%, about 1% to about 8%, about 1% to about 10%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10%. In some embodiments, the propylene glycol is present in an amount of about 1%, about 2%, about 4%, about 6%, about 8%, or about 10%. In some embodiments, the propylene glycol is present in an amount of at least about 1%, about 2%, about 4%, about 6%, or about 8%. In some embodiments, the propylene glycol is present in an amount of at most about 2%, about 4%, about 6%, about 8%, or about 10%. In some embodiments, propylene glycol is present in about the same amount as the vesicle forming lipid. In some embodiments, the ratio of propylene glycol to vesicle forming lipid in the composition is form about 2:1 to about 1:2 (w/w).

In some embodiments, the butanediol (e.g., 2,3-butanediol) is present in an amount of about 0.5% to about 25% (w/w) of the composition. In some embodiments, the butanediol is present in an amount of about 0.5% to about 2%, about 0.5% to about 5%, about 0.5% to about 8%, about 0.5% to about 10%, about 0.5% to about 12%, about 0.5% to about 15%, about 0.5% to about 20%, about 0.5% to about 25%, about 2% to about 5%, about 2% to about 8%, about 2% to about 10%, about 2% to about 12%, about 2% to about 15%, about 2% to about 20%, about 2% to about 25%, about 5% to about 8%, about 5% to about 10%, about 5% to about 12%, about 5% to about 15%, about 5% to about 20%, about 5% to about 25%, about 8% to about 10%, about 8% to about 12%, about 8% to about 15%, about 8% to about 20%, about 8% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25%. In some embodiments, the butanediol is present in an amount of about 0.5%, about 2%, about 5%, about 8%, about 10%, about 10.5%, about 12%, about 15%, about 20%, or about 25%. In some embodiments, the butanediol is present in an amount of at least about 0.5%, about 2%, about 5%, about 8%, about 10%, about 12%, about 15%, or about 20%. In some embodiments, the butanediol is present in an amount of at most about 2%, about 5%, about 8%, about 10%, about 12%, about 15%, about 20%, or about 25%. In some embodiments, the butanediol is present in an amount of about 1% to about 10%. In some embodiments, the butanediol is present in an amount of about 1% to about 2%, about 1% to about 4%, about 1% to about 6%, about 1% to about 8%, about 1% to about 10%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10%. In some embodiments, the butanediol is present in an amount of about 1%, about 2%, about 4%, about 6%, about 8%, or about 10%. In some embodiments, the butanediol is present in an amount of at least about 1%, about 2%, about 4%, about 6%, or about 8%. In some embodiments, the butanediol is present in an amount of at most about 2%, about 4%, about 6%, about 8%, or about 10%. In some embodiments, butanediol is present in about the same amount as the vesicle forming lipid. In some embodiments, the ratio of butanediol to vesicle forming lipid in the composition is form about 2:1 to about 1:2 (w/w).

In some embodiments, the oil phase of the lipid vesicle and/or the lipid vesicle portion of the composition comprises a sterol. In some embodiments, the sterol is a naturally derived sterol. In some embodiments, the sterol is a synthetic sterol. In some embodiments, the sterol is cholesterol. In some embodiments, the cholesterol may be plant-derived cholesterol. In some embodiments, the cholesterol may be synthetic cholesterol. In some embodiments the plant-derived cholesterol may be cholest-5-en-3-ol (e.g., PhytoChol®, SyntheChol®, Avanti #700100), or any other plant-derived cholesterol, or any combination thereof. In some embodiments, the sterol may be phytosterol or a derivative thereof. In some embodiments, the phytosterol or derivative thereof may be phytosterol MM, Advasterol™ 90 IP or 95 IP F, NET Sterol-ISO, Canola Sterols, sitosterol 700095, lanosterol-95, brassicasterol, or any combination thereof.

In some embodiments, the sterol is present in an amount of about 0.1% to about 5% (w/w) of the composition. In some embodiments, the sterol is present in an amount of about 0.1% to about 5%. In some embodiments, the sterol is present in an amount of about 0.1% to about 0.2%, about 0.1% to about 0.5%, about 0.1% to about 0.8%, about 0.1% to about 1%, about 0.1% to about 2%, about 0.1% to about 3%, about 0.1% to about 4%, about 0.1% to about 5%, about 0.2% to about 0.5%, about 0.2% to about 0.8%, about 0.2% to about 1%, about 0.2% to about 2%, about 0.2% to about 3%, about 0.2% to about 4%, about 0.2% to about 5%, about 0.5% to about 0.8%, about 0.5% to about 1%, about 0.5% to about 2%, about 0.5% to about 3%, about 0.5% to about 4%, about 0.5% to about 5%, about 0.8% to about 1%, about 0.8% to about 2%, about 0.8% to about 3%, about 0.8% to about 4%, about 0.8% to about 5%, about 1% to about 2%, about 1% to about 3%, about 1% to about 4%, about 1% to about 5%, about 2% to about 3%, about 2% to about 4%, about 2% to about 5%, about 3% to about 4%, about 3% to about 5%, or about 4% to about 5%. In some embodiments, the sterol is present in an amount of about 0.1%, about 0.2%, about 0.5%, about 0.8%, about 1%, about 2%, about 3%, about 4%, or about 5%. In some embodiments, the sterol is present in an amount of at least about 0.1%, about 0.2%, about 0.5%, about 0.8%, about 1%, about 2%, about 3%, or about 4%. In some embodiments, the sterol is present in an amount of at most about 0.2%, about 0.5%, about 0.8%, about 1%, about 2%, about 3%, about 4%, or about 5%. In some embodiments, the sterol is present in an amount of about 1% to about 1.5%, about 1% to about 2%, about 1% to about 2.5%, about 1% to about 3%, about 1% to about 4%, about 1% to about 5%, about 1.5% to about 2%, about 1.5% to about 2.5%, about 1.5% to about 3%, about 1.5% to about 4%, about 1.5% to about 5%, about 2% to about 2.5%, about 2% to about 3%, about 2% to about 4%, about 2% to about 5%, about 2.5% to about 3%, about 2.5% to about 4%, about 2.5% to about 5%, about 3% to about 4%, about 3% to about 5%, or about 4% to about 5% (w/w) of the composition. In some embodiments, the sterol is present in an amount of about 1%, about 1.5%, about 2%, about 2.5%, about 3%, about 4%, or about 5% (w/w) of the composition. In some embodiments, the sterol is present in an amount of at least about 1%, about 1.5%, about 2%, about 2.5%, about 3%, or about 4% (w/w) of the composition. In some embodiments, the sterol is present in an amount of at most about 1.5%, about 2%, about 2.5%, about 3%, about 4%, or about 5% (w/w) of the composition.

### Oil Phases

The lipid vesicle compositions provided herein comprise an oil-in-water emulsion. The oil component is selected such that the material is a liquid at operative temperatures (e.g., room temperature) and is non-miscible with water.

Any suitable oil may be used as the oil phase. In some embodiments, the oil comprises a naturally occurring oil. In some embodiments, the naturally occurring oil is derived from one or more plants or plant parts (e.g., seeds or nuts). In some embodiments, the oil is a naturally occurring oil such as olive oil, vegetable oil, sunflower oil, or other similar plant derived oil.

In some embodiments, the oil phase is selected from the group consisting of vegetable oils, mono-, di-, and triglycerides, silicone fluids, mineral oils, and combinations thereof. In some embodiments, the oil phase comprises an emollient. In some embodiments, the emollient comprises caprylic and/or capric triglycerides (e.g., Labrafac CC). In some embodiments, the emollient comprises a natural oil-soluble emollient, such an oil-soluble plant extracts, essential oils, vegetable oils, vegetable butters, or any combination thereof. In some embodiments, the natural oil-soluble emollient comprise oils from sunflowers. In some embodiments, the natural oil-soluble emollient comprise oils from avocados.

In some embodiments, the oil comprises a silicon oil or derivative, such as dimethicone. In some embodiments, the oil silicon oil comprises a siloxane polymer. In some embodiments, the siloxane polymer comprises C1-C3 substituents. In some embodiments, the siloxane is polydimethylsiloxane (PDMS). In some embodiments, the silicon oil is polymethylsilsesquioxane (e.g., Botanisil™ SP-360). In some embodiments, the oil is a mixture which comprises a silicon oil (e.g., dimethicone) as a smaller component. In some embodiments, the silicon oil is incorporated in order to enhance the feel of the resulting composition or as a moisturizer. In some embodiments, the oil comprises a silicon oil in an amount of up to about 5%, up to about 4%, up to about 3%, up to about 2%, or up to about 1%. In some embodiments, the silicon oil is present in an amount of from about 0.1% to about 2% (w/w) of the composition. In some embodiments, the silicon oil is present in an amount of from about 0.1% to about 0.5%, 0.1% to about 0.7%, 0.1% to about 1%, 0.1% to about 1.5%, 0.15% to about 2%, 0.5% to about 0.7%, 0.5% to about 1%, 0.5% to about 1.5%, 0.5% to about 2%, 0.7% to about 1%, 0.7% to about 1.5%, 0.7% to about 2%, 1% to about 1.5%, or 1% to about 2% (w/w) of the composition. In some embodiments, the silicon oil is present in an amount of about 0.1%, 0.5%, 0.6%, 0.7%, 1%, 1.5%, or 2% of the composition.

In some embodiments, the oils are present in an amount of about 1% to about 35% (w/w) of the composition. In some embodiments, the oils are present in an amount of about 1% to about 5%, about 1% to about 10%, about 1% to about 15%, about 1% to about 20%, about 1% to about 25%, about 1% to about 30%, about 1% to about 35%, about 5% to about 10%, about 5% to about 15%, about 5% to about 20%, about 5% to about 25%, about 5% to about 30%, about 5% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30%, about 25% to about 35%, or about 30% to about 35%. In some embodiments, the oils are present in an amount of about 1%, about 5%, about 10%, about 15%, about 20%, about 25%, about 30%, or about 35%. In some embodiments, the oils are present in an amount of at least about 1%, about 5%, about 10%, about 15%, about 20%, about 25%, or about 30%. In some embodiments, the oils are present in an amount of at most about 5%, about 10%, about 15%, about 20%, about 25%, about 30%, or about 35%. In some embodiments, the oils are present in an amount of about 5% to about 15%. In some embodiments, the oils are present in an amount of about 5% to about 8%, about 5% to about 9%, about 5% to about 10%, about 5% to about 11%, about 5% to about 12%, about 5% to about 13%, about 5% to about 14%, about 5% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or about 14% to about 15%. In some embodiments, the oils are present in an amount of about 5%, about 8%, about 9%, about 10%, about 11%, about 12%, about 13%, about 14%, or about 15%. In some embodiments, the oils are present in an amount of at least about 5%, about 8%, about 9%, about 10%, about 11%, about 12%, about 13%, or about 14%. In some embodiments, the oils are present in an amount of at most about 8%, about 9%, about 10%, about 11%, about 12%, about 13%, about 14%, or about 15%.

In some embodiments, the oil comprises one or more triglycerides. In some embodiments the triglyceride is a medium chain triglyceride. In some embodiments, the medium chain triglyceride comprises fatty acid esters having a chain length of C₆-C₁₂.

In some embodiments, the triglyceride is present in an amount of about 1% to about 35% (w/w) of the composition. In some embodiments, the triglyceride is present in an amount of about 1% to about 5%, about 1% to about 10%, about 1% to about 15%, about 1% to about 20%, about 1% to about 25%, about 1% to about 30%, about 1% to about 35%, about 5% to about 10%, about 5% to about 15%, about 5% to about 20%, about 5% to about 25%, about 5% to about 30%, about 5% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30%, about 25% to about 35%, or about 30% to about 35%. In some embodiments, the triglyceride is present in an amount of about 1%, about 1.5%, about 2%, about 2.5%, about 3%, about 3.5%, about 4%, about 4.5%, about 5%, about 10%, about 15%, about 20%, about 25%, about 30%, or about 35%. In some embodiments, the triglyceride is present in an amount of at least about 1%, about 1.5%, about 2%, about 2.5%, about 3%, about 3.5%, about 4%, about 4.5%, about 5%, about 10%, about 15%, about 20%, about 25%, or about 30%. In some embodiments, the triglyceride is present in an amount of at most about 1.5%, about 2%, about 2.5%, about 3%, about 3.5%, about 4%, about 4.5%, about 5%, about 10%, about 15%, about 20%, about 25%, about 30%, or about 35%.

In some embodiments, the oil phase of the lipid vesicle and/or the lipid vesicle portion of the composition comprises a sterol. In some embodiments, the sterol is cholesterol. In some embodiments, the cholesterol may be plant-derived cholesterol. In some embodiments, the plant-derived cholesterol may be PhytoChol®, SyntheChol®, or any other plant-derived cholesterol (e.g., Avanti #700100), or any combination thereof. In some embodiments, the sterol may be phytosterol or a derivative thereof. In some embodiments, the phytosterol or derivative thereof may be phytosterol MM, Advasterol™ 90 IP or 95 IP F, NET Sterol-ISO, canola sterols, sitosterol 700095, lanosterol-95, brassicasterol, or any combination thereof.

In some embodiments, the sterol is present in an amount of about 0.1% to about 5% (w/w) of the composition. In some embodiments, the sterol is present in an amount of about 0.1% to about 5%. In some embodiments, the sterol is present in an amount of about 0.1% to about 0.2%, about 0.1% to about 0.5%, about 0.1% to about 0.8%, about 0.1% to about 1%, about 0.1% to about 2%, about 0.1% to about 3%, about 0.1% to about 4%, about 0.1% to about 5%, about 0.2% to about 0.5%, about 0.2% to about 0.8%, about 0.2% to about 1%, about 0.2% to about 2%, about 0.2% to about 3%, about 0.2% to about 4%, about 0.2% to about 5%, about 0.5% to about 0.8%, about 0.5% to about 1%, about 0.5% to about 2%, about 0.5% to about 3%, about 0.5% to about 4%, about 0.5% to about 5%, about 0.8% to about 1%, about 0.8% to about 2%, about 0.8% to about 3%, about 0.8% to about 4%, about 0.8% to about 5%, about 1% to about 2%, about 1% to about 3%, about 1% to about 4%, about 1% to about 5%, about 2% to about 3%, about 2% to about 4%, about 2% to about 5%, about 3% to about 4%, about 3% to about 5%, or about 4% to about 5%. In some embodiments, the sterol is present in an amount of about 0.1%, about 0.2%, about 0.5%, about 0.8%, about 1%, about 2%, about 3%, about 4%, or about 5%. In some embodiments, the sterol is present in an amount of at least about 0.1%, about 0.2%, about 0.5%, about 0.8%, about 1%, about 2%, about 3%, or about 4%. In some embodiments, the sterol is present in an amount of at most about 0.2%, about 0.5%, about 0.8%, about 1%, about 2%, about 3%, about 4%, or about 5%. In some embodiments, the sterol is present in an amount of about 1% to about 1.5%, about 1% to about 2%, about 1% to about 2.5%, about 1% to about 3%, about 1% to about 4%, about 1% to about 5%, about 1.5% to about 2%, about 1.5% to about 2.5%, about 1.5% to about 3%, about 1.5% to about 4%, about 1.5% to about 5%, about 2% to about 2.5%, about 2% to about 3%, about 2% to about 4%, about 2% to about 5%, about 2.5% to about 3%, about 2.5% to about 4%, about 2.5% to about 5%, about 3% to about 4%, about 3% to about 5%, or about 4% to about 5% (w/w) of the composition. In some embodiments, the sterol is present in an amount of about 1%, about 1.5%, about 2%, about 2.5%, about 3%, about 4%, or about 5% (w/w) of the composition. In some embodiments, the sterol is present in an amount of at least about 1%, about 1.5%, about 2%, about 2.5%, about 3%, or about 4% (w/w) of the composition. In some embodiments, the sterol is present in an amount of at most about 1.5%, about 2%, about 2.5%, about 3%, about 4%, or about 5% (w/w) of the composition.

In some embodiments, the lipid vesicle compositions comprise one or more penetration enhancers. In some embodiments, a penetration enhancer acts to increase the amount of penetration of the anionic polymer material through one or more layers of skin when applied to the skin of an individual. In some embodiments, a penetration enhancer acts to increase the amount of penetration of one or more peptides through one or more layers of skin when applied to the skin of an individual.

In some embodiments, a composition comprising one or more penetration enhancers has an increase in penetration through one or more layers of the skin when applied to a skin sample or the skin of an individual by at least 5%, 10%, 15%, 20%, 25%, 30%, 40%, 50%, 60%, 70%, 80%, 90%, 100%, 120%, 150%, or 200% compared to an otherwise same or similar composition without the one or more penetration enhancers when applied one or more layers of the skin of a similar or same skin sample or skin of an individual. In some embodiments, a composition comprising one or more penetration enhancers has an increase in penetration through one or more layers of the skin when applied to a skin sample or the skin of an individual by greater than 5%, 10%, 15%, 20%, 25%, 30%, 40%, 50%, 60%, 70%, 80%, 90%, 100%, 120%, 150%, or 200% compared to an otherwise same or similar composition without the one or more penetration enhancers when applied one or more layers of the skin of a similar or same skin sample or skin of an individual. In some embodiments, a composition comprising one or more penetration enhancers has an increase in penetration through one or more layers of the skin when applied to a skin sample or the skin of an individual by about 5%, 10%, 15%, 20%, 25%, 30%, 40%, 50%, 60%, 70%, 80%, 90%, 100%, 120%, 150%, or 200% compared to an otherwise same or similar composition without the one or more penetration enhancers when applied one or more layers of the skin of a similar or same skin sample or skin of an individual.

In some embodiments, a composition comprising one or more non-ionic surfactants with an HLB value of about 10 or less has an increase in penetration through one or more layers of the skin when applied to a skin sample or the skin of an individual by at least 5%, 10%, 15%, 20%, 25%, 30%, 40%, 50%, 60%, 70%, 80%, 90%, 100%, 120%, 150%, or 200% compared to an otherwise same or similar composition without the one or more non-ionic surfactants with an HLB value of about 10 or less when applied one or more layers of the skin of a similar or same skin sample or skin of an individual. In some embodiments, a composition comprising one or more non-ionic surfactants with an HLB value of about 10 or less has an increase in penetration through one or more layers of the skin when applied to a skin sample or the skin of an individual by greater than 5%, 10%, 15%, 20%, 25%, 30%, 40%, 50%, 60%, 70%, 80%, 90%, 100%, 120%, 150%, or 200% compared to an otherwise same or similar composition without the one or more non-ionic surfactants with an HLB value of about 10 or less when applied one or more layers of the skin of a similar or same skin sample or skin of an individual. In some embodiments, a composition comprising one or more non-ionic surfactants with an HLB value of about 10 or less has an increase in penetration through one or more layers of the skin when applied to a skin sample or the skin of an individual by about 5%, 10%, 15%, 20%, 25%, 30%, 40%, 50%, 60%, 70%, 80%, 90%, 100%, 120%, 150%, or 200% compared to an otherwise same or similar composition without the one or more non-ionic surfactants with an HLB value of about 10 or less when applied one or more layers of the skin of a similar or same skin sample or skin of an individual.

In some embodiments, the penetration enhancer is included in the oil-in-water emulsion of the composition. In some embodiments, the penetration enhancer is included in the lipid bilayer of the composition.

There are many types of penetration enhancing agents that may be employed. In some embodiments, the penetration enhancing agent comprising an ionic surfactant, a nonionic surfactant, or a combination thereof. In some embodiments, an ionic surfactant, a non-ionic surfactant, or a combination thereof is not a penetration enhancing agent.

In some embodiments, the penetration enhancing agent comprises a non-ionic surfactant or a combination of non-ionic surfactants. In some embodiments, the penetration enhancing agent is a single non-ionic surfactant. In some embodiments, the penetration enhancing agent is a combination of at least 2, 3, 4, or more non-ionic surfactants. In some embodiments, the penetration enhancing agent is a combination 2 non-ionic surfactants. In some embodiments, the penetration enhancing agent is a combination 3 non-ionic surfactants.

In some embodiments, the non-ionic surfactant or combination of non-ionic surfactants is selected from polyethylene glycol ethers of fatty alcohols, sorbitan esters, polysorbates, and polyethylene glycol fatty acid esters, glycerol stearate and combinations thereof.

In some embodiments, the combination of non-ionic surfactants is a combination of a polyethylene glycol ether of a fatty alcohol and a sorbitan ester. In some embodiments, the combination of non-ionic surfactants is a combination of a polyethylene glycol ethers of fatty alcohol and a polysorbate. In some embodiments, the combination of non-ionic surfactants is a combination of a polyethylene glycol ethers of fatty alcohol and a sorbitan ester. In some embodiments, the combination of non-ionic surfactants is a combination of a polyethylene glycol ethers of fatty alcohol and a polyethylene glycol fatty acid ester. In some embodiments, the combination of non-ionic surfactants is a combination of a polyethylene glycol ether of a fatty alcohol, a sorbitan ester, and a polysorbate. In some embodiments, the combination of non-ionic surfactants is a combination of a polyethylene glycol ether of a fatty alcohol, a sorbitan ester, and a polyethylene glycol fatty acid ester. In some embodiments, the combination of non-ionic surfactants is a combination of a polyethylene glycol ether of a fatty alcohol, a polysorbate, and a polyethylene glycol fatty acid ester. In some embodiments, the combination of non-ionic surfactants is a combination of a polyethylene glycol ether of a fatty alcohol, a polysorbate, and glycerol stearate.

In some embodiments, the combination of non-ionic surfactants comprises a polyethylene glycol fatty acid ester and a sorbitan ester. In some embodiments, the combination of non-ionic surfactants comprises a polyethylene glycol fatty acid ester and a polysorbate. In some embodiments, the combination of non-ionic surfactants is a combination of a polyethylene glycol fatty acid ester, a polysorbate, and a sorbitan ester.

In some embodiments, the non-ionic surfactant comprises a polyethylene glycol (PEG) ether of a fatty alcohol. In some embodiments, the PEG ether of the fatty alcohol comprises from about 2 to about 8 PEG groups and a C₁₂-C₂₂ fatty alcohol. In some embodiments, the polyethylene glycol ether of a fatty alcohol comprises diethylene glycol hexadecyl ether, 2-(2-octadecoxyethoxy)ethanol, diethylene glycol monooleyl ether, polyoxyethylene (2) oleyl ether, polyoxyethylene (3) oleyl ether, or polyoxyethylene (5) oleyl ether, or any combination thereof. In some embodiments, the polyethylene glycol ether of a fatty alcohol comprises 2-(2-octadecoxyethoxy)ethanol. In some embodiments, the PEG ether of a fatty alcohol is super refined Brij® 02, or a derivative thereof. In some embodiments, the non-ionic surfactant is added to the aqueous phase of the composition.

In some embodiments, the PEG ether of the fatty alcohol is present in an amount of from about 0.5% to about 10% (w/w) of the composition. In some embodiments, the PEG ether of the fatty alcohol is present in an amount of about 0.5% to about 2.5%. In some embodiments, the PEG ether of the fatty alcohol is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the PEG ether of the fatty alcohol is present in an amount of about 0.5%, about 0.8%, about 1%, about 1.2%, about 1.5%, about 2%, or about 2.5%. In some embodiments, the PEG ether of the fatty alcohol is present in an amount of at least about 0.5%, about 0.8%, about 1%, about 1.2%, about 1.5%, or about 2%. In some embodiments, the PEG ether of the fatty alcohol is present in an amount of at most about 0.8%, about 1%, about 1.2%, about 1.5%, about 2%, or about 2.5%.

In some embodiments, the non-ionic surfactant comprises a sorbitan ester. In some embodiments, the sorbitan ester is a fatty acid ester. In some embodiments, the sorbitan ester is a C₁₂-C₂₂ fatty acid ester. In some embodiments, the sorbitan ester comprises sorbitan monolaurate, sorbitan monopalmitate, sorbitan monostearate, sorbitan monooleate, sorbitan trioleate, sorbitan sesquioleate, or sorbitan isostearate, or any combinations thereof. In some embodiments, the sorbitan ester comprises sorbitan monolaurate. In some embodiments, the sorbitan ester comprises sorbitan monopalmitate. In some embodiments, the sorbitan ester comprises sorbitan monostearate. In some embodiments, the sorbitan ester comprises sorbitan monooleate. In some embodiments, the sorbitan ester comprises sorbitan trioleate. In some embodiments, the sorbitan ester comprises sorbitan sesquioleate. In some embodiments, the sorbitan ester comprises sorbitan isostearate.

In some embodiments, the sorbitan ester is present in an amount of about 0.5% to about 2.5% (w/w) of the composition. In some embodiments, the sorbitan ester is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the sorbitan ester is present in an amount of about 0.5%, about 0.8%, about 1%, about 1.2%, about 1.5%, about 2%, or about 2.5%. In some embodiments, the sorbitan ester is present in an amount of at least about 0.5%, about 0.8%, about 1%, about 1.2%, about 1.5%, or about 2%. In some embodiments, the sorbitan ester is present in an amount of at most about 0.8%, about 1%, about 1.2%, about 1.5%, about 2%, or about 2.5%.

In some embodiments, the non-ionic surfactant comprises a polysorbate. In some embodiments, the polysorbate comprises polysorbate 20, polysorbate 21, polysorbate 40, polysorbate 60, polysorbate 80, polysorbate 85, or any combination thereof. In some embodiments, the polysorbate is polysorbate 80. In some embodiments, the polysorbate is polysorbate 20.

In some embodiments, the polysorbate is present in an amount of about 0.5% to about 2.5% (w/w) of the composition. In some embodiments, the polysorbate is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the polysorbate is present in an amount of about 0.5%, about 0.8%, about 1%, about 1.2%, about 1.5%, about 2%, or about 2.5%. In some embodiments, the polysorbate is present in an amount of at least about 0.5%, about 0.8%, about 1%, about 1.2%, about 1.5%, or about 2%. In some embodiments, the polysorbate is present in an amount of at most about 0.8%, about 1%, about 1.2%, about 1.5%, about 2%, or about 2.5%.

In some embodiments, the non-ionic surfactant comprises a polyethylene glycol (PEG) fatty acid ester. In some embodiments, the PEG fatty acid ester is a PEG chain of about 2-8 subunits comprising C₈-C₂₂ fatty acids affixed to each terminal hydroxyl to form the fatty acid ester. In some embodiments, the PEG fatty acid ester comprises PEG-8 dilaurate, PEG-4 dilaurate, PEG-4 laurate, PEG-8 dioleate, PEG-8 distearate, PEG-8 distearate, PEG-7 glyceryl cocoate, and PEG-20 almond glycerides, or any combination thereof. In some embodiments, the PEG fatty acid ester is PEG-4 dilaurate.

In some embodiments, the PEG fatty acid ester is present in an amount of about 0.5% to about 2.5% (w/w) of the composition. In some embodiments, the PEG fatty acid ester is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the PEG fatty acid ester is present in an amount of about 0.5%, about 0.8%, about 1%, about 1.2%, about 1.5%, about 2%, or about 2.5%. In some embodiments, the PEG fatty ester is present in an amount of at least about 0.5%, about 0.8%, about 1%, about 1.2%, about 1.5%, or about 2%. In some embodiments, the PEG fatty acid ester is present in an amount of at most about 0.8%, about 1%, about 1.2%, about 1.5%, about 2%, or about 2.5%.

In some embodiments, the non-ionic surfactant has a hydrophobic-lipophilic balance (HLB) of about 10 or less. In some embodiments, the composition comprises a plurality of non-ionic surfactants, each having an HLB of about 10 or less. In some embodiments, the non-ionic surfactant with an HLB of 10 or less is selected from the Table 1, or any combination thereof.

In some embodiments, the non-ionic surfactant or combination of non-ionic surfactants are present in an amount of about 0.5% to about 10% (w/w) of the composition. In some embodiments, the non-ionic surfactant or combination of non-ionic surfactants are present in an amount of about 0.5% to about 1%, about 0.5% to about 1.5%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 10%, about 1% to about 1.5%, about 1% to about 2%, about 1% to about 3%, about 1% to about 4%, about 1% to about 5%, about 1% to about 6%, about 1% to about 7%, about 1% to about 8%, about 1% to about 10%, about 1.5% to about 2%, about 1.5% to about 3%, about 1.5% to about 4%, about 1.5% to about 5%, about 1.5% to about 6%, about 1.5% to about 7%, about 1.5% to about 8%, about 1.5% to about 10%, about 2% to about 3%, about 2% to about 4%, about 2% to about 5%, about 2% to about 6%, about 2% to about 7%, about 2% to about 8%, about 2% to about 10%, about 3% to about 4%, about 3% to about 5%, about 3% to about 6%, about 3% to about 7%, about 3% to about 8%, about 3% to about 10%, about 4% to about 5%, about 4% to about 6%, about 4% to about 7%, about 4% to about 8%, about 4% to about 10%, about 5% to about 6%, about 5% to about 7%, about 5% to about 8%, about 5% to about 10%, about 6% to about 7%, about 6% to about 8%, about 6% to about 10%, about 7% to about 8%, about 7% to about 10%, or about 8% to about 10%. In some embodiments, the non-ionic surfactant or combination of non-ionic surfactants are present in an amount of about 0.5%, about 0.6%, about 0.7%, about 1%, about 1.5%, about 2%, about 3%, about 4%, about 5%, about 6%, about 7%, about 8%, or about 10%. In some embodiments, the non-ionic surfactant or combination of non-ionic surfactants are present in an amount of about 0.6% (e.g., 0.54% to about 0.66%). In some embodiments, the non-ionic surfactant or combination of non-ionic surfactants are present in an amount of about 0.7% (e.g, 0.63% to about 0.77%). In some embodiments, the non-ionic surfactant or combination of non-ionic surfactants are present in an amount of at least about 0.5%, about 0.7%, about 1%, about 1.5%, about 2%, about 3%, about 4%, about 5%, about 6%, about 7%, or about 8%. In some embodiments, the non-ionic surfactant or combination of non-ionic surfactants are present in an amount of at most about 0.7%, about 1%, about 1.5%, about 2%, about 3%, about 4%, about 5%, about 6%, about 7%, about 8%, or about 10%.

In some embodiments, the composition comprises a non-ionic surfactant in the oil-in-water emulsion, the lipid bilayer, or both. In some embodiments, the composition comprises a non-ionic surfactant in the oil-in-water emulsion. In some embodiments, the composition comprises a non-ionic surfactant in the lipid bilayer. In some embodiments, the composition comprises a non-ionic surfactant in the oil-in-water emulsion and the lipid bilayer, wherein the composition comprises two or more different non-ionic surfactants.

In some embodiments, the penetration enhancing agent comprises a salicylate ester or a nicotinate ester. In some embodiments, the ester is a C₁-C₆ alkyl ester or a benzyl ester. In some embodiments, the penetration enhancing agent may be a vasodilator. In some embodiments, the penetration enhancing agent comprises methyl salicylate or benzyl nicotinate. In some embodiments, the penetration enhancing agent is a nicotinate ester present in an amount of up to about 0.1%, 0.5%, 1%, 2%, or 3% (w/w) of the composition. In some embodiments, the nicotinate ester is present in an amount of from about 0.1% to about 3%, about 0.1% to about 2%, or about 0.1% to about 1%. In some embodiments, benzyl nicotinate is present at an amount of about 0.5%.

In some embodiments, the penetration enhancing agent comprises a fatty acid acylated amino acid. In some embodiments, the fatty acid acylated amino acid is lysine. In some embodiments, the lysine is mono-acylated with a fatty acid. In some embodiments, the penetration enhancing agent is monoloauryl lysine. In some embodiments, the lysine is di-acylated. In some embodiments, the penetration enhancing agent is dipalmitoyllysine. In some embodiments, the fatty acylated amino acid is present in an amount of up to about 1%, up to about 2%, up to about 3%, up to about 4%, or up to about 5% (w/w) of the composition. In some embodiments, the fatty acylated amino acid is present in an amount of from about 0.1% to about 5%, from about 0.1% to about 4%, from about 0.1% to about 3%, from about 0.1% to about 2%, from about 0.5% to about 5%, from about 0.5% to about 4%, from about 0.5% to about 3%, from about 0.5% to about 2%, from about 1% to about 5%, from about 1% to about 4%, from about 1% to about 3%, from about 1% to about 2%, or from about 1.5% to about 2.5%.

### Cationic Surfactants

In some embodiments, the composition further comprises a cationic surfactant. In some embodiments, the cationic surfactant is used to stabilize the water-in-oil emulsion (e.g., at the submicron emulsion stage prior to lipid vesicle formation). In some embodiments, the cationic surfactant is a mono-cationic surfactant. In some embodiments, the mono-cationic surfactant is net-mono-cationic (e.g., a phosphate salt comprising two side chains each with a single cationic functionality, which is partially neutralized by a phosphate anion).

In some embodiments, the mono-cationic surfactant is a fatty-amide derived propylene glycol-diammonium phosphate ester. In some embodiments, the mono-cationic surfactant is linoleamidopropyl PG-dimonium chloride phosphate (e.g., Arlasilk™ PTM, Arlasilk™ EFA).

In some embodiments, the fatty amide derived propylene glycol-diammonium phosphate ester is present in an amount of about 1% to about 20% (w/w) of the composition. In some embodiments, the fatty amide derived propylene glycol-diammonium phosphate ester is present in an amount of about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 11%, about 1% to about 12%, about 1% to about 13%, about 1% to about 14%, about 1% to about 15%, about 1% to about 16%, about 1% to about 17%, about 1% to about 18%, about 1% to about 19%, about 1% to about 20%, about 2% to about 3%, about 2% to about 4%, about 2% to about 5%, about 2% to about 6%, about 2% to about 7%, about 2% to about 8%, about 2% to about 9%, about 2% to about 10%, about 2% to about 11%, about 2% to about 12%, about 2% to about 13%, about 2% to about 14%, about 2% to about 15%, about 2% to about 16%, about 2% to about 17%, about 2% to about 18%, about 2% to about 19%, about 2% to about 20%, about 3% to about 4%, about 3% to about 5%, about 3% to about 6%, about 3% to about 7%, about 3% to about 8%, about 3% to about 9%, about 3% to about 10%, about 3% to about 11%, about 3% to about 12%, about 3% to about 13%, about 3% to about 14%, about 3% to about 15%, about 3% to about 16%, about 3% to about 17%, about 3% to about 18%, about 3% to about 19%, about 3% to about 20%, about 4% to about 5%, about 4% to about 6%, about 4% to about 7%, about 4% to about 8%, about 4% to about 9%, about 4% to about 10%, about 4% to about 11%, about 4% to about 12%, about 4% to about 13%, about 4% to about 14%, about 4% to about 15%, about 4% to about 16%, about 4% to about 17%, about 4% to about 18%, about 4% to about 19%, about 4% to about 20%, about 5% to about 6%, about 5% to about 7%, about 5% to about 8%, about 5% to about 9%, about 5% to about 10%, about 5% to about 11%, about 5% to about 12%, about 5% to about 13%, about 5% to about 14%, about 5% to about 15%, about 5% to about 16%, about 5% to about 17%, about 5% to about 18%, about 5% to about 19%, about 5% to about 20%, about 6% to about 7%, about 6% to about 8%, about 6% to about 9%, about 6% to about 10%, about 6% to about 11%, about 6% to about 12%, about 6% to about 13%, about 6% to about 14%, about 6% to about 15%, about 6% to about 16%, about 6% to about 17%, about 6% to about 18%, about 6% to about 19%, about 6% to about 20%, about 7% to about 8%, about 7% to about 9%, about 7% to about 10%, about 7% to about 11%, about 7% to about 12%, about 7% to about 13%, about 7% to about 14%, about 7% to about 15%, about 7% to about 16%, about 7% to about 17%, about 7% to about 18%, about 7% to about 19%, about 7% to about 20%, about 8% to about 9%, about 8% to about 10%, about 8% to about 11%, about 8% to about 12%, about 8% to about 13%, about 8% to about 14%, about 8% to about 15%, about 8% to about 16%, about 8% to about 17%, about 8% to about 18%, about 8% to about 19%, about 8% to about 20%, about 9% to about 10%, about 9% to about 11%, about 9% to about 12%, about 9% to about 13%, about 9% to about 14%, about 9% to about 15%, about 9% to about 16%, about 9% to about 17%, about 9% to about 18%, about 9% to about 19%, about 9% to about 20%, about 10% to about 11%, about 10% to about 12%, about 10% to about 13%, about 10% to about 14%, about 10% to about 15%, about 10% to about 16%, about 10% to about 17%, about 10% to about 18%, about 10% to about 19%, about 10% to about 20%, about 11% to about 12%, about 11% to about 13%, about 11% to about 14%, about 11% to about 15%, about 11% to about 16%, about 11% to about 17%, about 11% to about 18%, about 11% to about 19%, about 11% to about 20%, about 12% to about 13%, about 12% to about 14%, about 12% to about 15%, about 12% to about 16%, about 12% to about 17%, about 12% to about 18%, about 12% to about 19%, about 12% to about 20%, about 13% to about 14%, about 13% to about 15%, about 13% to about 16%, about 13% to about 17%, about 13% to about 18%, about 13% to about 19%, about 13% to about 20%, about 14% to about 15%, about 14% to about 16%, about 14% to about 17%, about 14% to about 18%, about 14% to about 19%, about 14% to about 20%, about 15% to about 16%, about 15% to about 17%, about 15% to about 18%, about 15% to about 19%, about 15% to about 20%, about 16% to about 17%, about 16% to about 18%, about 16% to about 19%, about 16% to about 20%, about 17% to about 18%, about 17% to about 19%, about 17% to about 20%, about 18% to about 19%, about 18% to about 20%, or about 19% to about 20%. In some embodiments, the fatty amide derived propylene glycol-diammonium phosphate ester is present in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% (w/w) of the composition. In some embodiments, the fatty amide derived propylene glycol-diammonium phosphate ester is present in an amount of at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%. In some embodiments, the fatty amide derived propylene glycol-diammonium phosphate ester is present in an amount of at most about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

### Additional Components

In some embodiments, the vesicle composition comprises additional components. In some embodiments, these additional components improve one or more properties of the vesicles without dramatically altering the delivery of an anionic polymer material, peptide, or a combination thereof.

In some embodiments, the vesicle composition further comprises one or more viscosity enhancing agents. In some embodiments, the viscosity enhancing agents thicken the composition for increased stability and/or feel to a user of the vesicle composition. In some embodiments, the viscosity enhancing agents also act as surfactants. In some embodiments, the viscosity enhancing agent comprises one or more of a fatty alcohol, a wax, a fatty ester of glycerol, or any combination thereof. In some embodiments, the fatty alcohol is a C₈-C₂₀ fatty alcohol. In some embodiments, the fatty alcohol is cetyl alcohol (e.g., Crodacol C₉₅). In some embodiments, the wax is a naturally occurring or synthetic wax. In some embodiments, the wax is beeswax. In some embodiment, the wax is synthetic beeswax. In some embodiments, the synethetic beeswax is Syncrowax™ BB4. In some embodiments, the synthetic beeswax is non-animal derived beeswax. In some embodiments, the non-animal derived beeswax is Syncrowax™ SB1. In some embodiments, the fatty ester of glycerol is a monoester. In some embodiments, the monoester is an ester of a C₈-C₂₄ fatty acid. In some embodiments, the fatty ester of glycerol is glycerol monostearate.

In some embodiments, the viscosity enhancing agents are present in an amount of from about 0.5% to about 10% (w/w) of the composition. In some embodiments, the viscosity enhancing agents are present in an amount of from about 0.5% to about 5%, about 0.5% to about 5%, about 0.5% to about 4%, about 0.5% to about 3%, or from about 0.5% to about 2% (w/w) of the composition. In some embodiments, the viscosity enhancing agents comprise a fatty alcohol in an amount of up to about 2%, a wax in an amount of up to about 2%, and a fatty ester of glycerol in an amount of up to about 5%. In some embodiments, the fatty alcohol is present in an amount of from about 0.1 to about 1.5%. In some embodiments, the fatty alcohol is present in an amount of about 0.4% (e.g., 0.36% to about 0.44%). In some embodiments, the wax is present in an amount of from about 0.1% to about 1%. In some embodiments, the wax is present in an amount of about 0.2% (e.g., about 0.18% to about 0.22%). In some embodiments, the wax is present in an amount of about 0.3% (e.g., about 0.27% to about 0.33%). In some embodiments, the fatty ester of glycerol is present in an amount of from about 0.5% to about 2%. In some embodiments, the fatty ester of glycerol is present in an amount of about 0.8% (e.g., 0.72% to about 0.88%). In some embodiments, the fatty ester of glycerol is present in an amount of about 0.9%.

In some embodiments, the vesicle composition further comprises one or more of a thickener, a preservative, a moisturizer, an emollient, a humectant, or any combination thereof. In some embodiments, the vesicle composition further comprises a thickener. In some embodiments, the vesicle composition further comprises a preservative. In some embodiments, the vesicle composition further comprises a moisturizer. In some embodiments, the vesicle composition further comprises an emollient. In some embodiments, the vesicle composition further comprises a humectant. In some embodiments, the vesicle composition further comprises a fragrance (e.g., Mentha piperita). In some embodiments, the fragrance (e.g., Mentha piperita) is present in an amount of about 0.01% to about 0.1%. In some embodiments, the fragrance (e.g., Mentha piperita) is present in an amount of about 0.05%.

In some embodiments, the vesicle composition further comprises a humectant. In some embodiments, the composition comprises glycerol. In some embodiments, the glycerol is present in an amount of from about 0.5% to about 25%, about 0.5% to about 20%, about 0.5% to about 15%, or about 0.5% to about 10% (w/w) of the composition. In some embodiments, the glycerol is present in an amount of about 1% to about 10%. In some embodiments, the glycerol is present in an amount of about 1% to about 2%, about 1% to about 4%, about 1% to about 6%, about 1% to about 8%, about 1% to about 10%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10%. In some embodiments, the glycerol is present in an amount of about 1%, about 2%, about 4%, about 6%, about 8%, or about 10%. In some embodiments, the glycerol is present in an amount of at least about 1%, about 2%, about 4%, about 6%, or about 8%. In some embodiments, the glycerol is present in an amount of at most about 2%, about 4%, about 6%, about 8%, or about 10%.

In some embodiments, the vesicle composition further comprises a preservative. In some embodiments, the preservative is a paraben ester. In some embodiments, the preservative is methylparaben or propylparaben, or a combination thereof. In some embodiments, the preservative is a cosmetic preservative, such as Euxyl® PE 9010 or Spectrastat®. In some embodiments, the preservative comprises a phenoxyethanol/ethylhexylglycerin mixture. In some embodiments, the preservative comprises a blend of caprylhydroxamic acid, caprylyl glycol, and glycerin. In some embodiments, the preservative is present in an amount of up to about 1%, up to about 0.9%, up to about 0.8%, up to about 0.7%, up to about 0.6%, up to about 0.5%, up to about 0.4%, up to about 0.3%, up to about 0.2% (w/w) of the composition. In some embodiments, the preservative is present in an amount of about 0.1%, 0.2%, 0.3%, 0.4%, 0.5%, 0.55%, 0.6%, 0.65%, 0.7%, 0.75%, 0.8%, 0.85%, 0.9%, 0.95%, 1%, 1.05%, 1.1%, 1.2%, 1.3%, 1.4%, or 1.5%.

In some embodiments, the composition further comprises material to promote a desirable change in the skin, such as smoothing wrinkles. In some embodiments, the composition comprises one or more carbohydrates or an acceptable salts thereof. In some embodiments, the one or more carbohydrates is a monosaccharide, disaccharide, or polysaccharide. In some embodiments, a carbohydrate or acceptable salt thereof comprises a glucosamine phosphate. In some embodiments, the glucosamine phosphate comprises disodium acetyl glucosamine phosphate, such as acetyl N-Acetyl-α-D-glucosamine 6-phosphate disodium salt (NovHyal™). In some embodiments, a carbohydrate comprises a saccharide isomerate (e.g., Hyanify™). In some embodiments, the one or more carbohydrates or acceptable salts thereof is in a liquid form. In some embodiments, the one or more carbohydrates or acceptable salts thereof is in a gel form. In some embodiments, the one or more carbohydrates or acceptable salts thereof is in a solid form. In some embodiments, the one or more carbohydrates or an acceptable salts thereof is present in the composition in an amount of from about 0.1% to about 1%. In some embodiments, the one or more carbohydrates or an acceptable salts thereof is present in the composition in an amount of from about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.4% to about 0.5%, about 0.4% to about 0.6%, about 0.4% to about 0.7%, about 0.4% to about 0.8%, about 0.4% to about 0.9%, about 0.4% to about 1%, about 0.5% to about 0.6%, about 0.5% to about 0.7%, about 0.5% to about 0.8%, about 0.5% to about 0.9%, about 0.5% to about 1%, about 0.6% to about 0.7%, about 0.6% to about 0.8%, about 0.6% to about 0.9%, about 0.6% to about 1%, about 0.7% to about 0.8%, about 0.7% to about 0.9%, about 0.7% to about 1%, about 0.8% to about 0.9%, about 0.8% to about 1%, or about 0.9% to about 1%. In some embodiments, the one or more carbohydrates or an acceptable salts thereof is present in the composition in an amount of from about 0.1%, about 0.2%, about 0.3%, about 0.4%, about 0.5%, about 0.6%, about 0.7%, about 0.8%, about 0.9%, or about 1%. In some embodiments, the one or more carbohydrates or an acceptable salts thereof is present in the composition in an amount of from at least about 0.1%, about 0.2%, about 0.3%, about 0.4%, about 0.5%, about 0.6%, about 0.7%, about 0.8%, or about 0.9%. In some embodiments, the one or more carbohydrates or an acceptable salts thereof is present in the composition in an amount of from at most about 0.2%, about 0.3%, about 0.4%, about 0.5%, about 0.6%, about 0.7%, about 0.8%, about 0.9%, or about 1%. In some embodiments, the one or more carbohydrates or an acceptable salts thereof is present in the composition in an amount of about 0.1%, 0.2%, 0.3%, 0.4%, 0.5%, 0.6%, 0.7%, 0.8%, 0.9%, and 1%. In some embodiments, the one or more carbohydrates or an acceptable salts thereof is present in the composition in an amount of at least about 0.1%, 0.2%, 0.3%, 0.4%, 0.5%, 0.6%, 0.7%, 0.8%, 0.9%, and 1%. In some embodiments, the one or more carbohydrates or an acceptable salts thereof is present in the composition in an amount of at most about 0.1%, 0.2%, 0.3%, 0.4%, 0.5%, 0.6%, 0.7%, 0.8%, 0.9%, and 1%.

In some embodiments, the composition comprises a polymer of an amino acid. In some embodiments, the polymer of an amino acid comprises a high MW and a low MW polymer of the amino acid. In some embodiments, the polymer of an amino acid comprises polyglutamate. In some cases, the polyglutamate comprises low MW polyglutamate (e.g., Hyfactor™ PGA LM) and/or high MW polyglutamate (e.g., Hyfactor™ PGA HM). In some embodiments, the polymer of an amino acid is present in an amount from about 0.01% to about 0.2%. In some embodiments, the polymer of an amino acid is present in an amount from about 0.01% to about 0.05%, about 0.01% to about 0.1%, about 0.01% to about 0.15%, about 0.01% to about 0.2%, about 0.05% to about 0.1%, about 0.05% to about 0.15%, about 0.05% to about 0.2%, about 0.1% to about 0.15%, about 0.1% to about 0.2%, or about 0.15% to about 0.2%. In some embodiments, the polymer of an amino acid is present in an amount of about 0.01%, about 0.05%, about 0.1%, about 0.15%, or about 0.2%. In some embodiments, the polymer of an amino acid is present in an amount of at least about 0.01%, about 0.05%, about 0.1%, or about 0.15%. In some embodiments, the polymer of an amino acid is present in an amount of at most about 0.05%, about 0.1%, about 0.15%, or about 0.2%.

In some embodiments, the composition further comprises collagen. In some cases, the collagen in human collagen. In some cases, the collage is vegan human collagen (e.g., HumaColl21®). In some embodiments, the collagen is in a solid form. In some embodiments, the collagen is in a gel form. In some embodiments, the collagen is in a liquid form. In some embodiments, the collagen in present in an amount from about 0.01% to about 0.2%. In some embodiments, the collagen is present in an amount from about 0.01% to about 0.02%, about 0.01% to about 0.03%, about 0.01% to about 0.05%, about 0.01% to about 0.1%, about 0.01% to about 0.15%, about 0.01% to about 0.2%, about 0.02% to about 0.03%, about 0.02% to about 0.05%, about 0.02% to about 0.1%, about 0.02% to about 0.15%, about 0.02% to about 0.2%, about 0.05% to about 0.1%, about 0.05% to about 0.15%, about 0.05% to about 0.2%, about 0.1% to about 0.15%, about 0.1% to about 0.2%, or about 0.15% to about 0.2%. In some embodiments, the collagen is present in an amount of about 0.01%, about 0.02%, about 0.03%, about 0.04%, about 0.05%, about 0.1%, about 0.15%, or about 0.2%. In some embodiments, the collagen is present in an amount of at least about 0.01%, about 0.02%, about 0.03%, about 0.04%, about 0.05%, about 0.1%, or about 0.15%. In some embodiments, the collagen is present in an amount of at most about 0.02%, about 0.03%, about 0.04%, about 0.05%, about 0.1%, about 0.15%, or about 0.2%.

In some embodiments, the composition further comprises material to promote a desirable change in the skin, such as reducing darkness in one or more areas of the skin. In some embodiments, the material comprises one or more biological extracts. In some embodiments, the one or more biological extracts are natural extracts or synthetic extracts. In some embodiments, the one or more biological extracts comprises a carbohydrate. In some embodiments, the carbohydrate comprises fucoidan. Examples of biological extracts comprise, but are not limited to, an algae extract, a marine extract, a biotechnology extract, or a flower extract (e.g., Seanactiv, Shadownyl Clear, Eyedeline, Eye'fective, or Meiview).

In some embodiments, the additional components comprise purified water. In some embodiments, purified water is present in an amount of about 50% to 80% (w/w). In some embodiments, purified water is present in an amount of about 50% to about 55%, about 50% to about 60%, about 50% to about 65%, about 50% to about 70%, about 50% to about 75%, about 50% to about 80%, about 55% to about 60%, about 55% to about 65%, about 55% to about 70%, about 55% to about 75%, about 55% to about 80%, about 60% to about 65%, about 60% to about 70%, about 60% to about 75%, about 60% to about 80%, about 65% to about 70%, about 65% to about 75%, about 65% to about 80%, about 70% to about 75%, about 70% to about 80%, or about 75% to about 80%. In some embodiments, purified water is present in an amount of about 50%, about 55%, about 60%, about 65%, about 70%, about 75%, or about 80%. In some embodiments, purified water is present in an amount of at least about 50%, about 55%, about 60%, about 65%, about 70%, or about 75%. In some embodiments, purified water is present in an amount of at most about 55%, about 60%, about 65%, about 70%, about 75%, or about 80%.

### Exemplary Compositions for Delivery of Unmodified Peptides

Provided below are exemplary compositions for the delivery of one or more peptides. In some embodiments, the one or more peptides are unmodified. In some embodiments, the one or more peptides comprise dipeptides, tripeptides, tetrapeptides, pentapeptides, hexapeptides, heptapeptides, octapeptides, or any combination thereof. In some embodiments, the exemplary compositions deliver one, two, three, four, five, six, seven, or eight peptides. The embodiments below may further comprise other ingredients or components, such as those provided herein. In some embodiment, the other ingredients or components may be an anionic polymer material. In some embodiments, the anionic polymer material may be hyaluronic acid. In some embodiments, the hyaluronic acid may be entrapped in the lipid bilayer, the oil-in-water emulsion, or a combination thereof.

Peptide Composition 1: In one aspect, provided herein, is a lipid vesicle composition comprising
(a) lipid vesicles each comprising a lipid bilayer comprising vesicle forming lipids, wherein the vesicle forming lipids are present in an amount of from about 5% to about 20%;
(b) an oil-in-water emulsion entrapped in the lipid vesicles, stabilized by one or more surfactants; and
(c) one or more peptides in an amount of from about 0.1 mg/mL to about 50 mg/mL entrapped in the lipid bilayer, the oil-in-water emulsion, or a combination thereof,
wherein the composition further comprises:
   a fatty amide derived propylene glycol-diammonium phosphate ester in an amount of from about 1% to about 10%; and
   a non-ionic surfactant in an amount of from about 0.1% to about 3%.

In some embodiments, the oil component is present in an amount of from about 2.5% to about 20%.

In some embodiments, the lipid vesicle composition comprises one or more peptides in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL. In some embodiments the lipid vesicle composition comprises one or more peptides in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the lipid vesicle composition comprises one or more peptides in an amount of about 1 mg/mL, about 2 mg/mL, about 3 mg/mL, about 4 mg/mL, or about 5 mg/mL.

In some embodiments, the one or more peptides of (c) comprises a tetrapeptide, pentapeptide, a hexapeptide, or any combination thereof. In some embodiments, the one or more peptides comprises an amino acid sequence at least about 50%, at least about 60%, at least about 70%, at least about 80%, or at least about 90% sequence homology to the amino acid sequence of any one of SEQ ID NO: 1-51. In some embodiments, the one or more peptides comprises at least two, three, four, five, or six peptides each with an amino acid sequence at least about 90% sequence homology to the amino acid sequence of any one of SEQ ID NO: 1-51. In some embodiments, the one or more peptides comprises an amino acid sequence at least about 50%, at least about 60%, at least about 70%, at least about 80%, or at least about 90% sequence homology to the amino acid sequence of any one of SEQ ID NO: 21, 24, 31, 36, 37, 39, or 40. In some embodiments, the one or more peptides comprises an amino acid sequence identical to the amino acids of any one of SEQ ID NO: 21, 24, 31, 36, 37, 39, or 40. In some embodiments, the one or more peptides comprises an amino acid sequence at least about 50%, at least about 60%, at least about 70%, at least about 80%, or at least about 90% sequence homology to the amino acid sequence of SEQ ID NO: 37. In some embodiments, the one or more peptides comprises an amino acid sequence identical to the amino acids of SEQ ID NO: 37.

In some embodiments, the composition further comprises a fatty acylated amino acid in an amount of from about 0.5% to about 3%. In some embodiments, the fatty acylated amino acid is monoloauryl lysine.

In some embodiments, the lipid vesicle composition further comprises viscosity enhancing agents in an amount of from about 0.5% to about 5%. In some embodiments, the viscosity enhancing agents comprise one or more of a fatty alcohol, a wax, a fatty ester of glycerol, or any combination thereof.

In some embodiments, the non-ionic surfactant comprises a PEG ether of a fatty alcohol.

Peptide Composition 2: In one aspect, provided herein, is a lipid vesicle composition comprising
(a) lipid vesicles each comprising a lipid bilayer comprising vesicle forming lipids, wherein the vesicle forming lipids are present in an amount of from about 2% to about 20%;
(b) an oil-in-water emulsion entrapped in the lipid vesicles, and stabilized by one or more surfactants;
(c) one or more peptides in an amount of from about 0.1 mg/mL to about 50 mg/mL entrapped in the lipid bilayer, the oil-in-water emulsion, or a combination thereof,
wherein the composition further comprises:
   a PEG fatty acid ester in an amount of from about 0.1% to about 2%;
   a polysorbate in an amount of from about 0.5% to about 3%; and
   a sorbate ester in an amount of from about 0.1% to about 2%.

In some embodiments, the oil component is present in an amount of from about 2.5% to about 20%.

In some embodiments, the lipid vesicle composition comprises one or more peptides in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL. In some embodiments the lipid vesicle composition comprises one or more peptides in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the lipid vesicle composition comprises one or more peptides in an amount of about 1 mg/mL, about 2 mg/mL, about 3 mg/mL, about 4 mg/mL, or about 5 mg/mL.

In some embodiments, the one or more peptides of (c) comprises a tetrapeptide, pentapeptide, hexapeptide, or a combination thereof. In some embodiments, the one or more peptides comprises an amino acid sequence at least about 50%, at least about 60%, at least about 70%, at least about 80%, or at least about 90% sequence homology to the amino acid sequence of any one of SEQ ID NO: 1-51. In some embodiments, the one or more peptides comprises at least two, three, four, five, or six peptides each with an amino acid sequence at least about 90% sequence homology to the amino acid sequence of any one of SEQ ID NO: 1-51. In some embodiments, the one or more peptides comprises an amino acid sequence at least about 50%, at least about 60%, at least about 70%, at least about 80%, or at least about 90% sequence homology to the amino acid sequence of any one of SEQ ID NO: 21, 24, 31, 36, 37, 39, or 40. In some embodiments, the one or more peptides comprises an amino acid sequence identical to the amino acids of any one of SEQ ID NO: 21, 24, 31, 36, 37, 39, or 40. In some embodiments, the one or more peptides comprises an amino acid sequence at least about 50%, at least about 60%, at least about 70%, at least about 80%, or at least about 90% sequence homology to the amino acid sequence of SEQ ID NO: 37. In some embodiments, the one or more peptides comprises an amino acid sequence identical to the amino acids of SEQ ID NO: 37.

In some embodiments, the lipid vesicle composition further comprises viscosity enhancing agents in an amount of from about 0.5% to about 5%. In some embodiments, the viscosity enhancing agents comprise one or more of a fatty alcohol, a wax, a fatty ester of glycerol, or any combination thereof.

In some embodiments, the PEG fatty acid ester comprises PEG4-dilaurate. In some embodiments, the polysorbate is polysorbate 80. In some embodiments, the sorbate ester is sorbitan palmitate.

Peptide Composition 3: In one aspect, provided herein, is a lipid vesicle composition comprising
(a) lipid vesicles each comprising a lipid bilayer comprising vesicle forming lipids, wherein the vesicle forming lipids are present in an amount of from about 5% to about 20%;
(b) an oil-in-water emulsion entrapped in the lipid vesicles, and stabilized by one or more surfactants;
(c) one or more peptides in an amount of from about 0.1 mg/mL to about 50 mg/ml entrapped in the lipid bilayer, the oil-in-water emulsion, or a combination thereof,
wherein the composition further comprises; and
   a fatty amide derived propylene glycol-diammonium phosphate ester in an amount of from about 1% to about 10%;
   a PEG ether of a fatty alcohol in an amount of from about 0.1% to about 3%;
   a polysorbate in an amount of from about 0.5% to about 3%; and
   a sorbate ester in an amount of from about 0.1% to about 2%.

In some embodiments, the oil component is present in an amount of from about 2.5% to about 20%.

In some embodiments, the lipid vesicle composition comprises one or more peptides in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL. In some embodiments the lipid vesicle composition comprises one or more peptides in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the lipid vesicle composition comprises one or more peptides in an amount of about 1 mg/mL, about 2 mg/mL, about 3 mg/mL, about 4 mg/mL, or about 5 mg/mL.

In some embodiments, the one or more peptides of (c) comprises a tetrapeptide, pentapeptide, hexapeptide, or any combination thereof. In some embodiments, the one or more peptides comprises an amino acid sequence at least about 50%, at least about 60%, at least about 70%, at least about 80%, or at least about 90% sequence homology to the amino acid sequence of any one of SEQ ID NO: 1-51. In some embodiments, the one or more peptides comprises at least two, three, four, five, or six peptides each with an amino acid sequence at least about 90% sequence homology to the amino acid sequence of any one of SEQ ID NO: 1-51. In some embodiments, the one or more peptides comprises an amino acid sequence at least about 50%, at least about 60%, at least about 70%, at least about 80%, or at least about 90% sequence homology to the amino acid sequence of any one of SEQ ID NO: 21, 24, 31, 36, 37, 39, or 40. In some embodiments, the one or more peptides comprises an amino acid sequence identical to the amino acids of any one of SEQ ID NO: 21, 24, 31, 36, 37, 39, or 40. In some embodiments, the one or more peptides comprises an amino acid sequence at least about 50%, at least about 60%, at least about 70%, at least about 80%, or at least about 90% sequence homology to the amino acid sequence of SEQ ID NO: 37. In some embodiments, the one or more peptides comprises an amino acid sequence identical to the amino acids of SEQ ID NO: 37.

In some embodiments, the lipid vesicle composition further comprises viscosity enhancing agents in an amount of from about 0.5% to about 5%. In some embodiments, the viscosity enhancing agents comprise one or more of a fatty alcohol, a wax, a fatty ester of glycerol, or any combination thereof.

In some embodiments, the polysorbate is polysorbate 80. In some embodiments, the sorbate ester is sorbitan palmitate. In some embodiments, the PEG ether of the fatty alcohol is diethylene glycol monooleyl ether.

Peptide Composition 4: In one aspect, provided herein, is a lipid vesicle composition comprising:
(a) lipid vesicles each comprising a lipid bilayer comprising vesicle forming lipids, wherein the vesicle forming lipids are present in an amount of from about 5% to about 20%;
(b) an oil-in-water emulsion entrapped in the lipid vesicles, and stabilized by one or more surfactants;
(c) one or more peptides in an amount of from about 0.001% to about 0.05% entrapped in the lipid bilayer, the oil-in-water emulsion, or a combination thereof,
wherein the composition further comprises:
   a fatty amide derived propylene glycol-diammonium phosphate ester in an amount of from about 1% to about 20%;
   a PEG ether of a fatty alcohol in an amount of from about 0.5% to about 2%;
   a polysorbate in an amount of from about 0.4% to about 1%; and
   a glyceryl stearate in an amount of from about 0.5% to about 2%.

In some embodiments, the lipid vesicle composition comprises one or more peptides each of the one or more peptides in an amount of about 0.001 mg/mL, about 0.005 mg/mL, about 0.01 mg/mL, about 0.02 mg/mL, about 0.05 mg/mL, about 0.08 mg/mL, or about 0.1 mg/mL. In some embodiments, the lipid vesicle composition comprises one or more peptides in total in an amount of about 0.001 mg/mL, about 0.005 mg/mL, about 0.01 mg/mL, about 0.02 mg/mL, about 0.05 mg/mL, about 0.08 mg/mL, about 0.1 mg/mL, about 0.2 mg/mL, about 0.3 mg/mL, about 0.4 mg/mL, or about 0.5 mg/mL.

In some embodiments, the one or more peptides of (c) comprise tetrapeptides, pentapeptides, hexapeptides, or any combination thereof. In some embodiments, the one or more peptides comprises an amino acid sequence at least about 50%, at least about 60%, at least about 70%, at least about 80%, or at least about 90% sequence homology to the amino acid sequence of any one of SEQ ID NO: 1-51. In some embodiments, the one or more peptides comprises at least two, three, four, five, or six peptides each with an amino acid sequence at least about 90% sequence homology to the amino acid sequence of any one of SEQ ID NO: 1-51. In some embodiments, the one or more peptides comprises an amino acid sequence at least about 50%, at least about 60%, at least about 70%, at least about 80%, or at least about 90% sequence homology to the amino acid sequence of any one of SEQ ID NO: 21-40. In some embodiments, the one or more peptides comprises at least two, three, four, five, or six peptides each with an amino acid sequence at least about 90% sequence homology to the amino acid sequence of any one of SEQ ID NO: 21-40. In some embodiments, the one or more peptides comprises an amino acid sequence at least about 50%, at least about 60%, at least about 70%, at least about 80%, or at least about 90% sequence homology to the amino acid sequence of any one of SEQ ID NO: 21, 24, 31, 36, 37, 39, or 40. In some embodiments, the one or more peptides comprises an amino acid sequence identical to the amino acids of any one of SEQ ID NO: 21, 24, 31, 36, 37, 39, or 40. In some embodiments, the one or more peptides comprises an amino acid sequence at least about 50%, at least about 60%, at least about 70%, at least about 80%, or at least about 90% sequence homology to the amino acid sequence of SEQ ID NO: 37. In some embodiments, the one or more peptides comprises an amino acid sequence identical to the amino acids of SEQ ID NO: 37. In some embodiments, the lipid vesicle composition comprises one, two, three, four, five, or six peptides with amino acid sequences of any one of SEQ ID NO: 21-40.

In some embodiments, the lipid vesicle composition further comprises viscosity enhancing agents in an amount of from about 0.5% to about 10%. In some embodiments, the viscosity enhancing agents comprise one or more of a fatty alcohol, a wax, a fatty ester of glycerol, or any combination thereof.

In some embodiments, the polysorbate is polysorbate 80. In some embodiments, the glyceryl stearate is a glyceryl monostearate. In some embodiments, the PEG ether of the fatty alcohol is diethylene glycol monooleyl ether.

### Methods of Use of Lipid Vesicle Compositions Provided Herein

The lipid vesicle compositions provided herein are contemplated for cosmetic uses in a subject, for indications including but not limited to the prevention or temporary improvement of the appearance of one or more of: skin wrinkles; skin laxity; moderate to severe glabellar lines associated with corrugator and/or procerus muscle activity; moderate to severe lateral canthal lines associated with orbicularis oculi activity (crow's feet lines); and moderate to severe forehead lines associated with frontalis muscle activity.

The lipid vesicle compositions provided herein are contemplated for pharmaceutical use in a subject, for indications including but not limited to: prevention or temporary improvement of the appearance of one or more of skin wrinkles, e.g., in the face, skin laxity, moderate to severe glabellar lines associated with corrugator and/or procerus muscle activity, moderate to severe lateral canthal lines associated with orbicularis oculi activity (crow's feet lines), and moderate to severe forehead lines associated with frontalis muscle activity.

In some embodiments, the subject is a mammal. In specific embodiments, the mammal is a human. In some embodiments, the human subject is a pediatric or adult subject, of any age.

### Methods for Using Cosmetic or Pharmaceutical Compositions

The present disclosure also relates to methods for using cosmetic or pharmaceutical compositions comprising one or more peptides or an anionic polymer material such as hyaluronic acid. In some embodiments, the disclosure relates to methods for using the cosmetic or pharmaceutical composition to prevent or temporarily improve the appearance in a subject of one or more of skin wrinkles, e.g., in the face or neck, skin laxity, moderate to severe glabellar lines associated with corrugator and/or procerus muscle activity, moderate to severe lateral canthal lines associated with orbicularis oculi activity (crow's feet lines), and moderate to severe forehead lines associated with frontalis muscle activity, comprising applying an effective amount of the cosmetic or pharmaceutical composition to the skin of the subject.

In some embodiments, the lipid vesicle composition is topically applied to a subject. Topical application as referred to herein can refer to application onto one or more surface, e.g., keratinous tissue. In some embodiments, the topical composition is applied to the skin of a subject. In some embodiments, the skin is the facial skin of the subject. In some embodiments, the skin comprises the neck of the subject. In some embodiments, the skin comprises the skin around the eyes of the subject. Topical application may relate to direct application to the desired area. A topical cosmetic or pharmaceutical composition or preparation can be applied by, e.g., pouring, dropping, or spraying, when present as a liquid or aerosol composition; smoothing, rubbing, spreading, and the like, when in ointment, lotion, cream, gel, or a like composition; dusting, when a powder; or by any other appropriate means.

In some embodiments, the lipid vesicle composition is formulated in a form suitable for topical administration. In some embodiments, the lipid vesicle composition is formulated as a cream, a lotion, a suspension, or an emulsion. In some embodiments, the lipid vesicle composition is formulated as a cream. In some embodiments, the lipid vesicle composition is formulated as a lotion. In some embodiments, the lipid vesicle composition is formulated as a suspension.

In some embodiments, topical composition is applied to a subject or the skin of the subject with an effective amount of the lipid vesicle composition one or more times, e.g., 1-3 times per day, 1-21 times per week, 1 time per day, 2 times per day, or 3 times per day. In some embodiments, the topical composition is applied to a subject or the skin of the subject with an effective amount of the lipid vesicle composition about 1 time per week to about 12 times per week. In some embodiments, the topical composition is applied to a subject or the skin of the subject with an effective amount of the lipid vesicle composition at least about 1 time per week. In some embodiments, the topical composition is applied to a subject or the skin of the subject with an effective amount of the lipid vesicle composition at most about 12 times per week. In some embodiments, the topical composition is applied to a subject or the skin of the subject with an effective amount of the lipid vesicle composition about 1 time per week to about 2 times per week, about 1 time per week to about 3 times per week, about 1 time per week to about 4 times per week, about 1 time per week to about 5 times per week, about 1 time per week to about 6 times per week, about 1 time per week to about 7 times per week, about 1 time per week to about 8 times per week, about 1 time per week to about 9 times per week, about 1 time per week to about 10 times per week, about 1 time per week to about 11 times per week, about 1 time per week to about 12 times per week, about 2 times per week to about 3 times per week, about 2 times per week to about 4 times per week, about 2 times per week to about 5 times per week, about 2 times per week to about 6 times per week, about 2 times per week to about 7 times per week, about 2 times per week to about 8 times per week, about 2 times per week to about 9 times per week, about 2 times per week to about 10 times per week, about 2 times per week to about 11 times per week, about 2 times per week to about 12 times per week, about 3 times per week to about 4 times per week, about 3 times per week to about 5 times per week, about 3 times per week to about 6 times per week, about 3 times per week to about 7 times per week, about 3 times per week to about 8 times per week, about 3 times per week to about 9 times per week, about 3 times per week to about 10 times per week, about 3 times per week to about 11 times per week, about 3 times per week to about 12 times per week, about 4 times per week to about 5 times per week, about 4 times per week to about 6 times per week, about 4 times per week to about 7 times per week, about 4 times per week to about 8 times per week, about 4 times per week to about 9 times per week, about 4 times per week to about 10 times per week, about 4 times per week to about 11 times per week, about 4 times per week to about 12 times per week, about 5 times per week to about 6 times per week, about 5 times per week to about 7 times per week, about 5 times per week to about 8 times per week, about 5 times per week to about 9 times per week, about 5 times per week to about 10 times per week, about 5 times per week to about 11 times per week, about 5 times per week to about 12 times per week, about 6 times per week to about 7 times per week, about 6 times per week to about 8 times per week, about 6 times per week to about 9 times per week, about 6 times per week to about 10 times per week, about 6 times per week to about 11 times per week, about 6 times per week to about 12 times per week, about 7 times per week to about 8 times per week, about 7 times per week to about 9 times per week, about 7 times per week to about 10 times per week, about 7 times per week to about 11 times per week, about 7 times per week to about 12 times per week, about 8 times per week to about 9 times per week, about 8 times per week to about 10 times per week, about 8 times per week to about 11 times per week, about 8 times per week to about 12 times per week, about 9 times per week to about 10 times per week, about 9 times per week to about 11 times per week, about 9 times per week to about 12 times per week, about 10 times per week to about 11 times per week, about 10 times per week to about 12 times per week, or about 11 times per week to about 12 times per week. In some embodiments, the topical composition is applied to a subject or the skin of the subject with an effective amount of the lipid vesicle composition about 1 time per week, about 2 times per week, about 3 times per week, about 4 times per week, about 5 times per week, about 6 times per week, about 7 times per week, about 8 times per week, about 9 times per week, about 10 times per week, about 11 times per week, about 12 times per week, about 13 times per week, or about 14 times per week.

In some embodiments, one or more layers of a lipid vesicle composition of the disclosure is applied to the skin of the subject at a given time. In some embodiments, a subsequent layer may be applied after a previous layer of the lipid vesicle composition is fully absorbed into the skin of the subject. In some embodiments, the lipid vesicle composition may take a couple of seconds (e.g., one second, two seconds, three second, five seconds, ten seconds, fifteen seconds, thirty seconds, etc.) to fully absorb into the skin of the subject. In some embodiments, one, two, three, four, five, six, or seven layers of the lipid vesicle composition is applied to the skin of the subject at a given time. In some embodiments, the lipid vesicle composition is applied to the skin of the subject one or more times a day (e.g., 1-3 times per day, 1 time per day, 2 times per day, 3 times per day, etc.). In some embodiments, the lipid vesicle composition is applied to the skin of the subject one or more times a week (e.g., 1-21 times per week, 1-14 times per week, 1-7 times per week, etc.). In some embodiments, the lipid vesicle composition is applied to the skin of the subject daily. In some embodiments, one or more layers of the lipid vesicle composition is applied to the skin of the subject once a day for one or more days. In some embodiments, two or more layers of the lipid vesicle composition is applied to the skin of the subject once a day for one or more days. In some embodiments, three or more layers of the lipid vesicle composition is applied to the skin of the subject once a day for one or more days. In some embodiments, one or more layers of the lipid vesicle composition is applied to the skin of the subject twice a day for one or more days. In some embodiments, two or more layers of the lipid vesicle composition is applied to the skin of the subject twice a day for one or more days. In some embodiments, three or more layers of the lipid vesicle composition is applied to the skin of the subject twice a day for one or more days. In some embodiments, the lipid vesicle composition is applied to the skin of the subject for at least one day, two days, three days, four days, five days, six days, one week, two weeks, three weeks, one month, two months, three months, six months, one year. In some embodiments, the lipid vesicle composition is applied to the skin of the subject for more than one day, two days, three days, four days, five days, six days, one week, two weeks, three weeks, one month, two months, three months, six months, nine months, or one year. In some embodiments, one or more layers of the lipid vesicle composition is applied to the skin of the subject twice a day for several days, and thereafter is applied three times a day. In some embodiments, five layers of the lipid vesicle composition is applied to the skin of the subject twice a day for five days (e.g., morning and night), and thereafter one to three layers of the lipid vesicle composition is applied to the skin of the subject three times a day (e.g., morning, noon and night).

In some embodiments, a lipid vesicle composition of the disclosure is applied to the skin of a subject, for indications including but not limited to: prevention or temporary improvement of the appearance of the skin, including, but not limited to, one or more of skin wrinkles on the face or the neck, e.g., skin laxity, moderate to severe glabellar lines associated with corrugator and/or procerus muscle activity, moderate to severe lateral canthal lines associated with orbicularis oculi activity (crow's feet lines), and moderate to severe forehead lines associated with frontalis muscle activity.

In some embodiments, a lipid vesicle composition of the disclosure is applied to a subject, for indications including but not limited to: temporary improvement of the appearance of the skin of the face. In some embodiments, a lipid vesicle composition of the disclosure is used with other skincare products, including, but not limited to sunscreen, moisturizers, face creams (e.g., BB cream, CC cream, night cream, etc.), mists, foundations, concealers, highlighters, primers, etc.

In some embodiments, a topical composition of the disclosure is self-applied by a subject. In some embodiments, a topical composition of the disclosure is not self-applied by a subject. In some embodiments, the topical composition is a topical cosmetic composition.

### Methods of Making Lipid Vesicle Compositions Provided Herein

Also provided herein are method of making lipid vesicle compositions. In some embodiments, compositions of the disclosure as described above are prepared by mixing oil components of the oil-in-water emulsion with aqueous components of the oil-in-water emulsion wherein either the oil components or aqueous components of the oil-in-water emulsion comprises one or more surfactants for emulsification of the oil component with the aqueous component of the oil-in-water emulsion. In an embodiment, the surfactant is mixed with the aqueous component and added to the oil for formation of an emulsion. The oil-in-water emulsion is then mixed with the solubilized vesicle-forming lipid and, if added, other lipid components under mixing conditions effective to form the lipid vesicles (e.g., multisomes).

In some embodiments, one or more penetration enhancing agents and the one or more compounds (e.g., anionic polymer material, one or more peptides, etc.) are added to oil component of the oil-in-water emulsion, to the aqueous component of the oil-in-water emulsion or both. Alternatively, or in addition to, the one or more penetration enhancing agents and/or the one or more compounds can be added to the lipid component.

In one aspect, provided herein, is a method of preparing a lipid vesicle composition provided herein, comprising a) preparing an oil-in-water emulsion comprising an active ingredient, by mixing oil components of the oil-in-water emulsion with aqueous components of the oil-in-water emulsion; b) solubilizing vesicle forming lipids in an acceptable solvent other than water; c) adding the oil-in-water emulsion to the solubilized vesicle forming lipids; and d) mixing the oil-in-water emulsion and the solubilized vesicle forming lipids under mixing conditions effective to form the lipid vesicles comprising a lipid bilayer comprising vesicle forming lipids, and an oil-in-water emulsion entrapped in the lipid vesicles. In some embodiments, the active ingredient is one or more peptides provided herein. In some embodiments, the one or more peptides are unmodified. In some embodiments, the one or more peptides comprise a dipeptide, tripeptide, tetrapeptide, pentapeptide, hexapeptide, heptapeptide, octapeptide, or a combination thereof. In some embodiments, the one or more peptides comprises an amino acid sequence at least about 50%, at least about 60%, at least about 70%, at least about 80%, or at least about 90% sequence homology to the amino acid sequence of any one of SEQ ID NO: 1-51. In some embodiments, the one or more peptides comprises an amino acid sequence identical to the amino acids of any one of SEQ ID NO: 1-51. In some embodiments, the one or more peptides comprises an amino acid sequence at least about 50%, at least about 60%, at least about 70%, at least about 80%, or at least about 90% sequence homology to the amino acid sequence of any one of SEQ ID NO: 21, 30, 35, 36, 37, 38, or 39. In some embodiments, the one or more peptides comprises an amino acid sequence identical to the amino acids of any one of SEQ ID NO: 21, 30, 35, 36, 37, 38, or 39. In some embodiments, the one or more peptides comprises an amino acid sequence at least about 50%, at least about 60%, at least about 70%, at least about 80%, or at least about 90% sequence homology to the amino acid sequence of SEQ ID NO: 37. In some embodiments, the one or more peptides comprises an amino acid sequence identical to the amino acids of SEQ ID NO: 37. In some embodiments, the active ingredient is an anionic polymer material provided herein. In some embodiments, the lipid vesicle may comprise of one or more active ingredients. In some embodiments, the one or more active ingredients comprise one or more peptides. In some embodiments, the one or more active ingredients comprise an anionic polymer material. In some embodiments, the one or more active ingredients comprise one or more peptides and an anionic polymer material. In some embodiments, the anionic polymer material is hyaluronic acid.

In some embodiments, the method further comprising adding one or more of the additional components provided herein (e.g., penetration enhancing agents, viscosity enhancing agents, etc.).

In some embodiments, mixing oil components of the oil-in-water emulsion with aqueous components of the oil-in-water emulsion vesicles of step a) and/or the mixing conditions of step e) comprises using agitation such as homogenization or emulsification, or micro-emulsion techniques which do not involve agitation. In an embodiment, the mixing comprises high pressure homogenizing. The high pressure homogenizing provides relatively precise control over the composition of the lipid vesicles. High pressure homogenizing is suitable for small molecules and peptides or proteins that are resistant to shearing. In an embodiment, the composition that is formed is any one of the lipid vesicle compositions described herein.

In some embodiments, other lipid components are added at any one of the steps.

### EXAMPLES

### Example 1. Preparation of Multisome Lipid Vesicle Compositions of Hyaluronic Acid

Biphasic vesicles with multiple/synergistic penetration enhancers (Multisomes) were formulated with three different molecular weight hyaluronic acid, 250K, 50K and 10K (Creative PEGWorks, Chapel Hill, NC) at with either 1 mg/mL or 1.5 mg/mL concentration. For formulation development unlabelled HA was used. For the diffusion cell experiments the vesicles were prepared with labelled HAs (Rhodamine-HA250K, FITC-HA50K and FITC-HA-10K; Creative PEGWorks).

The general procedure for multisome preparation was as follows:

1) The oil phase and aqueous phase ingredients were weighed out in separate beakers.

2) Both beakers were heated to ~70° C. to completely melt and incorporate all components.

3) The water phase was added to the oil, while stirring vigorously with a spatula to form an o/w crude emulsion, effectively yielding a homogenous milky solution (~2 to 6 min) in the ~70° C. water bath. The temperature of the solution was ~55 to 65° C.

4) The formulation was batch processed using the LV1 Microfluidizer or Nano DeBee homogenizer with Z5 module three times at ~20,000 psi.

Procedure for vesicle formation (applicable to all formulations):

1) The lipid phase components were weighed into a 20 mL glass vial.

2) The vial was heated to ~70° C. in a water bath to completely melt and incorporate all components.

3) The water phase (System A) was added to the liquid phase while stirring vigorously for ~10 to 20 min until the temperature of the solution was ~60° C.

In some cases, the mixture was intermittently vortexed and heated for 5 sec/5 sec for 8-10 cycles until a uniform creamy lotion formed.

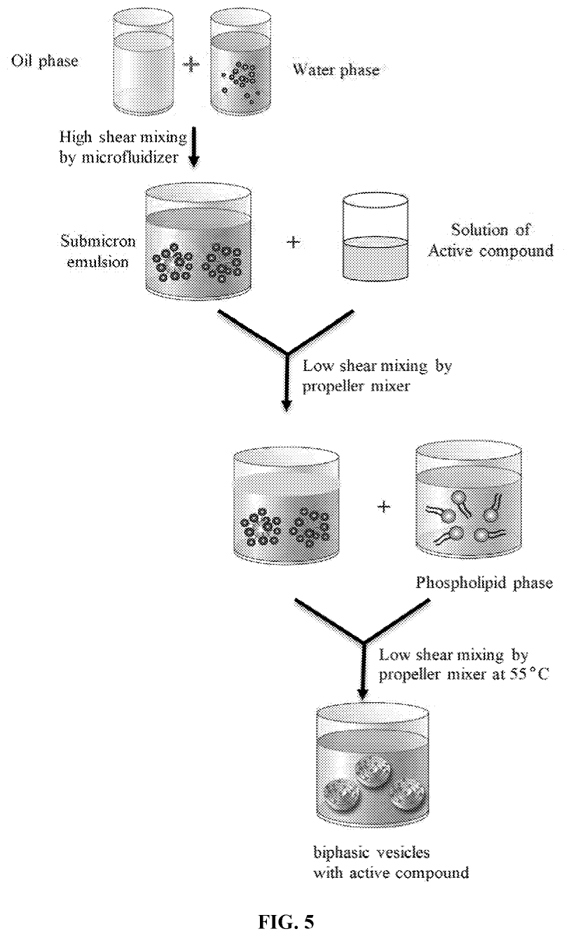

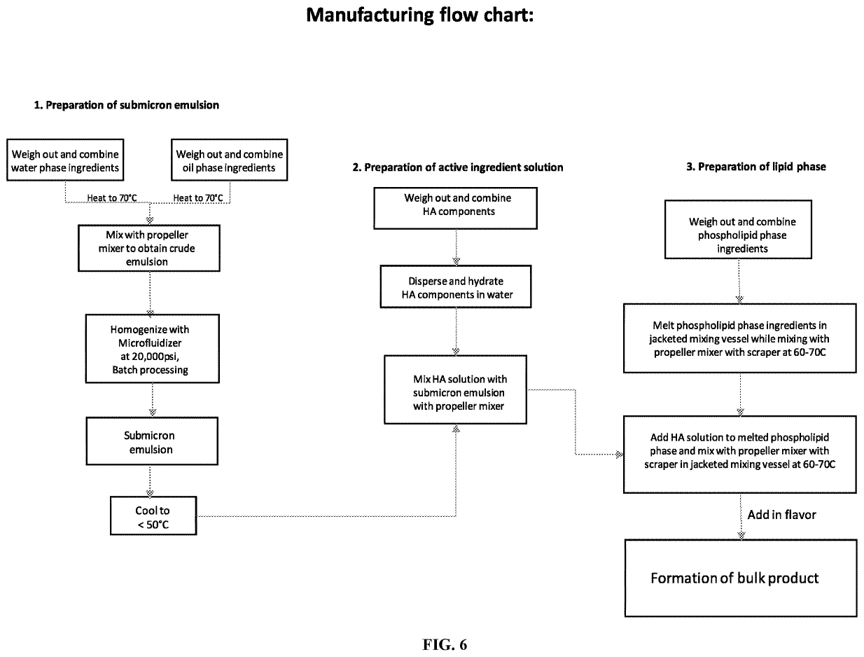

A pictorial representation of this process is shown in FIG. 5. A flow chart of this exemplary process is shown in FIG. 6.

The various lipid phases used throughout the experiments are described in Table A and the aqueous phases are described in Table B below.

TABLE A
Lipid Phases used in Formulations

| | | Concentration w/w |
|---|---|---|
| Lipid phase name | Ingredients | (final formulation) |
| F1SunL | Sunlipon 90H | 7% |
| | Cholesterol | 1.75% |
| | Propylene glycol | 7% |
| F1SunL1.5x | Sunlipon 90H | 10.5% |
| | Cholesterol | 2.7% |
| | Propylene glycol | 10.5% |
| F70SunL | Sunlipon 70H | 7% |
| | Cholesterol | 1.75% |
| | Propylene glycol | 7% |

TABLE B
Aqueous Phases used in Formulations

| Aqueous phases | | Concentration |
|---|---|---|
| (System A) | Ingredients | w/w |
| F4M | Labrafac CC | 5% |
| | Glyceryl monostearate NE | 1.2% |
| | Cetyl alcohol | 0.6% |
| | Syncrowax BB4 (beeswax) | 0.3% |
| | Arlasilk EFA | 5% |
| | Propylparaben | 0.05% |
| | Methylparaben | 0.15% |
| | Milli-Q Water | Qs to 100 |
| F2Gemini 12312 | Labrafac CC | 5% |
| | Glyceryl monostearate NE | 1.2% |
| | Cetyl alcohol | 0.6% |
| | Syncrowax BB4 (beeswax) | 0.3% |
| | Dicationic (gemini) surfactant | 0.1% |
| | 12-3-12 | |
| | Tween 80 | 0.5% |
| | Propylparaben | 0.05% |
| | Methylparaben | 0.15% |
| | Milli-Q Water | Qs to 100 |
| F3Plys1-5K | Labrafac CC | 5% |
| | Glyceryl monostearate NE | 1.2% |
| | Cetyl alcohol | 0.6% |
| | Syncrowax BB4 (beeswax) | 0.3% |
| | Polylysine (Sigma) 1-5K | 0.05% |
| | Tween 80 | 0.5% |
| | Propylparaben | 0.05% |
| | Methylparaben | 0.15% |
| | Milli-Q Water | Qs to 100 |
| F5-COSM4 | Labrafac CC | 5% |
| | Glyceryl monostearate NE | 1.2% |
| | Cetyl alcohol | 0.6% |
| | Syncrowax BB4 (beeswax) | 0.3% |
| | Dimethicone 200CST | 1% |
| | Lipovol GBT | 0.2% |
| | Lipocol O2 | 1% |
| | Arlasilk EFA | 5% |
| | Euxyl9010 | 0.8% |
| | Spectrastat | 0.5% |
| | Milli-Q Water | Qs to 100 |
| F6-COSM5 | Labrafac CC | 5% |
| | Glyceryl monostearate NE | 1.2% |
| | Cetyl alcohol | 0.6% |
| | Syncrowax BB4 (beeswax) | 0.3% |
| | Dimethicone 200CST | 1% |
| | Super refined Brij O2 | 1% |
| | Arlasilk EFA | 5% |
| | Euxyl9010 | 0.8% |
| | Spectrastat | 0.5% |
| | Milli-Q Water | Qs to 100 |
| FA3-2 | Labrafac CC | 5% |
| | Glyceryl monostearate NE | 1.2% |
| | Cetyl alcohol | 0.6% |
| | SyncrowaxBB4 | 0.3% |
| | Everguard Polylysine (3-5K) | 0.2% |
| | Tween 80 | 0.5% |
| | Milli-Q Water | Qs to 100 |
| F6-COSM6 | Labrafac CC | 5% |
| | Glyceryl monostearate NE | 1.2% |
| | Cetyl alcohol | 0.6% |
| | Syncrowax BB4 (beeswax) | 0.3% |
| | Dimethicone 200CST | 1% |
| | Lipovol GBT | 0.2% |
| | Super refined Brij O2 | 1% |
| | Everguard Polylysine (3-5K) | 0.2% |
| | Arlasilk EFA | 5% |
| | Tween80 | 0.5% |
| | Euxyl9010 | 0.8% |
| | Spectrastat | 0.5% |
| | Milli-Q Water | Qs to 100 |
| F6-COSM6BN | Labrafac CC | 5% |
| | Glyceryl monostearate NE | 1.2% |
| | Cetyl alcohol | 0.6% |
| | Syncrowax BB4 (beeswax) | 0.3% |
| | Dimethicone 200CST | 1% |
| | Lipovol GBT | 0.2% |
| | Super refined Brij O2 | 1% |
| | Benzyl nicotinate | 0.5% |
| | Everguard Polylysine (3-5K) | 0.2% |
| | Arlasilk EFA | 5% |
| | Tween80 | 0.5% |
| | Euxyl9010 | 0.8% |
| | Spectrastat | 0.5% |
| | Milli-Q Water | Qs to 100 |

### Example 2. Methods of Analysis and Characterization

The following methods were used to characterize the formulations described in the following examples as well as the performance of the formulations.

Physicochemical Characterization-Organoleptic observations, light microscopy and confocal microscopy (Zeiss 710 confocal laser scanning microscope (CLSM)) were carried out to characterize the formulations. Confocal microscopy images of the formulations were obtained using a Zeiss LSM 710 CLSM using argon-laser 488 and HeNe-laser 543 lines for FITC (495/525) and Rhodamine (570/590), and either the Plan-Apochromat 20×/0.80 dry objective or the 63×/1.40 oil immersion objective. Optical zoom selection was applied in selected cases. Laser intensity, pinhole and gain settings were kept consistent between sample sets to enable comparison of relative fluorescence intensity measurements between samples. Images were captured and processed using the Zen 2009 software.

Size (hydrodynamic diameter) and polydispersity index and zeta (ζ) potential measurements were carried out on formulations using the Nano ZS Zetasizer (Malvern Instruments, Worcestershire, UK) which measures the hydrodynamic diameter of particles using dynamic light scattering (DLS). Aliquots of formulations were diluted 20× in water and 100 μL and 1000 μL of each formulation were prepared for size and zeta potential measurements, respectively. Measurements were carried out in triplicates.

In vitro diffusion cell studies-Full thickness human breast skin was obtained from female donors undergoing elective mammoplasty surgeries at the Royal University Hospital, University of Saskatchewan (Saskatoon, SK, Canada). Approval for skin collection was granted by the Human Ethics Committee at the University of Saskatchewan. The skin was collected within 2 h following surgery, trimmed of subcutaneous fat, and stored at −20° C. until use. In-line Bronaugh Flow-through diffusion cells with a 9 mm orifice diameter (0.63 cm²) were mounted on a water insulated cell warmer (PermeGear, Inc., Hellertown, PA) and set to a constant temperature of 32° C. Precut 1 cm2 skin sections were placed in the diffusion cells with the stratum corneum side facing up. Perfusion buffer (100 mM phosphate buffer with 0.05% Na-azide) at 37° C. was circulated through the lower half of the diffusion cells at a rate of 1 mL/h using a peristaltic pump. The surface of the skin was dosed with 0.1 mL of the formulations. Following 24 h incubation, the skin samples were removed from the cells, cleansed and processed for analysis.

Skin analysis-After removing the skin samples from the diffusion cells, first, the formulation remaining on the skin surface was removed. Each skin sample was subjected to a cleansing protocol and a tape stripping protocol to remove residual bound cream and the stratum corneum as follows: the skin sample was washed with 3× 10 mL water, patted dry with a kimwipe and divided into 2 halves; one half of the skin was tape stripped two times (surface bound formulation removed), embedded into OCT compound on dry ice and cryosectioned The cryosections were examined by confocal microscopy.

Skin samples were cryosectioned with a Leica CM1850 cryostat into 10 μm sections. Sections on slides were left unstained. Confocal microscopy images of the skin sections were obtained using a Zeiss LSM 710 CLSM using argon-laser 488 and HeNe-laser 543 lines for FITC (495/525) and Rhodamine (570/590), and either the Plan-Apochromat 20×/0.80 dry objective or the 63×/1.40 oil immersion objective. Optical zoom selection was applied in selected cases. Laser intensity, pinhole and gain settings were kept consistent between sample sets to enable comparison of relative fluorescence intensity measurements between different applications. Images were captured and processed using the Zen 2009 software.

The ‘no application’ skin sample was used to confirm gain and pinhole settings to exclude noise and autofluorescence background before the analysis of the subsequent application samples.

### Example 3. Evaluation of Cationic Penetration Enhancers

The first strategy for formulating HA250K+HA10K 1 mg/mL combinations was to incorporate them into multisomes, i.e. next generation biphasic vesicles (synergistic enhancer type), with mono-, di- or polycationic building blocks to enhance the encapsulation and delivery of the negatively charged hyaluronic acid into skin layers. The components of these formulations are shown in Table C below.

TABLE C
Composition of formulations for delivery of HA250
+ 10 (1 mg/mL) using cationic excipients

| Formulation | Lipid phase | System A |
|---|---|---|
| F1 | F1 SunL | F4M |
| F2 | F1 SunL | F2G12312 |
| F3 | F1 SunL | F3Plys1-5K |

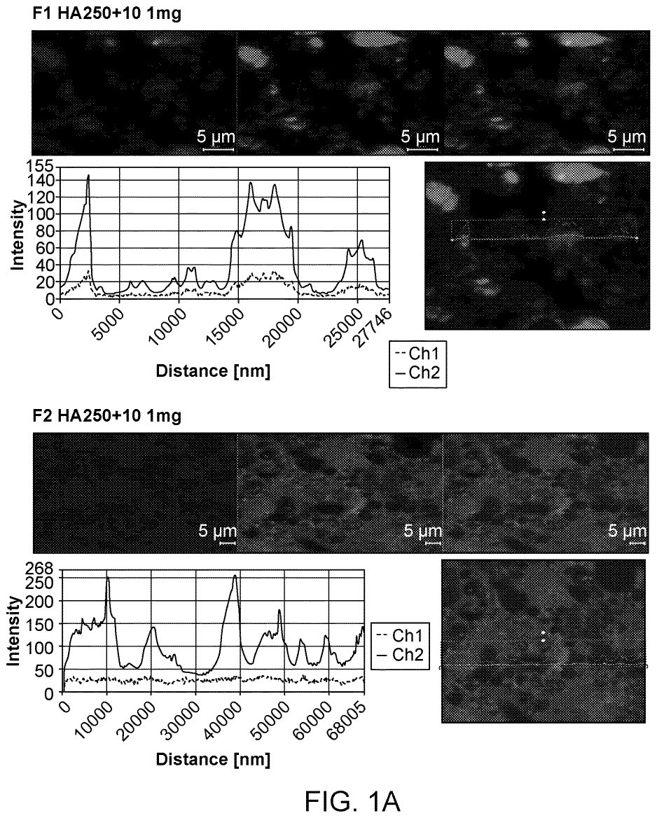

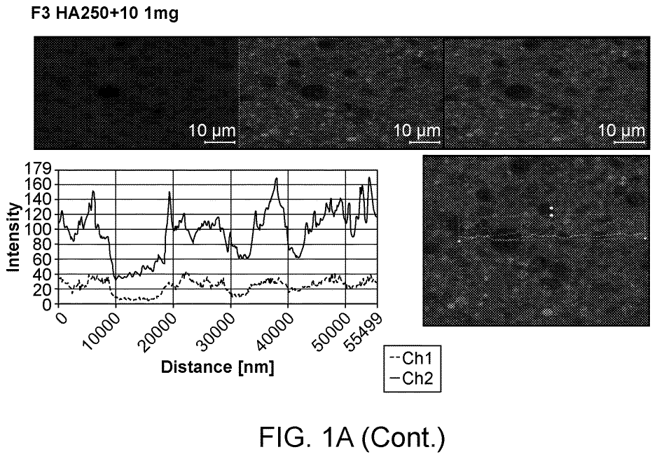

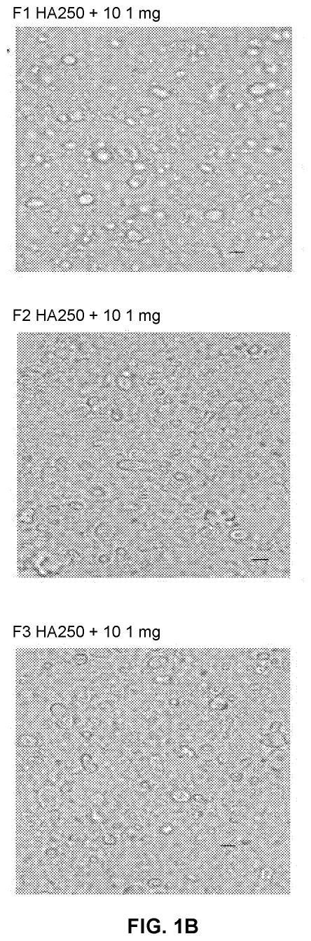

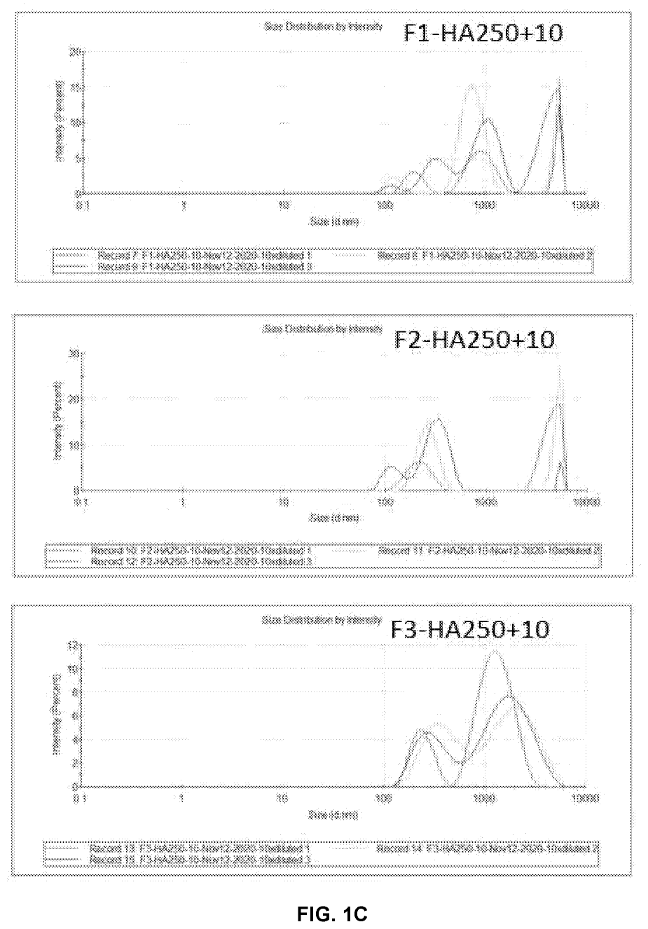

Confocal microscopic studies of the multisomes, showing the distribution of Rho-HA250K (red) and FITC-HA10K (green) fluorescence in the formulation, were used to analyze the HA encapsulation in the vesicles (FIG. 1A, images). The final concentration of HA in these samples was 1 mg/mL. Confocal microscopic profile tracings confirmed the association of red Rho-HA250K and green FITC-HA10K with the vesicles for formula F1, F2 and F3 (FIG. 1A, trace). The fluorescence intensity (FI) curves tracing the vesicles along the selected plane show the co-localization of the red and green fluorescence, indicating the co-encapsulation of the two different molecular weight HAs. Light microscopic images taken of formulations indicated the formation of multisomes (next generation biphasic vesicles) for each type of formulation (FIG. 1B). Zetasizer studies for the System A (submicron emulsion component and the biphasic vesicles were carried out (FIG. 1C). The formulations were shown to be polydisperse with vesicle sizes ranging generally between 0.3-10 μm. Zetasizer data show consistent results with the microscopic observations (FIG. 1B). This is typical of multisomes. The zeta potential for F1, F2 and F3 were +33.6±0.6, +13.0±0.71 and −5.78±0.31, respectively. Similar data was observed for the other formulations described in the examples that follow (data not shown).

The physicochemical properties of the multisome formulations were assessed for color, consistency, and homogeneity. All formulations were lotion or cream consistency suitable for topical application. The formulations were physically stable showing no separation, sedimentation or other signs of stability issues for >3 mo of storage at 4° C. Microscopic observations confirmed that the multisomes remain intact and uniformly distributed during storage. Similar observations were made for the other formulations described in the examples that follow (data not shown).

The cryosections of human skin samples to which topical formulations containing fluorescence labelled HA were applied in vitro in diffusion cells were evaluated for the presence of fluorescent protein. The enhancement of delivery of (negatively charged) HA compounds is shown with three basic vesicle formulations utilizing three cationic vesicle building blocks (Table C).

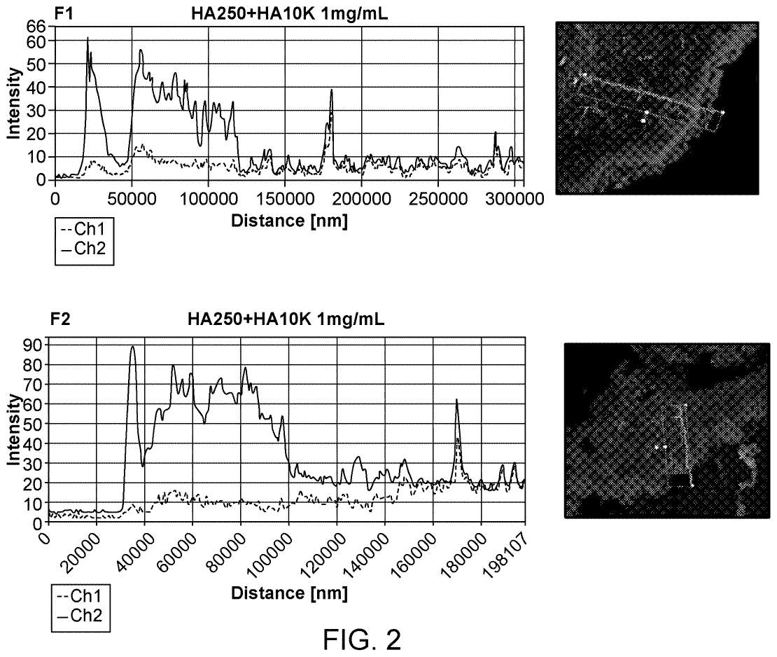

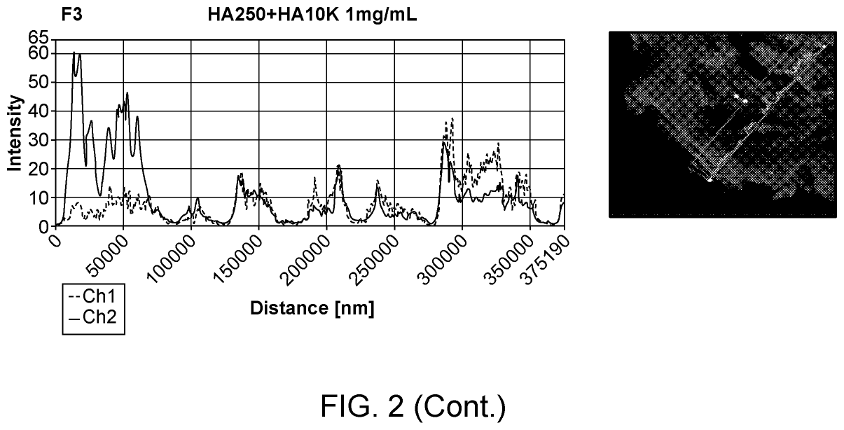

These studies indicated that all three cationic formulations increased HA delivery (Table D, FIG. 2). The order of enhancement followed the order dicationic>monocationic>polycationic formulations.

TABLE D
Analysis of the fluorescence intensity (FI) of F1,
F2 and F3 formulations applied to skin sections.

| | Average skin FI | | | Formulation FI |
|---|---|---|---|---|
| | (n = 3) | | Ratio | ratio |
| Formulation | Ch1 (FITC) | Ch2 (Rho) | Ch1/Ch2 ratio | Ch1/Ch2 ratio |
| F1 | 9 | 39 | 0.21 | 0.24 |
| F2 | 15 | 100 | 0.15 | 0.23 |
| F3 | 9 | 35 | 0.25 | 0.24 |

### Example 4. Evaluation of Hyaluronic Acid Concentration and Presence of Additional Cosmetic Components

Next, the effect of HA concentration was evaluated in the basic vesicle formulations. In order to assess this effect, 1 mg/mL and 1.5 mg/mL of 250 kDa and 10 kDa hyaluronic acid (total weight of combined HA, equal mass of each molecular weight) 1 mg/mL and 1.5 mg/mL of 250 kDa and 50 kDa hyaluronic acid (total weight of combined HA, equal mass of each molecular weight) were prepared in the formulations provided in Table E. Also prepared were solution or gel formulations of HA according to formulations E11 and E12 of Table E made from 1% hydroxypropyl methylcellulose (HPMC) gel.

TABLE E
Composition of formulations for delivery of
HA250 + 10 and HA250 + 50 combinations

| Formulation | | Lipid | |
|---|---|---|---|
| Number | Formulation | phase | System A |
| E1 | F4HA250 + 10 1 mg | F1SunL | F4M |
| E2 | F4HA250 + 10 1.5 mg | F1SunL | F4M |
| E3 | COSMF4HA250 + 10 1.5 mg | F1SunL | F5-COSM4 |
| E4 | F4HA250 + 50 1 mg | F1SunL | F4M |
| E5 | F4HA250 + 50 1.5 mg | F1SunL | F4M |
| E6 | COSMF4HA250 + 50 1.5 mg | F1SunL | F5-COSM4 |
| E7 | F3-2HA250 + 50 1.5 mg | F1SunL | FA3-2 |
| E8 | F1-F6-COSM6-HA250 + 10 1.5 mg | F1SunL | F6-COSM6 |
| E9 | F1-F6-COSM6-HA250 + 50 1.5 mg | F1SunL | F6-COSM6 |
| E10 | F1-F6-COSM6-HA250 + 50BN 1.5 mg | F1SunL | F6-COSM6BN |

| — | Gel Formulation | Vehicle |
|---|---|---|
| E11 | Gel-HA250 + 10 1.5 mg | 1% HPMC gel |
| E12 | Gel-HA250 + 50 1.5 mg | 1% HPMC gel |

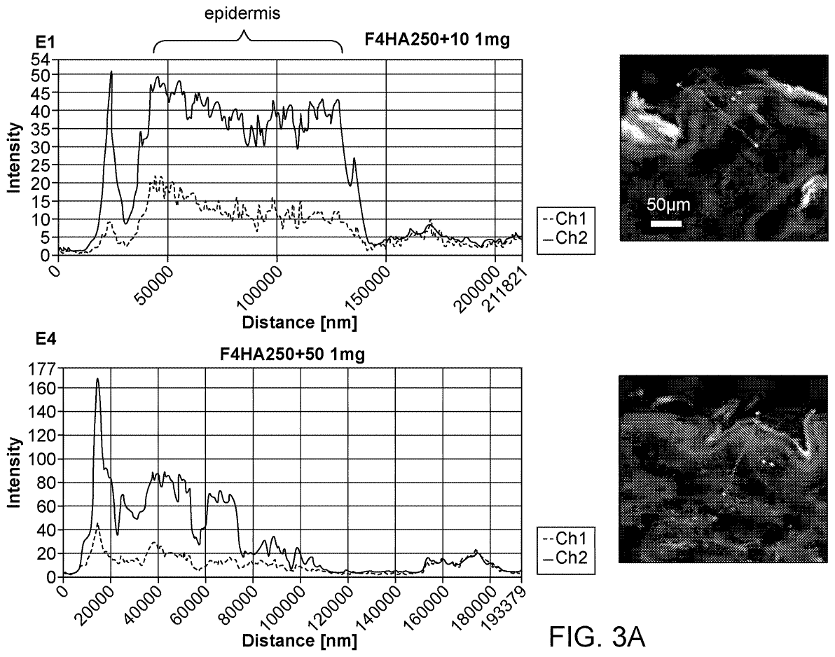

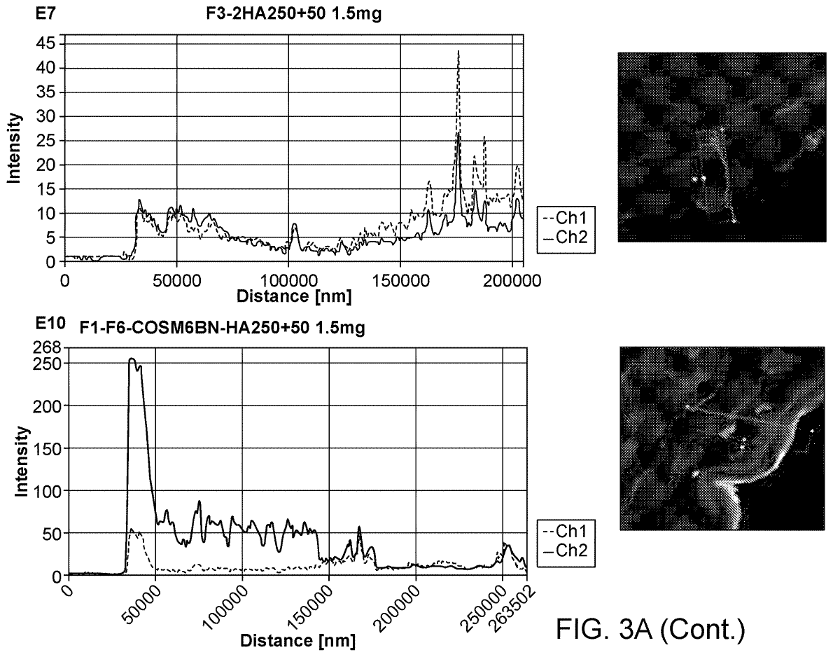

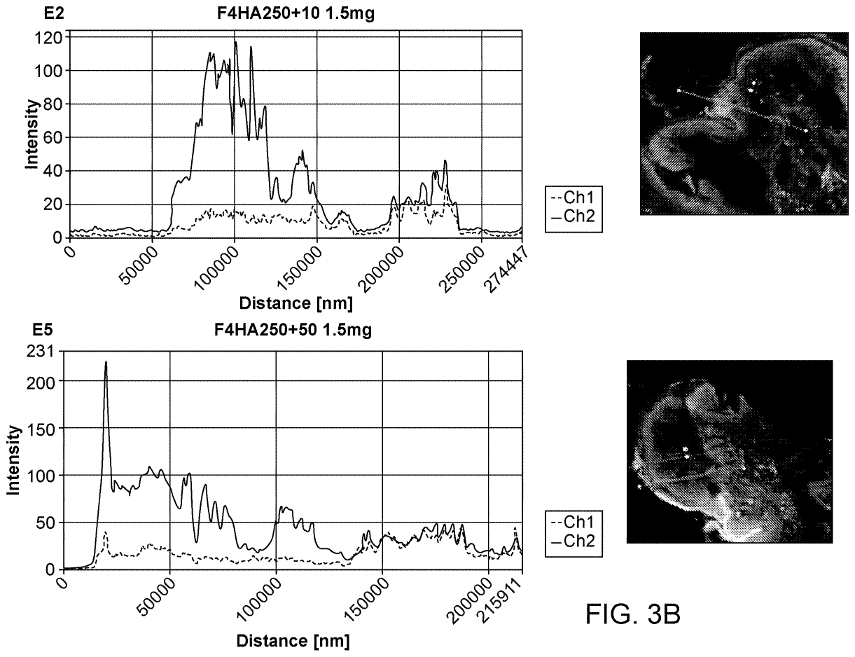

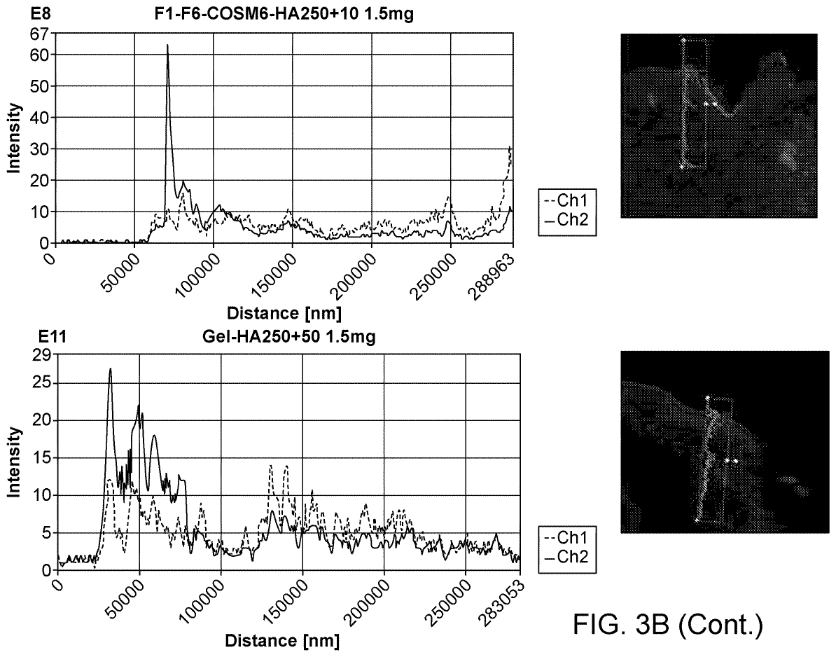

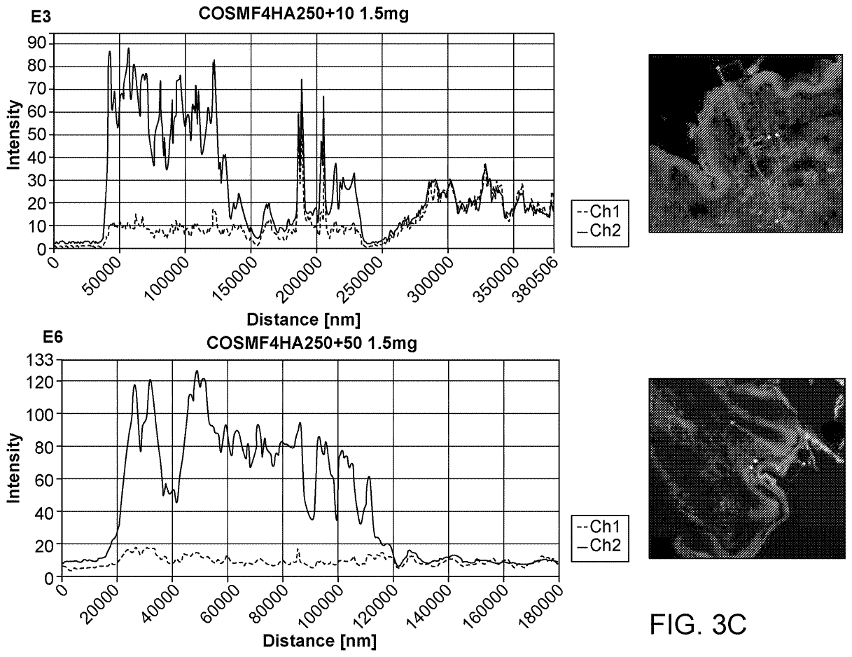

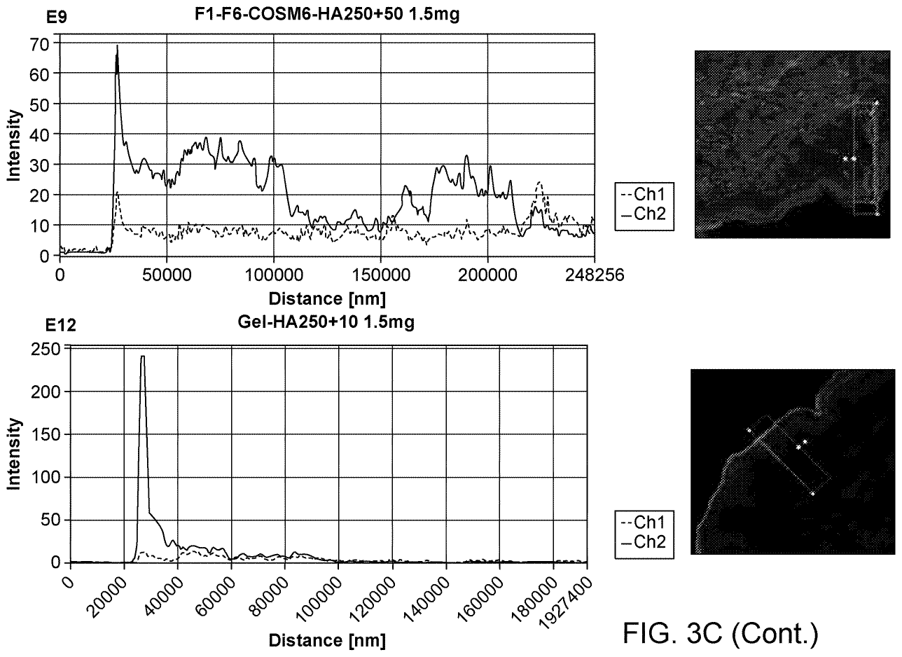

Upon administration to human skin as provided in Example 2, the results shown in Table F below were obtained. Increasing concentration in HA250/10K or HA250/50K total concentration from 1 mg/mL to 1.5 mg/mL vesicle formulation increased delivery, as shown by comparing formula E1 vs E2 and formula E4 vs E5 (Table F, FIGS. 3A-3C), especially evident from the increase of the HA250K component. Further optimization to obtain cosmetic vesicle formulations, indicated that these changes did not affect delivery, see formula E2 vs E3 and E5 vs E6 (Table F, FIGS. 3A-3C).

Comparing formulas E7, E8, E9 and E10 (Table F, FIGS. 3A-3C) indicate that these compositions did not achieve delivery enhancement compared to the other formulations. In formula E7 and E10 (Table F, FIGS. 3A-3C), the strategy of using polycationic and monocationic agents together decreased delivery compared to using monocationic agent alone. The gel formulations (formula E11 and E12 of Table F, FIGS. 3A-3C) where the HA250/10K or 250/50K was incorporated in ‘free’ (not encapsulated) form showed very low/negligible delivery levels.

All multiphasic vesicle formulations enhanced delivery compared to HA in solution or gel formulations. Formulation design differences of multiphasic vesicles indicated that HA delivery can be modulated.

TABLE F
Analysis of fluorescence intensities (FI)
of HA formulations applied to skin sections

| | | Average skin FI | | |
|---|---|---|---|---|
| | | (n = 3) | | Ratio |
| Formulation | | Ch1 | Ch2 | Ch1/Ch2 |
| Number | Formulation | (FITC) | (Rho) | ratio |
| E1 | F4HA250 + 10 1 mg | 15 | 45 | 0.33 |
| E2 | F4HA250 + 10 1.5 mg | 15 | 75 | 0.2 |
| E3 | COSMF4HA250 + 10 1.5 mg | 12 | 70 | 0.17 |
| E4 | F4HA250 + 50 1 mg | 30 | 85 | 0.35 |
| E5 | F4HA250 + 50 1.5 mg | 20 | 100 | 0.2 |
| E6 | COSMF4HA250 + 50 1.5 mg | 18 | 120 | 0.15 |
| E7 | F3-2HA250 + 50 1.5 mg | 10 | 10 | 1 |
| E8 | F1-F6-COSM6-HA250 + 10 1.5 mg | 10 | 15 | 0.66 |
| E9 | F1-F6-COSM6-HA250 + 50 1.5 mg | 10 | 35 | 0.28 |
| E10 | F1-F6-COSM6BN -HA250 + 50 1.5 mg | 8 | 60 | 0.13 |
| E11 | Gel-HA250 + 10 1.5 mg | 7 | 15 | 0.47 |
| E12 | Gel-HA250 + 50 1.5 mg | 12 | 15 | 0.8 |

Also assessed were multisome compositions having additional cosmetic properties, including formulations which included Lipovol GBT (tribehenin) or benzyl nicotinate. The effect on transdermal penetrations of these components on hyaluronic acids having combination molecular weights of 250/10 kDa and 250/50 kDa was assessed. The formulations tested are shown below in Table G.

TABLE G
Composition of formulations for delivery of HA250 +
10 and HA250 + 50 combinations (total HA concentration
1.5 mg/mL) with optimized cosmetic properties

| Formulation | | | |
|---|---|---|---|
| Number | Formulation | Lipid phase | System A |
| | HA250K + 10K | | |
| G1 | Formula F1-F5-COSM4-HA250 + 10 | F1SunL1.5x | F5-COSM4 |
| G2 | Formula F1-F5-COSM4- | F1SunL1.5x | F5-COSM4 |
| | HA250 + 10 + BN | | |
| G3 | Formula F1-F6-COSM5-HA250 + 10 | F1SunL1.5x | F6-COSM5 |
| G4 | Formula F1-F6-COSM5 - | F1SunL1.5x | F6-COSM5 |
| | HA250 + 10 + BN | | |
| | HA250K + 50K | | |
| G5 | Formula F1-F5-COSM4- HA250 + 50 | F1SunL1.5x | F5-COSM4 |
| G6 | Formula F1-F5-COSM4- | F1SunL1.5x | F5-COSM4 |
| | HA250 + 50 + BN | | |
| G7 | Formula F1-F6- COSM5 HA250 + 50- | F1SunL1.5x | F6-COSM5 |
| G8 | Formula F1-F6-COSM5- | F1SunL1.5x | F6-COSM5 |
| | HA250 + 50 + BN | | |
| | Other formulations | | |
| G9 | Formula F4-HA250 + 50-F70-1.5 mg | F70SunL | F4M |
| G10 | Formula F4-HA250 + 50 + 10-1.5 mg | F1SunL1.5x | F4M |

Upon administration to human skin as provided in Example 2, the results shown in Table H below were obtained.

TABLE H

| | | Average skin FI | | |
|---|---|---|---|---|
| | | (n = 3) | | Ratio |
| | | Ch1 | Ch2 | Ch1/Ch2 |
| | Formulation | (FITC) | (Rho) | ratio |
| G1 | Formula F1-F5-COSM4- | 12 | 70 | 0.17 |
| | HA250 + 10 | | | |
| G2 | Formula F1-F5-COSM4- | 15 | 124 | 0.12 |
| | HA250 + 10 + BN | | | |
| G3 | Formula F1-F6-COSM5- | 9 | 55 | 0.16 |
| | HA250 + 10 | | | |
| G4 | Formula F1-F6-COSM5 - | 18 | 112 | 0.16 |
| | HA250 + 10 + BN | | | |
| G5 | Formula F1-F5-COSM4- | 15 | 74 | 0.2 |
| | HA250 + 50 | | | |
| G6 | Formula F1-F5-COSM4- | 14 | 58 | 0.24 |
| | HA250 + 50 + BN | | | |
| G7 | Formula F1-F6- COSM5 | 12 | 70 | 0.17 |
| | HA250 + 50- | | | |
| G8 | Formula F1-F6-COSM5- | 14.2 | 100.8 | 0.14 |
| | HA250 + 50 + BN | | | |
| G9 | Formula F4-HA250 + 50-F70- | 16 | 35 | 0.45 |
| | 1.5 mg | | | |
| G10 | Formula F4-HA250 + 50 + 10- | 15 | 17 | 0.88 |
| | 1.5 mg | | | |

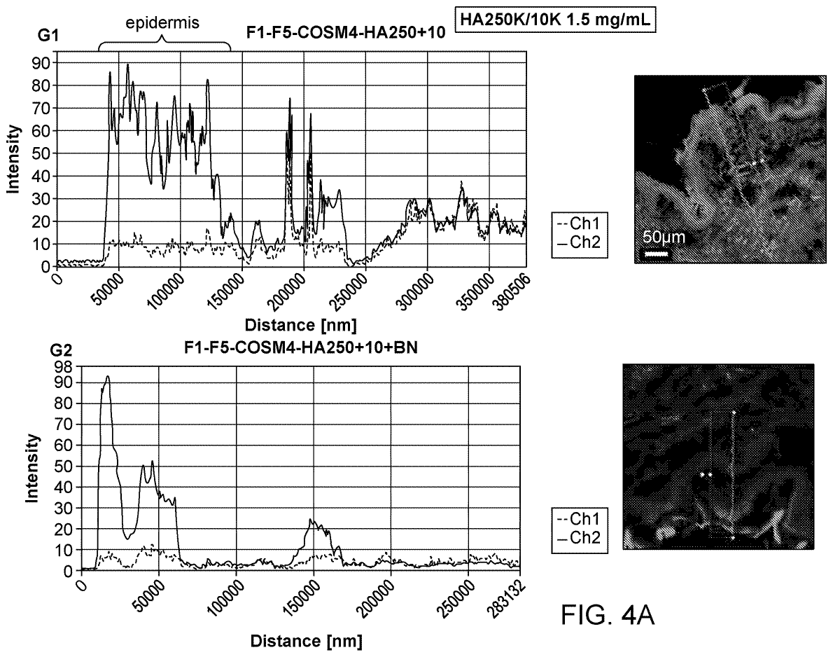

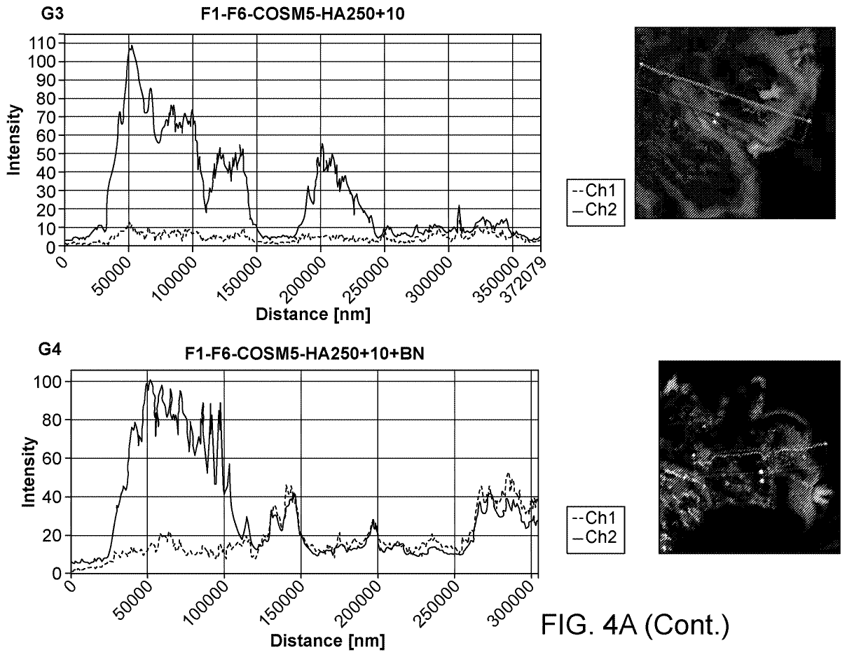

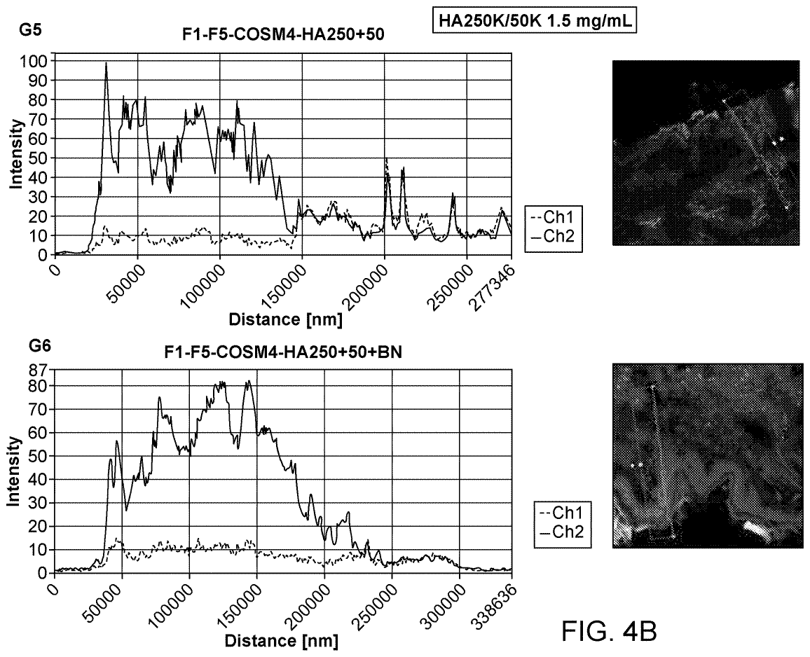

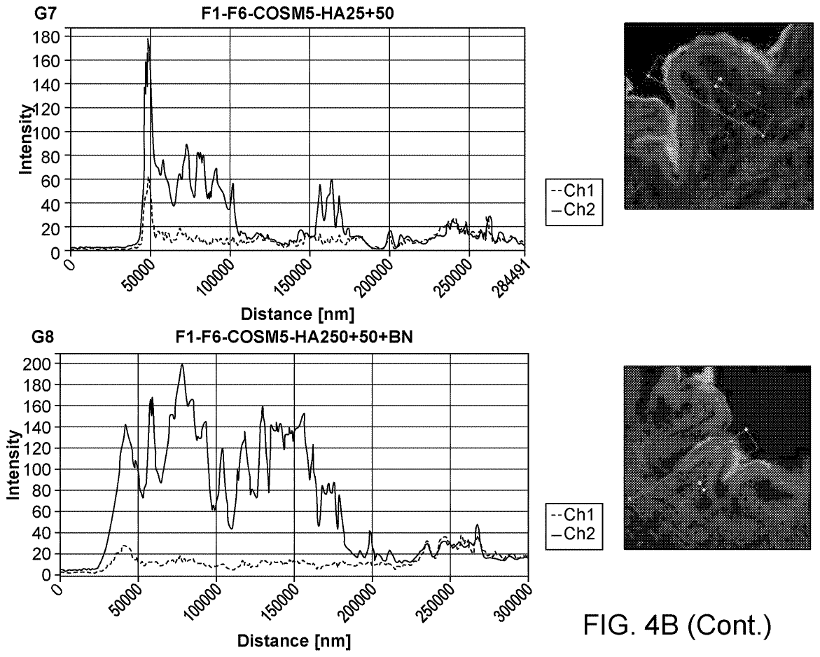

Comparing delivery efficiencies from compositions with ingredients added for cosmeceutical effects indicated that tribehenin and benzyl nicotinate (BN) influenced the delivery. For example, for the delivery of HA250/10K formulations G1 vs G2 (Table H) and formulas G3 and G4 (Table H and FIGS. 4A-4B) indicates that BN enhanced delivery whether T was present or absent.

When comparing formulas G5 and G7 (Table H) and formulas G6 and G8 (Table H and FIGS. 4A-4B) indicates that the presence or absence of tribehenin alone (without BN) did not affect delivery, but BN enhanced delivery when tribehenin was not present which may mean that tribehenin inhibits the effect of BN enhancing delivery. This is also noticeable when comparing formulas G7 and G8 (Table H and FIGS. 4A-4B) which shows that in the absence of tribehenin, BN increases delivery.

When comparing formulas G4 and G8 (Table H and FIGS. 4A-4B) it is noted that using BN but no tribehenin is the preferable formulations for delivering both HA250/10K or 250/50K combinations, with an overall 200% increased efficiency.

Another formulation composition tested but found to be less effective in delivering HA250/50K was formula G9 (Table H) when the phospholipid component was replaced with another type of phospholipid. Additionally, an increase in concentration of lipid phase components and the inclusion of each of 250 kDa, 50 kDa, and 10 kDa MW hyaluronic acid (formula G10; Table H) was also found to be less effective. Compare, e.g., G10 of Table H with E2 and E5 of Table F.

Additionally, there was no substantial difference in the delivery of the two different HA mwt combinations 250/10K or 250/50K from the different sets of equivalent formulations with the same overall composition. See, e.g., G1 vs. G5 and G3 vs. G7.

In Table F and H, the ratio of Ch1/Ch2 fluoresce values are also shown for the skin samples to which various formulations were applied. The similar ratio indicates that the delivery of FITC-HA10 or FITC-HA50K and Rho-HA250K is similar to the original ratio of these two actives in the multisome formulations, that is they are simultaneously delivered at the same extent. Ratios that are lower indicate that the HA250K component delivery is further enhanced relative to the smaller polymer.

### Example 5. Evaluation of a Multisome Formulation to Enhance Penetration of One or More Peptides

The objective of this work is to explore the skin penetration properties of one or more peptides in next generation biphasic vesicle formulations (multisomes). All of the peptide provided herein (e.g., SEQ ID NO: 1-51) possess similar properties for purposes of formulation a lipid vesicle delivery composition (e.g., similar size, conformation, charge, etc.).

Multisome type vesicles with different formulations are prepared with one or more peptides at loading concentration of about 2 mg/mL. The formulations are characterized for physicochemical properties.

An in vitro diffusion cell study is conducted with multiphasic vesicle formulations and transdermal fractions are collected for further analysis by mass spectrometry at a laboratory.

Overall, the results show that multiphasic vesicles cream formulations containing one or more suitable peptides can be prepared for intra/transdermal delivery.

The specific objectives for these experiments are to assess the skin delivery of one or more peptides from various formulations developed. The steps for accomplishing the specific objectives are as follows: 1) The uptake of one or more peptides through human skin is evaluated in experiments using diffusion cells (e.g., Bronaugh type in-line diffusion cells) and human skin. Formulations containing one or more peptides is applied to skin samples and the penetration through the skin is evaluated in the transdermal fractions by mass spectrometry; 2) As a reference, free peptides of the one or more peptides in solution is used for comparison, and blank vehicles are used as controls; 3) Transdermal fractions are collected periodically for analysis; 4) The fractions are pooled and concentrated by filtration (e.g., Pall filtration) and shipped for analysis at a laboratory.

### Materials and Methods

Formulations: Biphasic vesicles with multiple/synergistic penetration enhancers (multisomes)—Different vesicles are formulated, and select formulations are chosen to undergo further testing. For formulation development, one or more peptides including SEQ ID NO: 21-39 are used. For the diffusion cell experiments, multisomes without peptide and with one or more peptides are used. The pH of the formulations are between 6.0-7.0.

Physicochemical characterization—Organoleptic observations, light microscopy and confocal microscopy are carried out to characterize the formulations.

Size (hydrodynamic diameter), polydispersity index and zeta(s) potential measurements are carried out on formulations prepared with non-labelled peptides using a dynamic light scattering (DLS) instrument, for example, the Zetasizer NanoZS (Malvern Instruments, Worcestershire, UK). Aliquots of formulations are assessed for particle size distribution and subsequently diluted with water for zeta potential measurements. Measurements are carried out in triplicates.

In vitro diffusion cell study—Full thickness human skin samples are obtained from an approved vendor. The obtained skin samples are stored at about −20° C. until use.

Absorption of the one or more peptides from formulations into excised human skin in vitro is evaluated using diffusion cells, for example, 9 mm diameter Bronaugh-type teflon flow-through diffusion cells (PermeGear, Inc., Hellertown, PA) with an exposed surface area of about 0.6 cm². The cell holder is maintained at about 32° C. by a circulating water bath heater. A perfusion fluid is used in the diffusion cell, for example, degassed phosphate-buffered saline (PBS) buffer with 0.05% sodium-azide pH 7.2, maintained at 37° C., with a flow rate of 1 mL/h. Skin samples are removed from the freezer and cut into about 1 cm×1 cm square pieces and mounted into the diffusion cells epidermis facing up. The multisome formulations with one or more peptides or control blank formulations are applied to the skin at t=0 and the cells are covered with a teflon cap to provide occlusion. Application is performed periodically and transdermal fractions are collected into tissue culture tubes using a programmed fraction collector.

Transdermal fraction analysis—The transdermal fractions are collected periodically for analysis, for example hourly for 24 h. The samples are sent for analysis to a laboratory.

The skin samples from the diffusion cell study are cleansed by the usual protocol to remove residual bound cream, i.e. after the skin samples are removed from the diffusion cells and washed with water, and patted dry with a kimwipe. The cleansed skin discs are stored at about −20° C.

### Results and Discussion

### Multisome Formulation Optimization and Characterization

All formulations are lotion or cream consistency suitable for topical application. The formulations are physically stable showing no separation, sedimentation or other signs of stability issues for >1 mo of storage at 4° C.

These formulations are shown to be polydisperse with ranging vesicle sizes, as shown in light microscopic images. The microscopic observations confirm the formation of multisomes with the typical biphasic vesicle morphology and the uniform distribution of vesicles throughout the formulation for one or more peptide-containing and blank (no peptide) formulations.

Each of the one or more peptide-containing formulations are similar with respect to size distribution compared to their respective blank formulations, but overall, the blank formulations have narrower size distribution compared to the peptide formulations. Zetasizer data show consistent results with the microscopic observations, typical of multisomes.

Lipid vesicle formulations are prepared with one or more peptide and applied to skin samples. Blank versions of each formulation are prepared as controls, and a solution of the one or more peptides in water is prepared as an additional control. Each formulation is tested in triplicates and blank formulations, skin without any formulations applied, and the one or more peptide solution as free, non-encapsulated peptides are used for background fractions for the analysis.

### Diffusion Cell Study—Transdermal Delivery of Peptides

In this study the transdermal fractions are collected for further analysis by mass spectrometry by a laboratory. The total amount (Qt (24h)) of the one or more peptide delivered through the diameter skin disk applied in the diffusion cells and the delivery rates for each formulation are measured.

These studies show that all multisome formulations delivered the one or more peptides deeply into and through the human skin in vitro.

These studies further show that the one or more peptides in the multisome formulation are delivered deeper into and through the human skin in vitro compared to those that are not in the multisome formulation or are terminally modified, for example to include palmitoyl.

### Example 6. Evaluation of the Safety and Efficacy of Peptides Compared to Placebo for Facial Application

A lipid vesicle formulation of one or more peptides of the disclosure is tested for safety and efficacy for facial application of facial rhytids (skin wrinkles) and glabellar frown lines compared with placebo in in a randomized, double-blind human clinical trial. An amount of one or more peptides, or placebo (blank lipid vesicle) applied to the bilateral forehead and frown line areas of subjects on Day 1.

Primary outcome: Percentage of Participants Achieving a Score of None or Mild by Investigator-Assessment of Facial Wrinkle Scale With Photonumeric Guide (FWS) in Forehead Lines at Maximum Eyebrow Elevation.

On Day 30, the severity of the subject's forehead lines at maximum eyebrow elevation using the 4-point Facial Wrinkle Scale with Photonumeric Guide (FWS): 0=none, 1=mild, 2-moderate or 3=severe is assessed. The percentage of participants with a score of none or mild is determined.

Primary outcome: Percentage of Participants Achieving a Score of None or Mild by Subject-Assessment of Facial Wrinkle Scale With Photonumeric Guide (FWS) in Forehead Lines at Maximum Eyebrow Elevation.

Also on Day 30, the subjects assess the severity of their forehead lines at maximum eyebrow elevation using the 4-point Facial Wrinkle Scale with Photonumeric Guide (FWS): 0=none, 1=mild, 2-moderate or 3=severe and the percentage of participants with a score of none or mild is determined.

Secondary outcome: Percentage of Participants Achieving Satisfied or Very Satisfied by Subject Assessment of Satisfaction of Appearance of Forehead Lines (participant assessment).

On Day 30, participants rate their overall satisfaction with the appearance of the forehead line area using a 5-point scale: 1=very unsatisfied, 2-unsatisfied, 3=neutral, 4-satisfied or 5=very satisfied. The percentage of participants with a rating of satisfied or very satisfied is determined.

Secondary outcome: Percentage of Participants With a=1 Grade Improvement from Baseline by Investigator-Assessed FWS in Forehead Lines at Rest.

At baseline and on Day 30, the Investigator assesses the severity of the subject's forehead lines at rest using the 4-point FWS: 0=none, 1=mild, 2=moderate or 3=severe. The percentage of participants with a=1 grade improvement from baseline is determined.

Secondary outcome: Percentage of Participants With a=1 Grade Improvement from Baseline by Subject-Assessed FWS in Forehead Lines at Rest.

At baseline and on Day 30, the participant assesses the severity of their forehead lines at rest using the 4-point FWS: 0=none, 1=mild, 2=moderate or 3=severe. The percentage of participants with a=1 grade improvement from baseline is determined.

### Example 7. Lipid Vesicle Formulation Development

Biphasic vesicles with multiple/synergistic penetration enhancers (Multisomes) were formulated with different hyaluronic acid (250K and 50K) and various other high molecular weight functional ingredients, and peptides. Additional ingredients, such as those provided in the present disclosure, were also added. For formulation development unlabeled HA was used. For the diffusion cell experiments the vesicles were prepared with fluorescent labelled HAs (Rhodamine-HA250K and FITC-HA50K by Creative PEGWorks).

The general procedure for multisome preparation followed the procedure provided in Example 1. The general procedure for vesicle formulation followed the procedure provided in Example 1. Here, the water phase (System A) was added to the liquid phase in one quick addition. Depending on the batch size, the mixture was intermittently vortexed and heated for 5 sec/5 sec for 8-10 cycles until a uniform creamy lotion formed or mixed with a propeller mixer at high speed for 10-20 min followed by slower speed mixing until the product cools to room temperature.

Various functional ingredient combinations/packs (FIP) with hyaluronic acid (HA) (HA+FIP) were designed. In some embodiments, the HA comprises a HA solution with a high molecular weight and low molecular weight HA. In some embodiments, the HA solution included HA250 and HA50. In some embodiments, the weight ratio of HA250/50 was 2:1 or 1:1. In some embodiments, the HA solution comprising HA250/50 was present in a total amount of about 1 mg/g to about 3 mg/g. In some embodiments, the HA solution comprising HA250/50 was present in a total amount of about 1 mg/g to about 1.5 mg/g, about 1 mg/g to about 2 mg/g, about 1 mg/g to about 2.5 mg/g, about 1 mg/g to about 3 mg/g, about 1.5 mg/g to about 2 mg/g, about 1.5 mg/g to about 2.5 mg/g, about 1.5 mg/g to about 3 mg/g, about 2 mg/g to about 2.5 mg/g, about 2 mg/g to about 3 mg/g, or about 2.5 mg/g to about 3 mg/g. In some embodiments, the HA solution comprising HA250/50 was present in a total amount of about 1 mg/g, about 1.5 mg/g, about 2 mg/g, about 2.5 mg/g, or about 3 mg/g. In some embodiments, HA250 was present in an amount of about 0.5 mg/g to about 1.5 mg/g. In some embodiments, HA250 was present in an amount of about 0.5 mg/g to about 1 mg/g, about 0.5 mg/g to about 1.5 mg/g, or about 1 mg/g to about 1.5 mg/g. In some embodiments, HA250 was present in an amount of about 0.5 mg/g, about 1 mg/g, or about 1.5 mg/g. In some embodiments, HA50 was present in an amount of about 0.5 mg/g to about 1.5 mg/g. In some embodiments, HA50 was present in an amount of about 0.5 mg/g to about 1 mg/g, about 0.5 mg/g to about 1.5 mg/g, or about 1 mg/g to about 1.5 mg/g. In some embodiments, HA50 was present in an amount of about 0.5 mg/g, about 1 mg/g, or about 1.5 mg/g. In some embodiments, the FIP+HA further comprised low MW polyglutamate, high MW polyglutamate, or both (e.g., Hyafactor™ PGA). In some embodiments, the low MW polyglutamate, high MW polyglutamate, or both was present in an amount of about 0.5 mg/g to about 1.5 mg/g. In some embodiments, the low MW polyglutamate, high MW polyglutamate, or both was present in an amount of about 0.5 mg/g to about 1 mg/g, about 0.5 mg/g to about 1.5 mg/g, or about 1 mg/g to about 1.5 mg/g. In some embodiments, the low MW polyglutamate, high MW polyglutamate, or both was present in an amount of about 0.5 mg/g, about 1 mg/g, or about 1.5 mg/g. In some embodiments, the FIP+HA further comprised a carbohydrate or acceptable salt thereof comprising glucosamine phosphate (e.g., NovHyal™). In some embodiments, the carbohydrate or acceptable salt thereof comprising glucosamine phosphate was present in a liquid form. In some embodiments, the carbohydrate or acceptable salt thereof comprising glucosamine phosphate was present in an amount of about 1 mg/g to about 3 mg/g. In some embodiments, the carbohydrate or acceptable salt thereof comprising glucosamine phosphate was present in an amount of about 1 mg/g to about 2 mg/g, about 1 mg/g to about 3 mg/g, or about 2 mg/g to about 3 mg/g. In some embodiments, the carbohydrate or acceptable salt thereof comprising glucosamine phosphate was present in an amount of about 1 mg/g, about 2 mg/g, or about 3 mg/g. In some embodiments, the HA+FIP further comprised crosslinked HA (e.g., Hyacross™). In some embodiments, the cross-linked HA comprise a sodium hyaluronate crosspolymer. In some embodiments, an additive comprising the cross-linked HA further comprises water. In some embodiments, an additive comprising the cross-linked HA further comprises pentylene glycol. In some embodiments, the crosslinked HA was present as a gel. In some embodiments, the crosslinked HA was present in an amount of about 0.05 mg/g to about 5 mg/g. In some embodiments, the crosslinked HA was present in an amount of about 0.05 mg/g to about 0.1 mg/g, about 0.05 mg/g to about 0.2 mg/g, about 0.05 mg/g to about 0.5 mg/g, about 0.05 mg/g to about 1 mg/g, about 0.05 mg/g to about 5 mg/g, about 0.1 mg/g to about 0.2 mg/g, about 0.1 mg/g to about 0.5 mg/g, about 0.1 mg/g to about 1 mg/g, about 0.1 mg/g to about 5 mg/g, about 0.2 mg/g to about 0.5 mg/g, about 0.2 mg/g to about 1 mg/g, about 0.2 mg/g to about 5 mg/g, about 0.5 mg/g to about 1 mg/g, about 0.5 mg/g to about 5 mg/g, or about 1 mg/g to about 5 mg/g. In some embodiments, the crosslinked HA was present in an amount of about 0.05 mg/g, about 0.1 mg/g, about 0.2 mg/g, about 0.5 mg/g, about 1 mg/g, or about 5 mg/g. In some embodiments, the HA+FIP further comprised a saccharide isomerate (e.g., Hyanify™). In some embodiments, the saccharide isomerate was present in an amount of about 1 mg/g to about 10 mg/g. In some embodiments, the saccharide isomerate was present in an amount of about 1 mg/g to about 5 mg/g, about 1 mg/g to about 10 mg/g, or about 5 mg/g to about 10 mg/g. In some embodiments, the saccharide isomerate was present in an amount of about 1 mg/g, about 5 mg/g, or about 10 mg/g. In some embodiments, the HA+FIP further comprised collagen. In some embodiments, the collagen was human collagen. In some embodiments, the collagen was vegan collagen (e.g., HumaColl21®). In some embodiments, the collagen was in a powder form. In some embodiments, the collagen was present in an amount of about 0.1 mg/g to about 0.5 mg/g. In some embodiments, the collagen was present in an amount of about 0.1 mg/g to about 0.2 mg/g, about 0.1 mg/g to about 0.5 mg/g, or about 0.2 mg/g to about 0.5 mg/g. In some embodiments, the collagen was present in an amount of about 0.1 mg/g, about 0.2 mg/g, or about 0.5 mg/g. In some embodiments, the HA+FIP comprises an HA solution comprising HA250/50, low MW polyglutamate, and a carbohydrate or acceptable salt thereof comprising glucosamine phosphate. In some embodiments, the HA+FIP comprises an HA solution comprising HA250/50, low MW polyglutamate, high MW polyglutamate, crosslinked HA, and a saccharide isomerate. In some embodiments, the HA+FIP comprises an HA solution comprising HA250/50, low MW polyglutamate, a carbohydrate or acceptable salt thereof comprising glucosamine phosphate, and collagen. In some instances, the HA250/50 had a 1:1 ratio. In some instances, the HA250/50 had a 2:1 ratio. In some embodiments, the HA+FIP comprises an HA solution comprising HA250/50, low MW polyglutamate, high MW polyglutamate, a carbohydrate or acceptable salt thereof comprising glucosamine phosphate, and collagen. In some embodiments, the HA+FIP comprises an HA solution comprising HA250/50, low MW polyglutamate, high MW polyglutamate, a carbohydrate or acceptable salt thereof comprising glucosamine phosphate, crosslinked HA, and collagen. The different HA 250/50 ratios were evaluated, including unlabeled and fluorescent labelled formulations. Formulations with these FIPs were evaluated for vesicle formation and encapsulation of the various FIPs without peptides first, then with peptides, such as one or more of SEQ ID NOs: 1-51, were also included in the formulations. In some embodiments, the one or more peptides were not terminally modified. In some embodiments, one or more peptides were added in an amount of 0.001 to 0.1 mg/mL. In some embodiments, the one or more peptides was added in an amount of about 0.001 mg/mL to about 0.005 mg/mL, about 0.001 mg/mL to about 0.01 mg/mL, about 0.001 mg/mL to about 0.02 mg/mL, about 0.001 mg/mL to about 0.05 mg/mL, about 0.001 mg/mL to about 0.08 mg/mL, about 0.001 mg/mL to about 0.1 mg/mL, about 0.005 mg/mL to about 0.01 mg/mL, about 0.005 mg/mL to about 0.02 mg/mL, about 0.005 mg/mL to about 0.05 mg/mL, about 0.005 mg/mL to about 0.08 mg/mL, about 0.005 mg/mL to about 0.1 mg/mL, about 0.01 mg/mL to about 0.02 mg/mL, about 0.01 mg/mL to about 0.05 mg/mL, about 0.01 mg/mL to about 0.08 mg/mL, about 0.01 mg/mL to about 0.1 mg/mL, about 0.02 mg/mL to about 0.05 mg/mL, about 0.02 mg/mL to about 0.08 mg/mL, about 0.02 mg/mL to about 0.1 mg/mL, about 0.05 mg/mL to about 0.08 mg/mL, about 0.05 mg/mL to about 0.1 mg/mL, or about 0.08 mg/mL to about 0.1 mg/mL. In some embodiments, the one or more peptides was added in an amount of about 0.001 mg/mL, about 0.005 mg/mL, about 0.01 mg/mL, about 0.02 mg/mL, about 0.05 mg/mL, about 0.08 mg/mL, or about 0.1 mg/mL. In some embodiments, each of the one or more peptides was added in an amount of about 0.001 mg/mL, about 0.005 mg/mL, about 0.01 mg/mL, about 0.02 mg/mL, about 0.05 mg/mL, about 0.08 mg/mL, or about 0.1 mg/mL. In some embodiments, the one or more peptides was added in total in an amount of about 0.001 mg/mL, about 0.005 mg/mL, about 0.01 mg/mL, about 0.02 mg/mL, about 0.05 mg/mL, about 0.08 mg/mL, about 0.1 mg/mL, about 0.2 mg/mL, about 0.3 mg/mL, about 0.4 mg/mL, or about 0.5 mg/mL.

Various aqueous phases (System A) were designed for use in preparing the HA+FIP formulations. In some embodiments, the aqueous phase comprised caprylic and/or capric triglycerides (e.g., Labrafac™ CC). In some embodiments, the caprylic and/or capric triglyceride was present in an amount of about 1% to about 10%. In some embodiments, the caprylic and/or capric triglyceride was present in an amount of about 1% to about 2%, about 1% to about 5%, about 1% to about 8%, about 1% to about 10%, about 2% to about 5%, about 2% to about 8%, about 2% to about 10%, about 5% to about 8%, about 5% to about 10%, or about 8% to about 10%. In some embodiments, the caprylic and/or capric triglyceride was present in an amount of about 1%, about 2%, about 5%, about 8%, or about 10%. In some embodiments, the aqueous phase comprised a glyceryl monostearate (e.g., Glyceryl monostearate NE). In some embodiments, the glyceryl monostearate was present in an amount of about 0.5% to about 2%. In some embodiments, the glyceryl monostearate was present in an amount of about 0.5% to about 1%, about 0.5% to about 1.2%, about 0.5% to about 1.5%, about 0.5% to about 2%, about 1% to about 1.2%, about 1% to about 1.5%, about 1% to about 2%, about 1.2% to about 1.5%, about 1.2% to about 2%, or about 1.5% to about 2%. In some embodiments, the glyceryl monostearate was present in an amount of about 0.5%, about 1%, about 1.2%, about 1.5%, or about 2%. In some embodiments, the aqueous phase comprised a fatty alcohol. In some embodiments, the fatty alcohol was cetyl alcohol. In some embodiments, the fatty alcohol was present in an amount of about 0.1% to about 1%. In some embodiments, the fatty alcohol was present in an amount of about 0.1% to about 0.5%, about 0.1% to about 0.6%, about 0.1% to about 0.7%, about 0.1% to about 1%, about 0.5% to about 0.6%, about 0.5% to about 0.7%, about 0.5% to about 1%, about 0.6% to about 0.7%, about 0.6% to about 1%, or about 0.7% to about 1%. In some embodiments, the fatty alcohol was present in an amount of about 0.1%, about 0.5%, about 0.6%, about 0.7%, or about 1%. In some embodiments, the aqueous phase comprised a wax. In some embodiments, the wax was bees wax (e.g., Synchrowax™ BB4). In some embodiments, the wax was vegan wax (e.g., Synchrowax SB1™). In some embodiments, the wax was present in an amount of about 0.1% to about 0.6%. In some embodiments, the wax was present in an amount of about 0.1% to about 0.2%, about 0.1% to about 0.3%, about 0.1% to about 0.4%, about 0.1% to about 0.5%, about 0.1% to about 0.6%, about 0.2% to about 0.3%, about 0.2% to about 0.4%, about 0.2% to about 0.5%, about 0.2% to about 0.6%, about 0.3% to about 0.4%, about 0.3% to about 0.5%, about 0.3% to about 0.6%, about 0.4% to about 0.5%, about 0.4% to about 0.6%, or about 0.5% to about 0.6%. In some embodiments, the wax was present in an amount of about 0.1%, about 0.2%, about 0.3%, about 0.4%, about 0.5%, or about 0.6%. In some embodiments, the aqueous phase comprised a silicon oil. In some embodiments, the silicon oil was dimethicone (e.g., Dimethicone 200CST). In some embodiments, the silicon oil was present in an amount of about 0.5% to about 2%. In some embodiments, the silicon oil was present in an amount of about 0.5% to about 1%, about 0.5% to about 2%, or about 1% to about 2%. In some embodiments, the silicon oil was present in an amount of about 0.5%, about 1%, or about 2%. In some embodiments, the aqueous phase comprised a PEG ether of a fatty alcohol. In some embodiments, the PEG ether of a fatty alcohol was polyoxyethylene (2) oleyl ether (e.g., super refined Brij® 02). In some embodiments, the PEG ether of a fatty alcohol was present in an amount of about 0.5% to about 2%. In some embodiments, the PEG ether of a fatty alcohol was present in an amount of about 0.5% to about 1%, about 0.5% to about 2%, or about 1% to about 2%. In some embodiments, the PEG ether of a fatty alcohol was present in an amount of about 0.5%, about 1%, or about 2%. In some embodiments, the aqueous phase comprised cationic surfactant. In some embodiments, the cationic surfactant comprised linoleamidopropyl PG-dimonium chloride phosphate (e.g., Arlasilk™ EFA). In some embodiments, the cationic surfactant was present in an amount of about 1% to about 20%. In some embodiments, the cationic surfactant was present in an amount of about 1% to about 5%, about 1% to about 10%, about 1% to about 20%, about 5% to about 10%, about 5% to about 20%, or about 10% to about 20%. In some embodiments, the cationic surfactant was present in an amount of about 1%, about 5%, about 10%, or about 20%. In some embodiments, the aqueous phase comprised a preservative (e.g., Spectrastat). In some embodiments, the preservative was Caprylhydroxamic acid/caprylyl glycol. In some embodiments, the preservative was present in an amount of about 1% to about 2%. In some embodiments, the preservative was present in an amount of about 1% to about 1.5%, about 1% to about 2%, or about 1.5% to about 2%. In some embodiments, the preservative was present in an amount of about 1%, about 1.5%, or about 2%. In some embodiments, the aqueous phase comprised a polysorbate. In some embodiments, the polysorbate was polysorbate80 (e.g., Tween 80). In some embodiments, the polysorbate was present in an amount of about 0.5% to about 1%. In some embodiments, the polysorbate was present in an amount of about 0.5% to about 0.8%, about 0.5% to about 1%, or about 0.8% to about 1%. In some embodiments, the polysorbate was present in an amount of about 0.5%, about 0.8%, or about 1%. In some embodiments, the aqueous phase comprised a vitamin. In some embodiments, the vitamin was niacinamide. In some embodiments, the vitamin was a Vitamin C derivative (e.g., K3 Vita-C). In some embodiments, the vitamin was present in an amount of about 0.5% to about 4%. In some embodiments, the vitamin was present in an amount of about 0.5% to about 0.8%, about 0.5% to about 1%, about 0.5% to about 2%, about 0.5% to about 3%, about 0.5% to about 3.2%, about 0.5% to about 4%, about 0.8% to about 1%, about 0.8% to about 2%, about 0.8% to about 3%, about 0.8% to about 3.2%, about 0.8% to about 4%, about 1% to about 2%, about 1% to about 3%, about 1% to about 3.2%, about 1% to about 4%, about 2% to about 3%, about 2% to about 3.2%, about 2% to about 4%, about 3% to about 3.2%, about 3% to about 4%, or about 3.2% to about 4%. In some embodiments, the vitamin was present in an amount of about 0.5%, about 0.8%, about 1%, about 2%, about 3%, about 3.2%, or about 4%. In some embodiments, the aqueous phase comprised purified water. In some embodiments, the aqueous phase comprised a caprylic and/or capric triglyceride, a glyceryl monostearate, a fatty alcohol, a beeswax, a silicon oil, a PEG ether of a fatty alcohol, a cationic surfactant, a preservative, and purified water. In some embodiments, the aqueous phase comprised a caprylic and/or capric triglyceride, a glyceryl monostearate, a fatty alcohol, a vegan beeswax, a silicon oil, a PEG ether of a fatty alcohol, a polysorbate, a cationic surfactant, a preservative, and purified water. In some embodiments, the cationic surfactant was doubled in concentration. In some embodiments, the aqueous phase comprised a caprylic and/or capric triglyceride, a glyceryl monostearate, a fatty alcohol, a vegan beeswax, a silicon oil, a PEG ether of a fatty alcohol, a polysorbate, a cationic surfactant, vitamins, a preservative, and purified water. In some embodiments, the vitamins comprised two vitamins.

Various phospholipid phases were designed for use in preparing the HA+FIP formulations. Variations with a plant sterol and cholesterol, 2,3-butanediol and propylene glycol, and variations of phospholipid concentrations were designed. In some embodiments, the phospholipid phase comprised phospholipids. In some embodiments, the phospholipids were hydrogenated phosphatidylcholine (e.g., Sunlipon® 90H). In some embodiments, the phospholipid was present in an amount of about 5% to about 12%. In some embodiments, the phospholipid was present in an amount of about 5% to about 7%, about 5% to about 10%, about 5% to about 10.5%, about 5% to about 12%, about 7% to about 10%, about 7% to about 10.5%, about 7% to about 12%, about 10% to about 10.5%, about 10% to about 12%, or about 10.5% to about 12%. In some embodiments, the phospholipid was present in an amount of about 5%, about 7%, about 10%, about 10.5%, or about 12%. In some embodiments, the phospholipid phase comprised a sterol. In some embodiments, the sterol comprised cholesterol. In some embodiments, the sterol was a vegan sterol (e.g., Phytosterol MM). In some embodiments, the sterol was present in an amount of about 1% to about 3%. In some embodiments, the sterol was present in an amount of about 1% to about 1.5%, about 1% to about 1.7%, about 1% to about 2%, about 1% to about 2.4%, about 1% to about 2.6%, about 1% to about 3%, about 1.5% to about 1.7%, about 1.5% to about 2%, about 1.5% to about 2.4%, about 1.5% to about 2.6%, about 1.5% to about 3%, about 1.7% to about 2%, about 1.7% to about 2.4%, about 1.7% to about 2.6%, about 1.7% to about 3%, about 2% to about 2.4%, about 2% to about 2.6%, about 2% to about 3%, about 2.4% to about 2.6%, about 2.4% to about 3%, or about 2.6% to about 3%. In some embodiments, the sterol was present in an amount of about 1%, about 1.5%, about 1.7%, about 1.73%, about 2%, about 2.4%, about 2.6%, or about 3%. In some embodiments, the phospholipid phase comprised propylene glycol. In some embodiments, the propylene glycol was present in an amount of about 5% to about 12%. In some embodiments, the propylene glycol was present in an amount of about 5% to about 7%, about 5% to about 10%, about 5% to about 10.5%, about 5% to about 12%, about 7% to about 10%, about 7% to about 10.5%, about 7% to about 12%, about 10% to about 10.5%, about 10% to about 12%, or about 10.5% to about 12%. In some embodiments, the propylene glycol was present in an amount of about 5%, about 7%, about 10%, about 10.5%, or about 12%. In some embodiments, the phospholipid phase comprised a butanediol. In some embodiments, the butanediol was 2,3-butanediol (e.g., GreenDiol™). In some embodiments, the butanediol was present in an amount of about 5% to about 12%. In some embodiments, the butanediol was present in an amount of about 5% to about 7%, about 5% to about 10%, about 5% to about 10.5%, about 5% to about 12%, about 7% to about 10%, about 7% to about 10.5%, about 7% to about 12%, about 10% to about 10.5%, about 10% to about 12%, or about 10.5% to about 12%. In some embodiments, the butanediol was present in an amount of about 5%, about 7%, about 10%, about 10.5%, or about 12%. In some embodiments, the phospholipid phase comprised phospholipids, a vegan sterol, and propylene glycol. In some embodiments, the phospholipid phase comprised phospholipids, a vegan sterol, and 2,3-butenediol. In some embodiments, the phospholipid phase comprised phospholipids, a cholesterol, and propylene glycol. In some embodiments, the phospholipid phase comprised the same amount of phospholipid, sterol, propylene glycol, butanediol, or any combination thereof.

Various combinations of the phospholipid phase, aqueous phase (System A) and HA+FIP formulation were prepared. The various combination that were prepared in the experiment is provided in Table I.

TABLE I
Multisome (biphasic vesicle) compositions

| | Lipid | | |
|---|---|---|---|
| | phase | System A | FIPs |
| Developmental formulations: | | | |
| (Phytosterol formulas) | | | |
| F5P-7G-F6COSM5V-H2 | F5P-7G | F6COSM5V | HA2 |
| F5P-7G-F6COSM5V-H2 | F5P-7G | F6COSM5V | HA2 + P2 |
| F5P-7G-F6COSM5V-H4 | F5P-7G | F6COSM5V2 | HA4 |
| F5P-7G-F6COSM5V-H4 | F5P-7G | F6COSM5V2 | HA4 + P2 |
| F5P-G-F6COSM5V-H6 | F5P-G | F6COSM5V2 | H6 |
| F5P-G-F6COSM5V-H6 | F5P-G | F6COSM5V2 | H6 + P2 |
| F5P-F6COSM5V2-H8 | F5P | F6COSM5V2 | H8 |
| F5P-F6COSM5V2-H8 + P2 | F5P | F6COSM5V2 | H8 + P2 |
| Developmental formulations: | | | |
| (cholesterol formulas) | | | |
| F1-F6COSM5V2-H8 + P2 | F1 | F6COSM5V2 | H8 + P2 |
| F1-F6COSM5V2-H6 + P2 | F1 | F6COSM5V2 | H6 + P2 |
| F1-F6COSM5V2-H5-1 + P2 | F1 | F6COSM5V2 | H5-1 + P2 |
| F1-F6COSM5V2-H5-2 + P2 | F1 | F6COSM5V2 | H5-2 + P2 |
| Fluorescent formulations: | | | |
| F5P-F6COSM5V2-H6 + P2 | F5P2.6 | F6COSM5V2 | H6 + P2 |
| (phytosterol formula) | | | |
| F1-F6COSM5V2-H6 + P2 | F1 | F6COSM5V2 | H6 + P2 |
| (cholesterol formula) | | | |
| F5P-F6COSM5V2-H5-1 + P2 | F5P2.6 | F6COSM5V2 | H5-1 + P2 |
| (phytosterol formula) | | | |
| F1-F6COSM5V2-H5-1 + P2 | F1 | F6COSM5V2 | H5-1 + P2 |
| (cholesterol formula) | | | |
| F5P-F6COSM5V2-H5-2 + P2 | F5P2.6 | F6COSM5V2 | H5-2 + P2 |
| (phytosterol formula) | | | |
| F1-F6COSM5V2-H5-2 + P2 | F1 | F6COSM5V2 | H5-2 + P2 |
| (cholesterol formula) | | | |
| F5P-7G-F6COSM5V-H4 | F1-7 | F6COSM5V2 | HA4 |
| F5P-G-F6COSM5V-H6 | F5P2.6-G | F6COSM5V2 | HA6 |
| F5P-F6COSM5V2-H8 + P2 | F5P2.6 | F6COSM5V2 | H8 + P2 |
| (phytosterol formula) | | | |
| DIFF Cell Study: | | | |
| F5P-F6COSM5V2-H5-1 + P2 | F5P2.6 | F6COSM5V2 | H5-1 + P2 |
| F1-F6COSM5V2-H5-1 + P2 | F1 | F6COSM5V2 | H5-1 + P2 |
| F5P-F6COSM5V2-H5-2 + P2 | F5P2.6 | F6COSM5V2 | H5-2 + P2 |
| F1-F6COSM5V2-H5-2 + P2 | F1 | F6COSM5V2 | H5-2 + P2 |
| F5P-F6COSM5V2-H6 + P2 | F5P2.6 | F6COSM5V2 | H6 + P2 |
| F1-F6COSM5V2-H6 + P2 | F1 | F6COSM5V2 | H6 + P2 |
| F5P-F6COSM5V2-H8 + P2 | F5P2.6 | F6COSM5V2 | HA8 + P2 |
| F5PG-F6COSM5V2-H5-1 + P2 | F5P2.6-G | F6COSM5V2 | HA5-1 |
| F5PG-F6COSM5V2-H5-2 + P2 | F5P2.6-G | F6COSM5V2 | HA5-2 |
| Formulations with vitamins: | | | |
| F1-F6COSM5NC | F1 | F6COSM5NC | HA4 + P2 |

Table I shows the compositions for the various non-fluorescent and fluorescent formulations prepared and evaluated for cosmetic properties, encapsulation of FIPs and delivery of HA250/50 components into human skin in vitro.

### Characterization of Formulations

The methods of characterization and analysis of the formulations followed the methods generally outlined in Example 2, including the physiochemical characterization, in vitro diffusion cell studies, and skin analysis. The objectives were to encapsulate various combinations and number of HA and other functional ingredients and determine if there is a limit for loading into multisomes and evaluate possibilities for various combinations for a suitable cosmetic product.

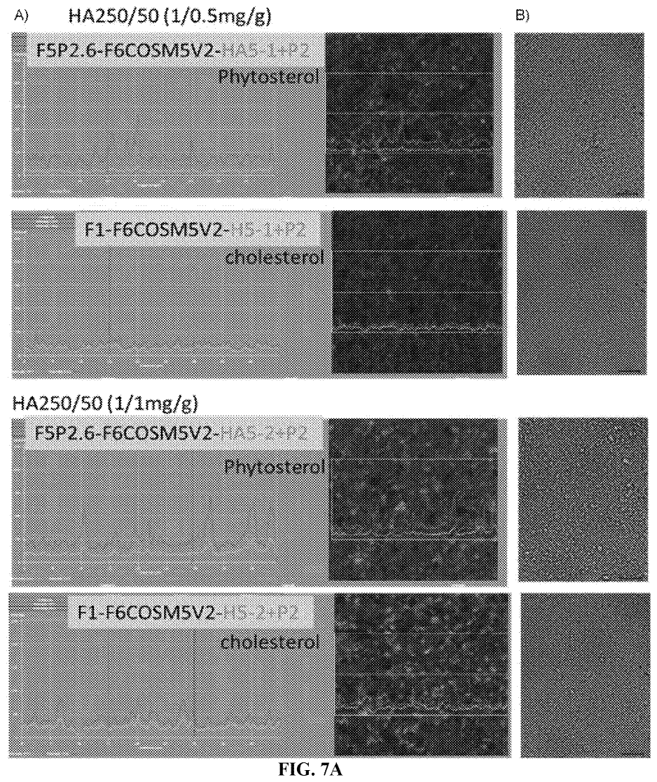

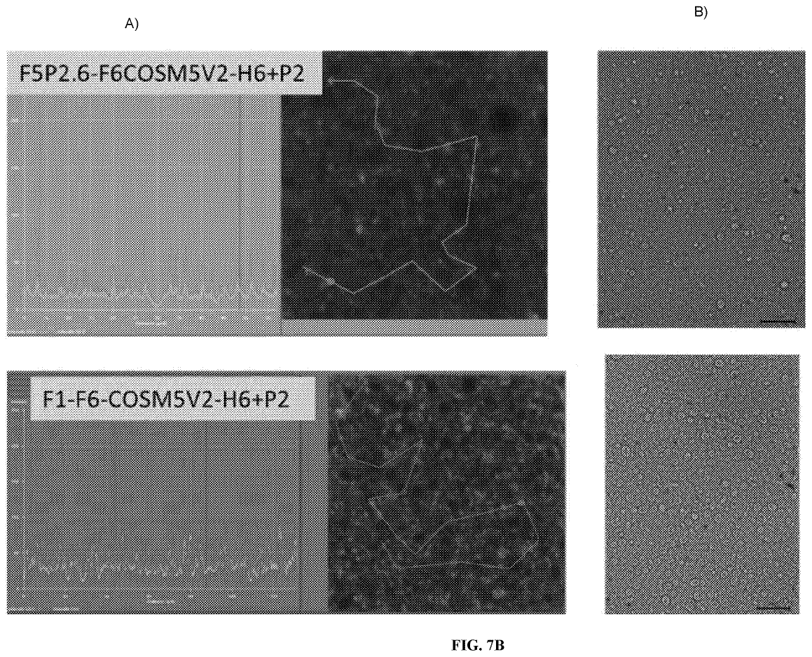

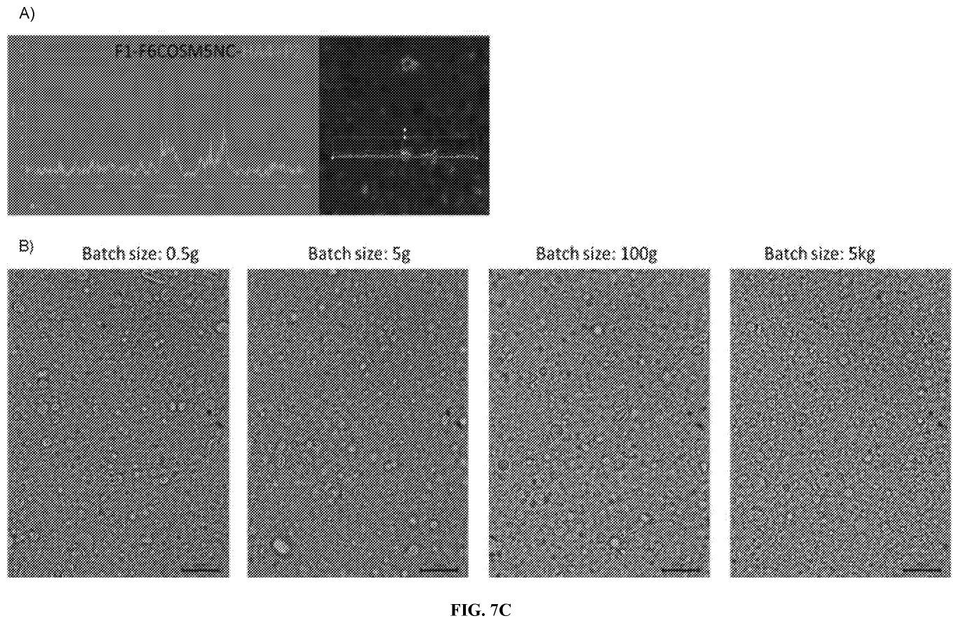

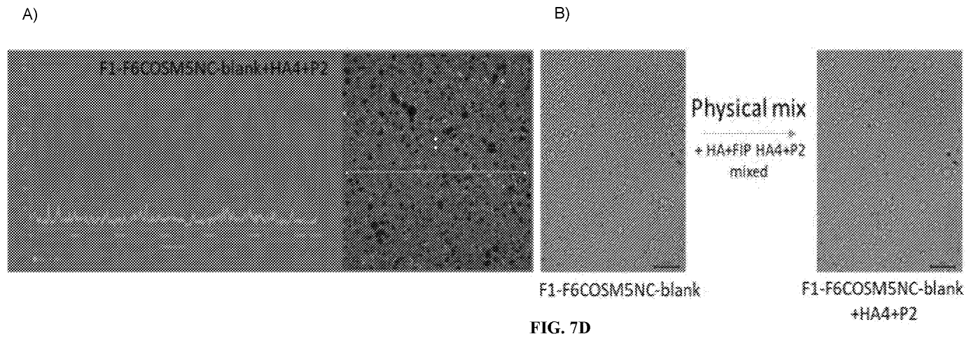

Confocal microscopic studies of the multisomes showing the distribution of Rho-HA250K (red) and FITC-HA50K (green) fluorescence in the formulations were used to analyze the HA encapsulation in the vesicles and determine if the presence of increasing amounts of other functional ingredients had an effect on encapsulation ratios. Confocal microscopic profile tracings confirmed the association of red Rho-HA250K and green FITC-HA50K with the vesicles (Panels A in FIGS. 7A-C). The fluorescence intensity (FI) curves tracing the vesicles along the selected plane showed the co-localization of the red and green fluorescence, indicating the co-encapsulation of the two different molecular weight HAs (HA250K and HA50K). FIG. 7D shows the simple mixing of the preformed biphasic vesicles with HA+FIP HA4+P2 as a reference for non-encapsulated HA+FIP profile of the formulation.

Light microscopic images are shown for each formulation next to the confocal microscopic fluoresce tracings (Panels B in FIGS. 7A-D). These images indicate the formation of multisomes (next generation biphasic vesicles) for each type of formulation. In a selected case of F1-F6COSM5NC, the scalability of the formulation is shown for various batch sizes (0.5 g, 5 g, 100 g, 5000 g) (FIG. 7C).

Zetasizer measurements for each System A (submicron emulsion component) used in the formulations were routinely performed to confirm the average particle size range of approximately <400 nm. The physicochemical properties of the multisome formulations were assessed for color, consistency, and homogeneity and biphasic vesicle formation. All formulations were lotion or cream consistency suitable for topical application. In general, the formulations were physically stable showing no separation, sedimentation or other signs of stability issues for >3 mo of storage at 4° C. and at room temperature. Microscopic observations confirmed that the multisomes remained intact and uniformly distributed during storage.

### Characterization of Delivery

The cryosections of human skin samples treated in vitro in diffusion cells with topical formulations containing fluorescence labelled HA250/50K were evaluated for the presence of fluorescent protein. The enhancement of delivery of (negatively charged) HA compounds is shown for various formulations. These studies indicated that overall multiphasic vesicles delivered significant amount of HA+FIPs into human skin. Several aspects of the delivery efficiency were evaluated.

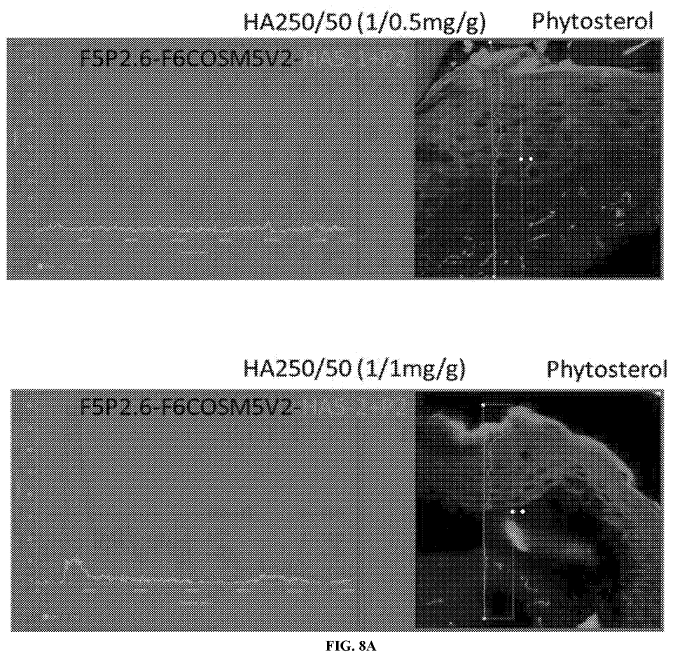

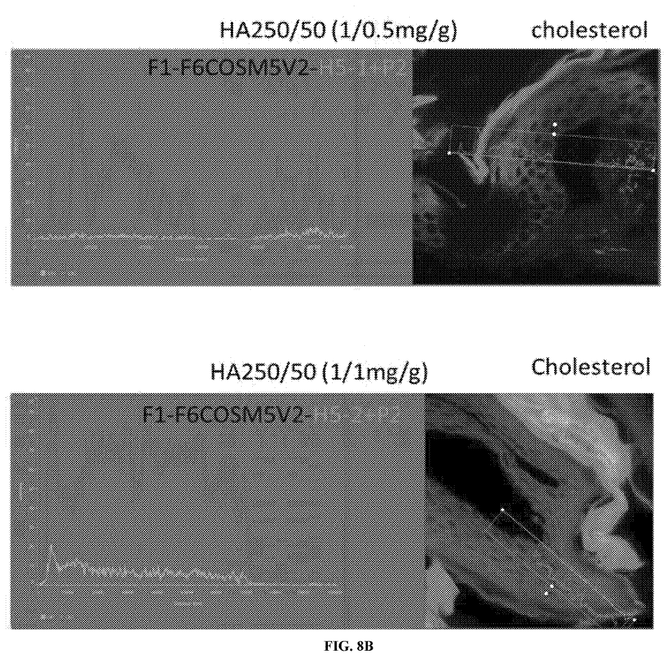

First, the effect of HA250/50 concentration was evaluated. The increase in HA250/50K total concentration from 1.5 mg/g (HA250/50 1/0.5 mg/g) to 2 mg/g (HA250/50 1/1 mg/g) vesicle formulation increased delivery of HAs for both Phytosterol and cholesterol containing formulations (FIGS. 8A-8B). The respective cholesterol containing formulations showed increased delivery compared to Phytosterol containing formulations for both ratios of HA.

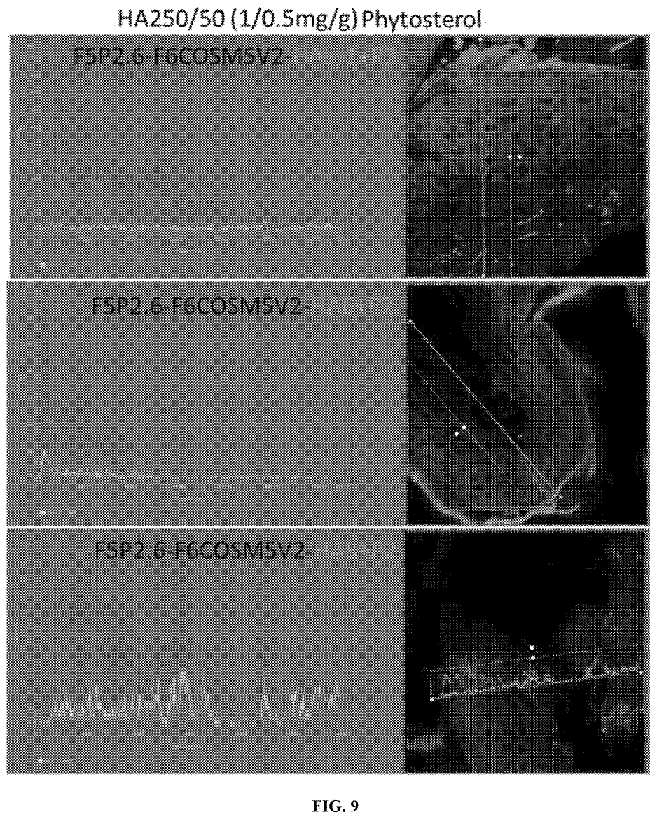

Increasing the number of FIPs decreased the delivery efficiency of the individual HAs as evident from comparing the fluorescence intensities in the skin after treatment with F5P2.5-F6COSM5V2 formulation encapsulating HA5-1+P2, HA6+P2 and HA8+P2 (FIG. 9). This is likely due to the proportional encapsulation of the increasing number of FIPs leading to an overall lower amount of each individual component.

FIG. 10 shows the effect of formulating multisomes with either propylene glycol (top) or 2,3-butenediol (GreenDiol™) (bottom). The formulation was F5P2.6-F6COSM5V2 with HA250/50 at 1/0.5 mg/g. FIG. 10 shows that the 2,3-butenediol containing formulation decreased HA delivery.

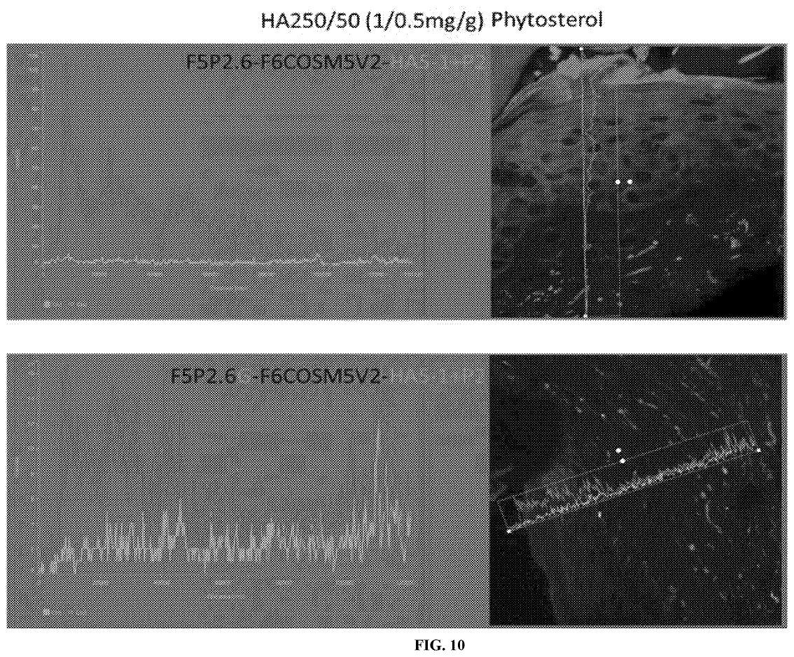

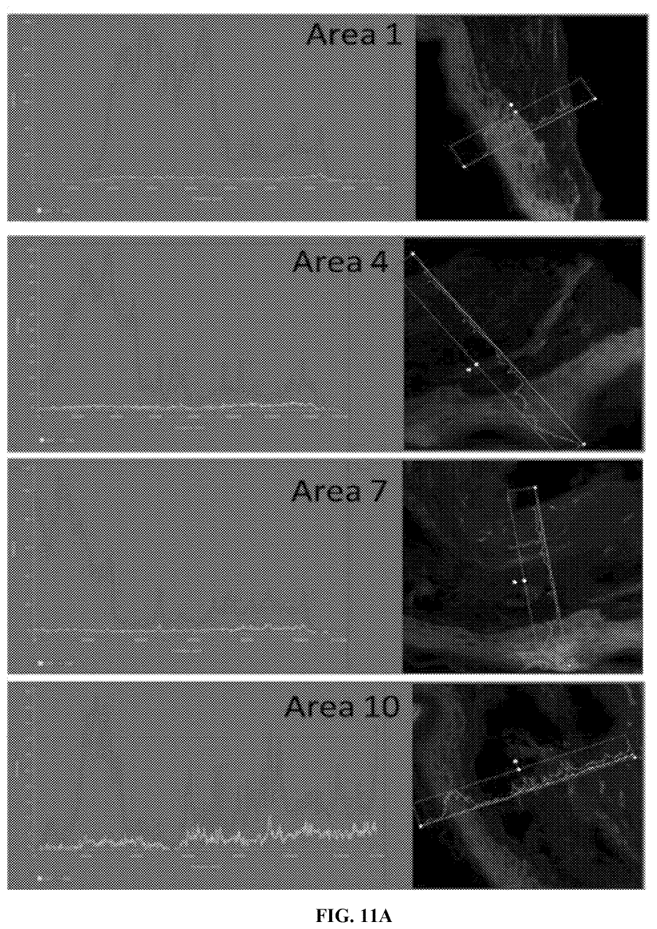

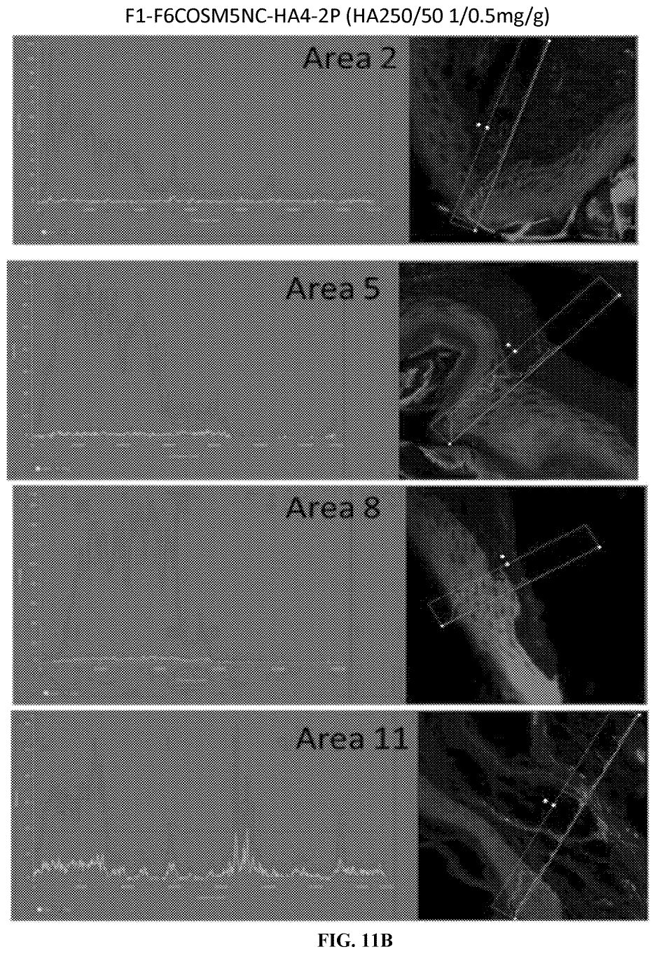

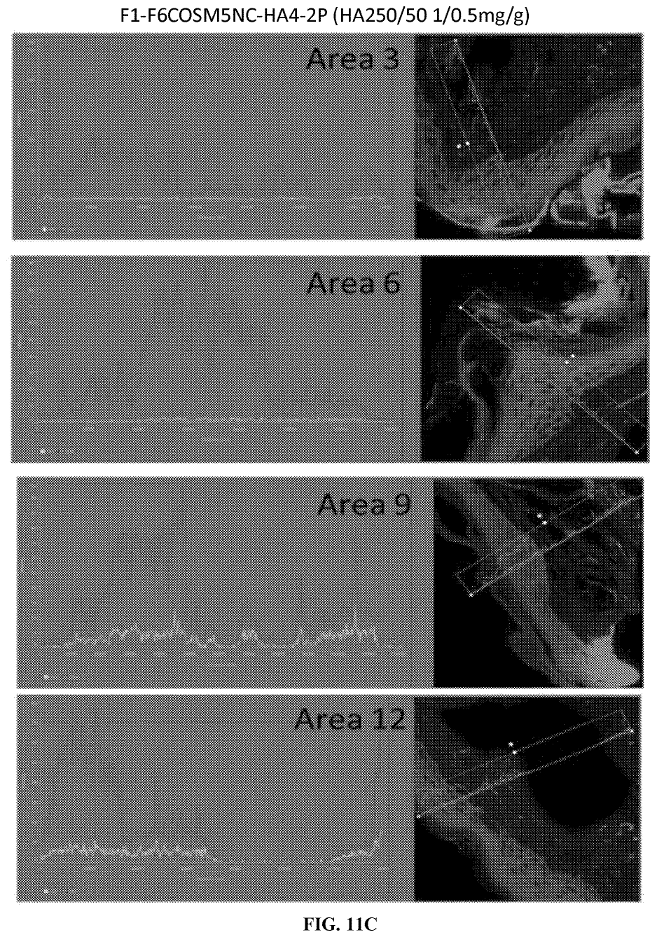

After the optimization of the HA+FIPs encapsulation and multisomes compositions, Formulation F1-F6COSM5NC-HA4-2P (with HA250/50 1/0.5 mg/g) containing additional vitamin components was developed and evaluated. This formulation showed consistently high HA+FIP delivery throughout various layers in the skin from the surface to the upper epidermis (FIGS. 11A-11C).

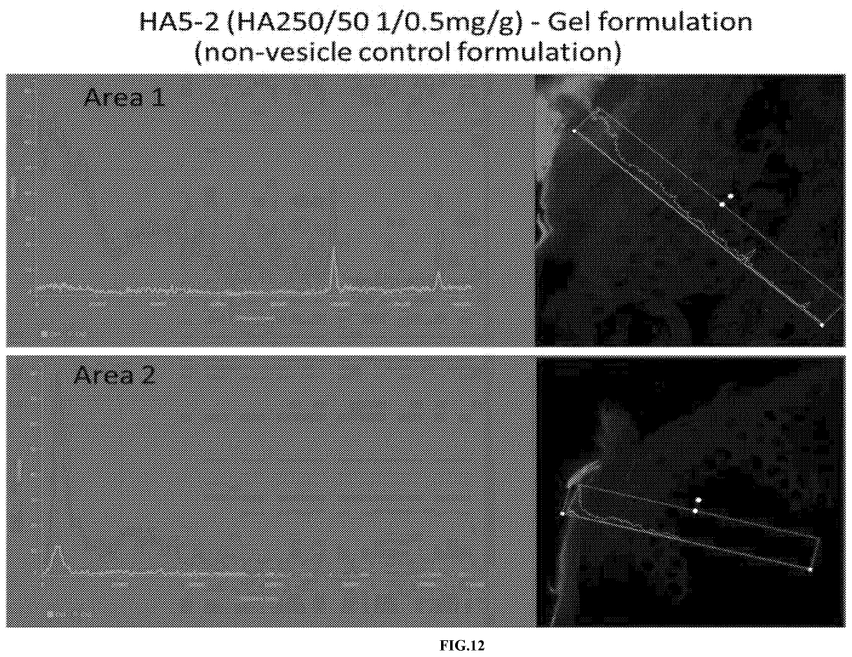

As a reference, a gel formulation (non-vesicle control) was also tested in vitro for HA250/50 delivery. The gel formulation contained HA+FIP HA5-2 (HA250/50 1/1 mg/g) in ‘free’ form. This formulation showed very low to negligible delivery levels, which is shown in two different areas of the skin in FIG. 12.

In Table J, the average Rho-HA250 and FITC-HA50 fluorescence intensities (FI) and ratios are shown for the skin samples treated with selected formulations. The original ratio based on a physical mix of HA+FIP HA2+P2 with vesicles (non-encapsulated) (Ch1/CH2 ratio 10/80) was 0.125. A similar ratio to the original ratio in the formulations indicates the approximate co-delivery of Rho-HA250K and FITC-HA50K. Ratios that are lower indicate that the HA250K component delivery is further enhanced.

All multiphasic vesicle formulations enhanced delivery compared to HA in gel formulation. Formulation design differences of multiphasic vesicles indicated that HA delivery can be modulated.

TABLE J
Average fluorescence intensities in skin treated
with selected multisome HA + FIP formulations

| | Fluorescence | |
|---|---|---|
| | Intensity | |
| | HA Rho250/ | Ch1/Ch2 |
| Formulation | FITC50 (Ch2/Ch1) | ratio |
| F5P2.6-F6COSM5V2-HA5-1 + P2 | 40/3 | 0.075 |
| F1-F6COSM5V2-H5-1 + P2 | 50/3 | 0.06 |
| F5P2.6-F6COSM5V2-HA5-2 + P2 | 20/3 | 0.15 |
| F1-F6COSM5V2-H5-2 + P2 | 75/10 | 0.13 |
| F5P2.6-F6COSM5V2-HA6 + P2 | 20/2 | 0.10 |
| F5P2.6-F6COSM5V2-HA8 + P2 | 12/2 | 0.17 |
| F5P2.6G-F6COSM5V2-HA5-1 + P2 | 12/3 | 0.25 |
| F1-F6COSM5NC-HA4-2P | 80/8 | 0.10 |

### Example 8. Lipid Vesicle Formulation Development for Application to Areas of the Skin

The formulations generally exemplified in Example 7 are developed for application to the skin around the eyes. The formulation comprises one or more peptides from Table 2, each in an amount of about 0.001% to about 0.01% of the formulation. In some examples, the formulation comprises four peptides from Table 2. In some examples, the formulation comprises five peptides from Table 2. In some examples, the formulation comprises butanediol, or a combination of propylene glycol or butanediol. In some examples, the formulations comprise a preservative (e.g., caprylhydroxamic acid/caprylyl glycol) in an amount of about 0.4% to about 0.6% of the formulation. In some examples, the formulation further comprises one or more biological extracts provided herein (e.g., flower extract, root extract, extract from fermented bacteria, etc.). In some examples, the formulation comprises at least one biological extracts. In some examples, the formulation comprises two biological extracts. In some examples, a formulation comprises one or more biological extracts in an amount of about 0.02% to 0.016% of the formulation.

In some instances, the formulations generally exemplified in Example 7 are further developed for application to the skin of the neck. In some examples, a formulation comprises one or more peptides from Table 2, each in an amount of about 0.001% to about 0.01% of the formulation.

### Example 9. Application of the Lipid Vesicles to the Skin

A formulation generally exemplified in Example 7 is applied to the skin of an individual. The formulation includes aqua/water/eau, propylene glycol, hydrogenated lecithin, caprylic/capric triglyceride, cholesterol, linoleamidopropyl PG-dimonium chloride phosphate, niacinamide, dimethicone, polysorbate 80, oleth-2, sodium hyaluronate, hyaluronic acid, aminopropyl ascorbyl phosphate, sodium polyglutamate, glyceryl stearate, cetyl alcohol, pentapeptide-4, sh-hexapeptide-9 SP acetate, disodium acetyl glucosamine phosphate, glycerin, synthetic beeswax, tocopheryl acetate, caprylyl glycol, and caprylhydroxamic acid.

The formulation is applied daily to the skin of the individual, in the morning and in the afternoon. In some examples, the formulation is applied after cleaning the skin of the individual, for example, using a cleanser. In some examples, the formulation is applied before moisturizing the skin of the individual, for example, using a moisturizer.

After about 30 minutes prior to application of the formulation, the skin where the formulation was applied feels more hydrated, moisturized, smoother, softer, or any combination thereof. After 2 weeks of applying the formulation twice a day to the skin, the skin where the formulation was applied feels more hydrated and/or supple, the skin looks more youthful and/or the facial volume is restored, the skin texture looks and/or feels smoother, the lines on the skin are reduced and/or smoothed out, the skin looks and/or feels healthier and/or more rejuvenated, or any combination thereof. In some examples, the formulation hydrates, visibly recovers volume, forms, and/or moisturizes the skin where the formulation is applied.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-20. (canceled)

21. A composition comprising at least two peptides, wherein the at least two peptides comprise amino acid sequences of SEQ ID NOs: 31 and 37.

22. The composition of claim 21, further comprising at least three peptides, wherein the at least three peptides comprise amino acid sequences of SEQ ID NOs: 21, 24, and 40.

23. The composition of claim 21, wherein the at least two peptides comprise unmodified peptides.

24. A composition comprising:
a. lipid vesicles each comprising a lipid bilayer comprising vesicle forming lipids;
b. an oil-in-water emulsion entrapped in the lipid vesicles; and
c. an unmodified peptide.

25. The composition of claim 24, wherein the composition comprises at least two lipid bilayers, and wherein the unmodified peptide is entrapped between adjacent bilayers of the at least two bilayers.

26. The composition of claim 24, wherein the unmodified peptide comprises the amino acid sequence consisting of SEQ ID NO: 31.

27. The composition of claim 24, wherein the unmodified peptide comprises the amino acid sequence consisting of SEQ ID NO: 37.

28. The composition of claim 24, wherein the composition further comprises at least one peptide comprising an amino acid sequence comprising SEQ ID NO: 21, 24, or 40.

29. The composition of claim 24, wherein the composition further comprises one or more vitamins or derivatives thereof.

30. A composition comprising:
a. lipid vesicles each comprising a lipid bilayer comprising vesicle forming lipids;
b. an oil-in-water emulsion entrapped in the lipid vesicles; and
c. a peptide comprising an amino acid sequence in Table 2.

31. The composition of claim 30, wherein the amino acid sequence comprises SEQ ID NO: 31.

32. The composition of claim 30, wherein the amino acid sequence comprises SEQ ID NO: 37.

33. The composition of claim 30, wherein the amino acid sequence comprises SEQ ID NO: 21.

34. The composition of claim 30, wherein the amino acid sequence comprises SEQ ID NO: 24.

35. The composition of claim 30, wherein the amino acid sequence comprises SEQ ID NO: 40.

36. The composition of claim 30, wherein the composition further comprises one or more vitamins or derivatives thereof.

37. The composition of claim 36, wherein the one or more vitamins or derivatives thereof comprises one or more of vitamin B, vitamin C, vitamin A, vitamin E, vitamin D, vitamin F, or vitamin K.

38. The composition of claim 30, wherein the composition comprises niacinamide.

39. The composition of claim 30, wherein the composition comprises a biological extract.

40. The composition of claim 30, wherein the composition comprises polyglutamate.


SEQUENCE LISTING (52 sequences)

SEQ ID NO: 1-20 - skipped (20 entries)

SEQ ID NO: 21 - protein, 4 aa, synthetic construct
  VARIANT 1: note = L-or D-Glycine
  VARIANT 2: note = L-or D-Glutamine
  VARIANT 3: note = L-or D-Proline
  VARIANT 4: note = L-or D-Arginine
GQPR

SEQ ID NO: 22 - protein, 4 aa, synthetic construct
  VARIANT 1: note = L-or D-Lysine
  VARIANT 2: note = L-or D-Threonine
  VARIANT 3: note = L-or D-Phenylalanine
  VARIANT 4: note = L-or D-Lysine
KTFK

SEQ ID NO: 23 - protein, 4 aa, synthetic construct
  VARIANT 1: note = L-or D-Alanine
  VARIANT 2: note = L-or D-Glutamine
  VARIANT 3: note = L-or D-Threonine
  VARIANT 4: note = L-or D-Arginine
AQTR

SEQ ID NO: 24 - protein, 4 aa, synthetic construct
  VARIANT 1: note = L-or D-Alanine
  VARIANT 2: note = L-or D-Histidine
  VARIANT 3: note = L-or D-Serine
  VARIANT 4: note = L-or D-Histidine
AHSH

SEQ ID NO: 25 - protein, 4 aa, synthetic construct
  VARIANT 1: note = L-or D-Arginine
  VARIANT 2: note = L-or D-Serine
  VARIANT 3: note = L-or D-Arginine
  VARIANT 4: note = L-or D-Lysine
RSRK

SEQ ID NO: 26 - protein, 4 aa, synthetic construct
  VARIANT 1: note = L-or D-Lysine
  VARIANT 2: note = L-or D-Aspartic acid
  VARIANT 3: note = L-or D-Valine
  VARIANT 4: note = L-or D-Tyrosine
KDVY

SEQ ID NO: 27 - protein, 4 aa, synthetic construct
  VARIANT 1: note = L-or D-Lysine
  VARIANT 2: note = L-or D-Threonine
  VARIANT 3: note = L-or D-Alanine
  VARIANT 4: note = L-or D-Lysine
KTAK

SEQ ID NO: 28 - protein, 4 aa, synthetic construct
  VARIANT 1: note = L-or D-Lysine
  VARIANT 2: note = L-or D-Phenylalanine
  VARIANT 3: note = L-or D-Tyrosine
  VARIANT 4: note = L-or D-Lysine
KFYK

SEQ ID NO: 29 - protein, 4 aa, synthetic construct
  VARIANT 1: note = L-or D-Lysine
  VARIANT 2: note = L-or D-Alanine
  VARIANT 3: note = L-or D-Tyrosine
  VARIANT 4: note = L-or D-Lysine
KAYK

SEQ ID NO: 30 - protein, 4 aa, synthetic construct
  VARIANT 1: note = L-or D-Threonine
  VARIANT 2: note = L-or D-Threonine
  VARIANT 3: note = L-or D-Lysine
  VARIANT 4: note = L-or D-Serine
TTKS

SEQ ID NO: 31 - protein, 5 aa, synthetic construct
  VARIANT 1: note = L-or D-Lysine
  VARIANT 2: note = L-or D-Threonine
  VARIANT 3: note = L-or D-Threonine
  VARIANT 4: note = L-or D-Lysine
  VARIANT 5: note = L-or D-Serine
KTTKS

SEQ ID NO: 32 - protein, 5 aa, synthetic construct
  VARIANT 1: note = L-or D-Lysine
  VARIANT 2: note = L-or D-Leucine
  VARIANT 3: note = L-or D-Alanine
  VARIANT 4: note = L-or D-Alanine
  VARIANT 5: note = L-or D-Lysine
KLAAK

SEQ ID NO: 33 - protein, 5 aa, synthetic construct
  VARIANT 1: note = L-or D-Lysine
  VARIANT 2: note = L-or D-Glycine
  VARIANT 3: note = L-or D-Glycine
  VARIANT 4: note = L-or D-Proline
  VARIANT 5: note = L-or D-Glycine
KGGPG

SEQ ID NO: 34 - protein, 5 aa, synthetic construct
  VARIANT 1: note = L-or D-Lysine
  VARIANT 2: note = L-or D-Alanine
  VARIANT 3: note = L-or D-Glycine
  VARIANT 4: note = L-or D-Glycine
  VARIANT 5: note = L-or D-Proline
KAGGP

SEQ ID NO: 35 - protein, 5 aa, synthetic construct
  VARIANT 1: note = L-or D-Glycine
  VARIANT 2: note = L-or D-Alanine
  VARIANT 3: note = L-or D-Glycine
  VARIANT 4: note = L-or D-Proline
  VARIANT 5: note = L-or D-Glycine
GAGPG

SEQ ID NO: 36 - protein, 6 aa, synthetic construct
  VARIANT 1: note = L-or D-Valine
  VARIANT 2: note = L-or D-Glycine
  VARIANT 3: note = L-or D-Valine
  VARIANT 4: note = L-or D-Alanine
  VARIANT 5: note = L-or D-Proline
  VARIANT 6: note = L-or D-Glycine
VGVAPG

SEQ ID NO: 37 - protein, 6 aa, synthetic construct
  VARIANT 1: note = L-or D-Glycine
  VARIANT 2: note = L-or D-Valine
  VARIANT 3: note = L-or D-Alanine
  VARIANT 4: note = L-or D-Proline
  VARIANT 5: note = L-or D-Glycine
  VARIANT 6: note = L-or D-Valine
GVAPGV

SEQ ID NO: 38 - protein, 6 aa, synthetic construct
  VARIANT 1: note = L-or D-Glycine
  VARIANT 2: note = L-or D-Lysine
  VARIANT 3: note = L-or D-Threonine
  VARIANT 4: note = L-or D-Threonine
  VARIANT 5: note = L-or D-Lysine
  VARIANT 6: note = L-or D-Serine
GKTTKS

SEQ ID NO: 39 - protein, 6 aa, synthetic construct
  VARIANT 1: note = L-or D-Glycine
  VARIANT 2: note = L-or D-Lysine
  VARIANT 3: note = L-or D-Threonine
  VARIANT 4: note = L-or D-Serine
  VARIANT 5: note = L-or D-Lysine
  VARIANT 6: note = L-or D-Serine
GKTSKS

SEQ ID NO: 40 - protein, 6 aa, synthetic construct
  VARIANT 1: note = L-or D-Phenylalanine
  VARIANT 2: note = L-or D-Valine
  VARIANT 3: note = L-or D-Alanine
  VARIANT 4: note = L-or D-Proline
  VARIANT 5: note = L-or D-Phenylalanine
  VARIANT 6: note = L-or D-Proline
FVAPFP

SEQ ID NO: 41 - protein, 6 aa, synthetic construct
  VARIANT 1: note = L-or D-Alanine
  VARIANT 2: note = L-or D-Glycine
  VARIANT 3: note = L-or D-Glycine
  VARIANT 4: note = L-or D-Alanine
  VARIANT 5: note = L-or D-Proline
  VARIANT 6: note = L-or D-Glycine
AGGAPG

SEQ ID NO: 42 - protein, 6 aa, synthetic construct
  VARIANT 1: note = L-or D-Lysine
  VARIANT 2: note = L-or D-Glycine
  VARIANT 3: note = L-or D-Glycine
  VARIANT 4: note = L-or D-Glycine
  VARIANT 5: note = L-or D-Proline
  VARIANT 6: note = L-or D-Glycine
KGGGPG

SEQ ID NO: 43 - protein, 6 aa, synthetic construct
  VARIANT 1: note = L-or D-Lysine
  VARIANT 2: note = L-or D-Alanine
  VARIANT 3: note = L-or D-Glycine
  VARIANT 4: note = L-or D-Glycine
  VARIANT 5: note = L-or D-Proline
  VARIANT 6: note = L-or D-Glycine
KAGGPG

SEQ ID NO: 44 - protein, 6 aa, synthetic construct
  VARIANT 1: note = L-or D-Tyrosine
  VARIANT 2: note = L-or D-Tyrosine
  VARIANT 3: note = L-or D-Arginine
  VARIANT 4: note = L-or D-Alanine
  VARIANT 5: note = L-or D-Aspartic acid
  VARIANT 6: note = L-or D-Alanine
YYRADA

SEQ ID NO: 45 - protein, 6 aa, synthetic construct
  VARIANT 1: note = L-or D-Glutamine
  VARIANT 2: note = L-or D-Glycine
  VARIANT 3: note = L-or D-Glutamine
  VARIANT 4: note = L-or D-Lysine
  VARIANT 5: note = L-or D-Proline
  VARIANT 6: note = L-or D-Glycine
QGQKPG

SEQ ID NO: 46 - protein, 6 aa, synthetic construct
  VARIANT 1: note = L-or D-Glutamine
  VARIANT 2: note = L-or D-Glycine
  VARIANT 3: note = L-or D-Valine
  VARIANT 4: note = L-or D-Lysine
  VARIANT 5: note = L-or D-Proline
  VARIANT 6: note = L-or D-Alanine
QGVKPA

SEQ ID NO: 47 - protein, 6 aa, synthetic construct
  VARIANT 1: note = L-or D-Proline
  VARIANT 2: note = L-or D-Glycine
  VARIANT 3: note = L-or D-Alanine
  VARIANT 4: note = L-or D-Tyrosine
  VARIANT 5: note = L-or D-Proline
  VARIANT 6: note = L-or D-Glycine
PGAYPG

SEQ ID NO: 48 - protein, 6 aa, synthetic construct
  VARIANT 1: note = L-or D-Proline
  VARIANT 2: note = L-or D-Lysine
  VARIANT 3: note = L-or D-Glycine
  VARIANT 4: note = L-or D-Serine
  VARIANT 5: note = L-or D-Proline
  VARIANT 6: note = L-or D-Glycine
PKGSPG

SEQ ID NO: 49 - protein, 7 aa, synthetic construct
  VARIANT 1: note = L-or D-Arginine
  VARIANT 2: note = L-or D-Glycine
  VARIANT 3: note = L-or D-Tyrosine
  VARIANT 4: note = L-or D-Tyrosine
  VARIANT 5: note = L-or D-Lysine
  VARIANT 6: note = L-or D-Lysine
  VARIANT 7: note = L-or D-Glutamic acid
RGYYKKE

SEQ ID NO: 50 - protein, 7 aa, synthetic construct
  VARIANT 1: note = L-or D-Cysteine
  VARIANT 2: note = L-or D-Glycine
  VARIANT 3: note = L-or D-Glycine
  VARIANT 4: note = L-or D-Proline
  VARIANT 5: note = L-or D-Glycine
  VARIANT 6: note = L-or D-Alanine
  VARIANT 7: note = L-or D-Glycine
CGGPGAG

SEQ ID NO: 51 - protein, 7 aa, synthetic construct
  VARIANT 1: note = L-or D-Glycine
  VARIANT 2: note = L-or D-Glycine
  VARIANT 3: note = L-or D-Glycine
  VARIANT 4: note = L-or D-Proline
  VARIANT 5: note = L-or D-Glycine
  VARIANT 6: note = L-or D-Alanine
  VARIANT 7: note = L-or D-Glycine
GGGPGAG

SEQ ID NO: 52 - protein, 8 aa, synthetic construct
  VARIANT 1: note = L-or D-Valine
  VARIANT 2: note = L-or D-Isoleucine
  VARIANT 3: note = L-or D-Glycine
  VARIANT 4: note = L-or D-Tyrosine
  VARIANT 5: note = L-or D-Lysine
  VARIANT 6: note = L-or D-Threonine
  VARIANT 7: note = L-or D-Threonine
  VARIANT 8: note = L-or D-Lysine
VIGYKTTK